(19) United States
(12) Patent Application Publication
(10) Pub. No.: US 2004/0106120 A1
(43) Pub. Date: Jun. 3, 2004

(54) Diagnosis and treatment of disease

(76) Inventors: Rachid Tazi-Ahnini, Sheffield (GB); Claes Bavik, Seattle, WA (US); Simon Ward, Sheffield (GB); Gordon Duff, Sheffield (GB); Michael Cork, Derbyshire (GB)

Correspondence Address: WORKMAN NYDEGGER (F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/433,234

(22) PCT Filed: Nov. 30, 2001
(86) PCT No.: PCT/GB01/05303

(30) Foreign Application Priority Data
Nov. 30, 2000 (GB) 0029225.0
Dec. 7, 2000 (GB) 0029879.4

(51) Int. Cl. C12Q 1/68

(52) U.S. Cl. 435/6

(57) ABSTRACT

We disclose a method of diagnosis of a disease, or susceptibility to a disease associated with abnormal cell-cell adhesion between epithelial cells, the method comprising detection of a mutation in a nucleic acid encoding an adhesion protein, a protease, or a protease inhibitor of an individual.

### FIELD

This invention relates to the diagnosis and treatment of diseases. More particularly, the invention relates to the treatment and diagnosis of epidermal or skin diseases, and agents for such treatment and/or diagnosis.

### BACKGROUND

The skin is a barrier that retains water within the body and prevents the penetration of environmental agents into the body. The barrier function of the skin is therefore essential to the maintenance of the internal homeostasis (Cork 1997). The epidermis is composed of layers of closely packed keratinocytes that are formed by division in the stratum basale (or germinative layer). As the keratinocytes move up through the prickle and granular layers, they differentiate and a rigid internal structure of keratin, microfilaments and microtubules is formed. The stratum corneum (horny layer) is composed of layers of flattened dead cells that have lost their nucleus, between which is a complex mixture of lipid and proteins.

The epidermal barrier is located in the stratum corneum and is dependent on strong adhesion between corneocytes (Egelrud, 2000). This adhesion is mediated by corneodesmosomes (Menton and Elisen, 1971 Chapman and Walsh, 1990; North et al, 1999). Corneodesmosin is a glycoprotein of corneodesmosomes.

The other component of the epidermal barrier is the lamellar lipids. The lipid is secreted into lamellar bodies in the stratum granulosum (Elias 1993). At the interface between the stratum granulosum and stratum corneum, the lipids are extracted from the granular cells into the intercorneoctye space. The lipids are then organised into highly organised multimellar bilayer structures (Landmann 1988). The stratum corneum can be visualised rather like a brick wall with the corneocytes forming the bricks and the lamellar lipids the mortar (Elias 1983). Corneocytes contain a water retaining substance, natural moisturising factor (NMF), which retains water within them. The high water content causes the corneocytes to swell, preventing the formation of fissures and cracks between them. The pliability and elasticity of the skin is directly related to its water content. Normal healthy stratum corneum has comparatively high water content.

Thus, the stratum corneum is a barrier that is continually being replaced by proliferation and differentiation of keratinocytes in the viable epidermis. In order to maintain a constant stratum corneum thickness at a given body site superficial parts of the stratum corneum must be continuously shed in the process of desquamation at a rate that balances their production. Impaired desquamation of corneocytes is characteristic of a number of diseases such as psoriasis, acne vulgaris, ichiosis and keratinosis pilaris, among others. In psoriasis, impaired desquamation of corneocytes causes an increased thickness of the stratum corneum and the barrier is usually enhanced. Acne vulgaris primary pathological event is a narrowing of the pilosebaceous unit, which arises as a result of impaired desquamation of corneocytes. The defect is combined with increased sebum production rate, and secondary events include stagnation of sebum.

### SUMMARY

We demonstrate that proteolysis of corneodesmosomal proteins is a key process involved in desquamation.

Accordingly, we have discovered that increased, impaired or otherwise abnormal proteolysis of corneodesmosomal proteins is involved in several skin disorders. In particular, we have found out that increased proteolysis of adhesion proteins is involved in diseases characterised by impaired barrier function, and decreased proteolysis of adhesion proteins is involved in diseases characterised by impaired desquamation of corneocytes. We further demonstrate that mutations in genes encoding adhesion proteins result in reduced adhesion between epithelial cells.

According to a first aspect of the present invention, we provide a method of diagnosis of a disease, or susceptibility to a disease associated with abnormal cell-cell adhesion between epithelial cells, the method comprising detection of a mutation in a nucleic acid encoding an adhesion protein, a protease, or a protease inhibitor of an individual.

There is provided, according to a second aspect of the present invention, a method of diagnosis of a Group I disease or susceptibility to a Group I disease in an individual, the method comprising detecting a presence or absence of a polymorphism in an adhesion protein, protease or protease inhibitor polypeptide, or a nucleic acid encoding such, in which the polymorphism is associated with a Group I disease.

Preferably, the adhesion protein is selected from the group consisting of adhesion proteins shown in Tables D2.1 and 5.1, preferably corneodesmosin, desmoglein I, desmoglein 3, plakoglobin, desmoplakin, desmocollin I and envoplakin, a serine rich protein, preferably a small proline-rich protein (SPRR), SPRR2A, SPRR1B, SPRK, SPRR2E, SPRR2F, SPRR2B, SPRR2D, SPRR2C, SPRR2G, SPRR1A, SPRR3, SPRR4, involucrin, or loricrin,

Preferably, the protease is selected from the group consisting of proteases shown in Tables D3.1 and 6.1, preferably stratum corneum chymotryptic enzyme (SCCE) or stratum corneum tryptic enzyme (SCTE).

Preferably, the protease inhibitor is selected from the group consisting of protease inhibitors shown in Tables D4.1 and 7.1, preferably Secretory Leukoprotease Inhibitor (SLPI), elafin protease inhibitor 3 (PI3 or SKALP) or cystatin A (CSTA).

Preferably, the Group I disease is selected from the group consisting of: atopic eczema, sebarrhoeic eczema, irritant contact dermatitis, allergic contact dermatitis, lung atopic asthma, post viral asthma, branchial hyper-reactivity, chronic obstruction pulmonary disease, Crohn's disease, ulcerative colitis, coeliac disease, peptic ulceration, impetigo, viral warts, Molluslum Contagiosum, bacterial meningitis, viral meningitis, peptic ulceration associated with penetration of Helicobacteria pylori, skin melanoma, squamous cell carcinoma, basal cell carcinoma, cutaneous lymphoma, a skin cancer, a malignancy of the gastrointestinal tract and a malignancy of the lung.

We provide, according to a third aspect of the present invention, a method of diagnosis of a Group I disease or susceptibility to a Group I disease in an individual, the method comprising detecting the presence, absence or a modulated level of an adhesion protein, protease or protease inhibitor, or a fragment thereof, in an individual.

As a fourth aspect of the present invention, there is provided a method of treatment or prophylaxis of a Group I disease, the method comprising up-regulating the expression and/or activity of an adhesion protein responsible for adhesion between the cells, or down-regulating the proteolysis of the adhesion protein.

Preferably, the method of treatment is one in which the expression and/or activity of the adhesion protein is up-regulated at the transcriptional or the translational level, or both.

Alternatively or in addition, the expression, activity and/or breakdown of a protease involved in proteolysis of the adhesion protein is down-regulated Furthermore, alternatively or in addition, the expression and/or activity of a protease inhibitor responsible for inhibiting the activity of a protease involved proteolysis of the adhesion protein is up-regulated, and/or in which the breakdown of the protease inhibitor is down-regulated.

Proteolysis of the adhesion protein may be reduced by one or more of the following: administration of a protease inhibitor or a fragment thereof; administration of an antagonist of a protease or a fragment thereof; administration of an agonist of a protease inhibitor; reducing the expression of a protease; reducing the activity of a protease; increasing the expression of a protease inhibitor; increasing the activity of a protease inhibitor.

The present invention, in a sixth aspect, provides a method of treatment or prophylaxis of a Group I disease, the method comprising administering to a patient suffering or likely to suffer from such a disease a therapeutically effective amount of a non-disease associated form of an adhesion protein, protease or protease inhibitor, or a fragment thereof.

Preferably, the method comprises administration of a protease inhibitor, or a fragment thereof, capable of inhibiting protease activity.

Preferably, the method comprises administration of a fragment of SLPI, preferably a peptide selected from the group consisting of: CGKS (SB7a) and CGKS CVSPVKA (SB7b); KIIDGA; GDKIIDGA; GDKIID; KII; KIID; KIIDG; KIIDGA; LDPVD (651); KRDLK (652); LDPVDTPNP (653); LDPVDTPNPTRRKPG (654); CGKSCVSPVKA (644); CVSPVKA (643), most preferably Peptide 643, Peptide 651 or Peptide 653.

Alternatively or in addition, the method comprises administration of TNF-α and/or IL-β.

Preferably, the Group I disease is eczema, preferably atopic eczema, or dermatitis, preferably dermatitis herpetiformis.

In a seventh aspect of the present invention, there is provided a method of diagnosis of a Group II disease or susceptibility to a Group II disease in an individual, the method comprising detecting a presence or absence of a polymorphism in an adhesion protein, protease or protease inhibitor polypeptide, or a nucleic acid encoding such, in which the polymorphism is associated with a Group II disease.

According to an eighth aspect of the present invention, we provide a method of treatment or prophylaxis of a Group II disease, the method comprising down-regulating the expression and/or activity of an adhesion protein for adhesion between the cells, or up-regulating the proteolysis of the adhesion protein.

Preferably, in which the expression and/or activity of the adhesion protein is down-regulated at the transcriptional or the translational level, or both.

Alternatively or in addition, the expression, activity and/or breakdown of a protease involved in proteolysis of the adhesion protein is up-regulated Furthermore, alternatively or in addition, the expression and/or activity of a protease inhibitor responsible for inhibiting the activity of a protease involved proteolysis of the adhesion protein is down-regulated, and/or in which the breakdown of the protease inhibitor is up-regulated.

Preferably, the proteolysis of the adhesion protein is increased by one or more of the following: administration of a protease or a fragment thereof; administration of an agonist of a protease; administration of an antagonist of a protease inhibitor; increasing the expression of a protease; increasing the activity of a protease; reducing the expression of a protease inhibitor; reducing the activity of a protease inhibitor.

We provide, according to a ninth aspect of the invention, a method of treatment or prophylaxis of a Group II disease, the method comprising administering to a patient suffering or likely to suffer from such a disease a therapeutically effective amount of a non-disease associated form of an adhesion protein, protease or protease inhibitor, or a fragment thereof.

Preferably, the method comprises administration of an adhesion protein, or a fragment thereof

Preferably the method comprises administration of a fragment of Desmocollin I, preferably a peptide comprising the sequence of Peptide 641, or a fragment of Desmoplakin, preferably a peptide comprising the sequence of Peptide 642.

There is provided, in accordance with a tenth aspect of the present invention, a monoclonal or polyclonal antibody capable of specifically reacting with an adhesion protein, protease or protease inhibitor, preferably a disease associated form of an adhesion protein, protease or protease inhibitor.

As an eleventh aspect of the invention, we provide a method for identifying a molecule capable of capable of binding to an adhesion protein, protease or protease inhibitor, the method comprising contacting an adhesion protein, protease or protease inhibitor polypeptide with a candidate compound and determining whether the candidate compound binds to the adhesion protein, protease or protease inhibitor.

We provide, according to a twelfth aspect of the invention, there is provided compound identified by a method according to the above aspect.

According to a thirteenth aspect of the present invention, we provide a method of identifying a molecule capable of modulating the activity of a protease, the method comprising: (a) providing an adhesion protein; (b) providing a protease; (c) exposing the adhesion protein to the protease in the presence of a candidate molecule; and (d) detecting cleavage or absence of cleavage of the adhesion protein by the protease.

There is provided, according to a fourteenth aspect of the present invention, use of a compound identified by a method according to the thirteenth aspect of the invention to treat a Group II disease, in which the compound is capable of enhancing the cleavage of the adhesion molecule by the protease.

We provide, according to a fifteenth aspect of the present invention, use of a compound identified by a method according to the thirteenth aspect of the invention to treat a Group I disease, in which the compound is capable of inhibiting the cleavage of the adhesion molecule by the protease.

As a sixteenth aspect of the present invention, there is provided a transgenic, non-human animal expressing a heterologous adhesion protein, protease or protease inhibitor, preferably a disease associated form of the adhesion protein, protease or protease inhibitor. In a seventh aspect of the present invention, there is provided a transgenic, non-human animal expressing a modulated level, preferably an up-regulated or down-regulated level of an adhesion protein, protease or protease inhibitor. According to an eighteenth aspect of the present invention, we provide a transgenic, non-human animal which substantially does not express an adhesion protein, protease or protease inhibitor. Such animals may be used as a model for a skin disease, preferably a Group I or a Group II disease.

### BRIEF DESCRIPTION OF THE FIGURES

FIG. 1 shows part of a chromatogram of the AE2 sequence that corresponds to Exon V of the SCCE gene, from an atopic eczema patient. The 4-bp repeat is indicated by an arrow.

FIG. 2 shows a part of the chromatogram of a Poly9 (control) sequence corresponding to FIG. 2, where the second repeat (AACC) is absent. The 4-bp single repeat is indicated by an arrow.

FIG. 3 shows electrophoresis gels of PCR products of ten DNA samples using primers F5 and I/D RII (first optimisation). The expected PCR product (457 bp) is indicated by an arrow.

FIG. 4 shows gel electrophoresis of products of PCR reactions, which include a “control” amplification product of 800 bp, the product of amplification of the whole exon 5 (first optimisation). This figure provides verification that results are valid using an internal control (800 bp-shown by an arrow).

FIG. 5 is a graph showing transcriptional activity of the cystatin A promoter in control and eczema patients. PCR products of promoter region are cloned in reporter vector (CAT). These include sequences p cstappoly7rCAT, p cstappoly3r, CAT pcstappoly4r and CAT pcstape8fCAT p cstape5rCAT p cstape7rCAT from controls and eczema patients respectively. Transfected SVHK cells are harvested and extracts are assayed for CAT activity.

FIG. 6 is a Western Blot probed with anti-corneodesmosin antibody, showing a proteolysis profiles of proteins extracted from psoriatic epidermis. PNL: psoriatic non-lesional skin, PL: psoriatic lesional skin, N: normal skin.

FIG. 7 is a Western Blot probed with anti-plakoglobin antibody, showing a proteolysis profiles of proteins extracted from psoriatic epidermis. PNL: psoriatic non-lesional skin, PL: psoriatic lesional skin, N: normal skin.

FIG. 8 is a Western Blot probed with anti-desmoplakin antibody, showing a proteolysis profiles of proteins extracted from psoriatic epidermis. PNL: psoriatic non-lesional skin, PL: psoriatic lesional skin, N: normal skin.

FIG. 9 is a Western Blot probed with anti-desmocollin I antibody, showing a proteolysis profiles of proteins extracted from psoriatic epidermis. PNL: psoriatic non-lesional skin, PL: psoriatic lesional skin, N: normal skin.

FIG. 10 is a Western Blot probed with anti-envoplakin antibody, showing a proteolysis profiles of proteins extracted from psoriatic epidermis. PNL: psoriatic non-lesional skin, PL: psoriatic lesional skin, N: normal skin.

FIG. 11 is a Western Blot probed with anti-SCCE antibody, showing a proteolysis profiles of proteins extracted from psoriatic epidermis. PNL: psoriatic non-lesional skin, PL: psoriatic lesional skin, N: normal skin.

FIG. 12 is a Western Blot probed with anti-SLPI antibody, showing a proteolysis profiles of proteins extracted from psoriatic epidermis. PNL: psoriatic non-lesional skin, PL: psoriatic lesional skin, N: normal skin.

FIG. 13 is a graph showing quantification of corneodesmosome density at the surface of the stratum corneum in skin conditions with altered skin barrier function

FIG. 14 is a graph showing quantification of corneodesmosome density in tape strips from psoriasis patients treated with or without protease enzymes

FIG. 15 shows a section of a untreated psoriatic biopsy. The numerous corneodesmosomes present in the thickened stratum corneum (SC) hold the layers of corneocytes tightly together. The viable cell layer underlying the stratum corneum is labelled (V). Transmission electron micrograph ×3300 magnification.

FIG. 16 shows a section of a psoriatic biopsy treated for 16 hours with 0.25% chymotrypsin. Splitting of the layers of corneocytes (acantholysis) is obvious due to degradation of the corneodesmosomes by chymotrypsin. Transmission electron micrograph ×3300 magnification.

FIG. 17 shows a section of an untreated psoriatic biopsy (stratum corneum). Numerous corneodesmosomes can be seen, some of these are indicated by the white arrows. Transmission electron micrograph, ×23000 magnification.

FIG. 18 shows a section of the stratum corneum of psoriatic biopsy treated for 16 hours with 0.25% chymotrypsin. Far fewer corneodesmosomes are visible after protease treatment (solid white arrows). Some remnants of degraded corneodesmosomes can also be seen (dashed white arrows). Transmission electron micrograph, ×23000 magnification.

FIG. 19 shows a section through reconstitued human epidermis cultures (skin equivalents). Left hand panel: treated with buffered salt solution (control), right hand panel: treated with 6 μM peptide 641.

FIG. 20 shows a section through reconstitued human epidermis cultures (skin equivalents). Left hand panel: treated with buffered salt solution (control), right hand panel: treated with 6 μM peptide 642.

FIG. 21 shows a section through reconstitued human epidermis cultures (skin equivalents). Left hand panel: treated with buffered salt solution (control), right hand panel: treated with 6 μM peptide 643.

FIG. 22 shows a section through reconstitued human epidermis cultures (skin equivalents). Left hand panel: treated with buffered salt solution (control), right hand panel: treated with 6 μM peptide 651.

FIG. 23 shows a section through reconstitued human epidermis cultures (skin equivalents). Left hand panel: treated with buffered salt solution (control), right hand panel: treated with 6 μM peptide 653.

FIG. 24 shows a section through reconstitued human epidermis cultures (skin equivalents). Left hand panel: treated with buffered salt solution (control), right hand panel: treated with 2.5 ng/ml TNF-α.

FIG. 25 shows a section through reconstitued human epidermis cultures (skin equivalents). Left hand panel: treated with buffered salt solution (control), right hand panel: treated with 2.5 ng/ml IL-1β.

### DETAILED DESCRIPTION

We demonstrate that regulated proteolysis of adhesion proteins is important for a healthy skin. We show that an underlying cause of various skin diseases is the breakdown in regulation of proteolysis of adhesion proteins, leading to an increased, decreased or otherwise abnormal adhesion between corneocytes. Such abnormal proteolysis may have various causes, including mutations in adhesion protein genes, mutations in proteases which act on adhesion proteins, and/or in protease inhibitors which act on such proteases and regulate their activity.

We show that treatment and prevention of such diseases may be achieved by modulating the proteolysis of adhesion proteins. We also demonstrate that detection of such mutations in adhesion proteins, proteases and/or protease inhibitors may be used to diagnose various skin diseases and/or susceptibility to such diseases.

Thus, for example, we show that mutations within genes encoding adhesion proteins, such as corneodesmosin (S gene) result in a reduced or increased adhesion between epithelial cells such as corneocytes, leading to disease. Furthermore, mutations within genes related to such adhesion proteins, for example, genes within the MHC epidermal gene cluster on chromosome 6p21 also result in reduced adhesion between epithelial cells. We identify specific mutations at various positions, including +1243, +180 and +619, and relate them to Group I and Group II diseases.

Furthermore, we show that mutations within genes encoding proteolytic enzymes (such as the stratum corneum chymotryptic enzyme (SCCE) and/or stratum corneum tryptic enzymes (SCTE) genes on chromosome 19 at the q13 band and related serine proteases genes in chromosome 17 result in an increased activity of these enzyme and as a result premature desquamation of corneocytes. In particular, we identify the association of an AACCAACC sequence with atopic eczema and other Group I diseases.

Furthermore, mutations in serine protease inhibitors genes such as SKALP and SLPI lead to failure of regulation of desquamation process. Thus, for example, mutations in the S, SCCE, SCTE, SKALP, SLPI genes or any combination of these genes result in impairment of the epidermal barrier function. Mutations in the genes occurring together increase the severity of the epidermal barrier function defect.

Accordingly, we provide for methods of diagnosis of a disease associated with abnormal cell-cell adhesion between epithelial cells by detecting such mutations. Furthermore, we also provide for methods of treatment of a disease associated with abnormal cell-cell adhesion between epithelial cells by regulating the expression of such protease and protease inhibitor genes.

Where reference is made to “treatment” of a disease, this should be taken to include reference to alleviation of a symptom of that disease. Preferably, substantially all of the symptoms of an individual having that disease are alleviated or removed. “Disease” should be taken to include any syndrome, as well as any condition affecting the health or well-being of an individual. Preferably, an individual is relieved of at least one symptom of the disease or condition using the methods and compositions described here. Preferably, the individual reverts substantially to the state of a normal unaffected individual. This may be assessed by a physician using a relevant clinical parameter.

Similarly, where reference is made to “diagnosis” of a disease, this should be taken to include both diagnosis of the disease itself, as well as susceptibility to the disease. The methods of diagnosis disclosed here may also be employed as methods of providing indications useful in the diagnosis of diseases.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference), chemical methods, pharmaceutical formulations and delivery and treatment of patients.

### Epidermal Barrier Function

The methods and compositions described here are primarily concerned with the diagnosis and/or treatment of diseases associated with or characterised by abnormal epidermal barrier function.

The skin is a barrier that retains water within the body and prevents the penetration of environmental agents into the body. This ability to resist penetration is essential to the maintenance of the internal homeostasis (Cork 1997). The epidermis is composed of layers of closely packed keratinocytes that are formed by division in the stratum basale (or germinative layer). As the keratinocytes move up through the prickle and granular layers, they differentiate and a rigid internal structure of keratin, microfilaments and microtubules is formed. Within the epidermis, the barrier function is typically performed by the stratum corneum and is dependent on strong adhesion between corneocytes (Egelrud, 2000) mediated by corneodesmosomes (Menton and Elisen, 1971, Chapman and Walsh, 1990; North et al, 1999).

The stratum corneum (horny layer) is composed of layers of flattened dead cells that have lost their nucleus, between which is a complex mixture of lipid and proteins. The stratum corneum is a barrier that is continually being replaced by proliferation and differentiation of keratinocytes in the viable epidermis. In order to maintain a constant stratum corneum thickness at a given body site superficial parts of the stratum corneum must be continuously shed in the process of desquamation at a rate that balances their production.

The “barrier function” of the epidermis, or the “epidermal barrier function”, as the terms are used in this document, is therefore intended to refer to the ability of an epidermal layer to resist the penetration of an external agent. Accordingly, the methods and compositions act by modulating the epidermal barrier function of an individual. In particular, the methods and compositions described here may be used to treat and/or diagnose diseases in which the epidermal barrier in individuals suffering from such diseases is modulated, i.e., enhanced or weakened, as compared to the epidermal barrier in normal individuals.

Measurement of barrier function may be done in various ways. For example, a Franz chamber and cadaver system (also known as a Franz chamber penetration system) may be used. This system measures barrier function by measuring the substances passing through it, and has the advantage in that it is a robust, quick and easy system.

Impaired Barrier Function

Impaired barrier function is a characteristic of Group I diseases, described in detail below.

An epidermis has an impaired barrier function when it is more permeable to an external agent than a normal, healthy epidermis from the same or another individual. Thus, an epidermis with impaired barrier function allows more penetration of an external agent than otherwise. Preferably, an epidermis with impaired barrier function is 20%, 40%, 60%, 80% or 100% or more permeable to an external agent than a normal epidermis. Preferably, a molecule which is substantially unable to cross a normal epidermal barrier is able to cross the barrier of an epidermis with impaired barrier function.

Increase in penetration or permeability is preferably reflected by increase of mass of agent or drug, activity of an agent or drug (such as a relevant chemical, biological or enzymatic activity) which is capable of passing through the epidermal layer. Thus, an epidermis with impaired barrier function will therefore preferably enable penetration of 5%, 10%, 20%, 30%, 40%, 50%, 60%, 70%, 80% 90% or 100% or more agent or drug than a normal epidermis.

Increase in permeability or penetration may also be reflected by an increased ratio of molecules which pass through compared to those which are retarded, or alternatively, as compared to those which are applied. This ratio, N, may be increased by 5%, 10%, 20%, 30%, 40%, 50%, 60%, 70%, 80% 90% or 100% or more with the methods and compositions as described here.

Measurement of epidermal barrier function may be done in various ways. Similarly, the level or degree of penetration of an agent or composition can be determined by techniques known to those of skill in the art. For example, radiolabeling of an active compound or a tracer compound, followed by measurement of the amount of radiolabelled compound absorbed by the skin enables one of skill in the art to determine levels of the composition absorbed using any of several methods of determining skin penetration of the test compound. Measurement of the amount of radiolabelled compound which crosses the skin layer may also be made.

In a preferred embodiment, a Franz chamber and cadaver system (also known as a Franz chamber penetration system) is used to measure penetration of an agent. This system measures barrier function by allowing the measurement of amount of radiolabelled compound which passes through a piece of skin to a receptacle fluid. The Franz chamber has the advantage in that it is a robust, quick and easy system, and is described in more detail below.

Enhanced Barrier Function

An epidermis which has increased barrier function is less permeable to an external agent than a normal, healthy epidermis from the same or other individual.

An epidermis has an increased or enhanced barrier function when it is less permeable to an external agent than a normal, healthy epidermis from the same or another individual. Thus, an epidermis with impaired barrier function allows less penetration of an external agent than otherwise. Preferably, such an epidermis has a permeability that is 90%, 80%, 70%, 60%, 40%, 20%, 10% 5% or less permeable to an external agent than a normal epidermis. Preferably, a molecule which is able to cross an epidermis with normal barrier function is substantially unable to cross an epidermis with increased or enhanced barrier function.

Decrease in penetration or permeability is preferably reflected by decrease of mass of agent or drug, activity of an agent or drug (such as a relevant chemical, biological or enzymatic activity) which is capable of passing through the epidermal layer. Thus, an epidermis with enhanced barrier function will therefore preferably enable penetration of, 90% or less, more preferably 80% or less, more preferably 70% or less, more preferably 60% or less, more preferably 50% or less, more preferably 40% or less, more preferably 30% or less, more preferably 20% or less, more preferably 10% or less, most preferably 5% or less, agent or drug than a normal epidermis.

Decrease in permeability or penetration may also be reflected by an decreased ratio of molecules which pass through compared to those which are retarded, or alternatively, as compared to those which are applied. This ratio, N, may be decreased by 5%, 10%, 20%, 30%, 40%, 50%, 60%, 70%, 80% 90% or 100% or more with the methods and compositions as described here.

### Group I Diseases: Associated with Decreased Cell-Cell Adhesion

In one embodiment, the methods disclosed here are suitable for treatment and diagnosis of diseases associated with decreased cell-cell adhesion, in particular of epithelial cells, in particular corneocytes. Such diseases are referred to in this document as “Group 1 diseases”, and are therefore diseases of impaired or reduced barrier function.

Within the Group 1 diseases, three sub-groups may be identified. In each group the impaired barrier function is permitting the penetration of a xenobiotic through an epithelial barrier. Once the xenobiotic has penetrated through the epithelial barrier, it interacts with the host's cells to produce the disease phenotype.

A first subgroup of Group 1 diseases (Subgroup 1.1) is characterised by an impaired barrier that permits the penetration of an irritant, allergen or other xenobiotic. These xenobiotics interact with the body's immune system to produce an abnormal inflammatory response, which in turn causes tissue destruction. The immune response to the xenobiotic may be enhanced as the result of an associated genetic predisposition. Examples of such diseases affecting the skin include atopic eczema, sebarrhoeic eczema, irritant contact dermatitis, and allergic contact dermatitis. Examples of such diseases affecting the lung include: atopic asthma, post viral asthma/branchial hyper-reactivity, and chronic obstruction pulmonary disease. Examples of such diseases affecting the bowel include: Crohns disease, ulcerative colitis, coeliac disease, and peptic ulceration.

A second subgroup of Group 1 diseases (Subgroup 1.2) is characterised by an impaired barrier that permits the penetration of bacteria, virus, other micro-organisms or micro-organism products e.g. superantigenic exotoxin. The micro-organism and/or the micro-organism product then leads to a disease process. Examples of such diseases include atopic eczema, contact dermatits, impetigo, viral warts, Molluslum Contagiosum, meningitis (bacterial and viral), and peptic ulceration caused by or associated with penetration of Helicobacteria pylori.

A third subgroup of Group I diseases (Subgroup 1.3) is characterised by an impaired barrier that permits the penetration of a carcinogen. The carcinogen can thereby gain access to the epithelial stem cell population and can induce transformation. The carcinogen may act as a co-carcinogen with other environmental agents e.g. ultraviolet radiation. Examples of such diseases affecting the skin include melanoma, squamous cell carcinoma, basal cell carcinoma, cutaneous lymphoma, and other skin cancers. Examples of such diseases affecting the bowel include malignancies of the entire gastrointestinal tract. Lung malignancies are examples of diseases affecting the lung.

Treatment of Group I Diseases

According to the methods and compositions described here, Group 1 diseases result from a decreased adhesion between epithelial cells, for example corneocytes in the skin. This can arise as a result of changes in the expression, activity and/or breakdown of adhesion proteins which modulate or are responsible for the adhesion between epithelial cells, for example, corneodesmosin, changes in the expression, activity and/or breakdown of proteases which break down the adhesion proteins, and/or changes in the expression, activity and/or breakdown of inhibitors of proteases which break down the adhesion proteins. We have discovered that a change in any, some or all of the above may result in decreased adhesion between epithelial cells such as corneocytes.

As an example, an adhesion protein whose structure is changed as a result of a genetic mutation may have reduced adhesion activity and/or be more vulnerable to degradation by proteolytic enzymes (proteases). Accordingly, therefore, treatment of Group 1 diseases may be affected by increasing the expression of an adhesion protein, for example, corneodesmosin. Alternatively or in conjunction treatment may be effected by enhancing the adhesion activity and/or protease resistance of the adhesion protein. Thus, the adhesion protein responsible for reduced cell-cell adhesion may be replaced or supplemented with another adhesion protein, or a variant of the first adhesion protein with enhanced adhesion activity and/or protease resistance.

Furthermore, an increase in the quantity, activity and/or bio-availability of proteases may cause increased break down of adhesion proteins. Accordingly, therefore, a Group 1 disease may be treated by reducing the expression and/or activity of a protease responsible for breaking down an adhesion protein. For example, the transcription and/or translation of such a protease may be down-regulated as a means to treat Group 1 diseases. Thus, the expression of proteases such SCCE and SCTE may be decreased at the transcriptional and/or translational level to treat a Group 1 disease.

Impaired barrier function as a result of reduced cell-cell adhesion may also happen where there is a decreased quantity, activity and/or bio-availability of an inhibitor of a protease which breaks down an adhesion protein. Thus, a combination of any, some or all of the above changes may result in a greater decrease in cell-cell adhesion and impaired barrier function.

Therefore, the expression and/or activity of a protease inhibitor which is capable of inhibiting a protease activity capable of breakdown of an adhesion protein may be up-regulated as a means to treat a Group 1 disease. The protease inhibitor whose expression and/or activity is up-regulated may be the natural protease inhibitor which physiologically inhibits the protease in question (i.e., the protease responsible for proteolysis of the adhesion protein). Such a protease inhibitor is referred to here as a “primary” protease inhibitor. Accordingly, we provide a method of treating a Group 1 disease by activating the transcription and/or translation of protease inhibitors (for example, SKALP and SLPI) to decrease the activity of proteases such as SCCE and SCTE. Furthermore, secondary protease inhibitors (i.e., protease inhibitors which are not normally involved in the physiological regulation of the protease activity) may also be used to supplement and/or replace the primary protease inhibitor activity.

Furthermore, the methods and compositions described here include the administration of a protease inhibitor or a fragment of a protease inhibitor to an individual suffering or likely to suffer from a Group I disease. The protease inhibitor and/or fragment may be administered in the form of a natural or synthethic peptide. Preferably, the peptide comprises an active portion of a protease inhibitor. Preferably, the peptide is capable of inhibiting one or more protease activities. Suitable peptides may be designed against any protease inhibitor, including Secretory Leukoprotease Inhibitor (SLPI), elafin protease inhibitor 3 (PI3 or SKALP) or cystatin A (CSTA).

Preferably, the peptide is selected from the group consisting of: CGKS (SB7a) and CGKS CVSPVKA (SB7b); KIDGA; GDKIIDGA; GDKID; KII; KIID; KIIDG; KIIDGA; LDPVD (651); KRDLK (652); LDPVDTPNP (653); LDPVDTPNPTRRKPG (654); CGKSCVSPVKA (644); CVSPVKA (643). In a preferred embodiment, the peptide comprises Peptide 643, Peptide 651 or Peptide 653.

Furthermore, where the natural inhibitors of proteases such as stratum corneum chymotrypsin/trypsin enzyme are found to be deficient or reduced in activity, these may be replaced or supplemented by secondary protease inhibitors such as chymotrypsin, soybean trypsin inhibitor, cathepsin G, or other protease inhibitors as known in the art. Primary protease inhibitors include antileukoprotease and elafin. Such primary and/or secondary protease inhibitors may be applied systemically, or preferably to the epithelial surface, in the form of a pharmaceutical composition as described in further detail below.

Using the skin as an example, a protease inhibitor may be applied to the epithelial surface, the protease inhibitor antagonising for example stratum corneum chymotrypsin enzyme. Such a protease inhibitor may be formulated in an emollient base. The protease inhibitor(s) will inhibit the degradation of adhesion proteins such as corneodesmosin and as a result increase the cohesion between the corneocytes and improve the structure and/or resistance of the epidermal barrier.

Expression of one or more genes coding for protease inhibitors, whether primary protease inhibitors or secondary protease inhibitors, may be used to increase protease inhibitor activity. For example, we provide for the subcutaneous (or otherwise) injection of expression vectors which express, for example, chymotrypsin, soybean trypsin inhibitor, cathepsin G, or other protease inhibitors in order to increase protease inhibitor activity to reduce proteolysis of the adhesion protein. Furthermore, as mentioned above, endogenous production of primary protease inhibitors may be enhanced by up-regulating transcription and/or translation of the relevant protease inhibitor genes, by means known in the art.

Furthermore, we provide for the deactivation of a pathogenic form of an epithelial or desmosomal protein, and/or the activation of non-pathogenic forms of epithelial or desmosomal proteins to treat Group 1 diseases. Thus, in heterozygote patients, the expression of a non-pathogenic form of a desmosomal protein, for example, corneodesmosin, may be up-regulated by means known in the art. Alternatively, or in conjunction, the expression of a pathogenic form of a desmosomal protein, for example, corneodesmosin, may be down-regulated by means known in the art. Pathogenic and non-pathogenic forms of such proteins may be identified by means known in the art such as genetic analysis and proteolysis profiles and as described in detail below.

Diagnosis of Group I Diseases

Mutations in adhesion proteins, proteases and/or protease inhibitors are associated with Group I diseases. Such mutations are disclosed in Examples A1 to A6 and B1 to B6. Any of these mutations may be detected in the relevant gene, nucleic acid or polypeptide in order to diagnose a Group I disease.

### Group II Diseases: Associated with Increased Cell-Cell Adhesion

In another embodiment, the methods and compositions described here are suitable for treatment and diagnosis of diseases characterised by increased cell-cell adhesion of epithelial cells, in particular corneocytes. Such diseases are referred to in this document as “Group 2 diseases”. Group 2 diseases are therefore diseases of increased or enhanced barrier function.

Group II Diseases

Examples of Group 2 diseases include psoriasis, the ichthyoses, acne vulgaris and keratoses pilaris.

The Group 2 diseases may result from increased adhesion between epithelial cells, for example corneocytes in the skin. Increased adhesion results in an increased thickness of the stratum corneum. For example, in psoriasis and the ichthyoses, there is a generalised thickening of the stratum corneum. In acne there is a localised focal thickening of the stratum corneum at the entrance to the pilosebaceous duct.

Increased adhesion can arise as a result of changes in the expression, activity and/or breakdown of adhesion proteins which modulate or are responsible for the adhesion between epithelial cells, for example, corneodesmosin, changes in the expression, activity and/or breakdown of proteases which break down the adhesion proteins, and/or changes in the expression, activity and/or breakdown of inhibitors of proteases which break down the adhesion proteins. We have discovered that a change in any, some or all of the above may result in increased adhesion between epithelial cells such as corneocytes.

As an example, an adhesion protein whose structure is changed as a result of a genetic mutation may be more adhesive and/or less vulnerable to degradation by proteolytic enzymes (proteases). Accordingly, the methods and compositions described here may be used to treat Group 2 diseases by down-regulating the activity and/or expression of a mutant adhesion protein with enhanced adhesion activity. Thus, pathogenic forms of corneodesmosin which are associated with reduced proteolytic degradation may be identified by methods known in the art. These may correspond to the full length form (52-56 kDa) of the corneodesmosin protein in the superficial layers of the lesional stratum envelope. Pathogenic forms of the corneodesmosin may be identified by comparing proteolysis profiles from normal and lesional skin. For example, Western blot using protein extract from skin strips and probed with an antibody which recognises corneodesmosin may be used. Pathogenic forms of the corneodesmosin protein may be also identified by genetic analysis using case-control and transmission disequilibrium test (TDT; see Examples). For example in psoriasis the most likely pathogenic form is the one encoded by CD2 haplotype (Jenisch et al, 1999). The transcription of such a pathogenic form may be blocked at the transcriptional and/or translational level. Patients heterozygous for a pathogenic form of corneodesmosin may be treated by activating the alternative allele encoding a non-pathogenic form of the protein.

Furthermore, a decrease in the quantity, activity and/or bio-availability of proteases may cause decreased break down of adhesion proteins. Accordingly, therefore, a Group 2 disease may be treated by increasing the expression and/or activity of a protease responsible for breaking down an adhesion protein. For example, the transcription and/or translation of such a protease may be up-regulated as a means of treating Group 2 diseases.

Increased cell-cell adhesion may happen where there is an increased quantity, activity and/or bio-availability of an inhibitor of a protease which breaks down an adhesion protein. Thus, a combination of any, some or all of the above changes may result in an increase in cell-cell adhesion.

Therefore, the expression and/or activity of a protease inhibitor which is capable of inhibiting a protease activity capable of breakdown of an adhesion protein may be down-regulated as a means to treat a Group I disease. The protease inhibitor whose expression and/or activity is down-regulated is typically a natural protease inhibitor which inhibits the protease in question (i.e., the protease responsible for proteolysis of the adhesion protein). Such a protease inhibitor is referred to as a “primary” protease inhibitor. Expression of the protease inhibitor may be effected at the transcriptional and/or the translational level.

Psoriasis

The compositions and methods described here are suitable for the treatment or alleviation of symptoms of psoriasis, and we further provide methods of diagnosis of psoriasis.

Psoriasis manifests itself as inflamed swollen skin lesions covered with silvery white scale. Characteristics of psoriasis include pus-like blisters (pustular psoriasis), severe sloughing of the skin (erythrodermic psoriasis), drop-like dots (guttate psoriasis) and smooth inflamed lesions (inverse psoriasis).

The causes of psoriasis are currently unknown, although it has been established as an autoimmune skin disorder with a genetic component. One in three people report a family history of psoriasis, but there is no pattern of inheritance. However, there are many cases in which children with no apparent family history of the disease will develop psoriasis. Whether a person actually develops psoriasis may depend on “trigger factors” which include systemic infections such as strep throat, injury to the skin (the Koebner phenomenon), vaccinations, certain medications, and intramuscular injections or oral steroid medications. Once something triggers a person's genetic tendency to develop psoriasis, it is thought that in turn, the immune system triggers the excessive skin cell reproduction.

Skin cells are programmed to follow two possible programs: normal growth or wound healing. In a normal growth pattern, skin cells are created in the basal cell layer, and then move up through the epidermis to the stratum corneum, the outermost layer of the skin. This normal process takes about 28 days from cell birth to death. When skin is wounded, a wound healing program (regenerative maturation) is triggered, in which cells are produced at a much faster rate, the blood supply increases and localized inflammation occurs. Lesional psoriasis is characterized by cell growth in the alternate growth program. Skin cells (keratinocytes) switch from the normal growth program to regenerative maturation, cells are created and pushed to the surface in as little as 2-4 days, and the skin cannot shed the cells fast enough. The excessive skin cells build up and form elevated, scaly lesions. The white scale (“plaque”) that usually covers the lesion is composed of dead skin cells, and the redness of the lesion is caused by increased blood supply to the area of rapidly dividing skin cells.

Psoriasis is a genetically determined disease of the skin characterized by two biological hallmarks. First, there is a profound epidermal hyperproliferation related to accelerated and incomplete differentiation. Second, there is a marked inflammation of both epidermis and dermis with an increased recruitment of T lymphocytes, and in some cases, formation of neutrophil microabcesses. Many pathologic features of psoriasis can be attributed to alterations in the growth and maturation of epidermal keratinocytes, with increased proliferation of epidermal cells, occurring within 0.2 mm of the skin's surface. Traditional investigations into the pathogenesis of psoriasis have focused on the increased proliferation and hyperplasia of the epidermis. In normal skin, the time for a cell to move from the basal layer through the granular layer is 4 to 5 weeks. In psoriatic lesions, the time is decreased sevenfold to tenfold because of a shortened cell cycle time, an increase in the absolute number of cells capable of proliferating, and an increased proportion of cells that are actually dividing. The hyperproliferative phenomenon is also expressed, although to a substantially smaller degree, in the clinically uninvolved skin of psoriatic patients.

A common form of psoriasis, psoriasis vulgaris, is characterized by well-demarcated erythematous plaques covered by thick, silvery scales. A characteristic finding is the isomorphic response (Koebner phenomenon), in which new psoriatic lesions arise at sites of cutaneous trauma.

Lesions are often localized to the extensor surfaces of the extremities, and the nails and scalp are also commonly involved. Much less common forms include guttate psoriasis, a form of the disease that often erupts following streptococcal pharyngitis, and pustular psoriasis, which is characterized by numerous sterile pustules, often 2 to 5 mm in diameter, on the palms and soles or distributed over the body.

Objective methods which are employed for establishing the effect of treatment of psoriasis patients include the resolution of plaques by visual monitoring and with photography. The visual scoring is done using PASI (Psoriasis Area and Severity Index) score (see Fredericksson, A J, Peterssonn B C Dermatologies 157:238-244 (1978)).

Psoriasis affects approximately 2% of the UK population and may be associated with arthritis (in 7-25%) and Crohn's disease. The effect upon the individual self confidence and social activity can be catastrophic. Currently therapies for psoriasis are only suppressive and systemic treatments have significant adverse effects. Psoriasis is a multifactorial disease and genome wide scans have demonstrated a significant linkage to several chromosomes including 6p21, 1q21, 4q, 19p and 17q. In 6p21 the strongest association is with the major histocompatibility region (MHC). The MHC S gene (corneodesmosin) is located 160 kb telomeric of HLA-C and is expressed in keratinocyte differentiation as a component of the corneodesmosomes.

Acne

The methods and compositions described here are also suitable for the treatment and/or diagnosis of acne.

Acne affects large patient populations and is a common inflammatory skin disorder which usually localizes on the face. Fortunately, the disease usually disappears and in the interval of months or years between onset and resolution, therapy, although not curative, can satisfactorily suppress the disease in the majority of patients.

A small number of acne patients with severe disease show little or no response to intensive therapeutic efforts including the use of high doses of oral tetracycline, dapsone, prednisone, and, in women, estrogen. In many cases, these drugs afford only a modest degree of control while the side effects of these agents severely restrict their usefulness. Patients with nodulocystic acne suffer from large, inflammatory, suppurative nodules appearing on the face, and frequently the back and chest. In addition to their appearance, the lesions are tender and often purulently exudative and hemorrhagic. Disfiguring scars are frequently inevitable.

Therapies for acne involve local and systemic administration of retinoids. Topical application of all-trans-retinoic acid (tretinoin) has been tried with some success, particularly against comedones or blackheads, but this condition frequently returns when the treatment is withdrawn.

Acne is one of the commonest skin disorders affecting 15% of adolescents clinically (acne major) and 85% physiologically. In 15% of patients with acne major lesions requiring therapy persist to age 25. Acne is a disease affecting the pilosebaceous follicle in which there are four major aetiological factors: increased sebum production, hypercornification of the pilosebaceous duct, abnormal bacterial function and production of inflammation (Cunliffe 1989). Mild acne can have a major psychological impact and in severe acne depression and even suicide may occur. Unemployment is increased in patients with acne to 16.5% compared with 9.25% in matched controls. There are problems with current therapies for acne; increasing resistance to antibiotics and major adverse effects from oral isotretinoin including teratogenicity, inhibition of bone growth and hyperlipidaemia Treatment of acne represents 6-10% of new patients seen in dermatology clinics.

### Adhesion Protein

Where the term “adhesion protein” is used in this document, this should be taken as reference to any protein, polypeptide or peptide which mediates adhesion between cells. Thus, an “adhesion protein” includes any protein which is involved in cell-cell adhesion.

Preferably, the adhesion protein mediates cell-cell interaction between epithelial cells, i.e., the adhesion protein is an epithelial cell adhesion protein. Preferably, the adhesion protein mediates cell-cell interaction between epidermal cells. More preferably, the adhesion protein mediates cell-cell adhesion between corneocytes. Preferably, the adhesion protein is located in a corneodesmosome. Most preferably, the adhesion protein is a desmosomal protein or a corneodesmosomal protein.

The adhesion protein may suitably be selected from the group consisting of adhesion proteins shown in Tables D2.1 and 5.1. Preferably, the adhesion protein is selected from the group consisting of: corneodesmosin (AF030130), desoplakin (XM_{—}004463), plakoglobin (NM_{—}002230); (NM_{—}021991), desmoglein 1 (XM_{—}008810), desmocollin 1 (MX_{—}008687), envoplakin (XM_{—}008135;U72543), plectin 1 (NM000445), S100A2 (AI539439;M87068), keratin 6A (L4261 1), keratin 17 (Z19574), S100A8 (AI126134), S100A7 (AA586894), S100A9), GB:W72424), SPRR2A), GB:M21302), SPRR1B (M19888), SPRK (AI923984), HCR (BAA81890), SEEK1 (BAA88130), SPR1 (BAB63315), STG (BAA88132), involucrin (NM_{—}005547), annexin A1/lipocortin (X05908), collagen, type VI, alpha 3 (COL6A3) (NM_{—}004369), trichohyalin (NM_{—}005547), and loricrin (XM_{—}048902). GenBank accession numbers are provided in brackets.

Particularly preferred adhesion proteins comprise any of the desmosomal proteins corneodesmosin (also known as S; AF030130), desmoglein 1 (XM_{—}008810), desmocollin 1 (MX_{—}008687), desmoplakins I and II (XM_{—}004463), plakoglobin (also known as PG; NM_{—}002230) and plakophilin (also known as PP).

Highly preferred adhesion proteins include corneodesmosin, desmoglein I, desmoglein 3, plakoglobin, desmoplakin, desmocollin I, envoplakin, a proline-rich protein, preferably a small proline-rich protein (SPRR), SPRR2A, SPRR1B, SPRK, SPRR2E, SPRR2F, SPRR2B, SPRR2D, SPRR2C, SPRR2G, SPRR1A, SPRR3, SPRR4, involucrin, or loricrin.

Mutations and polymorphisms in any of the above adhesion proteins which lead to or are associated with increased or decreased adhesion between epidermal cells may be detected by the methods described in detail in the Examples. Such changes may be detected as a means to identify or diagnose skin disease or susceptibility to such disease.

Corneodesmosin

Mutations in adhesion proteins may be detected as a means to diagnose skin disease or susceptibility to such diseases. In a highly preferred embodiment, the adhesion protein is corneodesmosin.

The corneodesmosin gene is very polymorphic. To date there are nineteen variants described in the literature (Kasahara et al 1996, Jenisch et al, 1999). These polymorphisms are highly conserved rather than sporadic and we believe that they have been selected during vertebrate evolution. Because of the barrier function of the skin some forms of the corneodesmosin giving a strong resistance and/or dynamic function to the skin have been selected. A strong association has been shown between corneodesmosin variant at position +1243 and chronic plaque psoriasis (Tazi-Ahnini et al, 1999b). This association is even stronger in guttate psoriasis (Tazi-Ahnini et al, 1999b). The substitution at position +1243 gives amino acid change L394S. We believe that this substitution interferes with the processing of the corneodesmosin, thus contributing to the disruption of desquamation. We have found that nine of the corneodesmosin polymorphisms giving amino acid change (Ser143/Asp, Ser153/-, Leu180/Phe, Ser202/Phe, Ser401/Gly, Ser408/Ala, Gly409/Val, Ser410/Leu, Asp527/Asn) have an important function in keratinocyte maturation and desquamation process. The screening method for these polymorphisms is described by Jenisch et 1999 and Guerrin et al, 2000. We show that there is a strong relationship between proteolysis process of the corneodesmosin and the sensitivity of normal skin, and also with diseases including psoriasis, acne, eczema in which the barrier function is disturbed.

We show in the Examples that mutations in corneodesmosin are associated with skin diseases, particularly diseases of decreased adhesion.

In particular, we demonstrate in the Examples that a T nucleotide in position +1243 of the corneodesmosin sequence (AF030130) is associated with diseases of decreased skin adhesion. The presence of a T nucleotide at this position leads to a threonine (T) residue at position 394 of the encoded corneodesmosin polypeptide. Detection of either or both may be used to diagnosis disease.

We therefore disclose the diagnosis of a disease of decreased skin adhesion (a Group I disease) or susceptibility to such a disease in an individual, by detecting the presence of a T at position +1243 of a corneodesmosin nucleic acid in an individual. We also provide for the diagnosis of such a disease, or susceptibility to it, by detection of a threonine (T) residue at position 394 of a corneodesmosin polypeptide in an individual. Group I diseases may also be diagnosed by detection of a CD5 or CD6 corneodesmosin allele in an individual; the sequence of the CD5 and CD6 alleles is described in Jenisch et al. 1999.

Preferably, the Group I disease is eczema or dermatitis. More preferably, the Group I disease is atopic eczema or dermatitis hyperformis. Other examples of Group I diseases are set out in a separate section in this document.

Other mutations are disclosed in the Examples at +619 and +180 of corneodesmosin nucleic acid; these and the corresponding polypeptide changes may be detected to diagnose a Group I or Group II disease. Other diagnosis and treatment methods employing corneodesmosin are disclosed in the Examples.

### Proteases

Proteases which may be used in the methods and compositions described here include the following: Apoptosis-related cysteine protease (CASP14) mRNA (NM_{—}012114), Transglutaminase 1 (TGM1) (M98447), TGM2 (XM_{—}009482), TGM4 (XM_{—}056203), TGM5 (XM_{—}007529), TGM7 (NM_{—}052955), TGM3 (L10386), phospholipases A(2) (BC013384), CD47 antigen (X69398), Kallilkrein 8 (AB008390), AD024 protein (XM_{—}002642), SCCE (XM_{—}009002), Defensin beta2 (AF0711216), Interferon a inducible protein 27 (X67325), Fatty acid binding protein FABP5 (M94856), SCTE (XM_{—}009000), kallikrein 1, renal/pancreas/salivary (KLK1) (XM_{—}047300), Homo sapiens kallikrein 2, prostatic (KLK2) (XM_{—}031757), kallikrein 3, (prostate specific antigen) (KLK3) (XM_{—}031768), kallikrein 6 (neurosin, zyme) (KLK6) (XM_{—}055658), kallikrein 4 (prostase, enamel matrix, prostate) (KLK4) (XM_{—}008997), membrane-type serine protease I (AF133086), Human skin collagenase (M13509), collagenase MMP-1 (LOC116389), collagenase MMP-12 (U78045), collagenase MMP-9 (NM_{—}004994), collagenase MMP-3 (U78045), collagenase MMP-28 (AF219624), caspase 7 (BC015799), Caspase 5 (NM_{—}004347), Caspase-14 (NM_{—}012114), ubiquitin specific protease USP-5 (NM_{—}003481), ubiquitin specific protease USP-11 (NM_{—}004651), ubiquitin specific protease USP 6 (NM_{—}004505), ubiquitin specific protease USP 26 (NM_{—}031907), ubiquitin specific protease (USP 28) (NM_{—}020886), 26S protease subunit 4, LILRB1 (AF004230), Signal transducer and activator of transcription 1, 91 kDa (STAT1) (977935), proteasome (prosome, macropain) subunit 6 (PSMA6) (X59417), TPS1 (NM_{—}003293), TPSB1 (XM_{—}016204), TPSG1 (XM_{—}008123), protease nexin-II (XM_{—}047793), Glia derived nexin precursor (P07093), 26S protease regulatory subunit S10B, and PCOLN3 (XM_{—}047524).

Preferably, the protease is one which is involved in or is capable of proteolysis of an adhesion protein, preferably an epidermal cell-cell adhesion protein such as corneodesmosin. Accordingly, in a preferred embodiment, the protease comprises a protease selected from the group consisting of: Stratum Corneum Chymotryptic Enzyme (SCCE), Stratum Corneum Tryptic Enzyme (SCCE) kallikrein 1, kallikrein 2, kallikrein 3, kallikrein 4, kallikrein 6 and kallikrein 8.

Stratum Corneum Chymotryptic Enzyme (SCCE)

Stratum Corneum Tryptic Enzyme (SCTE)

Stratum corneum chymotryptic enzyme (SCCE) or kallikrein 7 (KLK7) as well as stratum corneum tryptic enzyme (SCTE) or kallikrein 5 (KLK5) are members of large serine protease family. Analysis of their expression profiles suggests their skin specificity. Both enzymes are expressed in high suprabasal keratinocytes and interfollicular epidermis and have maximum activity at the physiologic pH of stratum corneum (Ekholm et al, 2000; Eugelrud, 1992, Ekholm and Eugelrud, 1998).

SCCE and SCTE are transported to the stratum corneum extracellular space during cornification. SCCE is produced as an inactive precursor and there is a need for an activating enzyme to confer to SCCE its proteolytic activity. SCTE is thought to be the enzyme involved in SCCE activation. A cluster of kallikrein genes including SCCE and SCTE has been mapped in chromosome 19q13.3-13.4. This includes at least 6 structurally and evolutionary-related members (KLK1, KLK2, KLK3, KLK4, SCCE and SCTE). Each of these kallikrein genes may be used in the methods and compositions described here.

Of those SCCE and SCTE are mainly expressed in skin. They are also expressed in other tissues such as brain, kidney, mammary and salivary glands (Yousef et al, 2000; Yousef and Diamandis, 1999).

We show that the presence of AACCAACC in SCCE nucleic acid is associated with diseases of decreased skin adhesion, in particular eczema (preferably atopic eczema). Therefore, we provide for the diagnosis of a disease of decreased skin adhesion (a Group I disease) or susceptibility to such a disease in an individual, by detecting the presence of an AACC repeat or the sequence AACCAACC of a SCCE nucleic acid in an individual.

Homologues of SCCE and SCTE, in particular, homologues which are localised in the skin and/or act on adhesion proteins, may also be used in the methods of diagnosis and treatment described here. Such homologues may be identified by conventional library screening using an SCCE and/or SCTE probe, as well as database searching using relevant sequences.

Examples of other proteases useful in the methods and compositions described here include aminopeptidase M, carboxypeptidase P, carboxypeptidase Y, caspase 1,4,5, caspase 2,3,7, caspase 6,8,9, chymotrypsin, Factor Xa, pepsin, TEV, thrombin, trypsin etc.

### Protease Inhibitors

We demonstrate that regulation of desquamation is not only controlled by proteases (for example., SCCE and SCTE) but also by their inhibitors. Protease inhibitors which may be used in the methods and compositions described here include the following:

Secretory Leukoprotease Inhibitor (SLPI) and Elafin

Several serine proteases are present in human epidermis including antileukoprotease (skin-derived antileukoprotease) and elafin. Antileukoprotease is a powerful inhibitor of SCCE. Elafin protease inhibitor 3 (PI3 or SKALP) is another serine proteases inhibitors produced by keratinocytes. It is over-expressed in the subcorneal layers of lesional psoriatic skin and in other skin disorders such as Behcet's syndrome, Sweet's syndrome, pyoderma gangrenosum and cutaneous allergic vasculitis Tanaka et al. 2000). We have found that changes of SKALP expression affects SCCE and SCTE activities and hence disturbs the structure of superficial layers of the epidermis in skin disorders such as psoriasis and eczema.

The terms “Secretory Leukoprotease Inhibitor”, “SLPr” and “antileukoprotease” as used in this document are intended to be synonymous with each other.

Elafin is also known as skin-derived antileukoprotease, SKALP and protease inhibitor 3 (PI3). Accordingly, these terms are intended to be synonymous to each other, as used in this document.

Cystatin A

The Examples demonstrate the presence of various polymorphisms in cystatin A, particularly in the promoter region of cystatin A. we therefore provide for the diagnosis of a disease, preferably a Group I and/or a Group II disease, by detecting the presence and/or absence of these polymorphisms.

We show in the Examples that cystatin A is highly expressed in disease with increase adhesion (e.g. acne and psoriasis) and down-regulated in diseases with defective skin barrier (e.g. eczema). Accordingly, activity or level of cystatin A may be up-regulated as a means to treat a Group I diesease. Cystatin A level and/or activity may be down-regulated as a means to treat a Group II diesease.

We therefore disclose the diagnosis of a Group I disease or susceptibility to a Group I disease (preferably eczema or susceptibility to eczema, preferably atopic eczema) in an individual, by detecting modulation, preferably down-regulation of expression of cystatin A in an individual.

We further provide for the diagnosis of a Group II disease or susceptibility to a Group II disease (preferably eczema and/or psoriasis) in an individual, by detecting modulation, preferably up-regulation of expression of cystatin A in an individual.

We provide for the treatment or prevention of a Group I disease (preferably eczema or susceptibility to eczema, preferably atopic eczema) in an individual, by modulating, preferably up-regulating expression of cystatin A in an individual.

We further provide for the treatment or prevention of a Group II disease or susceptibility to a Group II disease (preferably eczema and/or psoriasis) in an individual, by modulating, preferably down-regulating expression of cystatin A in an individual.

### Agonists and Antagonists

The methods and compositions described here rely, in some embodiments, on blocking the activity of proteases or protease inhibitors. Agents which are capable of increasing the activity of a protease or protease inhibitor are referred to as agonists of that activity. Similarly, antagonists reduce the activity of the protease or protease inhibitor.

The term “antagonist”, as used in the art, is generally taken to refer to a compound which binds to an enzyme and inhibits the activity of the enzyme. The term as used here, however, is intended to refer broadly to any agent which inhibits the activity of a molecule, not necessarily by binding to it. Accordingly, it includes agents which affect the expression of a protein such as a protease, or the biosynthesis of a molecule such as a protease inhibitor, or the expression of modulators of the activity of the protease or protease inhibitor. The specific activity which is inhibited may be any activity which is characteristic of the enzyme or molecule, for example, protease activity or protease inhibitor activity. Assays for protease activity and protease inhibitor activity are known in the art.

The antagonist may bind to and compete for one or more sites on the relevant molecule, for example, a protease enzyme, preferably, the catalytic site of the protease enzyme. Preferably, such binding blocks the interaction between the molecule and another entity (for example, the interaction between a protease enzyme and its substrate). However, the antagonist need not necessarily bind directly to a catalytic site, and may bind for example to an adjacent site, another protein (for example, a protein which is complexed with the enzyme) or other entity on or in the cell, so long as its binding reduces the activity of the enzyme or molecule.

Where antagonists of a enzyme such as a protease enzyme are concerned, an antagonist may include a substrate of the enzyme, or a fragment of this which is capable of binding to the enzyme. In addition, whole or fragments of a substrate generated natively or by peptide synthesis may be used to compete with the substrate for binding sites on the enzyme. Alternatively, or in addition, an immunoglobulin (for example, a monoclonal or polyclonal antibody) capable of binding to the protease enzyme may be used. The antagonist may also include a peptide or other small molecule which is capable of interfering with the binding interaction. Other examples of antagonists are set forth in greater detail below, and will also be apparent to the skilled person.

Blocking the activity of a protease or protease inhibitor may also be achieved by reducing the level of expression of the protease or inhibitor in the cell. For example, the cell may be treated with antisense compounds, for example oligonucleotides having sequences specific to the protease or protease inhibitor mRNA. The level of expression of pathogenic forms of adhesion proteins may also be regulated this way.

As used herein, in general, the term “antagonist” includes but is not limited to agents such as an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex.

The terms “antagonist” and “agent” are also intended to include, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an anti sense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, which bind to and occupy the active site of the polypeptide thereby making the catalytic site inaccessible to substrate such that normal biological activity is prevented, are also included. Examples of small molecules include but are not limited to small peptides or peptide-like molecules.

The antagonist or agent may itself be a protease which cleaves the protease or protease inhibitor. Examples of proteases include aminopeptidase M, carboxypeptidase P, carboxypeptidase Y, caspase 1,4,5, caspase 2,3,7, caspase 6,8,9, chymotrypsin, Factor Xa, pepsin, TEV, thrombin, trypsin etc.

### Antisence Compounds

As described above, the antagonist may comprise one or more antisense compounds, including antisense RNA and antisense DNA, which are capable of reducing the level of expression of the protease or protease inhibitor in the cell. Preferably, the antisense compounds comprise sequences complementary to the mRNA encoding the protease or protease inhibitor.

Preferably, the antisense compounds are oligomeric antisense compounds, particularly oligonucleotides. The antisense compounds preferably specifically hybridize with one or more nucleic acids encoding the protease or protease inhibitor. As used herein, the term “nucleic acid encoding protease” or “nucleic acid encoding protease inhibitor” encompasses DNA encoding the protease or protease inhibitor, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the protease or protease inhibitor. In the context of the present document, “modulation” means either an increase (stimulation) or a decrease (inhibition) in the expression of a gene. For example, the expression of a gene encoding an inhibitor of protease or protease inhibitor activity, or an inhibitor of expression of the protease or protease inhibitor, may be increased. However, preferably, inhibition of expression, in particular, inhibition of protease or protease inhibitor expression, is the preferred form of modulation of gene expression and mRNA is a preferred target.

Antisense constructs are described in detail in U.S. Pat. No. 6,100,090 (Monia et al), and Neckers et al., 1992, Crit Rev Oncog 3(1-2):175-231, the teachings of which document are specifically incorporated by reference.

### Polypeptides

The methods and compositions described here provide generally for a number of polypeptides, together with fragments, homologues, variants and derivatives thereof Suitably useful polypeptides include adhesion proteins, proteases or protease inhibitors, or fragments, homologues, varianst or derivatives thereof.

Thus, we disclose variants, homologues or derivatives of an amino acid sequence of an adhesion proteins, protease or protease inhibitor, as well as variants, homologues or derivatives of a nucleotide sequence encoding such amino acid sequences. Each of these may be used for the treatment or diagnosis of a Group I or Group II diesease.

Preferably, the polypeptides, variants, homologues, fragments and derivatives disclosed here comprise one or more properties of the adhesion protein, protease or protease inhibitor, preferably one or more biological activities. Thus, the variants, etc preferably comprise one or more activities including but not limited to adhesion, protease and protease inhibitor activity.

Homologues

The polypeptides disclosed include homologous sequences obtained from any source, for example related viral/bacterial proteins, cellular homologues and synthetic peptides, as well as variants or derivatives thereof. Thus polypeptides also include those encoding homologues of an adhesion protein, protease or protease inhibitor from other species including animals such as mammals (e.g. mice, rats or rabbits), especially primates, more especially humans. More specifically, homologues include human homologues.

In the context of this document, a homologous sequence is taken to include an amino acid sequence which is at least 15, 20, 25, 30, 40, 50, 60, 70, 80 or 90% identical, preferably at least 95 or 98% identical at the amino acid level, preferably over at least 50 or 100, preferably 200, 300, 400 or 500 amino acids with the relevant sequence.

In particular, homology should typically be considered with respect to those regions of the sequence known to be essential for protein function rather than non-essential neighbouring sequences, for example, sequences essential for proteolysis and/or adhesion. This is especially important when considering homologous sequences from distantly related organisms.

Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These publicly and commercially available computer programs can calculate % identity between two or more sequences. % identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local identity or similarity.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Altschul et al., 1990, J. Mol. Biol., 403410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence retains substantially the same activity as the unmodified sequence, preferably having at least the same activity as the polypeptides shown here.

Polypeptides having the amino acid sequence shown here, including in the Examples, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides described here. Functional domains of proteases include the protease domain and protease catalytic site. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Fragments

Polypeptides also include fragments of the full length sequence of any of the polypeptides described here (e.g., an adhesion protein, protease or protease inhibitor). Preferably fragments comprise at least one epitope. Methods of identifying epitopes are well known in the art. Fragments will typically comprise at least 6 amino acids, more preferably at least 10, 20, 30, 50 or 100 amino acids.

Included are fragments comprising, preferably consisting of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500, or more residues from a relevant nucleic acid sequence, e.g., a nucleic acid sequence encoding an adhesion protein, protease or protease inhibitor.

Adhesion proteins, proteases and protease inhibitors, and their fragments, homologues, variants and derivatives, may be made by recombinant means. However they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins may also be obtained by purification of cell extracts from animal cells.

The polypeptides, variants, homologues, fragments and derivatives disclosed here may be in a substantially isolated form. It will be understood that such polypeptides may be mixed with carriers or diluents which will not interfere with the intended purpose of the protein and still be regarded as substantially isolated. A variant, homologue, fragment or derivative may also be in a substantially purified form, in which case it will generally comprise the protein in a preparation in which more than 90%, e.g. 95%, 98% or 99% of the protein in the preparation is a protein.

The polypeptides, variants, homologues, fragments and derivatives disclosed here may be labelled with a revealing label. The revealing label may be any suitable label which allows the polypeptide , etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols. polypeptide, variant, homologue, fragment or derivative disclosed here, optionally labelled, my also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The adhesion protein, protease or protease inhibitor polypeptides, variants, homologues, fragments and derivatives disclosed here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which the adhesion protein, protease and protease inhibitor polypeptides, variants, homologues, fragments and derivatives disclosed here are expressed may be used in assay systems to identify candidate substances which interfere with or enhance the functions of the polypeptides in the cell.

### Fragments

We also provide for nucleic acids and polypeptides or peptides which are fragments of the adhesion protein, protease or protease inhibitor nucleic acids and polypeptides disclosed here.

Preferably such nucleic acid and polypeptide fragments comprise, preferably consist of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500, or more residues from a relevant sequence (i.e., any of the sequences disclosed here). Preferably, such fragments comprise biological activity, preferably adhesion, protease or protease inhibitor activity.

A nucleic acid of use in the methods and compositions described here may comprise a viral or non-viral DNA or RNA vector, where non-viral vectors include, but are not limited to, plasmids, linear nucleic acid molecules, artificial chromosomes, condensed particles and episomal vectors. Expression of heterologous genes has been observed after injection of plasmid DNA into muscle (Wolff J. A. et al., 1990, Science, 247: 1465-1468; Carson D. A. et al., U.S. Pat. No. 5,580,859), thyroid (Sykes et al., 1994, Human Gene Ther., 5: 837-844), melanoma (Vile et al., 1993, Cancer Res., 53: 962-967), skin (Hengge et al., 1995, Nature Genet., 10: 161-166), liver (Hickman et al., 1994, Human Gene Therapy, 5: 1477-1483) and after exposure of airway epithelium (Meyer et al., 1995, Gene Therapy, 2: 450-460).

As used herein, the term “nucleic acid” is defined to encompass DNA and RNA or both synthetic and natural origin which DNA or RNA may contain modified or unmodified deoxy- or dideoxy-nucleotides or ribonucleotides or analogues thereof. The nucleic acid may exist as single- or double-stranded DNA or RNA, an RNA/DNA heteroduplex or an RNA/DNA copolymer, wherein the term “copolymer” refers to a single nucleic acid strand that comprises both ribonucleotides and deoxyribonucleotides.

The term “synthetic”, as used herein, is defined as that which is produced by in vitro chemical or enzymatic synthesis.

Therapeutic nucleic acid sequences useful according to the methods and compositions described here include those encoding adhesion proteins, as well as proteases, protease inhibitors, etc. Therapeutic nucleic acid sequences also include sequences encoding nuclear proteins, cytoplasmic proteins, mitochondrial proteins, secreted proteins, plasmalemma-associated proteins, serum proteins, viral antigens, bacterial antigens, protozoal antigens and parasitic antigens. Therapeutic nucleic acid sequences also include sequences encoding proteins, lipoproteins, glycoproteins, phosphoproteins and nucleic acids (e.g., RNAs such as ribozymes or antisense nucleic acids). Ribozymes of the hammerhead class are the smallest known, and lend themselves both to in vitro synthesis and delivery to cells (summarised by Sullivan, 1994, J. Invest. Dermatol., 103: 85S-98S; Usman et al., 1996, Curr. Opin. Struct. Biol., 6: 527-533). The compounds which can be incorporated are only limited by the availability of the nucleic acid sequence encoding a given protein or polypeptide. One skilled in the art will readily recognise that as more proteins and polypeptides become identified, their corresponding genes can be cloned into the gene expression vector(s) of choice, administered to a tissue of a recipient patient or other vertebrate, and expressed in that tissue.

### Nucleic Acids

The methods and compositions described here provide generally for a number of nucleic acids, together with fragments, homologues, variants and derivatives thereof. Suitably useful nucleic acids include those which encode adhesion proteins, proteases or protease inhibitors, or fragments, homologues, varianst or derivatives thereof.

As used here in this document, the terms “polynucleotide”, “nucleotide”, and nucleic acid are intended to be synonymous with each other. “Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

We also provide nucleic acids which are fragments, homologues, variants or derivatives of the relevant nucleic acids (e.g., an adhesion protein, protease or protease inhibitor encoding nucleic acid).

Variants, Derivatives and Homologues

Nucleic acid variants, fragments, derivatives and homologues may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides may be modified by any method available in the art. Such modifications may be carried out in order to enhance the in vivo activity or life span of polynucleotides of interest.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleic acid from or to the sequence. Preferably said variant, homologues or derivatives code for a polypeptide having biological activity. Preferably, such fragments, homologues, variants and derivatives of the adhesion proteins, proteases and protease inhibitors comprise modulated enzymatic activity, for example, adhesion protein activity (e.g., ability to enable the adhesion between two cells), protease activity or protease inhibitor activity. Thus, for example, we provide fragments, homologues, variants and derivatives of the relevant polypeptides which comprise a lower adhesion protein, protease or protease inhibitor activity compared to unmodified polypeptides. In particular, we provide fragments, homologues, variants and derivatives of adhesion proteins, which display reduced or enhanced adhesion activity. Fragments, homologues, variants and derivatives of proteases and protease inhibitors are also provided, which display reduced or enhanced protease or protease inhibitor activity.

Assays for adhesion activity, protease activity and protease inhibitor activity are known in the art.

As indicated above, with respect to sequence identity, a “homologue” has preferably at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence shown in the sequence listings. Thus, in particular, we provide the use of homologues of adhesion proteins, proteases and protease inhibitors having such identity in the treatment of Group I and Group II diseases

More preferably there is at least 95% identity, more preferably at least 96% identity, more preferably at least 97% identity, more preferably at least 98% identity, more preferably at least 99% identity. Nucleotide identity comparisons may be conducted as described above. A prefeffed sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

We further describe nucleotide sequences that are capable of hybridising selectively to any of the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, may be at least 40% homologous, at least 45% homologous, at least 50% homologous, at least 55% homologous, at least 60% homologous, at least 65% homologous, at least 70% homologous, at least 75% homologous, at least 80% homologous, at least 85% homologous, at least 90% homologous, or at least 95% homologous to the corresponding nucleotide sequences presented herein. Preferably, such polynucleotides will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²p.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, we provide nucleotide sequences that can hybridise to the adhesion protein, protease or protease inhibitor nucleic acids, fragments, variants, homologues or derivatives under stringent conditions (e.g. 65° C. and 0.1×SSC {1×SSC=0.15 M NaCl, 0.015 M Na₃ Citrate pH 7.0).

Generation of Homologues, Variants and Derivatives

Polynucleotides which are not 100% identical to the sequences of the present invention but which are also included, as well as homologues, variants and derivatives of the adhesion proteins, proteases and protease inhibitors can be obtained in a number of ways. Other variants of the sequences may be obtained for example by probing DNA libraries made from a range of individuals, for example individuals from different populations. For example, homologues may be identified from other individuals, or other species. Further recombinant nucleic acids and polypeptides may be produced by identifying corresponding positions in the homologues, and synthesising or producing the molecule as described elsewhere in this document.

In addition, other viral/bacterial, or cellular homologues of adhesion proteins, proteases and protease inhibitors, particularly cellular homologues found in mammalian cells (e.g. rat, mouse, bovine and primate cells), may be obtained and such homologues and fragments thereof in general will be capable of selectively hybridising to a human adhesion protein, protease or protease inhibitor. Such homologues may be used to design non-human adhesion protein, protease and protease inhibitor nucleic acids, fragments, variants and homologues. Mutagenesis may be carried out by means known in the art to produce further variety.

Sequences of homologues may be obtained by probing cDNA libraries made from or genomic DNA libraries from other animal species, and probing such libraries with probes comprising all or part of any of the adhesion protein, protease or protease inhibitor nucleic acids, fragments, variants and homologues, or other fragments under conditions of medium to high stringency.

Similar considerations apply to obtaining species homologues and allelic variants of the polypeptide or nucleotide sequences disclosed here.

Variants and strain/species homologues may also be obtained using degenerate PCR which will use primers designed to target sequences within the variants and homologues encoding conserved amino acid sequences within the sequences of the nucleic acids encoding adhesion proteins, proteases or protease inhibitors. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments the sequences.

In addition, homologous sequences may be identified by searching nucleotide and/or protein databases using search algorithms such as the BLAST suite of programs.

Alternatively, such polynucleotides may be obtained by site directed mutagenesis of characterised sequences, for example, adhesion protein, protease and protease inhibitor nucleic acids, or variants, homologues, derivatives or fragments thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 8, 9, 10, or 15, preferably at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector Polynucleotides or primers may carry a revealing label. Suitable labels include radioisotopes such as ³²p or 35S, enzyme labels, or other protein labels such as biotin. Such labels may be added to polynucleotides or primers and may be detected using by techniques known per se. Polynucleotides or primers or fragments thereof labelled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected.

Suitable assay methods of this and other formats can be found in for example WO89/03891 and WO90/13667.

Tests for sequencing nucleotides, involve bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

### Antibodies

We further provide an antibody, either polyclonal or monoclonal, which specifically binds to epitopes on the polypeptide/protein encoded by the corneodesmosin gene or mutant epitopes. In preparing the antibody, the protein (with and without mutations) encoded by the corneodesmosin gene and polymorphisms thereof is used as a source of the immunogen. Peptide amino acid sequences isolated from the amino acid sequence or mutant peptide sequences may also be used as an immunogen.

The antibodies may be either monoclonal or polyclonal. Conveniently, the antibodies may be prepared against a synthetic peptide based on the sequence, or prepared recombinantly by cloning techniques or the natural gene product and/or portions thereof may be isolated and used as the immunogen. Such proteins or peptides may be used to produce antibodies by standard antibody production technology well known to those skilled in the art as described generally in Harlow and Lane, Antibodies: A Laboratory Manual, Cold Spring Harbor Laboratory Press, Cold Spring Harbor, N.Y., 1988.

For producing polyclonal antibodies a host, such as a rabbit or goat, is immunized with the protein or peptide, generally with an adjuvant and, if necessary, coupled to a carrier; antibodies to the protein are collected from the sera.

For producing monoclonal antibodies, the technique involves hyperimmunization of an appropriate donor, generally a mouse, with the protein or peptide fragment and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

The antibody may be bound to a solid support substrate or conjugated with a detectable moiety or be both bound and conjugated as is well known in the art. (For a general discussion of conjugation of fluorescent or enzymatic moieties see Johnstone and Thorpe, Immunochemistry in Practice, Blackwell Scientific Publications, Oxford, 1982.) The binding of antibodies to a solid support substrate is also well known in the art. (see for a general discussion Harlow and Lane Antibodies: A Laboratory Manual, Cold Spring Harbor Laboratory Publications, New York, 1988) The detectable moieties may include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

### Linkage Disequilibrium Analysis

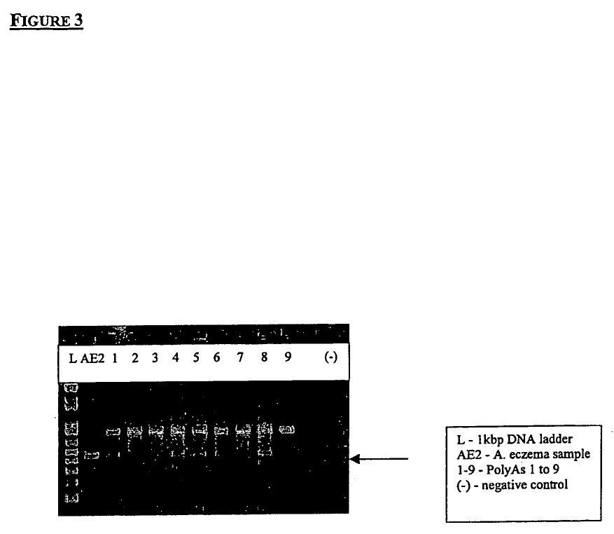

Linkage disequilibrium (LD) analysis is a powerful tool for mapping disease genes and may be particularly useful for investigating complex traits. LD mapping is based on the following expectations: for any two members of a population, it is expected that recombination events occurring over several generations will have shuffled their genomes, so that they share little in common with their ancestors. However, if these individuals are affected with a disease inherited from a common ancestor, the gene responsible for the disease and the markers that immediately surround it will likely be inherited without change, or IBD (“identical by descent”), from that ancestor. The size of the regions that remain shared (i.e. IBD) are inversely proportional to the number of generations separating the affected individuals and their common ancestor. Thus, “old” populations are suitable for fine scale mapping and recently founded ones are appropriate for using LD to roughly localize disease genes. (Houwen et al., 1994, in particular FIG. 3 and accompanying text). Because isolated populations have typically had a small number of founders, they are particularly suitable for LD approaches, as indicated by several successful LD studies conducted in Finland (de la Chapelle, 1993).

LD analysis has been used in several positional cloning efforts (Kerem et al., 1989; MacDonald et al., 1992; Petrukhin et al., 1993; Hastbacka et al., 1992 and 1994), but in each case the initial localization had been achieved using conventional linkage methods. Positional cloning is the isolation of a gene solely on the basis of its chromosomal location, without regard to its biochemical function. Lander and Botstein (1986) proposed that LD mapping could be used to screen the human genome for disease loci, without conventional linkage analyses. This approach was not practical until a set of mapped markers covering the genome became available (Weissenbach et al., 1992). The feasibility of genome screening using LD mapping is now demonstrated by the applicants.

Identification of the chromosomal location of a gene responsible for causing a disease associated with increased or reduced cell-cell adhesion in the epithelium can facilitate diagnosis, treatment and genetic counseling of individuals in affected families.

Due to the severity of the disorder and the limitations of a purely phenotypic diagnosis of such diseases, there is a tremendous need to genetically subtype individuals to confirm clinical diagnoses and to determine appropriate therapies based on their genotypic subtype.

### Diagnosis of Diseases

We provide for methods of diagnosis of diseases associated with abnormal epithelial cell-cell adhesion. Mutations in genes encoding adhesion proteins (for example, corneodesmosin) are shown to result in reduced adhesion between epithelial cells such as corneocytes. Some mutations in these genes may result in altered (typically reduced) expression of adhesion proteins, or in expression of adhesion proteins which have reduced adhesion activity or higher protease sensitivity. Other mutations in protease and protease inhibitor genes are also associated with disease, as shown in the Examples. Such mutations are typically associated with and may lead to Group 1 diseases, as the reduced cell-cell adhesion results in impaired barrier function of the epidermis.

Accordingly, a Group 1 disease may be diagnosed in a patient suffering or likely to suffer from the disease by determining the level of expression of an adhesion protein, in which a reduced level of expression of an adhesion protein is diagnostic of a Group I disease. Furthermore, we provide a method of diagnosis of a Group 1 disease by determining the adhesion activity of an adhesion protein involved in epithelial cell-cell adhesion, in which a reduced adhesion activity is diagnostic of a Group 1 disease. We further provide a method of diagnosis of a Group 1 disease by determining the protease sensitivity of an adhesion protein involved in epithelial cell-cell adhesion, in which an increased protease sensitivity is diagnostic of a Group 1 disease. We also disclose a method of diagnosis of a Group 1 disease, by determining the presence of a mutation which is associated with a reduced expression of an adhesion protein, or with expression of an adhesion protein with reduced adhesion activity and/or increased protease sensitivity.

Other mutations in genes encoding adhesion proteins such as corneodesmosin result in increased adhesion between epithelial cells such as corneocytes. Some mutations in these genes may result in enhanced expression of adhesion proteins, or in expression of adhesion proteins which have enhanced adhesion activity or reduced protease sensitivity. Mutations in protease and protease inhibitor genes are also disclosed which are associated with disease. These mutations are typically associated with and may lead to Group 2 diseases, as they lead to enhanced cell-cell adhesion.

Accordingly, a Group 2 disease may be diagnosed in a patient suffering or likely to suffer from the disease by determining the level of expression of an adhesion protein, in which a increased level of expression of an adhesion protein is diagnostic of a Group 2 disease. Furthermore, we describe a method of diagnosis of a Group 2 disease by determining the adhesion activity of an adhesion protein involved in epithelial cell-cell adhesion, in which a increased adhesion activity is diagnostic of a Group 2 disease. We further describe a method of diagnosis of a Group 2 disease by determining the protease sensitivity of an adhesion protein involved in epithelial cell-cell adhesion, in which an decreased protease sensitivity is diagnostic of a Group 2 disease. Also included is a method of diagnosis of a Group 2 disease, by determining the presence of a mutation which is associated with a increased expression of an adhesion protein, or with expression of an adhesion protein with increased adhesion activity and/or decreased protease sensitivity.

According to a preferred embodiment, diagnosis of a Group 1 or Group 2 disease is carried out by detection of the presence or absence of a Hph1 restriction enzyme site in the corneodesmosin gene.

Preferably, a Group 1 disease is diagnosed by detection of the absence of a Hph1 site in the corneodesmosin gene. Preferably, a Group 2 disease is diagnosed by the detection of the presence of a Hph1 site in the corneodesmosin gene. More preferably, the Group 1 disease diagnosed by the absence of an Hph1 site is eczema or Crohn's disease, and the Group 2 disease diagnosed by the presence of a Hph1 site is psoriasis or acne.

Alternatively or in conjunction, we provide for the diagnosis of a Group 1 or Group 2 disease by detection of the presence or absence of a T at position +1243 in the corneodesmosin gene. Preferably, a Group 1 disease is diagnosed by detection of the presence of a T at position at +1243 in the corneodesmosin gene. Preferably, a Group 2 disease is diagnosed by the detection of the absence of a T at position +1243 in the corneodesmosin gene. More preferably, the Group 1 disease diagnosed by the presence of a T at position +1243 is eczema or Crohn's disease, and the Group 2 disease diagnosed by the absence of a T at position +1243 is psoriasis or acne.

Preferably, the presence of T at position +1243 is associated with a C to T transition. Furthermore, the absence of T at position +1243 is associated with a T to C transition.

Any method of determining protease sensitivity of a protein as known in the art (Egelrud, 1993) may be used in the diagnostic methods presented above. Similarly, detection of mutations by means of, for example, SSCP, RFLP, SNP, etc analysis are known in the art and are described in further detail below.

Detection of abnormal (i.e., increased or decreased) proteolytic breakdown of an adhesion protein may also be used to diagnose Group 1 and Group 2 diseases. Thus, an increased level of expression and/or activity of a protease involved in proteolysis of an adhesion protein is characteristic (and hence diagnostic) of a Group 1 disease. Accordingly, we provide a method of diagnosis of a Group 1 disease, by detecting the level of expression of a protease involved in proteolysis of an adhesion protein. We further provide a method of diagnosis of a Group 1 disease, by detecting the level of activity of a protease involved in proteolysis of an adhesion protein, in which an increased level of activity of the protease is diagnostic of a Group 1 disease. The method is also suitable for diagnosis of a Group 2 disease. Thus, a method of diagnosis of a Group 2 disease comprises detection of the level of expression of a protease involved in proteolysis of an adhesion protein, in which a decreased expression of the protease is diagnostic of a Group 2 disease. Furthermore, we provide a method of diagnosis of a Group 2 disease, by detecting the level of activity of a protease involved in proteolysis of an adhesion protein, in which an decreased level of activity of the protease is diagnostic of a Group 2 disease.

The level of expression and/or activity of a protease inhibitor which is capable of inhibiting the proteolytic activity of a protease involved in breakdown of an adhesion protein may also be detected as a means of diagnosis of a Group 1 or Group 2 disease. Accordingly, an decreased level of expression and/or activity of a protease inhibitor is characteristic of a Group 1 disease. Therefore, a method of diagnosis of a Group 1 disease comprises detecting a decreased level of expression of a protease inhibitor in a patient. Similarly, a method of diagnosis of a Group 1 disease may also comprise detecting the level of activity of a protease inhibitor, in which an decreased level of activity of a protease inhibitor is diagnostic of a Group 1 disease.

In a further embodiment, a method of diagnosis of a Group 2 disease comprises detecting the level of expression of a protease inhibitor, in which an increased expression of a protease inhibitor is diagnostic of a Group 2 disease. Furthermore, a method of diagnosis of a Group 2 disease may comprise detecting the level of activity of a protease inhibitor, in which an increased activity of a protease inhibitor is diagnostic of a Group 2 disease.

It is clear that detection of expression and/or activity of proteases and/or protease inhibitors may also be undertaken at a genetic level, i.e., detecting mutations associated with increased or decreased protease/protease inhibitor activity.

Preferably, the adhesion protein which is detected or whose properties are determined in the above methods is corneodesmosin, and preferably, the gene is a gene encoding corneodesmosin. Preferably, the protease that is detected, etc is stratum corneum chymotryptic enzyme (SCCE) or stratum corneum tryptic enzyme (SCTE). Preferably, the protease inhibitor that is detected, etc, is anti-leukoprotease (SLPI) or elafin protease inhibitor 3 (PI3 or SKALP).

Preferably, the protease inhibitor that is detected, etc, is or elafin. Where the terms “higher”, “increased”, and “enhanced” are used with reference to activity, protease sensitivity, expression, etc, these are understood to be relative to the corresponding properties of a normal, un-diseased epithelium from the same patient or another individual. Similarly, the terms “lower”, “decreased” and “reduced” are to refer to a level relative to a normal, un-diseased epithelium.

Specific non-limiting examples of diagnostic methods suitable for the methods and compositions described here are set out below:

Genetic diagnostic tests may be performed by genotyping genes encoding corneodesmosomal proteins (e.g. corneodesmosome), proteases (SCCE, SCTE) and protease inhibitors (i.e. SKALP, SLPI) for SNPs associated with disease of interest. Primers are designed correspond to sequences which flank mutations and/or polymorphisms. The sequences of proteases (SCCE, SCTE) and protease inhibitors (i.e. SKALP, SLPI) genes may, for example, be used to design primers in the extremities of each exon using Gene Jockey II software (Biosoft, 49 Bateman Street, Cambridge CB2 1LR). After PCR amplification, the mutation can may be detected by allelic discrimination using restriction enzymes, TaqMan analysis (as described below) or simply by sequencing.

Briefly, 25 μl PCR reactions comprised 8% glycerol, 200 μM each dATP, dGTP and dCTP, 400 μM dUTP, 1.25 U AmplitaqGold (Perkin-Elmer, U.S.A), 1.25 U Uracil-N-Glycosylase (Perkin-Elmer, USA), 5 mM MgCl₂, 500-900 nM each primer. Allelic discrimination at these loci is performed using a 5′ nuclease assay (TaqMan™ allelic discrimination test), a method extensively validated. This test is based on 5′ nuclease activity of Taq polymerase and the detection by fluorescence-resonance energy transfer (FRET) of the cleavage of two probes designed to hybridise to either allele during PCR. Double fluorescent probes are provided by ABI-PE (Forster City, Calif.; Warrington, UK). Probe and primer sequences are designed and probes are labelled with carboxyfluorescein (FAM) and carboxy-4,7,2′,7′-tetrachlorofluorescein (TET) fluorescent dyes at the 5′ end, and with the quencher carboxytetramethylrhodamine (TAMRA) at the 3′ terminus. Concentrations of FAM and TET probes ranged between 20-50 nM and 50-350 nM respectively depending on the probes used. Plates are scanned in an LS50-B or a PE7200 fluorimeter (ABI/Perkin-Elmer).

As used herein the term “polymerase chain reaction” or “PCR” refers to the PCR procedure described in the patents to Mullis, et al., U.S. Pat. Nos. 4,683,195 and 4,683,202. The procedure basically involves: (1) treating extracted DNA to form single-stranded complementary strands; (2) adding a pair of oligonucleotide primers, wherein one primer of the pair is substantially complementary to part of the sequence in the sense strand and the other primer of each pair is substantially complementary to a different part of the same sequence in the complementary antisense strand; (3) annealing the paired primers to the complementary sequence; (4) simultaneously extending the annealed primers from a 3′ terminus of each primer to synthesize an extension product complementary to the strands annealed to each primer wherein said extension products after separation from the complement serve as templates for the synthesis of an extension product for the other primer of each pair; (5) separating said extension products from said templates to produce single-stranded molecules; and (6) amplifying said single-stranded molecules by repeating at least once said annealing, extending and separating steps.

Biochemical diagnostic tests include proteolysis profiles, screening for N/C terminals, screening for enzyme function and/or activity.

Proteolysis profiles may be conducted as follows. Polyclonal antibody capable of recognising the full and the mature forms of corneodesmosin are generated. Stratum corneum extract may be extracted from strips obtained from patients. Protein extracts are run on SDS-PAGE gel, transferred onto a membrane and hybridised with primary anti-corneodesmosin antibody and labelled secondary antibody. Molecular weight of processed corneodesmosin of patients are compared to the ones from controls.

Screening for N-/C-terminals may be conducted as follows. In the case of the presence of particular forms of desmosomal/corneodesmosomal proteins (e.g. corneodesmosin) in a patient group but not in controls or vice versa, N-/C-terminal specific antibodies antibody may be produced to detect the pathogenic forms of desmosomal/corneodesmosomal proteins (e.g. corneodesmosin). The identification of the pathogenic forms may be performed as follow. Different forms of corneodesmosin are purified from non denaturing hypotonic buffer extract (TEA buffer extract) of human epidermis by anion exchange and affinity chromatography (Simon et al, 1997). The eluted fractions of the affinity column are pooled, lyophilized, separated by SDS-PAGE. The bands corresponding to the pathogenisis forms of corneodesmosin are excised and characterized by internal and NH2-terminal amino acid sequence analysis.

Screening for enzyme function/activity may be conducted as follows.

Desmosomal/corneodesmosomal proteins (e.g. corneodesmosin) are radiolabelled during in vitro translation from cDNA and incubated with protease enzymes (SCCE, SCTE) at 37° C. for 1.5-3 h. SCCE and SCTE may be prepared using KCI extract of dissociated plantar corneocytes as described by Egelrud, 1993, and Brattsand and Egelrud, 1999 (Egelrud, 1993; Brattsand and Egelrud, 1999). SCCE may be purified by affinity chromatography on SBTI Affigel 15. An alternative method is to produce a fusion protein in vitro and purify using immunoaffinity columns (Ekholm et al, 2000). SDS-PAGE and autoradiography are used to analyze the pattern of hydrolytic peptides. The intensity of autoradiography bands is quantified by a densitometry and the fraction of peptides less that 56 kDa (full length) is calculated. Activity is expressed as mol substrate processed by g enzyme per second.

### Desmosomes and Corneodesmosomes

Desmosomes are symmetrical structures that form disc-shaped intercellular junctions between epithelial cells. In the epidermis the desmosomes mediate the adhesion between keratinocytes. In psoriasis, various ichtyoses and skin xerosis, the number of corneodesmosomes (desmosomes in upper layers of the epidermis) is increased in the stratum corneum. Immunoelectron microscopy has been used to show the define the interactions within corneodesmosomes between proteins of the extracellular core domain such as desmoglein and desmocollins and intracellular corneodesmosomal proteins including desmoplakins I and II, plakoglobin (PG) and plakophilins (PP) (Cowin and Burke, 1996). The importance of corneodesmosomal proteins in epidermal integrity is demonstrated by inherited disorders such as striate subtype of palmoplantar keratoderma caused by desmoplakin haploinsufficiency (Armstrong et al, 1999). Mutations in loricrin gene lead to keratoderma of Camisa. Corneodesmosin is a glycoprotein of corneodesmosomes. Three forms of the corneodesmosin with different weights 33-36 to 40-46 and 52-56 kDa have been isolated from the epidermis (Simon et al, 1997).

Mutations within the corneodesmosin/S gene and related genes within the MHC epidermal gene cluster (chromosome 6p21) result in a reduced cohesion between corneocytes.

### Screening Assays

The adhesion proteins, proteases and protease inhibitors described here may be employed in a screening process for compounds which bind the polypeptides and which activate (agonists) or inhibit activation of (antagonists) of the polypeptide. Thus, the adhesion proteins, proteases and protease inhibitors may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2):Chapter 5 (1991).

Adhesion proteins, proteases and protease inhibitors are responsible for many biological functions, including many pathologies such as skin diseases including inflammatory skin diseases such as Group I and Group II diseases. Accordingly, it is desirous to find compounds and drugs which stimulate adhesion proteins, proteases and protease inhibitors, or which can inhibit the function of the adhesion proteins, proteases and protease inhibitors on the other hand. In general, agonists and antagonists are employed for therapeutic and prophylactic purposes for any of the Group I and/or Group II diseases disclosed here.

Rational design of candidate compounds likely to be able to interact with adhesion proteins, proteases and protease inhibitors may be based upon structural studies of the molecular shapes of a polypeptide according to the invention. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

An alternative to rational design uses a screening procedure which involves in general producing appropriate cells which express the adhesion proteins, proteases or protease inhibitors on the surface thereof. Such cells include cells from animals, yeast, Drosophila or E. coli. Cells expressing the polypeptide (or cell membrane containing the expressed polypeptide) are then contacted with a test compound to observe binding, or stimulation or inhibition of a functional response. For example, Xenopus oocytes may be injected with mRNA encoding any one or more of adhesion proteins, proteases and protease inhibitors or polypeptide, and currents induced by exposure to test compounds measured by use of voltage clamps measured, as described in further detail elsewhere.

Where the candidate compounds are proteins, in particular antibodies or peptides, libraries of candidate compounds may be screened using phage display techniques. Phage display is a protocol of molecular screening which utilises recombinant bacteriophage. The technology involves transforming bacteriophage with a gene that encodes one compound from the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of binding to a polypeptide or peptide of the invention are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al., (1990) Proc. Nat'l Acad. Sci. USA 87;4007-4011, which describe sensitive methods to detect cellular responses. Competitive assays are particularly useful, where the cells expressing the library of compounds are contacted or incubated with a labelled antibody known to bind to a BACH polypeptide of the present invention, such as ¹²⁵I-antibody, and a test sample such as a candidate compound whose binding affinity to the binding composition is being measured. The bound and free labelled binding partners for the polypeptide are then separated to assess the degree of binding. The amount of test sample bound is inversely proportional to the amount of labelled antibody binding to the polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Another technique for candidate compound screening involves an approach which provides high throughput screening for new compounds having suitable binding affinity, e.g., to a polypeptide of the invention, and is described in detail in International Patent application no. WO 84/03564 (Commonwealth Serum Labs.), published on Sep. 13 1984. First, large numbers of different small peptide test compounds are synthesized on a solid substrate, e.g., plastic pins or some other appropriate surface; see Fodor et al. (1991). Then all the pins are reacted with solubilized polypeptide of the invention and washed. The next step involves detecting bound polypeptide. Compounds which interact specifically with the polypeptide will thus be identified.

Ligand binding assays provide a direct method for ascertaining pharmacology and are adaptable to a high throughput format. The purified ligand for a polypeptide may be radiolabeled to high specific activity (50-2000 Ci/mmol) for binding studies. A determination is then made that the process of radiolabeling does not diminish the activity of the ligand towards its binding partner. Assay conditions for buffers, ions, pH and other modulators such as nucleotides are optimized to establish a workable signal to noise ratio for both membrane and whole cell sources. For these assays, specific binding is defined as total associated radioactivity minus the radioactivity measured in the presence of an excess of unlabeled competing ligand. Where possible, more than one competing ligand is used to define residual nonspecific binding.

The assays may simply test binding of a candidate compound wherein adherence to the cells bearing the polypeptide is detected by means of a label directly or indirectly associated with the candidate compound or in an assay involving competition with a labeled competitor. Further, these assays may test whether the candidate compound results in a signal generated by binding to the polypeptide, using detection systems appropriate to the cells bearing the polypeptides at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing an adhesion protein protease or protease inhibitor polypeptide to form a mixture, measuring activity of the relevant protein in the mixture, and comparing the activity of the mixture to a standard.

CDNA encoding adhesion proteins, proteases and protease inhibitors, protein and antibodies to the proteins may also be used to configure assays for detecting the effect of added compounds on the production of mRNA and protein in cells. For example, an ELISA may be constructed for measuring secreted or cell associated levels of adhesion proteins, proteases and protease inhibitors using monoclonal and polyclonal antibodies by standard methods known in the art, and this can be used to discover agents which may inhibit or enhance the production of adhesion proteins, proteases or protease inhibitors (also called antagonist or agonist, respectively) from suitably manipulated cells or tissues. Standard methods for conducting screening assays are well understood in the art.

Examples of potential antagonists of adhesion proteins, proteases and protease inhibitors include antibodies or, in some cases, nucleotides and their analogues, including purines and purine analogues, oligonucleotides or proteins which are closely related to the ligand of the adhesion proteins, proteases or protease inhibitors, e.g., a fragment of the ligand, or small molecules which bind to the polypeptide but do not elicit a response, so that the activity of the polypeptide is prevented.

The present invention therefore also provides a compound capable of binding specifically to a adhesion protein, protease or protease inhibitor polypeptide and/or peptide of the present invention.

The term “compound” refers to a chemical compound (naturally occurring or synthesised), such as a biological macromolecule (e.g., nucleic acid, protein, nonpeptide, or organic molecule), or an extract made from biological materials such as bacteria, plants, fungi, or animal particularly mammalian) cells or tissues, or even an inorganic element or molecule. Preferably the compound is an antibody.

The materials necessary for such screening to be conducted may be packaged into a screening kit. Such a screening kit is useful for identifying agonists, antagonists, ligands, receptors, substrates, enzymes, etc. for adhesion protein, protease and protease inhibitor polypeptides or compounds which decrease or enhance the production of adhesion protein, protease and protease inhibitor polypeptides. The screening kit comprises: (a) an adhesion protein, protease or protease inhibitor polypeptide; (b) a recombinant cell expressing such a polypeptide; (c) a cell membrane expressing a such a polypeptide; or (d) antibody to such a polypeptide. The screening kit may optionally comprise instructions for use.

Substrates of proteases may be used to assay molecules capable of modulating protease activity. For example, an assay to identify a molecule capable of modulating activity of SCCE employs the substrate S-2586 (MeO-Suc-Arg-Pro-Tyr-pNA). S-2586 absorbs at 405 nm when it is cleaved. In the assay, SCCE and S-2586 are incubated with a candidate molecule, and the absorbance at 405 nm is detected to detect cleavage of the substrate. Suitable candidate molecules to be used as inhibitors of SCCE and other proteases include those which decrease the absorption at 405 nm.

### Expression Vectors

We provide vectors comprising an expression control sequence operatively linked to the nucleic acid sequence of an adhesion protein, protease or protease inhibitor gene, and portions thereof as well as mutant sequences which lead to the expression of forms of such proteins associated with Group 1 and/or Group 2 diseases. We further provide host cells, selected from suitable eucaryotic and procaryotic cells, which are transformed with these vectors.

Using the methods and compositions described here, it is possible to transform host cells, including E. coli, using the appropriate vectors so that they carry recombinant DNA sequences derived from the adhesion protein, protease or protease inhibitor transcript or containing the entire transcript in its normal form or a mutated sequence containing point mutations, deletions, insertions, or rearrangements of DNA. Such transformed cells allow the study of the function and the regulation of the relevant gene. Use of recombinantly transformed host cells allows for the study of the mechanisms of regulation of the adhesion proteins, proteases and protease inhibitors and, in particular it will allow for the study of gene function interrupted by the mutations in the adhesion protein, protease or protease inhibitor gene region.

Vectors are known or may be constructed by those skilled in the art and should contain all expression elements necessary to achieve the desired transcription of the sequences. Other beneficial characteristics may also be contained within the vectors such as mechanisms for recovery of the nucleic acids in a different form. Phagemids are a specific example of such beneficial vectors because they may be used either as plasmids or as bacteriophage vectors. Examples of other vectors include viruses such as bacteriophages, baculoviruses and retroviruses, DNA viruses, cosmids, plasmids and other recombination vectors. The vectors may also contain elements for use in either procaryotic or eucaryotic host systems. One of ordinary skill in the art will know which host systems are compatible with a particular vector.

The vectors may be introduced into cells or tissues by any one of a variety of known methods within the art. Such methods may be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa et al (1986) and include, for example, stable or transient transfection, lipofection, electroporation and infection with recombinant viral vectors. Introduction of nucleic acids by infection offers several advantages over the other listed methods. Higher efficiency may be obtained due to their infectious nature. See also U.S. Pat. Nos. 5,487,992 and 5,464,764. Moreover, viruses are very specialized and typically infect and propagate in specific cell types. Thus, their natural specificity may be used to target the vectors to specific cell types in vivo or within a tissue or mixed culture of cells. Viral vectors may also be modified with specific receptors or ligands to alter target specificity through receptor mediated events.

Recombinant methods known in the art may also be used to achieve the sense, antisense or triplex inhibition of a target nucleic acid. For example, vectors containing antisense nucleic acids may be employed to express protein or antisense message to reduce the expression of the target nucleic acid and therefore its activity.

A specific example of DNA viral vector for introducing and expressing antisense nucleic acids is the adenovirus derived vector Adenop53TK. This vector expresses a herpes virus thymidine kinase (TK) gene for either positive or negative selection and an expression cassette for desired recombinant sequences such as antisense sequences. This vector may be used to infect cells that have an adenovirus receptor. This vector as well as others that exhibit similar desired functions may be used to treat a mixed population of cells include, for example, an in vitro or ex vivo culture of cells, a tissue or a human subject.

Additional features may be added to the vector to ensure its safety and/or enhance its therapeutic efficacy. Such features include, for example, markers that may be used to negatively select against cells infected with the recombinant virus. An example of such a negative selection marker is the TK gene described above that confers sensitivity to the anti-viral gancyclovir. Negative selection is therefore a means by which infection may be controlled because it provides inducible suicide through the addition of antibiotic. Such protection ensures that if, for example, mutations arise that produce altered forms of the viral vector or sequence, cellular transformation will not occur. Features that limit expression to particular cell types may also be included. Such features include, for example, promoter and regulatory elements that are specific for the desired cell type.

Recombinant viral vectors are another example of vectors useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighbouring cells. The result is that a large area becomes rapidly infected, most of which was not initially infected by the original viral particles. This is in contrast to vertical-type of infection in which the infectious agent spreads only through daughter progeny. Viral vectors may also be produced that are unable to spread laterally. This characteristic may be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods described here will depend on desired cell type to be targeted. For example, a vector specific for epithelial cells may be used for treatment of skin diseases. Likewise, if diseases or pathological conditions of the lung are to be treated, then a viral vector that is specific for lung cells and their precursors, preferably for the specific type of lung cell, should be used.

Retroviral vectors may be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or may be constructed by one skilled in the art using well-known methodology.

If viral vectors are used, for example, the procedure may take advantage of their target specificity and consequently, do not have to be administered locally at the diseased site. However, local administration may provide a quicker and more effective treatment, administration may also be performed by, for example, intravenous or subcutaneous injection into the subject. Injection of the viral vectors into a spinal fluid may also be used as a mode of administration, especially in the case of neurodegenerative diseases. Following injection, the viral vectors will circulate until they recognize host cells with the appropriate target specificity for infection.

Transfection vehicles such as liposomes may also be used to introduce the non-viral vectors described above into recipient cells within the inoculated area. Such transfection vehicles are known by one skilled within the art.

### Transgenic Organisms

We further disclose the construction of transgenic and knockout organisms that exhibit the phenotypic manifestations of Group 1 and Group 2 diseases.

We therefore provide for transgenic corneodesmosin gene and mutant corneodesmosin gene animal and cellular (cell lines) models as well as for knockout models. The transgenic models include those carrying the corneodesmosin sequence, as well as mutations of the gene associated with Group 1 and Group 2 diseases. We further provide for animal and cellular (cell lines) models comprising and/or expressing transgenic and/or mutant adhesion protein, protease or protease inhibitor genes as well as for knockout models. The transgenic models include those carrying the sequence of an adhesion protein, protease or protease inhibitor, as well as mutations of the gene associated with Group 1 and Group 2 diseases. We These models are constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, (1991), Capecchi, (1989), Davies et al., (1992), Dickinson et al., (1993), Huxley et al., (1991), Jakobovits et al., (1993), Lamb et al., (1993), Rothstein, (1991), Schedl et al., (1993), Strauss et al., (1993). Further, patent applications WO 94/23049, WO 93/14200, WO 94/06908, WO 94/28123 also provide information. See also in general Hogan et al “Manipulating the Mouse Embryo” Cold Spring Harbor Laboratory Press, 2nd Edition (1994).

### Genetic Diagnosis

We disclose a method for diagnosing and detecting carriers of a defective an adhesion protein, protease or protease inhibitor gene responsible for causing Group 1 or Group 2 diseases, or associated with abnormal regulation of proteolysis of an adhesion protein such as corneodesmosin.

We further provide methods for detecting normal copies of the gene and its gene product. Identifying carriers either by their defective gene or by their missing or defective protein(s) encoded thereby, leads to earlier and more consistent diagnosis of gene carriers. Thus, since carriers of the disease are more likely to be prone to Group 1 or Group 2 diseases, better surveillance and treatment protocols may be initiated for them.

Briefly, the methods comprise the steps of obtaining a sample from a test subject, isolating the appropriate test material from the sample and assaying for the target nucleic acid sequence or gene product. The sample may be tissue or bodily fluids from which genetic material and/or proteins are isolated using methods standard in the art. For example, DNA may be isolated from blood.

More specifically, the method of carrier detection is carried out by first obtaining a sample of either cells or bodily fluid from a subject. Convenient methods for obtaining a cellular sample may include collection of either mouth wash fluids or hair roots. A cell sample could be amniotic or placental cells or tissue in the case of a prenatal diagnosis. A crude DNA could be made from the cells (or alternatively proteins isolated) by techniques well known in the art. This isolated target DNA is then used for PCR analysis (or alternatively, Western blot analysis for proteins) with appropriate primers derived from the gene sequence by techniques well known in the art. The PCR product would then be tested for the presence of appropriate sequence variations in order to assess genotypic disease status of the subject.

The specimen may be assayed for polypeptides/proteins by immunohistochemical and immunocytochemical staining (see generally Stites and Terr, Basic and Clinical Immunology, Appleton and Lange, 1994), ELISA, RIA, immunoblots, Western blotting, immunoprecipitation, functional assays and protein truncation test. In preferred embodiments, Western blotting, functional assays and protein truncation test (Hogervorst et al., 1995) are used. mRNA complementary to the target nucleic acid sequence may be assayed by in situ hybridization, Northern blotting and reverse transcriptase—polymerase chain reaction. Nucleic acid sequences may be identified by in situ hybridization, Southern blotting, single strand conformational polymorphism, PCR amplification and DNA-chip analysis using specific primers. (Kawasaki, 1990; Sambrook, 1992; Lichter et al, 1990; Orita et al, 1989; Fodor et al., 1993; Pease et al., 1994)

ELISA assays are well known to those skilled in the art. Both polyclonal and monoclonal antibodies may be used in the assays. Where appropriate other immunoassays, such as radioimmunoassays (RIA) may be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, 1992.

Current mutation data as described here indicate that Group 1 and Group 2 diseases are characterized by allelic heterogenicity. Thus, it is important for a successful mutation screen to be able to detect all possible nucleotide alterations in the corneodesmosin or other adhesion protein, protease or protease inhibitor gene, rather than being focused on a limited subset. Methods including direct sequencing of PCR amplified DNA or RNA or DNA chip hybridization (Fodor et al., 1993; Pease et al., 1994) may be applied along with other suitable methods known to those skilled in the art.

In order to use the methods for diagnostic applications, it is advantageous to include a mechanism for identifying the presence or absence of target polynucleotide sequence (or alternatively proteins). In many hybridization based diagnostic or experimental procedures, a label or tag is used to detect or visualize for the presence or absence of a particular polynucleotide sequence. Typically, oligomer probes are labelled with radioisotopes such as ³²P or ³⁵S (Sambrook, 1992) which may be detected by methods well known in the art such as autoradiography. Oligomer probes may also be labelled by non-radioactive methods such as chemiluminescent materials which may be detected by autoradiography (Sambrook, 1992). Also, enzyme-substrate based labelling and detection methods may be used. Labelling may be accomplished by mechanisms well known in the art such as end labelling (Sambrook, 1992), chemical labelling, or by hybridization with another labelled oligonucleotide. These methods of labelling and detection are provided merely as examples and are not meant to provide a complete and exhaustive list of all the methods known in the art.

The introduction of a label for detection purposes may be accomplished by attaching the label to the probe prior to hybridization.

An alternative method includes the step of binding the target DNA to a solid support prior to the application of the probe. The solid support may be any material capable of binding the target DNA, such as beads or a membranous material such as nitrocellulose or nylon. After the target DNA is bound to the solid support, the probe oligomers is applied.

Functional assays may be used for detection of Group 1 and Group 2 carriers or affected individuals.

We also provide a kit for diagnosis and detection of a defective corneodesmosin gene. The kit includes a molecular probe complementary to genetic sequences of a defective corneodesmosin gene which causes a Group 1 or a Group 2 disease and suitable labels for detecting hybridization of the molecular probe and the defective gene thereby indicating the presence of the defective gene. The molecular probe has a DNA sequence complementary to mutant sequences. Alternatively, the kit may contain reagents and antibodies for detection of mutant proteins.

A kit for detection and diagnosis of a defective adhesion protein, protease or protease inhibitor gene is also provided, comprising a molecular probe complementary to genetic sequences of a defective adhesion protein, protease or protease inhibitor gene which causes a Group 1 or a Group 2 disease and suitable labels for detecting hybridization of the molecular probe and the defective gene thereby indicating the presence of the defective gene. The molecular probe has a DNA sequence complementary to mutant sequences. Alternatively, the kit may contain reagents and antibodies for detection of mutant proteins.

A few different methods are commonly used to analyze DNA for polymorphisms or mutations. The most definitive method is to sequence the DNA to determine the actual base sequence (Maxam and Gilbert, 1977; Sanger et al., 1977). Although such a method is the most definitive it is also the most expensive and time consuming method.

Restriction mapping analysis may also be used in analyzing DNA for polymorphisms. If one is looking for a known polymorphism at a site which will change the recognition site for a restriction enzyme it is possible simply to digest DNA with this restriction enzyme and analyze the fragments on a gel or with a Southern blot to determine the presence or absence of the polymorphism. This type of analysis is also useful for determining the presence or absence of gross insertions or deletions. Hybridization with allele specific oligonucleotides is yet another method for determining the presence of known polymorphisms.

Thus, specific DNA sequences in an individual, for example, a gene encoding corneodesmosin, may undergo many different changes, such as deletion of a sequence of DNA, insertion of a sequence that was duplicated, inversion of a sequence, or conversion of a single nucleotide to another. Changes in a specific DNA sequence may be traced by using restriction enzymes that recognize specific DNA sequences of 4-6 nucleotides. Restriction enzymes cut (digest) the DNA at their specific recognized sequence, resulting in one million or so pieces. When a difference exists that changes a sequence recognized by a restriction enzyme to one not recognized, the piece of DNA produced by cutting the region are of a different size. The various possible fragment sizes from a given region therefore depend on the precise sequence of DNA in the region. Variation in the fragments produced is termed “restriction fragment length polymorphism” (RFLP). The different sized-fragments reflecting different variant DNA sequences may be visualized by separating the digested DNA according to its size on an agarose gel and visualizing the individual fragments by annealing to a radioactively labeled DNA “probe”. Each individual may carry two different forms of the specific sequence. When the two homologues carry the same form of the polymorphism, one band is seen. More than two forms of a polymorphism may exist for a specific DNA marker in the population, but in one family just four forms are possible; two from each parent. Each child inherits one form of the polymorphism from each parent. Thus, the origin of each chromosome region may be traced (maternal or paternal origin).

Furthermore, RT-PCR may be carried out, followed by restriction endonuclease fingerprinting (REF). REF is a modification of the single-strand conformation polymorphism (SSCP) method, and enables efficient detection of sequence alterations in DNA fragments up to 2 kb in length (Liu and Sommer, 1995).

The use of mass spectrometry to determine the presence of polymorphisms within known genes is disclosed in U.S. Pat. No. 5,869,242.

Single strand conformational polymorphism (SSCP) analysis is a rapid and efficacious method for detecting polymorphisms (Dean et al., Cell 61:863, 1990; Glavac and Dean, Hum. Mutation 2:404, 1993; Poduslo et al., Am. J. Hum. Genet. 49:106, 1992). In SSCP, abnormal strand motility on a gel is associated with mutational events in the gene.

Genetic analysis of polymorphisms is disclosed in detail in U.S. Pat. Nos. 5,552,28, 5,654,13, 5,670,33, 5,807,67, 5,858,66, 5,691,15, 5,922,57, 5,972,60, 6,136,53 and 5,955,26, among others.

Any of these methods described in the above references may be used in the diagnostic methods described here.

### Pharmaceutical Compositions

We further disclose pharmaceutical compositions comprising one or more agents for treating Group 1 or Group 2 disease.

Agents for treating Group 1 diseases include protease inhibitors, and fragments thereof, including those identified in the Examples as comprising anti-protease activity, for example primary protease inhibitors such as anti-leukoprotease (SLPI) and elafin protease inhibitor 3 (PI3 or SKALP) and/or secondary protease inhibitors, including chymotrypsin, soybean trypsin inhibitor, cathepsin G, etc as well as other protease inhibitors as known in the art. Other agents useful for treating Group 1 diseases include agonists of protease inhibitors, for example, agonists of any of the above protease inhibitors, as well as antagonists of proteases, including antagonists of stratum corneum chymotryptic enzyme (SCCE) and/or stratum corneum tryptic enzyme (SCTE).

Agents for treating Group 2 diseases include proteases such as stratum corneum chymotryptic enzyme (SCCE) and stratum corneum tryptic enzyme (SCTE), as well as agonists of proteases, including agonists of any of the above proteases. Agents for treating Group 2 diseases further include antagonists of protease inhibitors, for example antagonists of anti-leukoprotease (SLPD and elafin protease inhibitor 3 (PI3 or SKALP), etc.

While it is possible for the composition comprising the agent or agents to be administered alone, it is preferable to formulate the active ingredient as a pharmaceutical formulation. The composition may include the agent(s), a structurally related compound, or an acidic salt thereof. The pharmaceutical formulations comprise an effective amount of agent together with one or more pharmaceutically-acceptable carriers. An “effective amount” of an agent is the amount sufficient to alleviate at least one symptom of a Group 1 or a Group 2 disease, as the case may be. The effective amount will vary depending upon the particular disease or syndrome to be treated or alleviated, as well as other factors including the age and weight of the patient, how advanced the disease etc state is, the general health of the patient, the severity of the symptoms, and whether the agent is being administered alone or in combination with other therapies.

Suitable pharmaceutically acceptable carriers are well known in the art and vary with the desired form and mode of administration of the pharmaceutical formulation. For example, they can include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The pharmaceutical compositions include those suitable for topical and oral administration, with topical formulations being preferred where the tissue affected is primarily the skin or epidermis (for example, psoriasis and other epidermal diseases). The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated. As an example, an acute skin proliferation disease generally is treated with aqueous drying preparations, whereas chronic skin proliferation disease is treated with hydrating preparations. Soaks are the easiest method of drying acute moist eruptions. Lotions (powder in water suspension) and solutions (medications dissolved in a solvent) are ideal for hairy and intertriginous areas. Ointments or water-in-oil emulsions, are the most effective hydrating agents, appropriate for dry scaly eruptions, but are greasy and depending upon the site of the lesion sometimes undesirable. As appropriate, they can be applied in combination with a bandage, particularly when it is desirable to increase penetration of the agent composition into a lesion. Creams or oil-in-water emulsions and gels are absorbable and are the most cosmetically acceptable to the patient. (Guzzo et al, in Goodman & Gilman's Pharmacological Basis of Therapeutics, 9th Ed., p. 1593-15950 (1996)). Cream formulations generally include components such as petroleum, lanolin, polyethylene glycols, mineral oil, glycerin, isopropyl palmitate, glyceryl stearate, cetearyl alcohol, tocopheryl acetate, isopropyl myristate, lanolin alcohol, simethicone, carbomen, methylchlorisothiazolinone, methylisothiazolinone, cyclomethicone and hydroxypropyl methylcellulose, as well as mixtures thereof.

Other formulations for topical application include shampoos, soaps, shake lotions, and the like, particularly those formulated to leave a residue on the underlying skin, such as the scalp (Arndt et al, in Dermatology In General Medicine 2:2838 (1993)).

In general, the concentration of the agent composition in the topical formulation is in an amount of about 0.5 to 50% by weight of the composition, preferably about 1 to 30%, more preferably about 2-20%, and most preferably about 5-10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of agent must penetrate a patient's skin in order to obtain a desired pharmacological effect. It is generally understood that the absorption of drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of drug between the stratum corneum and the vehicle and the diffusion coefficient of drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (J. Pharm. Sci., 60:1175-1179 (1971) demonstrated that in vivo efficacy of topically applied steroids was proportional to the steroid penetration rate into dermatomed human skin in vitro.

A skin penetration enhancer which is dermatologically acceptable and compatible with the agent can be incorporated into the formulation to increase the penetration of the active compound(s) from the skin surface into epidemal keratinocytes. A skin enhancer which increases the absorption of the active compound(s) into the skin reduces the amount of agent needed for an effective treatment and provides for a longer lasting effect of the formulation. Skin penetration enhancers are well known in the art. For example, dimethyl sulfoxide (U.S. Pat. No. 3,711,602); oleic acid, 1,2-butanediol surfactant (Cooper, J. Pharm. Sci., 73:1153-1156 (1984)); a combination of ethanol and oleic acid or oleyl alcohol (EP 267,617), 2-ethyl-1,3-hexanediol (WO 87/03490); decyl methyl sulphoxide and Azone.RTM. (Hadgraft, Eur. J. Drug. Metab. Pharmacokinet, 21:165-173 (1996)); alcohols, sulphoxides, fatty acids, esters, Azone.RTM., pyrrolidones, urea and polyoles (Kalbitz et al, Pharmazie, 51:619-637 (1996));

Terpenes such as 1,8-cineole, menthone, limonene and nerolidol (Yamane, J. Pharmacy & Pharmocology, 47:978-989 (1995)); Azone.RTM. and Transcutol (Harrison et al, Pharmaceutical Res. 13:542-546 (1996)); and oleic acid, polyethylene glycol and propylene glycol (Singh et al, Pharmazie, 51:741-744 (1996)) are known to improve skin penetration of an active ingredient.

Levels of penetration of an agent or composition can be determined by techniques known to those of skill in the art. For example, radiolabeling of the active compound, followed by measurement of the amount of radiolabeled compound absorbed by the skin enables one of skill in the art to determine levels of the composition absorbed using any of several methods of determining skin penetration of the test compound. Publications relating to skin penetration studies include Reinfenrath, W G and G S Hawkins. The Weaning Yorkshire Pig as an Animal Model for Measuring Percutaneous Penetration. In:Swine in Biomedical Research (M. E. Tumbleson, Ed.) Plenum, N.Y., 1986, and Hawkins, G. S. Methodology for the Execution of In Vitro Skin Penetration Determinations. In: Methods for Skin Absorption, B W Kemppainen and W G Reifenrath, Eds., CRC Press, Boca Raton, 1990, pp.67-80; and W. G. Reifenrath, Cosmetics & Toiletries, 110:3-9 (1995).

For some applications, it is preferable to administer a long acting form of agent or composition using formulations known in the arts, such as polymers. The agent can be incorporated into a dermal patch (Junginger, H. E., in Acta Pharmaceutica Nordica 4:117 (1992); Thacharodi et al, in Biomaterials 16:145-148 (1995); Niedner R., in Hautarzt 39:761-766 (1988)) or a bandage according to methods known in the arts, to increase the efficiency of delivery of the drug to the areas to be treated.

Optionally, the topical formulations can have additional excipients for example; preservatives such as methylparaben, benzyl alcohol, sorbic acid or quaternary ammonium compound; stabilizers such as EDTA, antioxidants such as butylated hydroxytoluene or butylated hydroxanisole, and buffers such as citrate and phosphate.

The pharmaceutical composition can be administered in an oral formulation in the form of tablets, capsules or solutions. An effective amount of the oral formulation is administered to patients 1 to 3 times daily until the symptoms of the proliferative disease, cancer or photoageing etc are alleviated. The effective amount of agent depends on the age, weight and condition of a patient. In general, the daily oral dose of agent is less than 1200 mg, and more than 100 mg. The preferred daily oral dose is about 300-600 mg. Oral formulations are conveniently presented in a unit dosage form and may be prepared by any method known in the art of pharmacy. The composition may be formulated together with a suitable pharmaceutically acceptable carrier into any desired dosage form. Typical unit dosage forms include tablets, pills, powders, solutions, suspensions, emulsions, granules, capsules, suppositories. In general, the formulations are prepared by uniformly and intimately bringing into association the agent composition with liquid carriers or finely divided solid carriers or both, and as necessary, shaping the product. The active ingredient can be incorporated into a variety of basic materials in the form of a liquid, powder, tablets or capsules to give an effective amount of active ingredient to treat skin proliferation disease.

Other therapeutic agents suitable for use herein are any compatible drugs that are effective for the intended purpose, or drugs that are complementary to the agent formulation. As an example, the treatment with an formulation can be combined with other treatments such as a topical treatment with corticosteroids, calcipotrine, coal tar preparations, a systemic treatment with methotrexate, retinoids, cyclosporin A and photochemotherapy. The combined treatment is especially important for treatment of an acute or a severe skin proliferation disease. The formulation utilized in a combination therapy may be administered simultaneously, or sequentially with other treatment, such that a combined effect is achieved.

### Further Aspects of the Invention

Further aspects of the invention are now set out in the following numbered paragraphs; it is to be understood that the invention encompasses these aspects:

Paragraph 1. A method of treatment of a patient suffering from a disease associated with abnormal cell-cell adhesion between epithelial cells, the method comprising regulating the proteolysis of an adhesion protein responsible for adhesion between the cells.

Paragraph 2. A method according to Paragraph 1, in which the adhesion protein is a desmosomal protein.

Paragraph 3. A method according to Paragraph 1 or 2, in which the adhesion protein is corneodesmosin.

Paragraph 4. A method according to any preceding Paragraph, in which the epithelial cell is a corneocyte.

Paragraph 5. A method according to any preceding Paragraph, in which the regulation of proteolysis of the adhesion protein comprises regulation of the expression, activity and/or breakdown of a protease involved in proteolysis of the adhesion protein.

Paragraph 7. A method according to any preceding Paragraph, in which the regulation of proteolysis of the adhesion protein comprises regulation of expression, activity and/or breakdown of a protease inhibitor responsible for inhibiting the activity of a protease involved proteolysis of the adhesion protein.

Paragraph 8. A method of treatment of a patient suffering from a disease associated with abnormal cell-cell adhesion between epithelial cells, the method comprising regulating the expression and/or activity of an adhesion protein responsible for adhesion between the cells.

Paragraph 9. A method according to Paragraph 8, in which the expression of the adhesion protein is regulated at the transcriptional or the translational level, or both.

Paragraph 10. A method according to any preceding Paragraph, in which the disease is associated with decreased cell-cell adhesion, and the proteolysis of the adhesion protein is reduced by one or more of the following: administration of a protease inhibitor; administration of an antagonist of a protease; administration of an agonist of a protease inhibitor; reducing the expression of a protease; reducing the activity of a protease; increasing the expression of a protease inhibitor; increasing the activity of a protease inhibitor.

Paragraph 11. A method according to any of Paragraphs 1 to 9, in which the disease is associated with increased cell-cell adhesion, and the proteolysis of the adhesion protein is increased by one or more of the following: administration of a protease; administration of an agonist of a protease; administration of an antagonist a protease inhibitor; increasing the expression of a protease; increasing the activity of a protease; reducing the expression of a protease inhibitor; reducing the activity of a protease inhibitor.

Paragraph 12. A method of diagnosis of a disease associated with abnormal cell-cell adhesion between epithelial cells, the method comprising detection of a mutation in the corneodesmosin gene of an individual.

Paragraph 13. A method according to Paragraph 12, in which the disease is associated with decreased cell-cell adhesion, and the method comprises detecting the absence of a Hph1 restriction enzyme site in, or the presence of a T at position +1243 of, the corneodesmosin gene in an individual.

Paragraph 14. A method according to any of Paragraphs 1 to 10, 12 or 13, in which the disease is selected from the group consisting of: atopic eczema, sebarrhoeic eczema, irritant contact dermatitis, allergic contact dermatitis, lung atopic asthma, post viral asthma, branchial hyper-reactivity, chronic obstruction pulmonary disease, Crohn's disease, ulcerative colitis, coeliac disease, peptic ulceration, impetigo, viral warts, Molluslum Contagiosum, bacterial meningitis, viral meningitis, peptic ulceration associated with penetration of Helicobacteria pylori, skin melanoma, squamous cell carcinoma, basal cell carcinoma, cutaneous lymphoma, a skin cancer, a malignancy of the gastrointestinal tract and a malignancy of the lung.

Paragraph 15. A method according to Paragraph 12, in which the disease is associated with increased cell-cell adhesion, and the method comprises detecting the presence of a Hph1 site in, or the absence of a T at position +1243 of, the corneodesmosin gene in an individual.

Paragraph 16. A method according to any of Paragraphs 1 to 9, 11, 12, or 15, in which the disease is selected from the group consisting of: psoriasis, ichtyoses, acne vulgaris and keratoses pilaris.

### EXAMPLES

### Example A

### Adhesion Protein Polymorphisms

### Example A1

### Identification of Corneodesmosin (S) Gene Polymorphisms

Genomic DNA is extracted from whole blood according to standard protocols and stored at 100 ng/μl. One reported exonic polymorphism giving a T to C transition at position +1243 is analysed (Ishihara et al, 1996, Tazi-Ahnini et al, ¹⁹⁹⁹a, 1999b).

Primers S15 (5′ATTGCATTCCAGCCAGTGG3′) and S16 (5′AACTGGAGCTGCTGCTGAAGGA3′) are used to amplify the polymorphism locus (+1243). PCRs are prepared in bulk and aliquoted to 25 μl volumes comprising 50 mM KCL, 20 mM Tris-HCL, 1.5 mM MgCl₂, 200 μM each dNTP, 1.2 μM each primer, 1 U of Taq polymerase (Gibco BRL, Paisley, UK) and 200 ng of genomic DNA. Thermocycling conditions are 2 min at 95° C., 28 cycles of 1 min at 95° C., 1 min at 58° C. and 15 s at 72° C. The amplifications are ended by 15 min at 72° C.

Restriction digests are performed in 20 μl reactions containing 10 μl of PCR products and 2.5 U of HphI and appropriate manufacturer's buffer (New England Biolabs, Hitchin, UK) at 37° C. overnight. Allelic discrimination is performed by electrophoresis using 2% agarose. HphI digestion produces 123+89 bp for allele 1, while it does not cut allele 2 (212 bp).

Alleles

In the following Examples, “allele 1 ” refers to an allele in which the nucleotide residue at position +1243 is a C. The protein encoded by such an allele has a serine (S) residue at position 394 of the amino acid sequence employing the numbering of the polypeptide sequence having accession number L20815.

Similarly, “allele 2” refers to an allele in which the nucleotide residue at position +1243 is a T. The protein encoded by such an allele has a leucine residue at position 394 of a amino acid sequence, employing the numbering of the polypeptide sequence having accession number L20815.

Amino acid position numbering refers to the translation initiation codon (+1, ATG) of corneodesmosin sequence with GenBank accession numbers GB: L20815 or AF030130, as indicated. Thus, for example, a +1243 substitution C to T gives amino acid change from S to F at position 394 according to GB sequence L20815, while the same nucleotide substitution gives amino acid change from S to F at position 410 according to the GB sequence AF030130.

Statistical Analysis

Disease and control populations are compared using 2×3 tables, and the odds ratio is calculated by comparing individuals homozygotic for allele 1 with that for carriage of the alternative allele (allele 2) in control and patients population. A χ² test for allele 2 is also carried out, weighted by number of putative disease susceptibility alleles in each genotype group.

### Example A2

### Association of Corneodesmosin (S) Gene +1243 Polymorphisms With Atopic Eczema

In this Example, unless otherwise indicated, amino acid positions in the corneodesmosin sequence are provided with reference to the numbering in GenBank sequence L20815.

This Example demonstrates that corneodesmosin allele 2, in which the nucleotide residue of corneodesmosin at position +1243 is a T, leading to the presence of a leucine (T) residue at position 394 of the corneodesmosin polypeptide (L20815), is associated with atopic eczema.

The allelic distribution of +1243 polymorphism is assessed in both the atopic eczema and controls groups (n=154 and 550, respectively), as described above. Both controls and patients are in Hardy Weinberg equilibrium. Table A2.1 shows the observed (A) and the expected (B) values of each genotype in controls and atopic eczema patients groups.

TABLE A2.1
Allelic distribution of allele 11, 12 and 22
in control and atopic eczema patient groups.

| | | |
|---|---|---|
| 22 (T/T) | 12 (T/C) | 11 (C/C) |

(A) Observed values

| | | | |
|---|---|---|---|
| Controls | 150 | 284 | 116 |
| Patients | 52 | 74 | 28 |

(B) Expected values

| | | | |
|---|---|---|---|
| Controls | 157.8 | 279.7 | 112.5 |
| Patients | 44.2 | 78.3 | 31.5 |

A: Observed values;
B: Expected Values

The overall difference between expected and observed values in the controls and patients is not statistically significant. However, there is an increase of the rare allele (allele 2, 52/44.2=1.2). Allele 1 (common allele) appears to be protective from eczema, and for this reason we group individuals with alleles 12 and 11 in one subgroup and 22 in the other (see Table A2.2 below).

TABLE A2.2
Allelic distribution of allele 11 and 12/22 in
control and atopic eczema patient groups

| | 22 (TT) | 11/12 (CC/CT) |
|---|---|---|
| Patients | 52 | 102 |

A χ² test for allele 2 against carriage of allele 1 is carried out. A significant association is found between allele 2 of the corneodesmosin gene (+1243) polymorphism and atopic eczema [OR=1.36 (0.93, 1.99)].

We therefore disclose the diagnosis of a Group I disease or susceptibility to a Group I disease (preferably eczema or susceptibility to eczema, preferably atopic eczema) in an individual, by detecting the presence of a T at position +1243 of a corneodesmosin nucleic acid, or the presence of a leucine (L) residue at position 394 of a corneodesmosin polypeptide (L20815), or both, of an individual.

### Example A3

### Association of Corneodesmosin (S) Gene +1243 Polymorphisms With Dermatitis

In this Example, unless otherwise indicated, amino acid positions in the corneodesmosin sequence are provided with reference to the numbering in GenBank sequence L20815.

This Example demonstrates that corneodesmosin allele 2, in which the nucleotide residue of corneodesmosin at position +1243 is a T, leading to the presence of a leucine (L) residue at position 394 of the corneodesmosin polypeptide (L20815), is associated with dermatitis and might be the cause of the pathogenesis of dermatitis herpetiformis.

The allelic distribution of the +1243 polymorphism is assessed in both the dermatitis herpetiformis and controls groups (n=50 and 550, respectively), as described above. Both controls and patients are in Hardy Weinberg equilibrium.

TABLE A3.1
Allelic distribution of allele 11, 12 and 22 in
control and dermatitis herpetiformis groups

| | | |
|---|---|---|
| 22 (T/T) | 12 (T/C) | 11 (C/C) |

A) Observed values

| | | | |
|---|---|---|---|
| Controls | 150 | 284 | 116 |
| Patients | 26 | 23 | 1 |

B) Expected values

| | | | |
|---|---|---|---|
| Controls | 161.3 | 281.4 | 107.2 |
| Patients | 14.6 | 25.5 | 9.7 |

There is an increase of the rare allele (allele 2, 26/14.6=1.78). Only one patient is homozygous for allele 1 (a common allele). This allele seems to be a protective since it is very frequent in control population.

TABLE A3.2
Allelic distribution of allele 11 and 12/22 in
control and dermatitis herpetiformis groups

| | TT/CT (22/12) | CC (12/11) |
|---|---|---|
| Controls | 434 | 116 |
| Patients | 49 | 1 |

A χ² test for allele 1 against carriage of allele 2 is carried out. A significant association is found between allele 2 of S gene (+1243) polymorphism and dermatitis herpetiformis [OR=13.10 (1.79, 95.85); p<0.0001].

We therefore disclose the diagnosis of a Group I disease or susceptibility to a Group I disease (preferably dermatitis or susceptibility to dermatitis, preferably dermatitis herpetiformis) in an individual, by detecting the presence of a T at position +1243 of a corneodesmosin nucleic acid, or the presence of a leucine (L) residue at position 394 of a corneodesmosin polypeptide (L20815), or both, of an individual. This amino acid is at position 410 according to the GB sequence AF030130.

Allele 2 or T at position +1243 confers higher risk for dermatitis herpetiformis compared to atopic eczema because individual heterozygous at +1243 have also high risk of developing dermatitis.

### Example A4

### Linkage Disequilibrium Analysis

In this Example, unless otherwise indicated, amino acid positions in the corneodesmosin sequence are provided with reference to the numbering in GenBank sequence L20815.

It has previously been shown that C at position +1243 is in linkage disequilibrium with T and G at position +619 and +1240 respectively (Tazi-Ahnini et al, 1999a; Allen et al, 1999). The linkage disequilibrium is extended to other loci within the coding sequence of the corneodesmosin. Furthermore, using pedigree analysis with the transmission disequilibrium test (TDT), Jenisch and his colleagues identify 6 different alleles encoding 6 different amino acid sequences of the corneodesmosin gene (Jenisch et al, 1999).

Group I Diseases

We show that within allele 5 (CD5) and allele 6 (CD6), T (leu) at position +1243 is in strong linkage disequilibrium with A (asp), AGT (ser), T (phe), A (ser), T (ser), T (leu), G (asp) and T (asn) of CD5 and A (asp), deletion (−), T (phe), A (ser), T (ser), T (leu), G (asp) and C (asp) of CD6. These variants are at position +442, +468, +619, +1215, +1236, +1243, +1515 and +1593 respectively.

We find a strong association of these variants with atopic eczema in our collection. Therefore, detection of any of the changes listed above, and shown to be in linkage disequilibrium with T at position +1243 (i.e., allele 2), may be used in place of, or in addition to, detection of nucleotide T (+1243) or amino acid L (394 of L20815).

We therefore provide the diagnosis of a Group I disease or susceptibility to a Group I disease in an individual, preferably eczema or dermatitis, more preferably atopic eczema or dermatitis herpetiformis, or susceptibility to any of these diseases, by detecting any one or more of these changes.

Accordingly, we provide the diagnosis of a Group I disease, or susceptibility to a Group I disease, by detecting any one or more of the following residues at the relevant positions of a corneodesmosin nucleic acid, as shown in the Table A4.1 below.

TABLE A4.1
Corneodesmosin Polymorphisms and Group I Diseases.

| | Nucleotide Position | | | | | | | |
|---|---|---|---|---|---|---|---|---|
| | 442 | 468 | 619 | 1215 | 1236 | 1243 | 1515 | 1593 |
| Nucleic acid (s) | A | AGT | T | A | T | T | G | T |
| Residue | 127 | 137 | 186 | 385 | 392 | 394 | 485 | 511 |
| Position (1) | | | | | | | | |
| Residue | 143 | 153 | 202 | 401 | 408 | 410 | 501 | 527 |
| Position (2) | | | | | | | | |
| Residue | D | S/— | F | S | S | L | D | D/N |

(1): amino acid position numbering according to GenBank sequence L20815;
(2): amino acid position numbering according to GenBank sequence AF030130

Group II Diseases

We show that within allele 2 (CD2) of the corneodesmosin C (Ser) at position +1243 is in strong linkage disequilibrium with G (Ser), AGT (ser), T (phe), A (ser), T (ser), C (Ser), G (asp) and T (asn) of CD2. These variants are at position +442, +468, +619, +1215, +1236, +1243, +1515 and +1593 respectively.

We find a strong association of these variants with acne vulgaris in our collection. Therefore, detection of any of the changes listed above, and shown to be in linkage disequilibrium with T at position +1243 (i.e., allele 1), may be used in place of, or in addition to, detection of nucleotide C (+1243) or amino acid S (394 of L20815).

We therefore provide the diagnosis of a Group II disease or susceptibility to a Group II disease in an individual, preferably acne and/or psoriasis, more preferably acne vulgaris and/or psoriasis vulgaris, or susceptibility to any of these diseases, by detecting any one or more of these changes.

Accordingly, we provide the diagnosis of a Group H disease, or susceptibility to a Group II disease, by detecting any one or more of the following residues at the relevant positions of a corneodesmosin nucleic acid or polypeptide, as shown in the Table A4.2 below.

TABLE A4.2
Corneodesmosin Polymorphisms and Group II Diseases.

| | Nucleotide Position | | | | | | | |
|---|---|---|---|---|---|---|---|---|
| | 442 | 468 | 619 | 1215 | 1236 | 1243 | 1515 | 1593 |
| Nucleic acid (s) | G | AGT | T | A | T | C | G | T |
| Residue | 127 | 137 | 186 | 385 | 392 | 394 | 485 | 511 |
| Position (1) | | | | | | | | |
| Residue | 143 | 153 | 202 | 401 | 408 | 410 | 501 | 527 |
| Position (2) | | | | | | | | |
| Residue | S | S | F | S | S | S | D | D |

(1): amino acid position according to GenBank sequence L20815;
(2): amino acid position according to GenBank sequence AF030130.

The above nucleic acid changes lead to changes in the amino acid sequence of corneodesmosin. Accordingly, the detection of the nucleotide residues at the relevant nucleic acid positions may also be achieved by detecting their effects, i.e., detecting a corresponding change in the encoded polypeptide sequence. Thus, for example, instead of or in addition to detecting a polymorphism at position +1243 of the nucleic acid sequence using specific nucleic probe or digestive enzyme (e.g. HphI), monoclonal antibodies may be used which are able to discriminate between a L residue at position 394 and an S residue at position 394.

We also provide the diagnosis of a Group I disease, or susceptibility to a Group I disease, by detecting any one or more of the following residues at the relevant positions of a corneodesmosin polypeptide, as shown in the Table A4.2. We also provide the diagnosis of a Group II disease, or susceptibility to a Group II disease, by detecting any one or more of the following residues at the relevant positions of a corneodesmosin polypeptide, as shown in the Table A4.2.

Summary

We have shown an association of a marker within the corneodesmosin gene giving an increased susceptibility to atopic eczema and dermatitis herpetiformis. It should be mentioned here that the corneodesmosin allele having C at +1243 (allele 1) is associated with psoriasis and acne vulgaris, suggesting that the two alleles give rise to different susceptibilities. Allele 2 (L394) is associated to barrier dysfunction (e.g. dermatitis, Crohn's disease) and allele I (S394) to impaired desquamation (e.g. psoriasis, acne).

We show association of polymorphisms at +1243 of the corneodesmosin gene with atopic eczema and dermatitis. The substitution at position +1243 gives amino acid change L394S. Without wanting to be bound by theory, we believe that this substitution interferes with the processing of corneodesmosin, thus contributing to enhanced desquamation.

We find that the corneodesmosin polymorphisms giving amino acid changes (Ser143/Asp, Ser153/-, Ser202/Phe, Ser401/Gly, Ser408/Ala, S410/L, Asp527/Asn; position according to GB sequence AF030130) or (Ser127/Asp, Ser137/-, Ser186/Phe, Ser385/Gly, Ser392/Ala, S394/L, Asp511/Asn; position according to GB sequence L20815) have an important function in the keratinocyte maturation and desquamation process. The screening method for these polymorphisms is described by Jenisch et 1999 and Guerrin et al, 2001. We therefore demonstrate that there is a strong relationship between the proteolytic processing of the corneodesmosin and the sensitivity of normal skin, and the integrity of diseased skin where there is a disruption in the barrier function including psoriasis, acne and dermatitis.

### Example A5

### Corneodesmosin (S) Gene Polymorphism at Position 180

In this Example, unless otherwise indicated, amino acid positions in the corneodesmosin sequence are provided with reference to the numbering in GenBank sequence L20815.

Identification of +180 Corneodesmosin Gene Polymorphism

The S gene variant at position 180 is identified by automatic sequencing of a cloned S allele (Guerrin et al, 2001). Nucleic acid change from C to T at position +180 gives an amino acid change from L at position 40 (L20815) to F. This amino acid is very conserved in mammalian species. Sequence alignments of human, mouse and pig S sequences show that L40 is conserved between these species as detailed below in Table A5.1.

TABLE A5.1
Alignment of Corneodesmosin Sequences.
Amino acid number is provided with reference
to the numbering of the sequence in GenBank
accession number L20815. Nucleic acid
substitution C to T at position 180 gives
amino acid change L to F at position 56 of
the corneodesmosin protein according to GB
sequence AF030130.

| | |
|---|---|
| Mouse | ITSPNDPCLI |
| Pig1 | IASPNDPCLL |
| Pig2 | IASPSDPCLL |
| Human | ITSPNDPCL₄₀T |

Chymotrypsin Proteolysis

Peptides corresponding to fragments of corneodesmosin, and comprising amino acids at and around position 40 (L20815; corresponding to position 180 on the nucleotide sequence) are synthesised. The peptides are exposed to chymotrypsin in appropriate buffer, and the digestion products identified. Chymotrypsin is known to cleave the peptide bond C-terminal to a F, Y or W residue, but not C-terminal to an L residue.

Peptides P1 and P2 (size 38 amino acids, comprising L and F at position 40 of L20815 respectively) are exposed to chymotrypsin. Peptide P1 has the sequence PTRITSPNDPCL₄₀TGKGDSSGFSGSSSSGSSISSAR, while peptide P2 has the sequence PTRITSPNDPCF₄₀TGKGDSSGFSGSSSSGSSISSAR.

It is found that peptide P1 with L at position 40 of L208 15 gives two products of proteolysis: PTRITSPNDPCL₄₀TGKGDSSGF and SGSSSSGSSISSAR. However, peptide P2 with F at position 40 of L20815 gives three small peptides PTRITSPNDPCF, TGKGDSSGF and SGSSSSGSSISSAR. The above results are confirmed by using the ExPASy Molecular Biology Server at http://www.expasy.ch/. Peptides having the P1 and P2 sequences are predicted to be cleaved differently by chymotrypsin.

This demonstrates that the L to F amino acid change creates a new chymotrypsin site within corneodesmosin. This has an important effect on the corneodesmosin maturation during keratinocyte differentiation and desquamation. We therefore disclose an association of a L to F amino acid polymorphism in corneodesmosin and a disease phenotype.

We therefore disclose a method for the diagnosis of a Group I disease or susceptibility to a Group I disease (preferably dermatitis or susceptibility to dermatitis, preferably dermatitis herpetiformis, preferably eczema, preferably atopic eczema) in an individual, by detecting the presence of a protease cleavage site in a corneodesmosin polypeptide of an individual. Detection of the protease site may be done on the polypeptide sequence, or detecting a nucleic acid change which encodes a protease site. Preferably, the protease cleavage site is a cleavage site of SCCE or SCTE.

Group I Diseases

We find in particular that the amino acid substitution from L to F at position 40 is associated with Group I disease (e.g., eczema and/or dermatitis). This substitution is caused by a change from C to T at position 180 of the corneodesmosin nucleic acid.

We therefore provide for the diagnosis of a Group I disease or susceptibility to a Group I disease (preferably dermatitis or susceptibility to dermatitis, preferably dermatitis herpetiformis, preferably eczema, preferably atopic eczema) in an individual, by detecting the presence of an F at position 40 of a corneodesmosin polypeptide of an individual.

We further provide for the diagnosis of a Group I disease or susceptibility to a Group I disease (preferably dermatitis or susceptibility to dermatitis, preferably dermatitis herpetiformis, preferably eczema, preferably atopic eczema) in an individual, by detecting the presence of a T at position 180 of a corneodesmosin nucleic acid of an individual.

Group II Diseases

We also find that the amino acid substitution from F to L at position 40 is associated with Group II disease (e.g., psoriasis and/or acne). This substitution is caused by a change from T to C at position 180 of the corneodesmosin nucleic acid. The alternative allele L at position 40 is therefore associated with diseases with increased adhesion (Group II) such as acne and psoriasis. This because the presence of L at position 40 of the corneodesmosin polypeptide make the protein resistant to the proteolysis process by proteases.

We therefore provide for the diagnosis of a Group II disease or susceptibility to a Group II disease (preferably psoriasis or acne or susceptibility to psoriasis or acne) in an individual, by detecting the presence of an L at position 40 of a corneodesmosin polypeptide of an individual.

We further provide for the diagnosis of a Group II disease or susceptibility to a Group II disease (preferably psoriasis or acne or susceptibility to psoriasis or acne) in an individual, by detecting the presence of a C at position 180 of a corneodesmosin nucleic acid of an individual.

### Example A6

### Identification of +619 Corneodesmosin Gene Polymorphism

We demonstrate that the nucleic acid substitution C to T at position +619 gives an amino acid change from S to F at position 186 according to the numbering in L20815 and position 202 according to the numbering in AF030130. This creates a new chymotryptic site within the corneodesmosin protein. We find that the serine at position 186 is very conserved in mammalian species.

We further demonstrate that F at position 186 is associated with defective skin barrier such as in eczema and dermatitis. F at position 186 is found in CD5 and CD6 of the corneodesmosin polypeptide.

Group I Diseases

We therefore provide for the diagnosis of a Group I disease or susceptibility to a Group I disease (preferably dermatitis or susceptibility to dermatitis, preferably dermatitis herpetiformis, preferably eczema, preferably atopic eczema) in an individual, by detecting the presence of an F at position 186 of a corneodesmosin polypeptide of an individual.

We further provide for the diagnosis of a Group I disease or susceptibility to a Group I disease (preferably dermatitis or susceptibility to dermatitis, preferably dermatitis herpetiformis, preferably eczema, preferably atopic eczema) in an individual, by detecting the presence of a T at position 619 of a corneodesmosin nucleic acid of an individual.

Group II Diseases

We also find that the amino acid substitution from F to S at position 186 is associated with Group II disease (e.g., psoriasis and/or acne). This substitution is caused by a change from T to C at position 619 of the corneodesmosin nucleic acid.

We therefore provide for the diagnosis of a Group II disease or susceptibility to a Group II disease (preferably psoriasis or acne or susceptibility to psoriasis or acne) in an individual, by detecting the presence of an S at position 186 of a corneodesmosin polypeptide of an individual.

We further provide for the diagnosis of a Group II disease or susceptibility to a Group II disease (preferably psoriasis or acne or susceptibility to psoriasis or acne) in an individual, by detecting the presence of a C at position 619 of a corneodesmosin nucleic acid of an individual.

Treatments

Polypeptides derived from or comprising a corneodesmosin or other adhesion protein, preferably peptides with additional protease cleavage sites, may be used to treat patients with defective skin barrier (Group I diseases). Such peptides preferably are capable of penetrating the skin barrier and preferably have molecular weights of less than or about <800 Da The peptides may preferably comprise a SPNDPCF₄₀TGKGDSS or a QSSSSSQTF₁₈₆GVSSSGQSV sequence.

These peptides can become the target of proteases (e.g. SCCE and SCTE) because they mimic the native corneodesmosin containing F at position 40 or the native corneodesmosin containing F at position 186, as the case may be.

Therefore, peptides comprising or derived from the above sequence can act as competitive inhibitors of proteolysis of corneodesmosin and other adhesion proteins, and thereby restore normal skin barrier.

### Examples B

### Polymorphisms in Protease and Protease Inhibitor Genes

### Example B1

### Identification of Stratum Corneum Chymotrypsin Enzyme (SCCE) Polymorphisms

The DNA sequence encoding the stratum corneum chymotryptic enzyme (SCCE) is retrieved from the NCBI fileserver (www.ncbi.nlm.nih.gov/) under the accession number AF166330. From the same resource, the five exon sequences of the SCCE DNA sequence are obtained and specific primers are designed, using the GeneJockey program (BIOSOFT®, UK).

Identification of Polymorphisms

The five exons of the SCCE gene are screened for mutations and/or polymorphisms. Based on these initial findings, it is decided to focus on exons I and V, since nothing is initially detected in the former and the latter reveals the presence of a insertion of an AACC duplication.

Therefore, to facilitate polymorphism analysis we amplify the two exons of the SCCE gene from 9 healthy (control) individuals in nine separate polymerase chain reaction products using specific primers, F1, R1, F5 and R5.

The primer sequences are as follow. F1: 5′ CACTAGCTCTCCC ATTAGTCCCC 3′; R5: 5′ TCGTTGTGCC AAGCAGAC 3′; F5: 5′ CACTAGCTCTCCC ATTAGTCCCC 3′; R5: 5′ TCGTTGTGCC AAGCAGAC 3′ (see also Table B1.1 below). Due to differences in sequence composition of each exon (e.g. their GC content), different conditions are used to amplify the two exons; these conditions are also specified in the table.

For exon I, genomic DNA amplification is achieved using 20-μl PCR reactions, comprising 2 μl Taq polymerase buffer (×1) (Gibco; ×10), 0.8 μl MgCl₂ (2 mM) (Gibco; 50 mM), 1.6 μl dNTPs (10 mM) (Promega), 0.1 μl W-1 (Gibco; 1%), 0.1 μl Taq polymerase (Gibco; 0.5μ), 1 μl primer F1 (2 μM; initial conc. 20 μM), 1 μl primer R1 (2 μM; initial conc. 20 μM), 1 μl DNA template (100 ng/μl) and 12.4 μl of sterile H₂O.

For exon V, genomic DNA is amplified by PCR in 20μl reactions, comprising of 2 μl Pfx polymerase buffer (×1) (Gibco; >10), 0.8μl MgSO₄ (2 mM) (Gibco; 50 mM), 1.6 μl dNTPs (10 mM) (Promega), 2 μM PCR enhancer solution (×1) (Gibco; ×10 ), 0.06 μl Pfx polymerase (Gibco; 250 u), 1 μl primer F5 (2 μM; initial conc. 20 μM), 1 μl primer R5 (2 μM; initial conc. 20 μM), 1 μl DNA template (100 ng/μl) and 10.54 μl of sterile H₂O.

PCR amplification is carried out in a 40-well thermocycler (TECHNE-GENIUS; Scientific Laboratories Supp. Ltd), under the following conditions: 98° C. for 5 min (1 cycle), 97° C. for 1 min, either 57° C. (exon V) or 60° C. (exon I) for 30 s, 72° C. for 1 min (35 cycles), 74° C. for 5 min and 15° C. hold. A summary of the primers and the PCR conditions, employed for the amplification of the two exons, is shown in Table B1.1.

TABLE B1.1
Primers and conditions used in amplification and sequencing of
exons I and V of the SCCE gene.

| | Forward | Reverse | | Annealing | | |
|---|---|---|---|---|---|---|
| Exon | Primer | Primer | Size (bp) | Temp. (° C.) | Cycles | Mg⁺⁺ |
| I | F1 | R1 | 382 | 60 | 35 | 2 mM |
| V | F5 | R5 | 800 | 57 | 35 | 2 mM |

Agarose Gel Electrophoresis

Amplified products are separated by agarose gel electrophoresis. The following method is based on a standard gel electrophoresis protocol for a 1.5% agarose gel in 1× TAE (4,900 ml of sterile H₂O plus 100 ml 50× TAE stock: 242 g Tris base, glacial acetic acid 57.1 ml, adjusted to pH8 with 100 ml 0.5M EDTA).

1.5 g of agarose is fully dissolved in 100 ml 1×TAE, by heating in a microwave oven and after adding 4 μl of ethidium bromide, the solution is mixed and allowed to cool carefully under cold water. At the same time, a gel tray is prepared by taping the ends on the tray and a comb is placed to form the wells at the desired location. The molten agarose is then poured into the gel tray and allowed to solidify at room temperature. First the comb and then the tape are removed and the gel in its tray is inserted into a horizontal gel tank, where it is covered (by a few mm) with 1× TAE buffer.

The DNA samples containing 10% (v/v) loading buffer (0.25% bromophenol blue, 0.25% xylene cyanol and 25% ficoll), to increase their density, are then loaded into the formed wells in the gel. Normally the wells are loaded with as much sample as possible for more clearer results. The positive (red) and negative (black) terminals of the tank are then connected to the power pack (Bio-Rad, Pac 300) and the gel is allowed to run at a constant voltage (100V). The DNA migrates from the negative electrode to the positive one and after approximately 40 min, the DNA bands are visualised under ultraviolet light (UV; 306ₙₘ).

Extraction and Purification of DNA From TAE Agarose Gels

The initial step, prior to the extraction of the DNA bands from the gel, is the preparation of the glasswool the material necessary for the purification of DNA from agarose contaminants. Using strictly glass equipment, an appropriate amount of glasswool (Sigma) is soaked in a φml solution, containing 2 ml dimethylchlorositane and 98ml chloroform, and left in a fume cupboard overnight. The following day, the soaked glasswool is washed once in methanol and three times in pure water and blot-dried, prior to its use.

Having the dry glasswool already stored in a tube, the DNA bands of interest are identified and they are excised carefully (containing as little as possible excess agarose) from the gel using a clean (washed in ethanol) scalpel and placed in 0.5 ml Eppendorf tubes. These tubes are pierced at the bottom with a small syringe and filled with a reasonable (covering ⅔ of the tube) amount of siliconised glasswool. The 0.5 ml Eppendorf tubes containing the glasswool and the sliced DNA bands are then placed in 1.5 ml Eppendorf tubes and centrifuged for 15 min at 6,000 rpm.

Subsequently the 0.5 ml Eppendorf tube is discarded and the eluted DNA is ethanol precipitated, by adding 0.1 volume (in relation to the volume of the eluted DNA) of 3M sodium acetate, 2 volumes of 100% ethanol and 1 μl of glycogen in each eluted DNA solution. The solutions are then left to precipitate at −70° C. for 60 min or at 4° C. overnight The precipitated solutions are then centrifuged at 14,000 rpm for 15 min, the supernatant is discarded and the pellets are further washed in 200 μl of 70% ethanol. The latter solutions are centrifuged at 14,000 rpm for 5 min, the supernatant is again discarded and the pellets are left to dry in room temperature for a couple of hours. When the pellets are completely dried (no traces of ethanol detected), they are resuspended in 10 μl of H₂O.

Sequencing Analysis of Purified PCR Products

Before the purified PCR products are sent for sequencing, it is necessary to determine whether a sufficient amount of DNA (i.e. >500 ng) is present in the resuspended samples. For that purpose, 1 00 μl of spec solution is prepared containing 2 μl from each resuspension and 98 μl of sterile water and its absorbance is measured by a spectrophotometer against a blank sample. The DNA concentration is then calculated by using the following equation:

DNA Conc. (ng)=O.D×0.05×Dilution factor×μl of remained DNA solution

The resuspension samples that are found to have the appropriate amount of DNA are sent for di-deoxy sequencing, using the BigDye™, which is a Termination Cycle Sequencing Ready Reaction, with resolution being made on a ABI Prism™ 377 DNA sequencer (Applied Biosystems™). Finally, in order to detect possible polymorphisms, the resulting sequences are compared to each other and to the corresponding sequence of SCCE (accession no. AF166330), using the nucleotide multi-alignment program obtained from the GeneJockey program (BIOSOFT®, UK).

Recruitment of Patient and Control Individuals

The 20 atopic eczema individuals who form the basis of this study are recruited from dermatology clinics in Sheffield, UK. Each patient is individually examined by an experienced dermatologist to confirm the diagnosis of atopic eczema.

DNA from healthy controls, ethnically matched to the disease population (white, Northern English), used in this study are obtained from blood donors from the Trent Blood Transfusion service (Sheffield). Genomic DNA is extracted from whole blood, obtained from the above individuals, according to standard protocols and stored in 99-well microtitre plates, with each well containing 500 μl of different DNA solution (100 ng/μl).

DNA Analysis and PCR-Based Assay

For the allelic discrimination of the 4-bp (AACC) insertion/deletion polymorphism detected (see below), two different primers are designed, one (I/D R1) composed of the one AACC repeat and the other (I/D R2) with the two AACC repeat.

Forward: 5′ CAC TAG CTC TCC CAT TAG TCC CC 3′; I/D R I: 5′ GGT TTA TCA ACA GGG CAT GAG GTT TAA AT 3′ and I/D R2: 5′ GGT TTA TCA ACA GGG CAT GAG GTT GGT T 3′ (Table B1.2).

TABLE B1.2
PCR-based assay - Primers and conditions applied

| | | | | Annealing | | |
|---|---|---|---|---|---|---|
| | Forward | Reverse | Size | Temp. | | |
| Allele | Primer | Primer | (bp) | (° C.) | Cycles | Mg⁺⁺ |
| Allele 1 (One | F5 | I/D R1 | 453 | 60 | 35 | 2.5 mM |
| AACC repeat) | | | | | | |
| Allele 2 (Two | F5 | I/D R2 | 457 | 61 | 35 | 2.5 mM |
| AACC repeat) | | | | | | |

Preparation of Primers

Since those primers, used as reverse primers in two separate PCR reactions, are obtained in dry solution it is necessary for them to be ethanol precipitated, prior to their use. Each primer is resuspended in 200 μl TE buffer and after thorough mixing, 100 μl of primer solution is inserted into a 1.5 ml Eppendorf tube. In this tube, 10 μl of 3M sodium acetate (pH4.8) and 330 μl of 100% Ethanol are added and the whole solution is left to precipitate at −70° C. for 60 min or overnight. After precipitation is completed, the solution is microfuged hard for 5 min, the supernatant is discarded and the pellet is washed in 200 μl of 70% ethanol. The latter is microfuged hard for 2 min, the supernatant is removed and the pellet is left to dry. The next step is to resuspend the pellet in 1 00 μl of H₂O. As before spectroscopic analysis is employed to test the absorbance of the DNA within the solution. The following equation is used to calculate the μmoles/l of the primer solution:

(O.D₂₆₀×Dilution factor×0.033)/MolWt(of the primer)×10⁶=μmoles/l

The number obtained is divided by 20 to give a dilution factor of primer solution, leading to 20 μl solution. Each of the reverse primers is used in combination with the forward primer (F5) employed in the amplification of the whole exon V, utilising the same pattern of PCR reaction mixture and thermocycling conditions used in exon V, with modifications regarding Annealing temperature and Mg⁺⁺ concentration, detailed in Table B1.2 above.

Statistical Analysis

Disease and control groups are compared using 2×3 tables. In the control group the allelic distribution of SCCE Allele I/Allele II polymorphism is in Hardy-Weimberg equilibrium. To investigate the possibility of a dose effect, odds ratios (Ors) for the heterozygotes and homozygotes are calculated separately by comparing their risk with that for individuals homozygous for the alternative allele.

A dose effect is evident if the odd ratios (ORs) for the individual homozygous for the rare allele (Allele II) is greater than individual heterozygous for the same allele. Therefore, a χ² analysis for trend is carried out, weighted by the number of putative susceptibility alleles in each genotype group and Fisher's exact p-value is calculated.

### Example B2

### Association of Stratum Corneum Chymotrypsin Enzyme (SCCE) Polymorphisms With Atopic Eczema

Identification of Intron IV and Exon V SNP Polymorphisms

We screened exons I and V of the SCCE gene, for the presence of mutations or polymorphisms. In order to detect those polymorphisms, suitable primers are designed and the appropriate thermocycling conditions are optimised to achieve ideal amplification. The size-expected bands (382 bp and 800 bp for exons I and V, respectively) are selected and purified, as described in an Example above and the corresponding sequences are subsequently obtained from an ABI DNA sequencer, employing the same primers used for their amplification.

Using the multi-alignment program, from the GeneJockey software package (BIOSOFT, UK), we performed an alignment of exon V DNA sequences from several normal and diseased individuals. The results are shown below.

| | |
|---|---|
| | 200 210 220 230 240 250 |
| | \| \| \| \| \| \| |
| Contig# 1 | ATCTGCAAGACAAAGACCGATAACTGAGGAATGTATGAGAATCAGTTGGGCTTTGATCTGAAGC |
| Concensus5 | ATCTGCAAGACAAAGACCGATAACTGAGGAATGTATGAGAATGAGTTGGCCTTTGATCTGCAGC |
| AE2 | ATCTGCAAGACAAAGACCGATAACTGAGGAATGTATGAGAATGAGTTGGGCTTTGATCTGXAGC |
| KLK7exon5AE3/F | ATCTGCAAGACAAAGACCGATAACTGAGGAATGTATGAGAATGAGTTGGGCTTTGATCTGAAGC |
| KLK7ex5AE7/F | ATCTGCAAGACAAAGACCGATAACTGAGGAATGTATGAGAATGAGTTGGGCTTTGATCTGXAGC |
| KLKex5-3F | ATCTGCAAGACAAAGACCGATAACTGAGGAATGTATGAGAATGAGTTGGGCTTTGATCTGAAGC |
| KLKex5-7/F | ATCTGCAAGACAAAGACCGATAACTGAGGAATCTATGAGAATGAGTTGGGCTTTGATCTGAAGC |
| KLKex5-9/F | ATCTGCAAGACAAAGACCGATAACTGAGGAATGTATGAGAATGAGTTGGGCTTTGATCTGAAGC |
| | 260 270 280 290 300 310 320 |
| | \| \| \| \| \| \| \| |
| Contig# 1 | CAAAGTTAATCTCCGGCTCTATTCCCTCTACGGTGACTCAGGGGGACCGTTGGTGTGCAGACGN |
| Concensus5 | CAAAGTTAATCTCCGGCTCTATTCCCTCTAGCGTGACTCAGGGGGACCGTTGGTGTGCAGACGT |
| AE2 | CAAAGTTAATCTCCGGCTCTATTCCCTCTAGGGTGACTCAGGGGGACCGTTGGTGTGCAGAGG- |
| KLK7exon5AE3/F | CAAAGTTAATCTCCGGCTCTATTCCCTCTAGGGTGACTCAGGGGGACCGTTGGTGTGCAGAGG- |
| KLK7ex5AE7/F | CAAAGTTAATCTCCGGCTCTATTCCCTCTAGGGTCACTCAGGGGGACCGTTGGTGTGCAGAGG- |
| KLKex5-3F | CAAAGTTAATCTCCGGCTCTATTCCCTCTAGGGTGACTCAGGGGGACCGTTGGTGTGCAGAGGT |
| KLKex5-7/F | CAAAGTTAATCTCCGGCTCTATTCCCTCTAGGGTGACTCAGGGGGACCGTTGGTGTGCAGAGG- |
| KLKex5-9/F | CAAAGTTAATCTCCGGCTCTATTCCCTCTAGGGTGACTCAGGGGGACCGTTGGTGTGCAGAGG- |
| | 330 340 350 360 370 380 |
| | \| \| \| \| \| \| |
| Contig# 1 | ACCCTGCAAGGTCTGGTGTCCTGGGGAACTTTCCCTTGCGGCCAACCCAATGACCCAGGAGTCT |
| Concensus5 | ACCCTGCAAGGTCTGGTGTCCTGGGGAACTTTCC-TTGCGGCCAACCCAATGACCCAGGAGTCT |
| AE2 | ACCCTGCAAGGTCTGGTGTCCTGGGGAACTTTCCCTTGCGGCCAACCCAATGACCCAGGAGTCT |
| KLK7exon5AE3/F | TCCCTGCAAGGTCTGGTGTCCTGGGGAACTTTCCCTTGCGGCCAACCCAATGACCCAGGAGTCT |
| KLK7ex5AE7/F | ACCCTGCAAGGTCTGGTGTCCTGGGGAACTTTCCCTTGCGGCCAACCCAATGACCCAGGAGTCT |
| KLKex5-3F | ACCCTGCAAGGTCTGGTGTCCTGGGGAACTTTCCCTTGCGGCCAACCCAATGACCCAGGAGTCT |
| KLKex5-7/F | ACCCTGCAAGGTCTGGTGTCCTGGGGAACTTTCCCTTGCGGCCAACCCAATGACCCAGGAGTCT |
| KLKex5-9/F | ACCCTGCAAGGTCTGGTGTCCTGGGGAACTTTCCCTTGCGGCCAACCCAATGACCCAGGAGTCT |
| | 390 400 410 420 430 440 |
| | \| \| \| \| \| \| |
| Contig# 1 | ACACTCAAGTGTGCAAGTTCACCAAGTGGATAAATGACACCATGAAAAAGCATCGCTAACGCCA |
| Concensus5 | ACACTCAAGTCTGCAAGTTCACCAAGTGGATAAATGACACCATGAAAAAGCATC-TGAACGCCA |
| AE2 | ACACTCAAGTGTGCAAGTTCACCAAGTGGATAAATGACACCATGAAAAAGCATCGCTAACGCCA |
| KLK7exon5AE3/F | ACACTCAAGTGTGCAAGTTCACCAAGTGGATAAATGACACCATGAAAAAGCATCGCTAACGCCA |
| KLK7ex5AE7/F | ACACTCAAGTGTGCAAGTTCACCAAGTGGATAAATGACACCATGAAAAAGCATCGCTAACGCCA |
| KLKex5-3F | ACACTCAAGTGTGCAAGTTCACCAAGTGGATAAATGACACCATGAAAAAGCATCGCTAACGCCA |
| KLKex5-7/F | ACACTCAAGTGTGCAAGTTCACCAAGTGGATAAATGACACCATGAAAAAGCATCGCTAACGCCA |
| KLKex5-9/F | ACACTCAAGTGTGCAAGTTCACCAAGTGGATAAATGACACCATGAAAAAGCATCGCTAACGCCA |
| | 450 460 470 480 490 500 510 |
| | \| \| \| \| \| \| \| |
| Contig# 1 | CACTGAGTTAATTAACTGTGTGCTTCCAACAGAAAATGCACAGGAGTGAGGACGCCGATGACCT |
| Concensus5 | CACTGAGTTAATTAACTGTGTGCTTCCAACAGAAAATGCACAGGAGTGAGGACGCCGATGACCT |
| AE2 | CACTGAGTTAATTAACTGTGTGCTTCCAACAGAAAATGCACAGGAGTGAGGACGCCGATGACCT |
| KLK7exon5AE3/F | CACTCAGTTAATTAACTGTGTGCTTCCAACAGAAAATGCACAGGAGTGAGGACGCCGATGACCT |
| KLK7eX5AE7/F | CACTGAGTTAATTAACTGTGTGCTTCCAACAGAAAATGCACAGGAGTGAGGACGCCGATGACCT |
| KLKex5-3F | CACTGAGTTAATTAACTGTGTGCTTCCAACAGAAAATGCACAGGAGTGAGGACGCCGATGACCT |
| KLKex5-7/F | CACTGAGTTAATTAACTGTGTGCTTCCAACAGAAAATGCACAGGAGTGAGGACGCCGATGACCT |
| KLKex5-9/F | CACTGAGTTAATTAACTGTGTGCTTCCAACAGAAAATGCACAGGAGTGAGGACGCCGATGACCT |
| | 520 530 540 550 560 570 |
| | \| \| \| \| \| \| |
| Contig# 1 | ATGAANGTCAAATTTGACTTTACCTTTCCTCAAAGATATATTTAAACCNACCTCATGCCCTGTT |
| Concensus5 | ATGAA-GTCAAATTTGACTTTACCTTTCCTCAAAGATATATTTAAACCAACCTCATGCCCTGTT |
| AE2 | ATGAA-GTCAAATTTGACTTTACCTTTCCTCAAAGATATATTTAAACCAACCTCATGCCCTGTT |
| KLK7exon5AE3/F | ATGAA-GTCAAATTTCACTTTACCTTTCCTCAAAGATATATTTAA----ACCT |
| KLK7ex5AE7/F | ATGAA-GTCAAATTTGACTTTACCTTTCCTCAAAGATATATTTAA----ACCTCATGCCCTGTT |
| KLKex5-3F | ATGAAAGTCAAATTTGACTTTACCTTTCCTCAAACATATATTTAAACC----TCATGCCCTGTT |
| KLKex5-7/F | ATGAA-GTCAAATTTGACTTTACCTTTCCTCAAAGATATATTTAA----ACCTCATGCCCTGTT |
| KLKex5-9/F | ATGAA-GTCAAATTTGACTTTACCTTTCCTCAAAGATATATTTAA----ACCTCATGCCCTGTT |
| | 580 590 600 610 620 630 640 |
| | \| \| \| \| \| \| \| |
| Contig# 1 | GATAAACCAATCAAATTGGTAAAGACCTAAAACCAAAACAAATAAAGAAACACAAAACCCTCAG |
| Concensus5 | GATAAACCAATCAAATTGGTAAAGACCTAAAACCAAAACAAATAAAGAAACACAAAACCCTCAX |
| AE2 | GATAAACCAATCAAATTGGTAAAGACCTAAAACCAAAACAAATAAAGAAACACAAAACCCTCAG |
| KLK7ex5AE7/F | GATAAACCAATCAAATTGGTAAAGACCTAAAACCAAAACAAATAAAGAAACACAAAACCCTCAG |
| KLKex5-3F | GATAAACCAATCAAATTGGTAAAGACCTAAAACCAAAACAAATAAAGAAACACAAAACCCTCAG |
| RLKex5-7/F | XGTXAACCAATCAAATTGGTTGAGACCTAAXTCCAAACCAAATAAAGAAACACAAAACCCTCAC |
| KLKex5-9/F | GATAAACCAATCAAATTGGTAAAGACCTAAAACCAAAACAAATAAAGAAACACAAAACCCTCAG |

AE2, KLKex5-3F and KLK7ex5AE7/F correspond to atopic eczema patients, whereas 3, 7 and 9, are obtained from control individuals and sequenced using the F5 primer. Significant nucleotide differences, suggesting possible SNPs are highlighted in bold, whereas the AACC insertion or deletion is underlined.

No SNPs are detected in exon 1 by sequencing 3 control samples. Alignments show significant differences between specific nucleotides of sequences for the SCCE gene from different individuals, suggesting the presence of SNPs (see Table B2.1 below). For example, SNP (A→C) is found at base 253 of the consensus region of intron IV. Two more SNPs (G→T and A→C) are identified at bases 580 and 607 respectively, of the consensus sequence of exon V. They both lead to an amino acid change in the mRNA encoded by the SCCE gene.

TABLE B2.1
Positions of SNPs within exons I and V of the SCCE gene.

| | | | Position in | | Nucleic | Amino |
|---|---|---|---|---|---|---|
| | | | Consensus | | acid | acid |
| Exon | Intron | Genomic | Sequence | cDNA | change | change |
| | 4 | 23,857 | 253 | | A→C | |
| 5 | | 24,174 | 580 | 7,650 | G→T | L→F |
| 5 | | 24,205 | 607 | 7,680 | A→C* | K→N |

Identification of an Exon V AACC Insertion/Deletion Polymorphism in an Atopic Eczema Sample

The sequence alignments as shown above demonstrate the presence of a 4-bp repeat (AACC) in exon V of the SCCE sequence (Genomic location: 7,634-7,637 bp) of an atopic eczema patient (designated as AE2), which corresponds to the sequence of the SCCE obtained from the database.

However, this repeat is not detected in some of the other aligned sequences, suggesting that it might be an insertion or a deletion, depending on the sequence one is referring to.

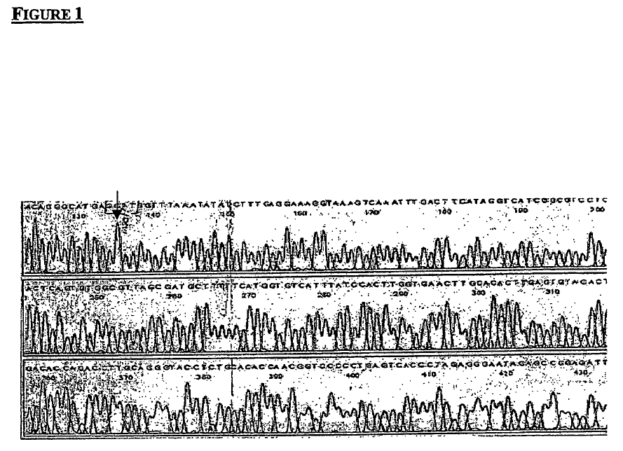

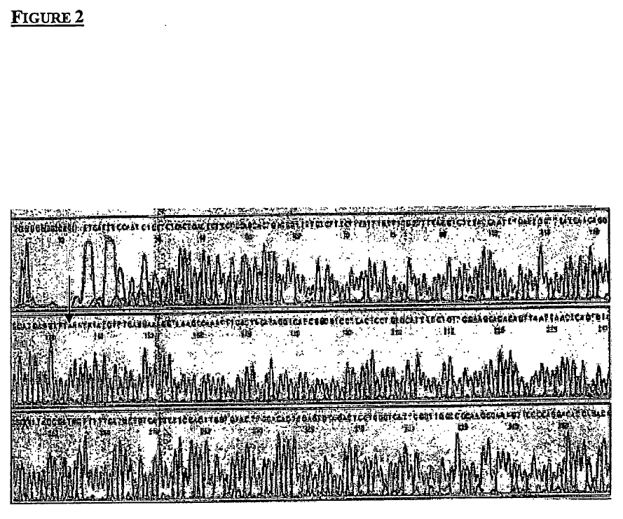

FIG. 1 shows a chromatogram of part of the AE2 sequence corresponding to Exon V of the SCCE gene, showing this insertion/deletion. A corresponding chromatogram obtained from a control individual is shown in FIG. 2. The absence of the second repeat (AACC) shown as GGTT is clear from the sequence of the chromatogram.

Case Control Study

We further designed a PCR-based assay using specific primers to discriminate between individuals that harbour the one-repeat (AACC) allele and individuals who harbour the two-repeat (AACCAACC) allele. The primers are designed so that they are complementary to the DNA sequence. The primers have the same sequence except that the second repeat (AACC) is not present in one primer (see Example B1 above).

Thus, if the sequence of the sample under investigation lacks the one repeat, hybridisation would be incomplete and amplification would not occur. In that case, amplification of that sequence would occur using the alternative allele (i.e. the one with the two-repeats in its sequence). Amplification with both primers indicates that the individual that corresponds to the examined sample is heterozygous for both alleles. For the purpose of simplicity we designate the allele that harbours the one-repeat in its sequence Allele I and the one with the two repeats (AACCAACC) Allele 2.

Optimization of PCRs That Differentiate Between the Two Alleles

In order to determine the allelic distribution of allele 1 (the one-repeat allele) and allele 2 (the two-repeat allele), we designed a direct PCR assay, using specific primers (see Example B1 above). Prior to the screening of healthy and patient individuals, the optimization of the PCR conditions is necessary to yield accurate and efficient results.

First Optimisation

For the first optimization, primers F5 and I/D RII are used under the thermocycling conditions for Exon V, with the specific conditions of 2.5 mM Mg⁺⁺ and 60° C. (Annealing Temperature). For this purpose, ten DNA samples are studied, one of which is the AE2 sequence (already shown to contain the two-repeat allele in its sequence—see FIG. 1) and nine control individuals, designated as Poly1-9, with Poly9 already found to contain the one-repeat allele in its sequence (see FIG. 2).

The results show absence of amplification in samples 2, 3, 7 and 9. However, it is a possibility that this is due not to absence of the second repeat in those sequences but due to bad DNA quality or to non-specific PCR conditions. In order to exclude this possibility, we performed another set of PCR reactions, which include a “control” amplification product of 800 bp, the product of amplification of the whole exon 5 (i.e. primers F5 and R5, 2 mM Mg⁺⁺ and 57° C.). The resulting gel is shown in FIG. 3, and demonstrates that our previous optimization is accurate and the allele discrimination results are valid.

Second Optimization

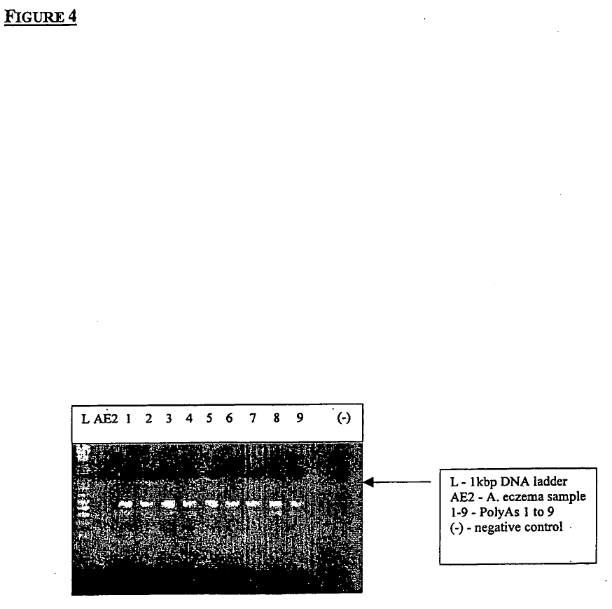

The second optimisation involves the use of the same DNA samples, primers F5 and I/D RI, and PCR conditions of 2.5 mM Mg⁺⁺ and 61° C. As expected there is no band in the AE2 lane, whereas there are bands for samples 2,3,7 and 9 for which amplification previously did not occur. The results for the second optimisation are shown in FIG. 4.

Genotyping of Control and Atopic Eczema Samples Using Optimised Conditions

By means of a direct PCR method, using the above optimised conditions for both primers, we screened healthy and atopic eczema samples in order to elucidate the allelic distribution of the two distinct alleles of the SCCE gene.

Results of reactions in which a control band is present along with the actual screening results are shown in Table B2.2 below.

TABLE B2.2
Allelic distribution of SCCE AACC/AACCAACC
polymorphism in atopic eczema and control groups

| | Allele 1 | Heterozygotes | Allelle 2 |
|---|---|---|---|
| Control | 8 | 7 | 5 |
| Atopic Eczema | 4 | 2 | 14 |
| Total | 12 | 9 | 19 |

Statistical Analysis

The allelic distribution of the SCCE AACC/AACCAACC polymorphism in both atopic eczema and control groups is shown in Table B2.2 above. There is a significant increase in allele frequency of the rare allele (Allele 2) in the patient group compared to the control group. The rare allele in the control group (ferquency=0.43) is the more common allele in the disease group (frequency=0.75). As a dose effect is evident for this polymorphism, the odds ratio (ORs) for the rare allele is significantly greater than of the heterozygous. Therefore a χ² test is carried out. A strong association is found between SCCE-Allele II variant and Atopic eczema [p=0.0104, OR=7.00 (95% confidence interval (1.74, 28.17))].

Conclusion

We show that the SCCE allele with the insertion AACCAACC is common in the patient cohort. 80% of eczema patients carry one or two SCCE AACCAACC alleles and 70% are homozygous for this allele. However, in the control group, 60% are heterozygotes for SCCE AACCAACC allele and only 25% are homozygotes for this allele. An SCCE allele comprising AACCAACC is therefore shown to be associated with eczema.

We surmise that the insertion in 3′UTR has a strong effect on the stability of the mRNA. Therefore the quantity of SCCE produced changes, which affects the process of corneodesmosomal proteolysis. This breaks skin barrier homeostasis and lead to an eczematous clinical phenotype.

The SCCE insertion may be used to diagnose patients with sensitive skin (e.g. eczema). This diagnosis is extremely useful for therapeutic treatments. For example, if the insertion is associated with less SCCE protein production, the treatments are focused in increasing SCCE physiological concentration (e.g. inhibition of SLPI or use of SCCE in a pharmaceutical lotion to the skin). However, if the insertion in SCCE gene is associated with an increase in SCCE concentration in the skin, the treatments are focused on decreasing the endogenous concentration of SCCE by applying for example, SLPI and/or SLPI peptides and/or SCCE antibody in a lotion to the skin.

We therefore disclose the diagnosis of a Group I disease or susceptibility to a Group I disease (preferably eczema or susceptibility to eczema, preferably atopic eczema) in an individual, by detecting the presence of an AACCAACC sequence in an SCCE nucleic acid of an individual. We further disclose such diagnosis by detection of an AACC sequence at positions corresponding to positions 7634-7637 in an SCCE genomic sequence (GB: AF166330). Preferably, the diagnosis is conducted on mRNA or genomic DNA of an individual. Preferably an AACC sequence is detected at positions 7634-7637 of an SCCE genomic sequence (GB: AF166330).

The detection of the absence of AACCAACC at the specified positions may also be used to diagnose or detect susceptibility to a Group II disease, preferably psoriasis and/or acne.

### Example B3

### Identification of Secretory Leukoprotease Inhibitor (SLPI) Polymorphisms

Detection of Mutations in the Promoter of SLPI

We screened approximately 1500 bp of the promoter region of SLPI, including the TATA signal, CAAT signal and the two CRE-like sequences.

Ten samples are obtained from different ethnical groups and with no history of skin diseases, and 10 samples are obtained from patients with each of atopic eczema, acne vulgaris and psoriasis vulgaris. Each of these samples is screened by PCR using appropriate primers. First, the SLPI promoter sequence (Gene Bank accession number; M74444) is screened for regulatory elements. Two CRE (cyclic AMP response element)-like sequences are found. Cyclic AMP response elements (CRE) are recognised by transcription factors such as NF-IL6 and CREB (Tsukada et al, 1994). Mutation points in CRE significantly decrease IL1-beta stimulation (Potter et al, 2000). It is known that SPLI is up-regulated by both IL-1beta and TNF-alpha (Tanaka et al, 2000, see also treatment section in this study).

The samples are screened around the SLPI promoter region containing TATA signal (−27/−21), CAAT signal (−87/−83) (as reported by Abe et al, 1991) and the two CRE-like 1 (ttgctgtca; −907/−898) and CRE-like 2 (tacgtacgtca; −1157/−1146) sequences. The positions are given by reference to the position transcription initiation point (+1; C at position 1374 in the sequence). The sequence of the promoter region of SLPI is shown below:

| | |
|---|---|
| 1 | gaattccaag catgaagata atgagtcaag agcttggagt ttgtagctag atgagctttg |
| 61 | gttgaatttt attttatttt atttttttaa gacagggtat cgctctgtcc cccaagctgg |
| 121 | aatgcagtgg cacaatcatg gctcactgca gcctcaaact cctgggctaa agcgatcctc |
| 181 | ctggctcagc ctcccaagta gctgggacta caggcatacg tacgtcatca tgcctggctg |
| 241 | attttttaca tttttttgta gagatggggt ctcaatatgt ggccagggct ggtctcaaac |
| 301 | tcctactctc aaggaatcca tacacctcag cctcctgggc agctgagaca gcaagtgtgc |
| 361 | gaccctacac tcagctatgg gctgaatttt agagataatg gtcgctctct ttataattag |
| 421 | aagcaaccta tgcagactgg gtagcaaata gaatgggttt aattttttgc tgteatgtga |
| 481 | gatctgtaag ggattttggg gaattttagg aagcaatcct ctaagatctc aaattatctc |
| 541 | acagctaaat gtagattaca gtgactgatg agctgctttc cccctttatc tcagattcat |
| 601 | ttcaattctc tttagtggga agggatacta ttcatttgtt cttttcattc agagtccctt |
| 661 | catgccctta atttcataac cctctgagaa gggctgactt gttagtatca tttcatttca |
| 721 | cagctgagac aactgagctc cagagagatt tgtggagagc ggagctcttc ttcagctttc |
| 781 | atttgtgagt gcttttcctg tgtcaggcac agaacaggca ctggggatat aacggtgtaa |
| 841 | atatttcagg gaactaagta tcagttggtt gaacgagctg aacttttgag aaagaaactg |
| 901 | cattgagtaa tcagcagagt ttcacaatgc ctgagagtcc agtaatgtga gaatcagaat |
| 961 | tagcaatgtg agaatagaat gtattgcaca aagtctcagc agggagtctg tgtctggttt |
| 1021 | tagttccagg tccgggtagc acctttgcaa ttgaccactt cttccctctc tccacctata |
| 1081 | aggctaatgg cctgggatct tgtgatgttt agggctcaga tggacactga gatggcctct |
| 1141 | ttaatcaacc aacttcccag gccaatctct tccctttctt ttctgatagt tgctgtgttg |
| 1201 | gcctcatagc cttacctggc ataggaaaga taaacaatct ccttggtgtc aggatttctg |
| 1261 | gtctctggct acgtttcctg cttatgcaat agtagctggg agaggccgaa agaattctgg |
| 1321 | tggggccaca cccactggtg aaagaataaa tagtgaggtt tggcattggc catcagagtc |
| 1381 | actcctgcct tcaccatgaa gtccagcggc ctcttcccct tcctggtgct gcttgccctg |
| 1441 | ggaactctgg caccttgggc tgtggaaggc tc |

CRE-like positions are shown in bold. TATA-box and ATG are underlined.

PCR conditions are as follow: 1 cycle of 94° C. /3 minutes; 45 cycles of 94° C./30 seconds; Annealing temperature/30 seconds; 72° C. /45 seconds; 1 cycle of 72° C./10 minutes; 4° C. for ever. Primers used for PCR are shown in Table B3.1 below.

TABLE B3.1

| Fragment | Forward Primer | Reverse Primer |
|---|---|---|
| SLPI 1 | GCT AAA GCG ATC CTC CTG | CAG ACT CCC TGC TGA GAC |
| SLPI 2 | GTC TCA GCA GGG AGT CTG | CAG ATT AGA CAG TGA CTC C |

Optimised concentrations of Mg²⁺ and annealing temperatures for amplifying each fragment of SLPI promoter are provided in Table B3.2 below.

TABLE B3.2

| Fragment | Mg²⁺ Concentration (mM) | Annealing Temperature |
|---|---|---|
| SLPI 1 | 1.5 | 57 |
| SLPI 2 | 1.5 | 56 |

Using the PCR screening procedure, we identify mutations in SLPI, as shown in Table B3.3 below.

TABLE B3.3
Mutations in the human SLPI sequence.

| | | | | Nucleic | |
|---|---|---|---|---|---|
| | | | Nucleotide | Acid | Sequence of 30 bases |
| Acesssion | Exon | Intron | Position*** | Change | around the mutation. |
| M74444 | | Promoter | 217-227 | CRE-like 2 | |
| | | | | (no change) | |
| | | Promoter | 240 | G - A | CATCATGCCTGGCTG |
| | | | | | ATTTTTTACATTTTT |
| | | Promoter | 240/241 | G insert | CATCATGCCTGGCTG |
| | | | | | ATTTTTTACATTTTT |
| | | Promoter | 279 | G - T | GGGGTCTCAATATGT |
| | | | | | GGCCAGGGCTGGTCT |
| | | Promoter | 279/280 | T or G | GGGGTCTCAATATGT |
| | | | | insert | GGCCAGGGCTGGTCT |
| | | Promoter | 280 | T - A | GGGGTCTCAATATGT |
| | | | | | GGCCAGGGCTGGTCT |
| | | Promoter | 291 | G - T | TATGTGGCCAGGGCT |
| | | | | | GGTCTCAAACTCCTA |
| | | Promoter | 292/293 | G insert | TATGTGGCCAGGGCT |
| | | | | | GGTCTCAAACTCCTA |
| | | Promoter | 345 | G - T | CCTCCTGGGCAGCTG |
| | | | | | AGACAGCAAGTGTGC |
| | | Promoter | 467-475 | CRE-like 1 | |
| | | | | (no change) | |
| | | Promoter | 762 | G - A | TGTGGAGAGCGGAGC |
| | | | | | TCTTCTTCAGCTTTC |
| | | Promoter | 815 | C - A | TGTCAGGCACAGAAC |
| | | | | | AGGCACTGGGGATAT |
| | | Promoter | 816 | G - A | TGTCAGGCACAGAAC |
| | | | | | AGGCACTGGGGATAT |
| | | Promoter | 1235/ | C insert | ATGCTCTTTGCAGATA |
| | | | 1236** | | TCTAGAGCTACGCC |
| | | Promoter | 1287-1291 | CAAT | |
| | | | | signal (no | |
| | | | | change) | |
| | | Promoter | 1300** | G - C* | CTGATTAGCGGAATG |
| | | | | | GGAGCAGGCGGGGCA |
| | | Promoter | 1325** | G - A | GAGCAGGCGGGGCAG |
| | | | | | ACAGGAGAAGATCGC |
| | | Promoter | 1347-1352 | TATA | |
| | | | | signal (no | |
| | | | | change) | |
| | | Promoter | 1384/ | C Insert | GAGAAGATCGCCAAA |
| | | | 1385** | | CCTTGACCCTGAGAA |
| | | Promoter | 1387** | A - G | GAGAAGATCGCCAAA |
| | | | | | CCTTGACCCTGAGAA |
| | | Promoter | 1418** | C - T | AATTTTTCCATGGCCT |
| | | | | | TCCTGGAGGCCCAG |
| X04502 | | Promoter | 1481** | A - G | GGCCCAGTCCTCTGA |
| | | | | | TCTTCAGGGAAGAAA |
| | | Promoter | 1481/ | G Insert | GGCCCAGTCCTCTGA |
| | | | 1482** | | TCTTCAGGGAAGAAA |
| | | Promoter | 1509/ | G Insert | AGCCCCTGGGAAGTC |
| | | | 1510** | | AGTGTTCCAGAGCA |
| | | Promoter | 1549/ | G/C Insert | AAGAAACAGGCCCTG |
| | | | 1550** | | AAAATCCGCAGGGTC |
| | | Promoter | 1567/ | T Insert | TCCAGAGCACTGGGC |
| | | | 1568** | | AACCTGGGGGCAGGT |
| | | Promoter | 1596/ | T Insert | GCAACCTGGGGGCAG |
| | | | 1597** | | GTGGCAGAGCCTGGT |

The polymorphism positions are underlined
**These are from the second PCR fragment which was used.
***See the reference sequence at Annex B.

Disease association between the various polymorphisms and alleles identified are shown in Table B3.4 below.

TABLE B3.4
Disease association in SLPI.

| Nucleotide | Nucleic Acid | C mmon allele in: | | | |
|---|---|---|---|---|---|
| Position | Change | Healthy | Acne | Eczema | Psoriasis |

NCBI M74444

| | | | | | |
|---|---|---|---|---|---|
| 240 | G - A | G | A | None | None |
| 240/241 | G insert | G | — | None | — |
| 279 | G - T | G*** | G | None | None |
| 279/280 | T or G insert | — | — | None | None |
| 280 | T - A | T | T | T*** | None |
| 291 | G - T | G*** | G | None | G*** |
| 292/293 | G insert | — | — | G*** | —*** |
| 345 | G - T | G*** | G | None | G*** |
| 762 | G - A | G | | | |
| 815 | A - C | A | | | |
| 816 | G - A | None | | | |
| 1235/1236** | C insert | C | — | C*** | — |
| 1300** | G - C | G | G*** | G | G |
| 1325** | G - A | G | G | G | G*** |
| 1384/1385** | C or A Insert | C | None | —*** | A |
| 1387** | A - G | None | None | None | A |
| 1418** | C - T | C*** | C*** | C | C*** |

NCBI X04502

| | | | | | |
|---|---|---|---|---|---|
| 9** | A - G | G | G*** | G*** | G*** |
| 9/10** | G Insert | G | None | G*** | — |
| 37/38** | G Insert | — | —*** | None | — |
| 77/78** | G/C Insert | — | None | None | — |
| 95/96** | T Insert | — | T | —*** | —*** |
| 124/125** | T Insert | —*** | None | None | None |

None: Both alleles are present at the same frequency.
Blank: No samples of that category present at that position.
***Only this allele present in this position and in this sample set.

Detection of SLPI Polymorphisms for Diagnosis of Disease

We provide in general a method of diagnosis of disease, preferably an inflammatory skin disease, by detection of any of the polymorphisms in SLPI as identified in Table B3.4 above.

In particular, we provide a method of diagnosis of disease, preferably an inflammatory skin disease, by detection of any of the polymorphisms in SLPI as shown in Table B3.5 below:

TABLE B3.5

| Disease | Polmorphisms to Detect |
|---|---|
| Acne | 1300G, 1418C |
| Eczema | 280T, 292/293G, 1235/1236C, 1384/1385- |
| Psoriasis | 291G, 292/293-, 1325G, 1418C |

In this and each of the next three subsections, position numbers are preferably made in reference to accession number M74444, or the reference SLPI sequence in Annex B.

Diagnosis of Acne

In particular, we disclose a method of diagnosis of a Group II disease, preferably acne, in an individual, the method comprising detectin the presence of a G residue at position 1300 of SLPI or the presence of a C residue at position 1418 of SLPI, or both.

Diagnosis of Psoriasis

We disclose a method of diagnosis of a Group II disease, preferably psoriasis, in an individual, the method comprising detecting the presence of one or more polymorphisms selected from the group consisting of: the presence of a G residue at position 291 of SLPI, the absence of a G residue at position 292/293 of SLPI, the presence of a G residue at position 1325 of SLPI, the presence of a C residue at position 1418 of SLPI.

Diagnosis of Eczema

We disclose a method of diagnosis of a Group I disease, preferably eczema, more preferably atopic eczema, in an individual, the method comprising detecting the presence of one or more polymorphisms selected from the group consisting of: the presence of a T residue at position 280 of SLPI, the presence of a G residue at position 292/293 of SLPI, the presence of a C residue at position 1235/1236 of SLPI, the absence of a C or A residue at position 1384/1385 of SLPI.

### Example B4

### Identification of Cystatin A (CSTA) Promoter Polymorphisms

Cystatin A (CSTA) is a cysteine proeinase inhibitor expressed in the stratum corneum located at chromosomal location 3q21. This region contains the susceptibility loci of both psoriasis, atopic dermatitis and other autoimmune diseases (Cookson et al, 2001, Lee et al, 2000, Becker et al, 1998).

Detection of Mutations in the Promoter of Human Cystatin A

We screened 500-1000 bp of the promoter region of Cystatin A (CSTA) including TATA signal and CAAT sequences in ten samples from different ethnical groups and with no history of skin diseases, 10 samples from patients with each of atopic eczema, acne vulgaris and psoriasis vulgaris.

PCR conditions are as follow: 1 cycle of 94° C./3 minutes; 45 cycles of 94° C./30 seconds; Annealing temperature/30 seconds; 72° C./45 seconds; 1 cycle of 72° C./10 minutes; 4° C. for ever. Primers used for PCR are shown in Table B4.1 below

TABLE B4.1

| Region | Forward Primer | Reverse Primer |
|---|---|---|
| Cystatin Promoter | GAA GAC ACA TCC AGC | CTG GAT TTC TGG |
| | CAA G | AGT GGC G |
| Cystatin Exon 1 | CAG ATG ATG CAA CAG | GCT CAA GGT CAC |
| | GAT G | ACT CAC AG |
| Cystatin Exon 2 | GGT ACA TTG CAT ACA | TGA GAG TCC ACC |
| | TGG | ACT TG |
| Cystatin Exon 3 | GAC CTG TGG CTC TCT | CAG TTG CAT TAG |
| | CAC | GCT TGA C |

Optimised concentrations of Mg²⁺ and annealing temperatures for amplifying each of the four regions are shown in Table B4.2 below.

TABLE B4.2

| Region | Mg²⁺ Concentration (mM) | Annealing Temperature |
|---|---|---|
| Cystatin Promoter | 1.5 | 58 |
| Cystatin Exon 1 | Not Optimised | |
| Cystatin Exon 2 | 1.5 | 55 |
| Cystatin Exon 3 | 1.5 | 58 |

Using the PCR screening procedure, we identify mutations in the Human Cystatin A regions, as shown in Table B4.3 below.

TABLE B4.3
Mutations in the Human Cystatin A sequence.

| Intron | Exon | Position** | Nucleic Acid Change |
|---|---|---|---|
| Promoter | | 1; −375 | T deletion |
| Promoter | | 1; −373 | A - G |
| Promoter | | 1; −334 | T deletion |
| Promoter | | 1; −284 | T deletion |
| Promoter | | 1; −271/1; −270 | G insertion |
| Promoter | | 1; −256/1; −257 | G insertion |
| Promoter | | 1; −235/1; −234 | G insertion |
| Promoter | | 1; −190 | T - C |
| Promoter | | 1; −186 | T - G |
| Promoter | | 1; −135 | A - G |
| Promoter | | 1; −134/1; −133 | A insertion |
| Promoter | | 1; −130 & 1; −129 | GC deletion |
| Promoter | | 1; −124 | A - G |
| Promoter | | 1; −123 | A - G |
| Promoter | | 1; −122 & 1; −121 | AC deletion |
| Promoter | | 1; −111/1; −110 | G insertion |
| Promoter | | 1; −104 | T - C |
| Promoter | | 1; −96 | C - G |
| Promoter | | 1; −92 | T - A |
| Promoter | | 1; −85 | T deletion |
| Promoter | | 1; −77 & 1; −76 | TG deletion |
| Promoter | | 1; −73 | G deletion |
| Promoter | | 1; −72 | A deletion |
| Promoter | | 1; −71 | A deletion |
| Promoter | | 1; −65 & 1; −64 | AG deletion |
| Promoter | | 1; −60 | T deletion |
| Promoter | | 1; −59 & 1; −58 | CT deletion |
| Promoter | | 1; −57 | A deletion |
| Promoter | | 1; −48 | G - C/deletion |
| Promoter | | 1; −47 | C - T |
| Promoter | | 1; −26 | A deletion |
| Promoter | | 1; −25 | C - T |
| Promoter | | 1; −24 | A - G |
| Promoter | | 1; −23 | G - A |
| Promoter | | 1; −20 | G - T |
| Promoter | | 1; −17 | T - C |
| Promoter | | 1; −15 | C deletion |
| Promoter | | 1; −14 | A deletion |
| Promoter | | 1; −13 | C deletion |
| Promoter | | 1; −10 | T - C |
| Promoter | | 1; −6 | C deletion |
| Promoter | | 1; −5 | T deletion |
| Promoter | | 1; −4 | G deletion |
| Promoter | | 1; −2 | T - G/deletion |
| | Exon 1 | 1; 1 | A deletion |
| | Exon 1 | 1; 2 | C deletion |
| | Exon 1 | 1; 3 | T deletion |
| | Exon 1 | 1; 4 | T deletion |
| | Exon 1 | 1; 7 | G deletion |
| | Exon 1 | 1; 8 | T deletion |
| | Exon 1 | 1; 55 | G - T* |
| | Exon 2 | 2; 2410 | T - A |
| | Exon 2 | 2; 2403/2; 2404 | G insertion |
| | Exon 2 | 2; 2446 | T - C |
| | Exon 2 | 2; 2480 | G - C |
| | Exon 3 | 2; 6329 | A - C |
| | Exon 3 | 2; 6398 | A - T |
| | Exon 3 | 2; 6419 | C - T |
| | Exon 3 | 2; 6492 | A - G |
| | Exon 3 | 2; 6511 | G - A |

**The positions given in this table use the 5′ end of exon 1 as position 1. All other positions are relative to this.

As there is a gap in the published sequence between the 5′ and 3′ sections of intron 1, so numbering of nucleotide positions are made with reference to two Cystatin A sequences, CystA.1 and CystA.2. Sequence CystA.2, position 1 is at the start of the published 3′ section of intron 1. The sequence used for the position numbering is indicated by “1” (for CystA.1) or “2” (for CystA.2) before the semicolon. Therefore, for example, “2;2446” indicates the position 2446 in sequence for CystA.2. The reference sequences for CystA.1 and for CystA.2 are shown in Annex A below.

Disease association between the various polymorphisms and alleles identified are shown in Table B4.4 below.

TABLE B4.4
Disease association in cystatin A alleles.

| | Nucleic Acid | Common allele in: | | | |
|---|---|---|---|---|---|
| Position** | Change | Healthy | Acne | Eczema | Psoriasis |
| 1; −1744 | G - T | G | | | |
| 1; −1650 | T - C | G | | | |
| 1; −1616 | G - C | G | | | |
| 1; −1558 | A - C | A | | | |
| 1; −1489 | A - T | A | | | |
| 1; −1468 | C - T | C | | | |
| 1; −1445 | A - G | A | | | |
| 1; −1376 | G - A | G | | | |
| 1; −375 | T deletion | | T | T | T |
| 1; −373 | A - G | | A | A | A |
| 1; −334 | T deletion | | — | T | T |
| 1; −284 | T deletion | | — | None | T |
| 1; −271/1; −270 | G insertion | | G | — | —*** |
| 1; −256/1; −257 | G insertion | | — | — | — |
| 1; −235/1; −234 | G insertion | | — | G | — |
| 1; −190 | T - C | | T | T | T |
| 1; −186 | T - G | | T | T | T |
| 1; −135 | A - G | A | A | G | None |
| 1; −134/1; −133 | A insertion | — | A | None | A |
| 1; −130 & | GC deletion | GC | GC | GC | GC |
| 1; −129 | | | | | |
| 1; −124 | A - G | A | None | G | G |
| 1; −123 | G - A | G | None | Hetero | Hetero |
| 1; −122 & | AC deletion | AC*** | — | —*** | — |
| 1; −121 | | | | | |
| 1; −111/1; −110 | G insertion | —*** | —*** | G*** | G |
| 1; −104 | T - C | T*** | None | None | T |
| 1; −96 | C - G | C*** | C*** | None | C |
| 1; −92 | T - A | T*** | A | None | A |
| 1; −85 | T deletion | T*** | T | T*** | T |
| 1; −77 & 1; −76 | TG deletion | TG*** | TG*** | — | — |
| 1; −73 | G deletion | G*** | —*** | G*** | G*** |
| 1; −72 | A deletion | A*** | — | —*** | — |
| 1; −71 | A deletion | A*** | A*** | — | — |
| 1; −65 & 1; −64 | AG deletion | AG*** | AG | — | — |
| 1; −60 | T deletion | T*** | — | —*** | — |
| 1; −59 & 1; −58 | CT deletion | CT*** | — | CT | CT |
| 1; −57 | A deletion | A*** | — | A | A |
| 1; −48 | G - C/ | G*** | G*** | — | None |
| | deletion | | | | |
| 1; −47 | C - T | C*** | C | C | C |
| 1; −26 | A deletion | A*** | A | A | — |
| 1; −25 | C - T | C*** | C | C | C |
| 1; −24 | A deletion | A*** | —*** | A | — |
| 1; −23 | C deletion | C*** | C | C | None |
| 1; −20 | G - T | G*** | G*** | None | None |
| 1; −17 | T - C | T*** | T*** | C | C |
| 1; −15 | C deletion | C*** | — | —*** | —*** |
| 1; −14 | A deletion | A*** | None | —*** | — |
| 1; −13 | C deletion | C*** | C*** | —*** | — |
| 1; −10 | T - C | T*** | T*** | Hetero | Hetero |
| 1; −6 | C deletion | C*** | —*** | —*** | —*** |
| 1; −5 | T deletion | T*** | T | —*** | — |
| 1; −4 | G deletion | G*** | G | — | — |
| 1; −2 | T - G/ | T*** | — | G | G |
| | deletion | | | | |
| 1; 1 | A deletion | A | — | — | — |
| 1; 2 | C deletion | C | — | C | C |
| 1; 3 | T deletion | T*** | — | T | T |
| 1; 4 | T deletion | T*** | — | T*** | T*** |
| 1; 7 | G deletion | G*** | G | —*** | —*** |
| 1; 8 | T deletion | T*** | T*** | — | — |
| 2; 2410 | T - A | T | None | | |
| 2; 2403/2; 2404 | G insertion | G | — | | |
| 2; 2446 | T - C | C | None | | |
| None: Both alleles are present at the same frequency. | | | | | |
| Blank: No samples of that category present at that position. | | | | | |
| ***Only this allele present in this position and in this sample set. | | | | | |
| **The positions given in this table use the 5′ end of exon 1 as position 1. All other positions are relative to this. As there is a gap in the published sequence between the 5′ and 3′ sections of intron 1, so numbering of nucleotide positions are made with reference to two Cystatin A sequences, CystA.1 and CystA.2. | | | | | |
| # Sequence CystA.2, position 1 is at the start of the published 3′ section of intron 1. The sequence used for the position numbering is indicated by “1” (for CystA.1) or “2” (for CystA.2) before the semicolon. Therefore, for example, “2; 2446” indicates the position 2446 in sequence for CystA.2. | | | | | |
| # The reference sequences for CystA.1 and for CystA.2 are shown in Annex A below. | | | | | |

Detection of Cystatin A Polymorphisms for Diagnosis of Disease

We provide in general a method of diagnosis of a disease, preferably an inflammatory skin disease, by detection of any of the polymorphisms in Cystatin A identified in Table B4.4 above.

Diagnosis of Acne

In particular, we disclose a method of diagnosis of a Group II disease, preferably acne, in an individual, the method comprising detecting any one or more polymorphisms selected from the group consisting of: the presence of a G residue at position 110 of CystA.1, the presence of a C residue at position 96 of CystA.1 , the presence of an A residue at position 71 of CystA.1, the presence of a G residue at position 20 of CystA.1, the presence of a T residue at position 17 of CystA.1, the presence of a C residue at position 13 of CystA.1, the presence of a T residue at position 10 of CystA.1, or the presence of a T residue at position 8 of CystA.1.

We further disclose a method of diagnosis of a Group II disease, preferably acne, in an individual, the method comprising detecting any one or more polymorphisms selected from the group consisting of: the absence of a G residue at position 73 of CystA.1, the absence of a TG at positions 76 and 77 of CystA.1, and the absence of a C residue at position 6 of CystA.1.

The diagnosis methods in the two above paragraphs may be combined.

Diagnosis of Psoriasis

We disclose a method of diagnosis of a Group II disease, preferably psoriasis, in an individual, the method comprising detecting the presence of one or more polymorphisms selected from the group consisting of: the absence of a G residue at position 270 of CystA.1, the presence of a G residue at position 73 of CystA.1, the absence of a C residue at position 15 of CystA.1, the absence of a C residue at position 6 of CystA.1, the absence of a G residue at position 4 of CystA.1, and the absence of a G residue at position 7 of CystA.1.

Diagnosis of Eczema

We disclose a method of diagnosis of a Group I disease, preferably eczema, more preferably atopic eczema, in an individual, the method comprising detecting the presence of one or more polymorphisms selected from the group consisting of: the absence of AC at positions 122 and 121 of CystA.1, the absence of a G residue at position 110 of CystA.1, the presence of a t residue at position 85 of CystA.1, the presence of a G residue at position 73 of CystA.1, absence of an A residue at position 72 of CystA.1, the absence of a T at position 60 of CystA.1, the absence of a C at position 15 of CystA.1, the absence of an A residue at position 14 of CystA.1, the absence of a C residue at position 13 of CystA.1, the absence of a C residue at position 6 of CystA.1, the absence of a T residue at position 5 of CystA.1, the absence of a G residue at position 4 of CystA.1, and the absence of a G residue at position 7 of CystA.1.

### Example B5

### Identification of Cystatin A (CSTA) Coding Sequence Polymorphisms

Detection of Mutations in the Coding Sequence of Human Cystatin A

The Cystatin A coding sequence is screened using PCR for polymorphisms, as described above. Identified polymorphisms are shown in Tables B4.3 and Table B4.4 above.

### Example B6

### Analysis of Promoter Activity

We use the sequence of Exon 1 to perform a search in the human genome databases and identify a CSTA promoter region in RP11-299J3 clone (AC083798). We identify potential TATA and CAAT signals in that sequence. These signals are indicated in bold in the CSTA 5′ sequence below.

Four regulatory regions are located in the promoter sequence: three TPA responsive elements (TRE-1, TRE-2 and TRE-3) and one Ap-2 site. TPA or 12-O-tetradecanoylphorbol-13-acetate is a potent protein kinase C activator and AP-2 is an enhancer-binding protein.

5′region of CSTA
ttttatagttcatcaagtcaccgtgcccagcctccaattatttttatatttgtatgtgtaaatgaatgtctctgaag
gatacagaagaagttggtgccaataattgtctctggagagtagaagggtgtctgaggaattaagtgagtgaataatt
ccttatattgtaaagcctggttccttccaaacatttttatcctgtgtatgtattatatattcccaaaattgaataaa
ataatatacataaatattcacacaatgtggccattttgcttctagaccagaaacaacgaaaatcgtcatcaatagag
aaatgagtatgtaaaatatgaactagaatatagcaattagataggtagactaaatagataggtctagcataaaatag
catatcacagttaaaggaaatgaacaggattgatctatctgtggattgatctatgtgtatatcaacatagaaaggct
caaaaacatgttgaatagaaaaaggaacataacatagaatatatttagcatacaatttaagtgaaattttaaagaca
caccaagtaagacatattttaattttaaagacacacatataaaaatggcctggaaggatattaattcaccatgtact
ttgccttctggaaggcaaggttgcgtggggtgtggggcttcctccatatctgtaatattttatttcctaaaaataac
tacaaaaataaaaaacagcaagcaaatatgacaaaagggttaaaagttttaattctgagtgatagaaatatagatgt
ttgttattttattctttgtgttttaccgtatgttaaacatttccagatatttaaataagagtaaagaagacacatcc
agccaaggtcctccagatagatccttttgctttctttctaaagtcaagtaaattctaaactaaccttgacattatta
gtaagttttgctttaaaaaaaataaaattttgtgttagaagttttaaaacatttggaaattctagttgcggcttcag
atttcataattcagatgatgcaacaggatggaaccattgtcaaagagaatgcagggacgtttgatgcttgttaggac
atgactcctgtacttgcccatttgttcatcctccaacccctctttcttccaaattccatgtagcatattctctccag
gaagcaagaagacttgcctggcggcatactcattttccccatgcctctttgctgtttgtggaaaataaagcattcta
taggcggagctagtgaacgcctcttttaaaacacgagtctccacacttccctgttc(−1)

The sequences of the promoter region 1 of CSTA from controls and patients are aligned, and shown below.

| | | * * |
|---|---|---|
| cstape2f | 45 | -GTAAAT-CTAAACTAACCTTGAC-ATATTAGTAAGTTTT-GCTTTAAAAAAAATAAA |
| cstapp6f | 1 | --TAANT-CTAAACTAACCTTGAC-TTATTAGTAAGTTTT-GCTTTAAAAAAAATAAA |
| cstape1f | 27 | -GTAAAT-CTAAACTAACCT-GACATTATTAGTAAGTTTT-GCTTTAAAAAAAATAAA |
| cstapp8f | 3 | -GTAANT-CTAAACTAACCTTGACATTATTAGTAAGTTTTCGCTTTAAAAAAAATAAA |
| cstapcon | 61 | -GTAAATTCTAAACTAACCTTGACATTATTAGTAAGTTTT-GCTTTAAAAAAAATAAA |
| cstape8f | 44 | -NTAANT-CTAAACTAACCTTGGCATTATTAGTAAGTTTT-GCTTTAAAAAAAATAAA |
| | | * |
| cstape2f | 99 | ATTT-GTGTTAGAAGTTTTAAAACATTTGGAAATTCTAGTTGCGGCTTCAGATT-CAT |
| cstapp6f | 54 | ATTT-GTGTTAGAAGTTTTAAAACATTTGGAAATTCTAGTTGCGGCTTCAGATTTCAT |
| cstape1f | 61 | ATTTTGTGTTAGAAGTTTTAAAACATTTGGAAATTCTAGTTGCCGCTTCAGATTTCAT |
| cstapp8f | 59 | ATTT-GTGTTAGAAGTTTTAAAACATTTGGAAATTCTAGTTGCGGCTTCAGATTTCAT |
| cstapcon | 117 | ATTTTGTGTTAGAAGTTTTAAAACATTTGGAAATTCTAGTTGCGGCTTCAGATTTCAT |
| cstape8f | 99 | ATTTTGTGTTAGAAGTTTTAAAACATTTGGAAATTCTAGTTGCGGCTTCAGATTTCAT |
| | | TRE-2 |
| | | * * * |
| cstape2f | 155 | AATTCAGATG-ATGCAACAGGATGG-AACCATT-GTCAAAGAGAATGCAGGGACGTTTG |
| cstapp6f | 111 | AATTCAGATG-ATGCAACAGGATGG-AACCATT-GTCAAAGAGAATGCAGGGACGTTTG |
| estape1f | 139 | AATTCAGATG-ATGCAACAGGATGG-AACCATT-GTCAAAGAGAATGCAGGGACGTTTG |
| cstapp8f | 116 | AATTCAGATG-ATGCAACAGGATGG-AACCATT-GTCAAAGAGAATGCAGGGACGTTTG |
| cstapcon | 175 | AATTCAGATG-ATGCAACAGGATGG-AACCATT-GTCAAAGAGAATGCAGGGACGTTTG |
| cstape8f | 157 | AATTCAGATGGATGCAACAGGATGGGAACCATT-GTCAAAGAGAATGCAGGGCCGTTTG |
| | | * TRE-1 |
| cstape2f | 211 | -ATG-CTTGTTAGGACATGACTCCTG-TACTTGCCCATTTGTTCATCCTCCAACCCCTCT |
| cstapp6f | 167 | -ATG-CTTGTTAGGACATGACTCCTG-TACTTGCCCACTTGNTCATCCTCCAACCCCTCT |
| cstape1f | 195 | GATG-CTTGTTAGGACATGACTCCTG-TACTTGCCCATTTGTTCATCCTCCAACCCCTCT |
| cstapp8f | 172 | GNTG-CTTGTTAGGACATGACTCCNG-TACTTGCCCATTTGTTCATCCTCCAACCCCTCT |
| cstapcon | 231 | -ATG-CTTGTTAGGACATGACTCCTG-TACTTGCCCATTTGTTCATCCTCCAACCCCTCT |
| cstape8f | 215 | GATG-CTTGTTNGGACATGNCTCCTG-TNCTTGCCCATTTGGTCATCCTCCAACCCCNCT |
| cstape2f | 268 | TTCTTCCAAATTCCATG-TAGCATATTCTCTCCAGGAAGCAAGAAGACTTGCCTGGCGCC |
| cstapp6f | 224 | TTCTTCCAAATTCCATG-TAGCATATTCTCTCCAGGAAGCAAGAAGACTTGCCTGGCGGC |
| cstape1f | 253 | TTCTTCCAAATTCCATG-TAGCATATTCTCTCCAGGAAGCAAGAAGACTTGCCTGGCGGC |
| cstapp8f | 230 | TTCTTCCAAATTCCATG-TAGCATATTCTCTCCAGGAAGCAAGAAGACTTGCCTGGCGGC |
| cstapcon | 288 | TTCTTCCAAATTCCATG-TAGCATATTCTCTCCAGGAAGCAAGAAGACTTGCCTGGCGGC |
| cstape8f | 273 | TTCTTCCAAANNCCATG-TNGCNTNTTCTCTCCAGGAAGCANGACGACTTGCCTGGCGGG |

In the above alignment, TRE-1 and TRE-2 sequences are underlined. * indicates mutation positions. Cstaconr is sequence from GB AC083798 sequence.

Cstape2f, cstape1rf and cstape8f are sequences from eczema patients. Cstapp6f and cstapp8f are sequences from psoriasis patients.

Promoter Region 2

The sequences of the promoter region 2 of CSTA from controls and patients are aligned, and shown below.

| | | ** |
|---|---|---|
| cstappoly4r | 35 | CCA-TGT-AGCATATTCT-CTCCA-GG-AAGCAGAAGACTTGCCTGGCGG-GCATAC--T |
| cstappoly7r | 7 | CCNATGT-AGCATATTCT-CTCCA-GG-AAGCAAGAAGACTTGCCTGGCG-GCATAC--T |
| cstappoly3r | 31 | CCA-TGT-AGCATATTCT-CTCCA-GG-AAGCAAGAAGACTTGCCTGGCG-GCATAC--T |
| CSTAconr | 300 | CCA-TGT-AGCATATTCT-CTCCA-GG-AAGCAAGAAGACTTGCCTGGCG-GCATAC--T |
| cstappoly6r | 5 | CCA-TGT-AGCATATTCT-CTCCA-GN-AAGCAAGAAGACTTGCCTGGCG-GCATAC--T |
| cstapa2r | 75 | CCA-TGT-AGCATATTCT-CTCCAAGGAAAGCAAGAAN--T-GCCTGGCG-GCATAC--T |
| Cstapa1r | 42 | CCG-AGT-AGCATATTCT-CTCCAAGG-AAGC--GAAAACT-GCCTGGCG-GCATNACTN |
| cstape5r | 14 | CNATAGNGANGATATTCTTCTGCGAGG-AAGNA-GAGN--TTGCNTGGNGAGCAAGN--- |
| cstape7r | 1 | ---ATGT-AACAAATTCT-CTCCA-GG-AAACA--AAGN-TTGCNTGNCAGGCATAN--N |
| cstapp4r | 10 | CCNATGT--ACATATTCT-CTCCNAGG-AAGCAA-AAN--TTGCCTGGCGGGCATAC--T |
| cstapp1r | 1 | --------------------------------AACAGAGCTTGCCTGACGGGCATAAC-T |
| cstape9r | 25 | CCGATGT-AGCATATTCT-CTCCGAGG-AA-CAAGAGN--TTGCCTGCCGGGCATAC--T |
| cstapp6r | 82 | CCNATGT-AGCATATTCT-CTCCNAGG-AA--NANAGN--TTGCCTGGCGGGCATAC--T |
| cstapp7r | 3 | CCA-TGT-AGCATATTCT-CTCCNAGG-AAGCAAGAGN--T--CCTGGNG-GCATAC-TC |
| | | AP2-site |
| | | ** ** ** ** * * * |
| cstappoly4r | 87 | CATTTTCCCCATGCCTCT-TTGCTGTTTGTGGAAAATAAAGCATTCTATAGGCGGAGCT |
| cstappoly7r | 60 | CATTTTCCCCATGCCTCT-TTGCTGTTTGTGGAAAATAAAGCATTCTATAGGCGGAGCT |
| cstappoly3r | 83 | CATTTTCCCCATGCCTCT-TTGCTGTTTGTGGAAAATAAAGCATTCTATAGGCGGAGCT |
| CSTAconr | 352 | CATTTTCCCCATGCCTCT-TTGCTGTTTGTGGAAAATAAAGCATTCTATAGGCGGAGCT |
| cstappoly6r | 57 | CATTTTCCCCATGCCTCT-TTGCTGTTTGTGGAAAATAAAGCATTCTATAGGCGGAGCT |
| cstapa2r | 126 | CATTTTCCCCAAGCCTCT-TTGCTGTTTGTG--AAATAAAGCAT----TNAGCGGAGCT |
| Cstapa1r | 94 | CATTTTCCCCNAGCCTCTATTGCTGTTTGTG--AAANAAAGCAT----TNAGCGGAGCT |
| cstape5r | 67 | NGTTTTCGCC-AGCCTCT-TTGCTGTT--TGG-AAATAA--CAT-CTAT-GGCGGA-CT |
| cstape7r | 49 | CATTTTCNCCATGCCTCT-TTGCTGTT--TGG--AATAA--CAT-CTAT-GGCGGA-CT |
| cstapp4r | 61 | CATTTTCCCCGAGCCTCT-TTGCTGTT--TGGGAAAAAA---CT-CTAT-NGCGGA-CT |
| cstapp1r | 28 | CATTTTCNCCATGCCTCT-TTGCTGTT--TGG--AATAA--NAT-CTAT-GGCGGNCTA |
| cstape9r | 77 | CATTTTCCCCATGCCTCT-TTGCTGTT--TGG--AATAA--CAT-CTAT-GGCGGGCTT |
| cstapp6r | 133 | CATTTTCCCC-AGCCTCT-TTGCTGTT--TGG-AAATAA--NAT-CTAT-GGCGGACTA |
| cstapp7r | 53 | CATTTTCCCC-AGCCTCT-TTGCTGTTTGTGG--AATAA-GCAT--T-NAGGCG-AGCT |
| | | ** *** *** * ** |
| cstappoly4r | 145 | AGTGAACG-CCTCTTTTAAAACACGAG-TCTCCACNACTTCCCTGTTCA-TTTGGTTCCA |
| cstappoly7r | 118 | AGTGAACG-CCTCTTTTAAAACACGAG-TCTCCACA-CTTCCCTGTTCA-TTTGGTTCCA |
| cstappoly3r | 141 | AGTGAACG-CCTCTTTTAAAACACGAG-TCTCCACA-CTTCCCTGTTCA-TTTGGTTCCN |
| CSTAconr | 410 | AGTGAACG-CCTCTTTTAAAACACGAG-TCTCCACA-CTTCCCTGTTCACTTTGGTTCCA |
| cstappoly6r | 115 | AGTGAACG-CCTCTTTTAAAACACGAG-TCTCCACA-CTTCCCTGTTCCATTTGGTTCCG |
| cstapa2r | 178 | ATGAAACG-CCTCTTTTAAANT-CGAG-TCTC-ACA-CTTCC-TGTCC----TGGTTC-A |
| Cstapa1r | 147 | ATGGAACG-CCTCTTTTAAAAC-NGAG-TCTC-NCA-CTTCC-TGTCC----TGGTTC-- |
| cstape5r | 116 | AGTGAACG-CCTCTTTTAAA-CANGAC-TCCC---A-CNTCC---TGCC-TTTG--TCCC |
| cstape7r | 98 | AGTGAACG-CCTCTTTTAAA-CACG-G-TCCC---A-CNTCC----GCC-TTTG--TCCA |
| cstapp4r | 111 | AGTGAACG-CCTCTTTTAANNN--GAT-TCCC---A-CNTCC---TGCN-TTTG--TCCG |
| cstapp1r | 78 | AGTGAACG-CCTCTTTTAAA-CACGAN-TCCC---A-CNTCC---TGCC-TTTG--TCCN |
| cstape9r | 127 | AGTGAACG-CCTCTTTTAAA-CANGAGCTCCC---A-CNTCC---TGCCTTTTG--TCCN |
| cstapp6r | 183 | AGTGAACG-CCTCTTTTAAACN--GNN-TCCC---A-CNTCC---TGCC-TTTG--TCCG |
| cstapp7r | 103 | A-TGAACG-CCTCTTTTAAAAN-CGAG-TCTC--CA-CTTCC-TGTCC---TTG-TTCC- |

In the above alignment, the AP-2 sequence is underlined. * represents mutation positions. Cstappoly7r, cstapolyp3r, cstappoly4r, cstappoly6r are sequences from controls from different ethnic groups. Cstaconr is sequence from GenBank AC083798 sequence. Cstapa2r and cstapa1r are sequences from acne patients. Cstape5r, cstape7r and cstape9r are sequence from eczema patients. Cstapp7r, cstapp6r and cstapp4r are sequences from psoriasis patients.

It is clear that the promoter region of Cystatin A contains several mutation points that are present only in patients but not in control samples. Samples from acne and eczema present the greatest variations in the promoter sequence; therefore this region is altered in patients compare to normal. There is insertion of G in the TRE-2 region in cystatin A promoter of acne (cstapa3f) and eczema (cstape8) patients, suggesting that the CSTA promoter activity is altered in diseases with abnormal barrier (e.g. acne, eczema).

We therefore provide for a method of diagnosis of a disease, preferably a skin disease, preferably a skin inflammatory disease, the method comprising detecting the presence of a G residue in a TRE-2 region of a cystatin A nucleic acid.

Promoter Activity

The following experiment is conducted to identify the effects of these promoter and coding sequence polymorphisms in expression of cystatin A.

PCR Cloning Strategy

We designed two primers in the promoter region Forward 5′GCTTCCTCCATATCTGTA3′ and reverse 5′AGATAAGCCTCCAGGTATC3′, and performed PCR using DNA from normal (cstappoly4r, cstappoly3r and cstappoly7r) and eczema (cstape8f, cstape5r, cstape7r) patients. The cloning strategy is similar to the one described by Takahashi et al, 1998. PCR products are subcloned in pCR™ 2.1 vector (Invitrogen, San Diego, Calif.) and the HindIII/XbaI-digested fragments from recombinant plasmids are inserted into the promotersless 0-CAT plasmids.

5 μg of Fragments-CAT plasmids are used to transfect 10⁵ SVHK cells by liposome method using Lipofectin. After 48 h, cells are collected and a CAT assay is performed.

Results

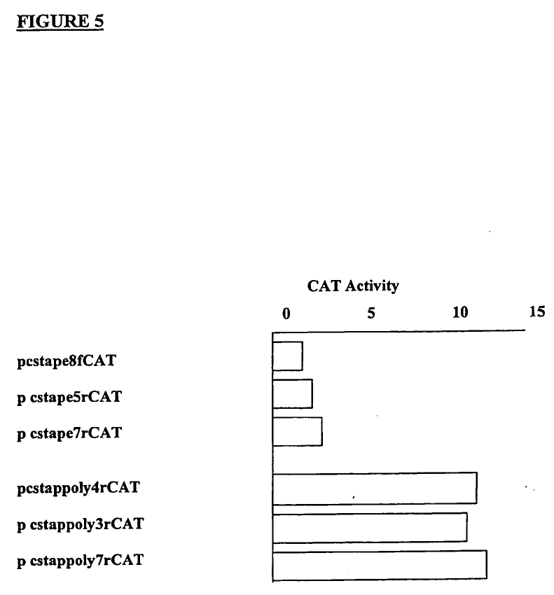

The results are shown in FIG. 5.

There is significant decrease in the activity of Cystatin promoter of eczema patients compared to control promoter (see FIG. 5). These results suggest that CSTA is involved in the skin barrier homeostatis. CSTA is down-regulated in diseases with defective skin barrier (e.g. eczema). Up-regulation of CSTA could also induce an increase in the adhesion between epithelial cells. This suggests that CSTA could be also involved in diseases with enhance skin barrier.

Detection of mutations within the promoter region of CSTA may therefore be used for diagnostic of patients with abnormal skin barrier (e.g eczema or any Group I disease). This diagnosis is an important step for identifying the appropriate treatment. For example detection of mutations (e.g. G insertion in TRE-2 sequence TGGATGCA) in eczema patients suggest that CSTA has very weak activity and application of one or combination of peptides that mimic the inhibition activity of CSTA to the patient skin would be the most appropriate treatment in this case.

### Examples C

### Proteolysis profiles/Protease Mediated Maturation of Desmosomal and Corneodesmosomal Proteins

### Example C1

### Production of Recombinant SCTE

Stratum Corneum Tryptic Enzyme (SCTE) is cloned and expressed in an active form such that it has full enzymatic activity.

Expression Vectors Used

Expression is achieved using different vector systems in order to achieve protein expression at a high level, and enable purification. Three vector systems are used

Insect Cell System: No His-Tag at terminus; Invitrogen vector-pMT/V5-HisA, B or C are used, depending on the reading frame. Expression in S2 insect (Drosophila) cells; purification as described in Hansson et al., 1994.

Retroviral System: No His-Tag; Clontech retroviral vectors (pLNCX2) Protein is expressed in murine mammary cells, and purified as described in Hansson et al., 1994.

pcDNA3.1 System: His-Tag at the C-terminal; Invitrogen vectors (pcDNA3.1, ABC with three ORFs) used. Expression of protein is achieved in COS-7 cells (Monkey African Green Kidney Cells).Reverse Transcription-Polymerase Chain Reaction (RT-PCR)

PCR Primers, covering the open reading frame, are designed manually i.e. by sight using SCTE sequence (GB: AF168768). All primers are from Invitrogen

| | |
|---|---|
| Forward (5′ TO 3′): | GGA AAT CAG GTG GAG CG |
| Reverse (5′ TO 3′): | GAT GAG TCA GGA GTT GGC |

Human epidermis total RNA ISreverse transcribed to cDNA with the Applied Biosystems Gold RNA PCR Kit. The RT Cycle comprises hybridisation for 10 mins at 25° C., followed by Reverse Transcription for 12mins at 42 ° C. PCR is performed using Applied Biosystems Gold Taq Polymerase with 2.0 mM Mg²⁺, 40 cycles. Each PCR Cycle consists of Taq Activation 95° C. for 10 mins, Denaturation: 94 for 30 sec, Annealing: 58.8° C. for 1 min, Extension: 72 ° C. for 20 seconds and Annealing/Extension: 72° C. for 7 mins

PCR amplification is verified on a 1.5% agarose gel. PCR products are purified from the other PCR mixture components using the Stratagene Strataprep PCR Purification Kit. The purified SCTE PCR product is used as the template for restriction enzyme PCRRestriction Enzyme Site Flanked PCR

The insect cell vector pMT/V5-His (Invitrogen, Cat #K4120-20) is chosen for cloning. This vector is available in 3 reading frames, A, B and C. Each reading frame facilitates cloning with expression of the C-terminal peptide. The correct reading frame is chosen with respect to the start codon of the SCTE DNA insert.

Restriction enzyme (RE) sites around the multiple cloning site of the vector, which are in frame with the SCTE start codon, are chosen. The SCTE DNA sequence is screened for the presence of restriction enzyme sites using the Webcutter program found on the NIH web site (www.nih.gojp/˜jun/research/anal.html). Those restriction enzyme sites not found in the SCTE sequence, but found on the vector are potentially available for use.

The SCTE start codon is found to be in frame with the pMT/V5-HisC vector. Kpn1 is chosen as the first restriction enzyme, at the start codon. Not1 is chosen as the second primer, after the stop codon. Restriction enzyme sites are incorporated into the forward and reverse SCTE PCR primers in order to generate a SCTE PCR product flanked by Kpn1 and Not1. These sites enable the sequence to be inserted into the vector:

SCTE Forward Restriction Enzyme Primer (5′ to 3′)
ATTAGTA-GGGTAG-ATGGCTACAGCAAGACCC
Random Sequence-Kpn1 Restriction Enzyme Sequence-SCTE Start codon sequence
SCTE Forward RE Primer (5′ to 3′)
ATTAGTA GGTACC ATGGCTACAGCAAGACCC
• Random SequenceKpn1 RE Seq SCTE Start codon sequence
SCTE Reverse RE Primer (3′ to 5′)
1) TCCAGGCCAACTC CTGA GCGGCCGC ATATTA
2) SCTE Stop codon sequence Not1 RE Seq Random Sequence
SCTE Reverse RE Primer (5′ to 3′) (compliment)
TAATATGCGGC CGCTCAGGAG TTGGCCTGGA

SCTE PCR product from standard primer PCR is used as the template for Restriction Enzyme-primer PCR. PCR conditions are 2.5 mM Mg²⁺ at any annealing temperature between 57° C. and 67° C. The SCTE-Restriction Enzyme PCR product is digested with the corresponding restriction enzymes (Kpn1 and Not1) according to the manufacturers recommended protocol (Promega). The digested SCTE-Restriction Enzyme PCR product is purified from the restriction enzyme mix by agarose gel electrophoresis and the DNA purified from the gel using the Qiagen QiaexII Agarose Gel Extraction Kit. The SCTE insert is now ready for ligation to the prepared vector.

PMT/V5-HisC Vector Propagation and Restriction Enzyme Digestion

The pMT/V5-HisC vector is propagated in Invitrogen TOP10 chemically modified E. Coli Cells (Invitrogen, Cat #C4040-10). The protocol is described in the TOP10 information booklet: Add 1 μl of the vector to a vial of TOP10 cells (which have been thawed on ice). Gently mix. Incubate for 5 to 30 mins on ice. Heat-shock cells for 30 seconds at 42 ° C., without shaking. Immediately transfer back to ice. Add 250 μl of room temperature SOC buffer. Cap the tube tightly and shake to tube horizontally at 37° C. for 1 hour. Evenly spread 50 μl of transformant mix onto a fresh agar plate containing ampicillin. Incubate overnight at 37° C. Single colonies are picked up and used to inoculate ampicillin-LB, and the culture incubated overnight at 37° C. Glycerol stocks are made of the cultures. The propagated pMT/V5-HisC vector is purified from the bacteria using the Qiagen DNA Mini-Prep Kit, according to the manufacturers recommended protocol.

Purified vector is digested with Kpn1 and Not1, as for the SCTE-Restriction Enzyme PCR product. Digested vector is also purified from the restriction enzyme mix by agarose gel electrophoresis and the DNA purified from the gel using the Qiagen QiaExII Agarose Gel Extraction Kit. The vector is now ready for ligation to the prepared SCTE insert.

pMT/V5-HisC and SCTE Vector Ligation

The SCTE is ligated to the pMT/V5-HisC vector using the enzyme T4-DNA Ligase (Gibco, Cat #15224-017). Ligation performed at a ratio of 5:1, DNA insert:vector according to the following protocol: DNA Ligase (1 U/μl) 1 μl, 5× Ligation Buffer 4 μl, SCTE Insert (X μg/μl), pMT vector ((X μg/μl), Water (up to 20 μl). The ligation reaction is incubated at 16° C. overnight (floating on icey water at 16° C.). The ligation reaction is assessed by 0.7% agarose gel (compared to unligated control samples). Competent E-Coli cells are transformed with ligated SCTE/Vector construct for propagation.

Propagation of SCTE/Vector Construct in E-Coli CelisTOP10 cells are transformed with the construct according to the manufacturers protocol, described above. The transformed cells are streaked onto ampicillin-agar plates and incubated overnight at 37° C. Single colonies are used to inoculate ampicillin-LB and the culture incubated overnight at 37 ° C. Glycerol stocks have been made of the cultures. The construct is purified using the Qiagen DNA Mini-Prep Kit.

Lipid Transfection of S2 Insect Cells

S2 Insect cells (drosophila, from Invitrogen, Cat #R690-07) are cultured as recommended by the manufacturer—in Drosophila Expression Medium (DES, Cat #Q500-01) supplemented with foetal calf serum and penicillin-streptomycin. Transfection is performed using Cellfectin (Invitrogen, Cat #10362-010), a liposome formulation, again according to the manufacturers protocol.

Induction of SCTE Protein Expression/Formation of Stable Cell Lines

The pMT/V5-HisC vector has a metal-inducible (inducible with copper sulphate) promoter, metallothionein (MT), which enables high level expression of the target gene in S2 cells. Protein expression in the cells is induced with copper sulphate, added to a final concentration of 500 μM. Cells are incubated for 1-4 days. Following induction, cells are harvested and stored until further analysis. Polyacrylamide gel electrophoresis (PAGE) analysis is used to determine whether recombinant protein expression has occurred by comparing the total protein of induced cells and non-induced cells.

Formation of Stable Cell Lines

After demonstrating that the SCTE protein is expressed in S2 cells, expression is scaled up by creating stable cells lines. Cells produce blasticidin, which is a potent translational inhibitor in prokaryotic and eukaryotic systems. Resistance to blasticidin is conferred after co-transfection of the SCTE/pMT/V5-HisC construct and pCoBlast selection vector (Invitrogen, Cat #1 K5150-01) into S2 cells. The protocol is found in the Drosophila expression system (DES) manual, which accompanies all DES reagents. Blasticidin (25 μg/ml) is used to select stable transfectants.

Scale-Up of Protein Expression

Expression is scaled up for protein purification by using larger volumes/flasks. The Invitrogen manual enclosed with the S2 cells provides details of a protocol for doing this. Purification is performed by FPLC. Different fractions are collected and analysed for the presence of the recombinant protein.

### Example C2

### Production of Recombinant SCCE

Stratum Corneum Chymotryptic Enzyme (SCCE) is cloned and expressed in an active form such that it has full enzymatic activity.

Reverse Transcription-Polymerase Chain Reaction (RT-PCR)

PCR Primers, covering the open reading frame, are designed manually i.e. by sight using SCCE sequence (GB: NM005046). All primers are from Invitrogen.

| | |
|---|---|
| Forward (5′`TO 3′): | CGG GCT CCA TGG CAA GAT C |
| Reverse (5′`TO 3′): | GCG TCC TCA CTC CTG TGC |

Human epidermis total RNA reverse transcribed to cDNA with the Applied Biosystems Gold RNA PCR Kit. RT Cycle is as previously described for SCTE, using 2.0 mM Mg²⁺, 40 cycles, PCR Cycle is as described for SCTE, except that annealing is done at 60° C. for 1 min.

Restriction Enzyme Site Flanked PCR

The correct reading frame and Restriction Enzyme sites around the multiple cloning site is chosen with respect to the start codon of the SCCE DNA insert. The SCCE DNA sequence is screened for the presence of restriction enzyme sites using the Webcutter program, and restriction enzyme sites chosen as described previously. The SCCE start codon is found to be in frame with the pMT/V5-HisA vector. EcoR1 is chosen as the first restriction enzyme, at the start codon. Xho1 is chosen as the second primer, after the stop codon.

Restriction enzyme sites are incorporated into the forward and reverse SCCE PCR primers in order to generate a SCCE PCR product flanked by EcoR1 and Xho1. These sites enable the sequence to be inserted into the vector:-SCCE Forward

SCCE Forward Restriction Enzyme Primer (5′ to 3′)
GCC AGC-GAA TTC-ATGGCA AGA TCC CTT CTC
Random Sequence—EcoRl Restriction Enzyme Sequence—
SCCE Start codon sequence
SCCE Reverse Restriction Enzyme Primer (3′ to 5′)
ATGAAAAAGCATCGCTAA-CTCGAG-AGCACT
SCCE Stop codon sequence—Xho1 Restriction Enzyme
Sequence—Random Sequence
SCCE Reverse Restriction Enzyme Primer (5′ to 3′)
(compliment)
AGTGCTCTCG AGTTAGCGAT GCTTTTTCAT

SCCE PCR product from standard primer PCR is used as the template for Restriction Enzyme-primer PCR. PCR conditions are 2.0 mM Mg²⁺ at an annealing temperature of 62° C. The SCCE-Restriction Enzyme PCR product is digested with the corresponding Restriction Enzyme's (EcoR1 and Xho1)—according to the manufacturers recommended protocol (Promega). The digested SCCE-Restriction Enzyme PCR product is purified from the restriction enzyme mix by agarose gel electrophoresis and the DNA purified from the gel using the Qiagen QiaexII Agarose Gel Extraction Kit. The SCCE insert is now ready for ligation to the prepared pMT/V5-HisA vector.

Expression is scaled up for protein purification by using larger volumes/flasks. The Invitrogen manual enclosed with the S2 cells provides details. Purification is performed by FPLC. Different fractions are collected and analysed for the presence of the recombinant protein. Further details for expression and purification of SCCE are disclosed in Hansson et al., 1994 (J. Biol. Chem. 269 (30), 19420-19426), which describes the purification of the protease enzyme SCCE.

### Example C3

### Cleavage of Adhesion Proteins with Recombinant SCCE and SCTE

Recombinant ProSCCE is produced and purified as described above. Recombinant ProSCCE is activated with agarose-bound trypsin, as described in Brasttsand & Egelrud, 1999.

Proteins are extracted from human epidermis in the presence of a detergent (TENP-40 buffer extract) and incubated with active SCCE for 37 C. for 4 h. The reactions are stopped by boiling for 2 mins after Laemmli's sample buffer is added. The proteins are immunoblotted with antibodies against corneodesmosomal proteins.

Cleavage of Adhesion Proteins by SCCE

We demonstrate with this Example that recombinant SCCE is able to cleave adhesion proteins, in particular, Corneodesmosin, Plakoglobin Desmoglein, Desmoplakin, Envoplakin and Desmocollin. Therefore, these adhesion proteins are substrates of this protease.

SCCE and Corneodesmosin: Corneodesmosin is found to be proteolysed by recombinant SCCE. The native form of corneodesmosin has a molecular weight of between 50 and 56 kDa. After 2 h incubation the major forms of Corneodesmosin are 36 and 46-43 kDa.

SCCE and Plakoglobin: Plakoglobin is found to be proteolysed by recombinant SCCE. The native form of plakoglobin has a molecular weight of between 85 kDa and 75 kDa After 2-4 h incubation the major form of Plakoglobin is 70 kDa

SCCE and Desmoglein: Desmoglein is found to be proteolysed by SCCE. After 2-4 h incubation the major forms of Desmoglein are 95 and 80 kDa which are the proteolytic products of the 160 kDa form.

SCCE and Desmoplakin: Desmoplakin is found to be proteolysed by SCCE. The native form of Desmoplakin has a molecular weight of between 190-250 kDa. After 2-4 hour incubation the major forms of desmoplakin are 120-180 kDa and 75-80 kDa.

SCCE and Envoplakin: Envoplakin is found to be proteolysed by SCCE. The native form of Envoplakin has a molecular weight of between 124-209 kDa. After 2-4 hour incubation the major forms of envoplakin are 100-120 kDa, 60-80 kDa and 50-55 kDa.

SCCE and Desmocollin: Desmocollin is found to be proteolysed by SCCE. The native form of Desmocollin has a molecular weight of between 70-80 kDa. After 2-4 hour incubation the major forms of desmocollin are 60-70 kDa and 50-60 kDa.

Cleavage of Adhesion Proteins by SCTE

We demonstrate with this Example that recombinant SCTE is able to cleave adhesion proteins, in particular, Corneodesmosin, Plakoglobin Desmoglein, Desmoplakin, Envoplakin and Desmocollin. Therefore, these adhesion proteins are substrates of this protease.

SCTE and Corneodesmosin: Corneodesmosin is found to be proteolysed by recombinant SCTE. After 2 h incubation the major forms of Corneodesmosin are 36 and 46-43 kDa

SCTE and Plakoglobin: Plakoglobin is found to be proteolysed by recombinant SCTE. After 2-4 h incubation the major form of Plakoglobin is 70 kDa

SCTE and Desmoglein: Desmoglein is found to be proteolysed by SCTE. After 24 h incubation the major forms of Desmoglein are 95 and 80 kDa which are the proteolytic products of the 160 kDa form.

SCTE and Desmoplakin: Desmoplakin is found to be proteolysed by SCTE. After 2-4 hour incubation the major forms of desmoplakin are 120-180 kDa and 75-80 kDa.

SCTE and Envoplakin: Envoplakin is found to be proteolysed by SCTE. After 24 hour incubation the major forms of envoplakin are 100-120 kDa, 60-80 kDa and 50-55 kDa.

SCTE and Desmocollin: Desmocollin is found to be proteolysed by SCTE. After 2-4 hour incubation the major forms of desmocollin are 60-70 kDa and 50-60 kDa.

### Example C4

### Proteolysis Profiles Materials and Methods

Polyclonal Antibody Production

Antibodies to corneodesmosin, SCCE, envoplakin, desmoplakin, desmocollin 1 and SLPI are produced in rabbits by injection of synthetic peptides conjugated with keyhole limpet hemocyanin (KLH).

Peptides are coupled to a keyhole limpet hemocyanin (KLH) using standard procedures. The peptides are designed comprising the following amino acid sequences, derived from the relevant GenBank sequences as shown in Table C4.1 below:

TABLE C4.1
Bold C residues are synthesised for coupling and
do not exist in the native sequence.

| Protein | Peptide Sequence | Name | GenBank Sequence |
|---|---|---|---|
| Corneodesmosin | FTKENPVKGSPGVC | SB2 | GB: AF030130 |
| SCCE | INDTMKKHR | SB1 | GB: XM_009002 |
| SCCE | RRAQRIKASKS | SB4 | GB: XM_009002 |
| Envoplakin | SASPTVPRSLR | SB3 | GB: XM_008135 |
| Desmoplakin | SGKRDKSEEVQC | 642 | GB: XM_004463 |
| Desmocollin 1 | MENSLGPFPQC | 641 | GB: XM_004463 |
| SLPI | CGKSCVSPVKA | 644 | GB: X04502 |

Rabbits are injected with peptide coupled to KLH Rabbits at day 1. A brief protocol follows. Day 1: mix approximately 150 μl (containing 300 μg of conjugate) with an equal volume of Freunds Complete Adjuvant by passing several times through a 23 G needle until an emulsion (which does not separate on standing) is formed. Inject subcutaneously into the rabbit using a 25 G needle. Day 22, repeat the above but use Freunds Incomplete Adjuvant. Day 43, repeat the above exactly. Day 53, take first test bleed from ear vein. Boost and re-bleed as necessary.

Monoclonal Antibodies

Monoclonal antibodies against adhesion protein antigens are obtained commercially from Biotechnic, Germany. These comprise anti-plakoglobin antibody directed against PG5.1 (Plakoglobin (NM-021991)) and anti-desmoglein 1 (XM-008810) antibody directed against Dsg1 and Dsg2.

Protein Extraction from the Epidermis

Proteins are extracted from biopsies in the form of “dog ears”, which are triangular pieces of skin removed to produce a neat linear scar. This is not skin removed from the mastectomy specimen sent to the pathologists for diagnostics. All samples are from female patients attending clinics at the University of Sheffield undergoing mastectomy for breast carcinoma. Informed consent is obtained from the patients.

The epidermis is separated from the dermis by incubating a breast biopsy from breast surgery with trypsin solution A (Life Technology, France) for 18 h at 4 C. The epidermis is divided on two parts. One part is used for protein extraction and the other for RNA extraction (see below). The peeled epidermis is heated 5 mins in phosphate-buffuered saline at 56 C. (Simon et al, 1997). The epidermis is homogenized on ice on in equal volume of the following buffers (three times in each buffer): 40 mM Tris-HCl, pH 7.5, 10 mM EDTA, 0.25 mM phenylmethylsulfonyl fluoride, and 2 μg/ml each of aprotinin, pepstatin A, and leupeptin (TE buffer), TE buffer containing 0.5% Nonidet P40 (TE-Nonidet P-40 buffer).

The pellet is then divided into three parts that are extracted in one-third of the original volume of TE buffer containing various concentrations of urea (4, 6, and 8 M) (TEU buffers). After each extraction, the homogenates are centrifuged for 15 mins at 15,000×g, and supernatants are kept at −30 C. until used. Finally, the pellet corresponding to the last extraction in 8 M urea is homogenized in 35 mM Tris-HCl, pH 6.8, 8M urea, 50 mM dithiothreitol, 5% glycerol, 0.25 mM phenylmethylsulfonyl fluoride, and 2 μg/ml each of aprotinin, pepstatin A, and leupeptin (TUDTT buffer), incubated at 95 C. for 30 mins, and centrifuged as described above (this method is originally described by Simon et al, 1997). Protein concentrations are measured using the Coomassie Plus protein assay (Pierre Chemical Co, Rockford, Ill.).

Protein Extraction From Stratum Corneum

Adhesion proteins are extracted from the stratum corneum according to a method described by Guerrin et al, 1998. Briefly, a tape strip is applied to a biopsy from normal skin or lesional and non-lesional of a patient with psoriasis. The tape strips are incubated in acetone and the tissue is recovered by centrifugation (500×g, 1 min), washed in acetone, and air-dried. The powder is boiled for 10 mins in 62.5 mM Tris-HCl, pH 6.8, containing 2% SDS and 50 μM DTT, and the solution is centrifuged (10,000×g, 10 mins).

Proteins are extracted from the stratum corneum of normal and psoriatic patient groups, as described above, and analysed by Western blots using specific antibodies against corneodesmosomal proteins. We show that the profile of epidermally extracted proteins differs between normal and diseased (psoriatic) individuals.

Western Blot

Proteins from the epidermal and stratum corneum biopsies (˜1 μg) are separated by 10% SDS-polyacrylamide gel electrophoresis (SDS-PAGE). After electrophoresis the proteins are electrotransferred to nitrocellulose membranes. Membranes are blocked overnight at 4° C. in Blotto (3% milk powder, 2% BSA, 0.1% Tween 20 in TBS). The membranes are probed with the primary antibodies described below for 3 hours at room temperature with agitation. Mouse monoclonal antibodies directed against plakoglobin and desmoglein are obtained from Progen (Heidelberg, Germany) and used at a 5 μg/ml concentration, diluted in Blotto. Rabbit polyclonal antibodies directed against peptides designed from protein sequences of desmocollin, desmoplakin, SLPI, SCCE, S protein and envoplakin (Antibody Resource Centre, Sheffield University, UK) are used at a 1:250 dilution. Membranes are washed for 3× 5mins in Blotto and incubated in the presence of the secondary antibody, either anti-mouse or anti-rabbit IgG HRP obtained from Santa Cruz Biotechnology, Inc. (California, USA) at a 1:1000 dilution for 1 hour at room temperature, with agitation. The membranes are washed in TBS-Tween 20 (0.1%) for 3×5 mins and proteins detected using the ECL+Plus™ western blotting detection system (Amersham Pharmacia Biotech, Little Chalfont, UK).

### Example C5

### Psoriasis Proteolysis Profiles of Proteins Extracted from Epidermis

Proteins are extracted from the epidermis of normal and psoriatic patient groups (from lesional and non-lesional skin), as described above, and analysed by Western blots using specific antibodies against corneodesmosomal proteins. We show that the profile of epidermally extracted proteins differs between normal and psoriatic lesional and non-lesional skin.

Corneodesmosin Proteolysis Profile

The deficiency in proteolysis of desmo/corneodesmosomal proteins is reflected in a significant decrease in the quantity of mature forms of corneodesmosome proteins in psoriatic skin compared to normal. Proteolysis deficit causes the cells to stick together in This consists of a significant decrease in the quantity of mature forms of corneodesmosome proteins in psoriatic skin compared to normal. Proteolysis deficit causes the cells to stick together in the surface of the epidermis preventing cell shedding.

For example, the 36 and 46-43 kDa forms of corneodesmosin are the major forms in the stratum corneum in normal skin and the 52-56 kDa form is very rare in normal stratum corneum. The corneodesmosin proteolysis profile in psoriatic skin is different. Thus, in psoriatic skin, 52-56 kDa form of corneodesmosin is dominant form in the epidermis of psoriatic patients and persists in the stratum corneum. This suggests that there is a deficit in proteolysis in psoriatic skin.

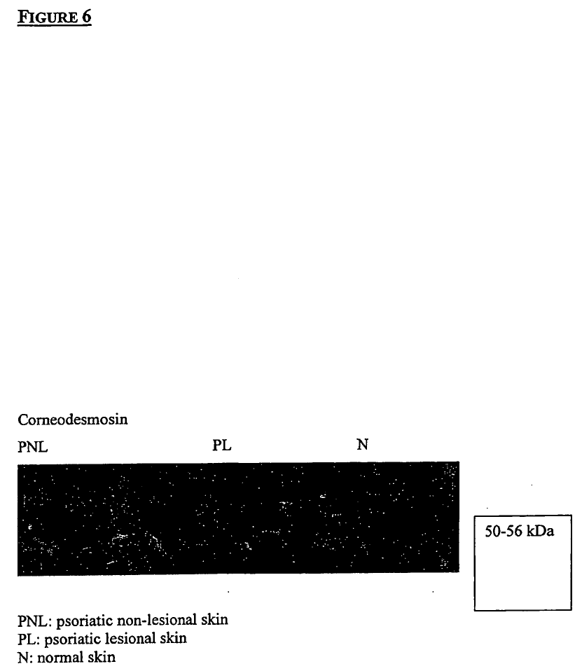

The results are shown in FIG. 6.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of one or more 52-56 kDa corneodesmosin polypeptides in an individual. We also disclose the diagnosis of a Group II disease or susceptibility to such a disease (preferably psoriasis or susceptibility to psoriasis) in an individual, by detecting the absence of or a modulated level, preferably a lower level of one or both of 36 kDa and 46-43 kDa corneodesmosin polypeptides in an individual.

The diagnosis may also be achieved by assaying the relative abundance of the 36 kDa, 46-43 kDa and 52-56 kDa polypeptides.

Preferably, the relevant polypeptides are detected in an epidermis of an individual, whether in vivo or ex-vivo (e.g., in a biopsy).

Desmoglein I Proteolysis Profile

Similar results are obtained with desmoglein I (DG I). In the epidermis of normal skin the most abundant forms of DG I are 95 and 80 kDa which are the proteolytic products of the 160 kDa form. However, in the stratum corneum of psoriatic skin the most abundant form is the 160 kDa. This suggests that there is a deficit of proteolysis of DG I in psoriatic skin.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of a 160 kDa desmoglein I polypeptide in an individual. Such diagnosis may also be done by detecting the absence of or a modulated level, preferably a lower level of one or both of a 95 and 80 kDa desmoglein I polypeptide in an individual.

The diagnosis may also be achieved by assaying the relative abundance of the 80 kDa, 95 kDa and 160 kDa polypeptides. The diagnosis may be done by detecting lack of proteolysis of a 60kDa desmoglein I polypeptide in an individual.

Preferably, the relevant polypeptides are detected in an epidermis of an individual, whether in vivo or ex-vivo (e.g., in a biopsy).

Desmoglein 3 Proteolysis Profile

Desmoglein 3 (Dsg 3) also shows a decrease in proteolysis. The bands corresponding to the two proteolysis fragments 55 kDa and 100 kDa are weaker in the stratum corneum of psoriatic skin compared to the stratum corneum of normal skin. Both fragments react with the same antibody directed against the cytoplasmic domain. Another band of 80 kDa appears as a proteolysis product of Dsg 3 more strongly in normal compared to psoriatic epidermis.

We therefore disclose the diagnosis of a Group II disease or susceptibility to such a disease (preferably psoriasis or susceptibility to psoriasis) in an individual, by detecting any of the changes described above, preferably the absence of or a modulated level, preferably a lower level of any one, pair or all of a 55 kDa desmoglein 3 polypeptide, an 80 kDa desmoglein 3 polypeptide and a 100 kDa desmoglein 3 polypeptide in an individual.

Preferably, the relevant polypeptides are detected in an epidermis of an individual, whether in vivo or ex-vivo (e.g., in a biopsy).

Plakoglobin Proteolysis Profile

Plakoglobin also shows a significant decrease in proteolysis in the epidermis of psoriatic patients compared to normal epidermis.

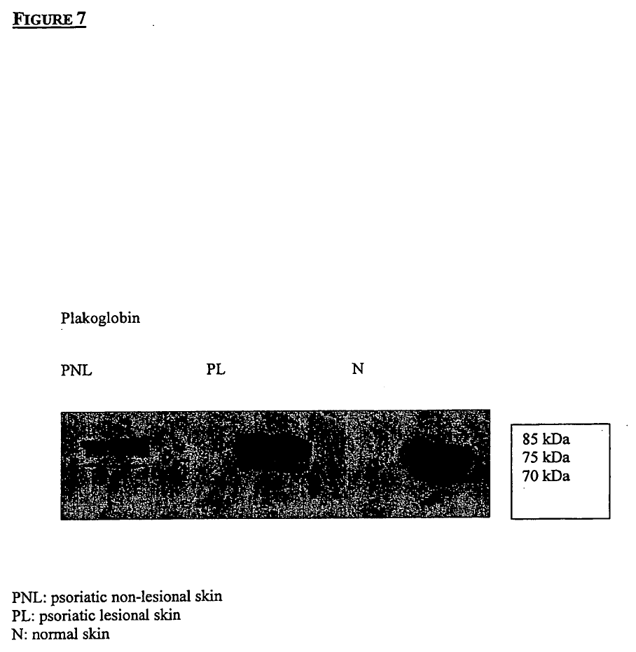

The results are shown in FIG. 7.

Antibody to plakoglobin reveals three bands 85, 75 and 70 kDa. The 70 and 75 kDa are proteolytic forms of the native protein (85 kDa) which is cleaved by proteases such as SCCE and SCTE during keratinocyte differentiation. The 70 kDa is very strong in psoriatic skin (both lesional and non-lesional) and is almost absent in the normal epidermis. Psoriatic and normal skin have two different profiles. The 70 kDa band can be use for diagnosis of psoriasis.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of a 70 kDa plakoglobin polypeptide in an individual. We also disclose the diagnosis of a Group II disease or susceptibility to such a disease (preferably psoriasis or susceptibility to psoriasis) in an individual, by detecting the absence of or a modulated level, preferably a lower level of an 75 kDa plakoglobin polypeptide.

The diagnosis may also be achieved by assaying the relative abundance of the 85 kDa, 75 kDa and 70 kDa polypeptides.

Preferably, the relevant polypeptides are detected in an epidermis of an individual, whether in vivo or ex-vivo (e.g., in a biopsy).

Desmoplakin Proteolysis Profile

Desmoplakin is a component of cytoplasmic plaque of the corneodesmosomes. It is glycoprotein thought to be involved in cohesion between keratinocytes and tranduction of the signal.

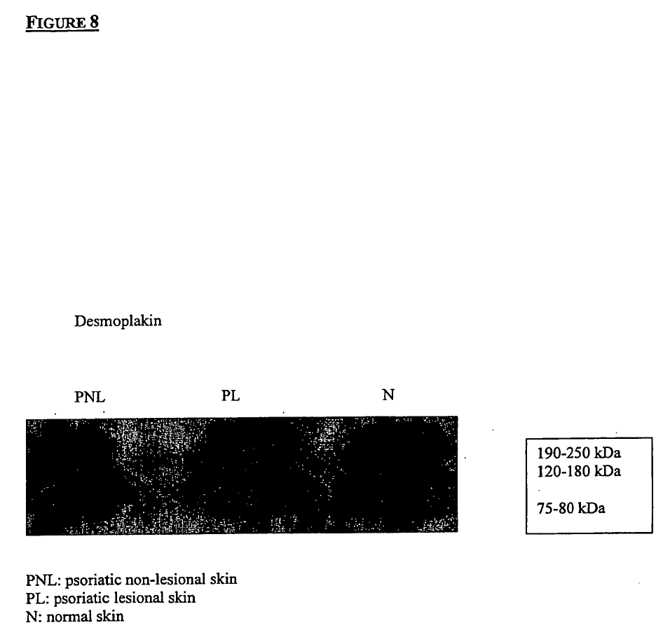

The results are shown in FIG. 8.

Using polyclonal antibody to desmoplakin we see three bands 190-250; 120-180 and 75-80 kDa. 190-250 and/or 120-180 are strongly expressed in normal compared to the disease. The 75-80 kDa could be the proteolysis form of 190-250 and/or 120-180, suggesting that desmoplakin is not proteolysed properly in psoriatic skin (lesional and non-lesional skin). These profiles can be used to identified diseases with increase adhesion such as psoriasis.

We therefore disclose the diagnosis of a Group II disease or susceptibility to such a disease (preferably psoriasis or susceptibility to psoriasis) in an individual, by detecting any of the changes described above, preferably the absence of or a modulated level, preferably a lower level of a either or both of 190-250 kDa and 120-180 kDa polypeptides of desmoplakin in an individual. The diagnosis may also be achieved by assaying the relative abundance of the 75-80 kDa, 190-250 and/or 120-180 polypeptides. The diagnosis may be done by detecting lack of proteolysis of a 85kDa desmoplakin polypeptide in an individual.

Preferably, the relevant polypeptides are detected in an epidermis of an individual, whether in vivo or ex-vivo (e.g., in a biopsy).

Desmocollin 1 Proteolysis Profile

Desmocollin 1 (Dsc1) is strongly expressed in the suprabasal layers of the normal epidermis.

The results are shown in FIG. 9.

Antibody to desmoscollin reveals three Dsc1 isoforms in normal epidermis at molecular weigh 70-80; 60-70 and 50-60 kDa. Only two bands are detected in the epidermis of psoriatic skin including lesional and non-lesional skin, suggesting that the proteolysis process is impaired in psoriatic skin. The absence of a 50-60 kDa band could be used for proteomic diagnosis of disease with enhance adhesion such as psoriasis.

We therefore disclose the diagnosis of a Group II disease or susceptibility to such a disease (preferably psoriasis or susceptibility to psoriasis) in an individual, by detecting any of the changes described above, preferably the absence of or a modulated level, preferably a lower level of a 50-60 kDa Desmocollin 1 polypeptide in an individual.

The diagnosis may also be achieved by assaying the relative abundance of the 70-80 kDa, 60-70 kDa and 50-60 kDa polypeptides.

Preferably, the relevant polypeptides are detected in an epidermis of an individual, whether in vivo or ex-vivo (e.g., in a biopsy).

Envoplakin Proteolysis Profile

Envoplakin is protein of the cornified envelope. It is important protein in the cohesion between keratinocytes.

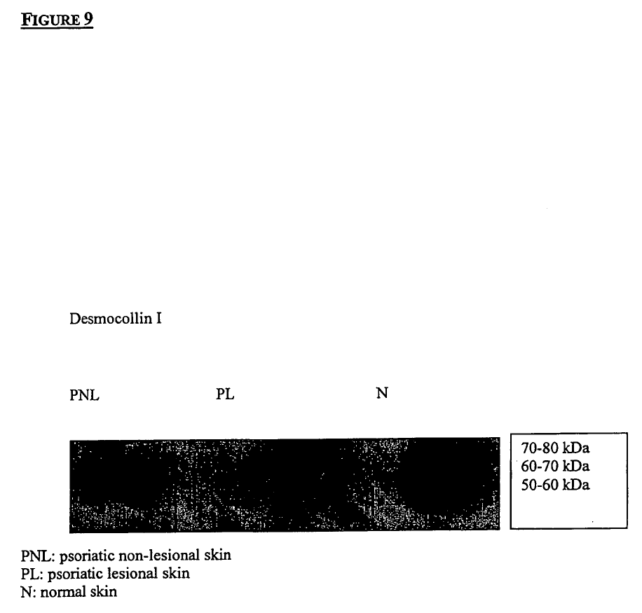

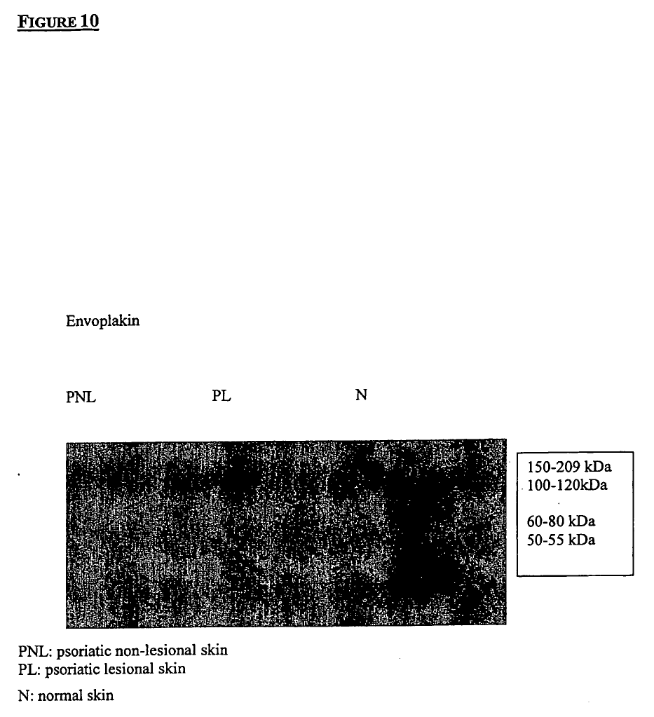

The results are shown in FIG. 10.

Antibody to envoplakin revealed four bands 124-209; 100-120; 60-80 and 50-55 kDa 100-120; 60-80 and 50-55 kDa could correspond to proteolysis form of the native envoplakin. The psoriatic skin (lesional) expresses only the 60-80 and 50-55 kDa bands. These two forms could be used for diagnosis of diseases with enhance adhesion such as psoriasis.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of an 60-80 and/or 50-55 kDa envoplakin polypeptide in an individual. We also disclose the diagnosis of a Group II disease or susceptibility to such a disease (preferably psoriasis or susceptibility to psoriasis) in an individual, by detecting any of the changes described above, preferably the absence of or a modulated level, preferably a lower level of an 124-209 kDa and/or an 100-120 kDa envoplakin polypeptide in an individual.

The diagnosis may also be achieved by assaying the relative abundance of the 124-209 kDa, 100-120 kDa, 60-80 kDa and 50-55 kDa envoplakin polypeptides.

Preferably, the relevant polypeptides are detected in an epidermis of an individual, whether in vivo or ex-vivo (e.g., in a biopsy).

SCCE Isoforms

SCCE is differentially expressed in stratum corneum of psoriatic skin compared to normal skin.

Antibody to SCCE reveals two strong bands in normal skin at 80-90 and 70-75 kDa. In psoratic lesional skin only a 70-75 kDa band is detected and an extra band (124-209) at higher molecualar weight is detected in psoriatic lesional skin. The expected size of SCCE is about 30 kDa. SCCE could be inserted into the cornified envelope by transglutaminases. This gives to SCCE higher MW than expected. The 124-209 kDa band can be used in the diagnostic of disease with increase adhesion such as psoriasis.

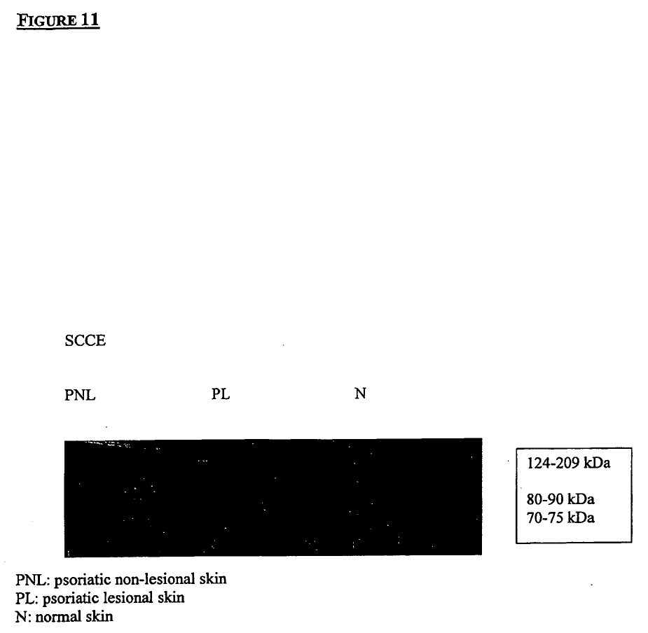

The results are shown in FIG. 11.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of an 124-209 kDa SCCE polypeptide in an individual. We also disclose the diagnosis of a Group II disease or susceptibility to such a disease (preferably psoriasis or susceptibility to psoriasis) in an individual, by detecting any of the changes described above, preferably the absence of or a modulated level, preferably a lower level of a 80-90 kDa SCCE polypeptide in an individual.

The diagnosis may also be achieved by assaying the relative abundance of the 80-90 kDa and 70-75 kDa polypeptides.

Preferably, the relevant polypeptides are detected in an epidermis of an individual, whether in vivo or ex-vivo (e.g., in a biopsy).

SLPI Profile

SLPI is strongly expressed in stratum corneum of psoriatic skin compared to normal stratum corneum.

Antibody to SLPI revealed two bands at 90-100 kda and 70-80 kDa.70-80 kDa shows that SLPI is up-regulated in psoriatic skin mostly in the lesional skin.

The results are shown in FIG. 12.

The expected Mw of SLPI protein is 20 kDa. Again SLPI could be inserted into the cornified envelope and cross-linked to other corneodesmosomal proteins by transglutaminases. The 90-100 kDa band is detected only in psoriatic skin including lesional and non lesional skin. This could be used for diagnosing diseases with increase adhesion such as psoriasis.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of an 90-100 kDa SLPI polypeptide in an individual. We also disclose the diagnosis of a Group II disease or susceptibility to such a disease (preferably psoriasis or susceptibility to psoriasis) in an individual, by detecting any of the changes described above, preferably the absence of or a modulated level, preferably a lower level of a 20 kDa SLPI polypeptide in an individual.

The diagnosis may also be achieved by assaying the relative abundance of the 90-100 kDa and 20 kDa polypeptides.

Preferably, the relevant polypeptides are detected in an epidermis of an individual, whether in vivo or ex-vivo (e.g., in a biopsy).

### Example C6

### Psoriasis Proteolysis Profiles of Proteins Extracted From the Stratum Corneum

Proteins are extracted from the stratum corneum of normal and psoriatic patient groups (from lesional and non-lesional skin), as described above, and analysed by Western blots using specific antibodies against corneodesmosomal proteins. We show that the profile of epidermally extracted proteins differs between normal and psoriatic lesional and non-lesional skin.

Corneodesmosin Proteolysis Profile The deficiency in proteolysis of desmo/corneodesmosomal proteins is reflected in a significant decrease in the quantity of mature forms of corneodesmosome proteins in psoriatic skin compared to normal. Proteolysis deficit causes the cells to stick together in the surface of the epidermis preventing cell shedding. For example, the 36 and 46-43 kDa forms of corneodesmosin are the major forms in the stratum corneum in normal skin and the 52-56 kDa form is very rare in normal stratum corneum. The corneodesmosin proteolysis profile in psoriatic skin is different. Thus, in psoriatic skin, 52-56 kDa form of corneodesmosin is abondant in the epidermis of psoriatic patients and persist in the stratum corneum. This suggests that there is a deficit in proteolysis in psoriatic skin.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of one or more 52-56 kDa corneodesmosin polypeptides in an individual. We also disclose the diagnosis of a Group II disease or susceptibility to such a disease (preferably psoriasis or susceptibility to psoriasis) in an individual, by detecting the absence of or a modulated level, preferably a lower level of one or both of 36 kDa and 46-43 kD corneodesmosin polypeptides in an individual.

The diagnosis may also be achieved by assaying the relative abundance of the 36 kDa, 46-43 kDa and 52-56 kDa polypeptides.

Preferably, the relevant polypeptides are detected in the stratum corneum of an individual, whether in vivo or ex-vivo (e.g., in a biopsy).

Desmoglein I Proteolysis Profile Similar results are obtained with desmoglein I (DG I). In the epidermis of normal skin the most abundant forms of DG I are 95 and 80 kDa which are the proteolytic products of the 160 kDa form. However, in the stratum corneum of psoriatic skin the most abundant form is the 160 kDa. This suggests that there is a deficit of proteolysis of DG I in psoriatic skin.

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of a 160 kDa desmoglein I polypeptide in an individual. Such diagnosis may also be done by detecting the absence of or a modulated level, preferably a lower level of one or both of a 95 and 80 kDa desmoglein I polypeptide in an individual.

The diagnosis may also be achieved by assaying the relative abundance of the 80 kDa, 95 kDa and 160 kDa polypeptides. The diagnosis may be done by detecting lack of proteolysis of a 160 kDa desmoglein I polypeptide in an individual.

Preferably, the relevant polypeptides are detected in an stratum corneum of an individual, whether in vivo or ex-vivo (e.g., in a biopsy).

Desmoglein 3 Proteolysis Profile

Desmoglein 3 (Dsg 3) also shows a decrease in proteolysis. The bands corresponding to the two proteolysis fragments 55 kDa and 100 kDa are weaker in the stratum corneum of psoriatic skin compared to the stratum corneum of normal skin. Both fragments react with the same antibody directed against the cytoplasmic domain. Another band of 80 kDa appears as a proteolysis product of Dsg 3 more strongly in normal compared to psoriatic epidermis.

We therefore disclose the diagnosis of a Group II disease or susceptibility to such a disease (preferably psoriasis or susceptibility to psoriasis) in an individual, by detecting the absence of any one or more of a 55 kDa desmoglein 3 fragment, an 80 kDa desmoglein 3 fragment and a 100 kDa desmoglein 3 fragment in an individual.

Preferably, the relevant polypeptides are detected in an stratum corneum of an individual, whether in vivo or ex-vivo (e.g., in a biopsy).

Desmoplakin Proteolysis Profile

Using polyclonal antibody to desmoplakin we revealed three bands 190-250; 120-180 and 75-80 kDa 190-250- and/or 120-180 are strongly expressed in normal compared to the disease. The 75-80 kDa could be proteolysis form of 190-250- and/or 120-180, suggesting that desmoplakin is not proteolysed properly in psoriatic skin (lesional and non-lesional skin). These profiles can be used to identified diseases with increase adhesion such as psoriasis

We therefore disclose the diagnosis of a Group II disease or susceptibility to such a disease (preferably psoriasis or susceptibility to psoriasis) in an individual, by detecting any of the changes described above, preferably the absence of or a modulated level, preferably a lower level of a either or both of 190-250 kDa and 120-180 kDa polypeptides of desmoplakin in an individual. The diagnosis may also be achieved by assaying the relative abundance of the 75-80 kDa, 190-250 and/or 120-180 polypeptides. The diagnosis may be done by detecting lack of proteolysis of a 85 kDa desmoplakin polypeptide in an individual.

Preferably, the relevant polypeptides are detected in an stratum corneum of an individual, whether in vivo or ex-vivo (e.g., in a biopsy).

Plakoglobin Proteolysis Profiles

Antibody to plakoglobin revealed three bands 85, 75 and 70 kDa The 70 and 75 kDa are proteolytic forms of the native protein (85 kDa) which is cleaved by proteases such as SCCE and SCTE during keratinocytes differenciation. The 70 kDa is the major form of the plakoglobin observed in the stratum corneum, is very strong in psoriatic skin (both lesional and non-lesional) and is almost absent in the normal epidermis. Psoriatic and normal skin have two different profiles. The 70 kDa band can be use for diagnosis of disease with increase adhesion (e.g. psoriasis and acne).

We therefore disclose the diagnosis of a Group II disease or susceptibility to such a disease (preferably psoriasis or susceptibility to psoriasis) in an individual, by detecting any of the changes described above, preferably the presence of or a modulated level, preferably a higher level of a 70 kDa plakoglobin polypeptide in an individual. We also disclose the diagnosis of a Group II disease or susceptibility to such a disease (preferably psoriasis or susceptibility to psoriasis) in an individual, by detecting the absence of or a modulated level, preferably a lower level of an 75 kDa plakoglobin polypeptide.

The diagnosis may also be achieved by assaying the relative abundance of the 85 kDa, 75 kDa and 70 kDa polypeptides. The diagnosis may be done by detecting lack of proteolysis of a 85 kDa plakoglobin polypeptide in an individual.

Preferably, the relevant polypeptides are detected in an stratum corneum of an individual, whether in vivo or ex-vivo (e.g., in a biopsy).

Desmocollin 1 Proteolysis Profile

Desmocollin 1 (DGIV/V) is strongly expressed in the suprabasal layers of the normal epidermis.

Antibody to desmoscollin reveals three Dsc1 isoforms in normal epidermis at molecular weigh 70-80; 60-70 and 50-60 kDa. Only two bands are detected in the epidermis of psoriatic skin including lesional and non-lesional skin, suggesting an abnormal proteolysis of the desmocollin I in psoriasis skin. The absence of 50-60 kDa band could be used for proteomic dignostic of disease with enhance adhesion such as psoriasis.

We therefore disclose the diagnosis of a Group II disease or susceptibility to such a disease (preferably psoriasis or susceptibility to psoriasis) in an individual, by detecting any of the changes described above, preferably the absence of or a modulated level, preferably a lower level of a 50-60 kDa Desmocollin 1 polypeptide in an individual.

The diagnosis may also be achieved by assaying the relative abundance of the 70-80 kDa, 60-70 kDa and 50-60 kDa polypeptides.

Preferably, the relevant polypeptides are detected in an stratum corneum of an individual, whether in vivo or ex-vivo (e.g., in a biopsy).

### Example C7

### Eczema Proteolysis Profiles of Proteins Extracted From the Stratum Corneum

Proteins are extracted from the stratum corneum of normal and eczematous patient groups, as described above, and analysed by Western blots using specific antibodies against corneodesmosomal proteins. We show that the profile of epidermally extracted proteins differs between normal and diseased (eczema) individuals.

Corneodesmosome Proteolysis Profile

The increase in proteolysis of desmo/corneodesmosomal proteins in eczema skin is reflected in a significant increase in the quantity of immature forms of corneodesmosome proteins in eczematous skin compared to normal. Increased proteolysis causes the cells to stick together in the surface of the epidermis preventing cell shedding.

For example, the 36 and 46-43 kDa forms of corneodesmosin are the major forms in the stratum corneum in normal skin and the 52-56 kDa forms are very rare in the stratum corneum. There is an increase of corneodesmosin proteolysis in eczematous skin, so that the 36 and 4643 kDa forms of corneodesmosin are dominant forms in the stratum corneum of eczema patients. This shows that there is an increase in proteolysis in eczematous skin.

We therefore disclose the diagnosis of a Group I disease or susceptibility to a Group I disease (preferably eczema or susceptibility to eczema) in an individual, by detecting any of the changes described above, preferably the presence of or a modulated level, preferably a higher level of one or more 36, 46-43 kDa corneodesmosin polypeptides in an individual. We also disclose the diagnosis of a Group I disease or susceptibility to such a disease (preferably eczema or susceptibility to eczema) in an individual, by detecting the absence of or a modulated level, preferably a lower level of 52-56 kDa corneodesmosin polypeptides in an individual.

The diagnosis may also be achieved by assaying the relative abundance of the 36 kDa, 4643 kDa and 52-56 kDa polypeptides.

Preferably, the relevant polypeptides are detected in an stratum corneum of an individual, whether in vivo or ex-vivo (e.g., in the form of a tape strip).

Desmoglein I Proteolysis Profile

Similar results are obtained with desmoglein I (DG I). In the stratum corneum of normal skin the most abundant forms of DG I are 95 and 80 kDa which are the proteolytic products of the 160 kDa form. In the stratum corneum of eczema skin there is an increase of 95 and 80 kDa forms, suggesting that the proteolysis process of DG I is enhanced in eczematous skin.

We therefore disclose the diagnosis of a Group I disease or susceptibility to a Group I disease (preferably eczema or susceptibility to eczema) in an individual, by detecting any of the changes described above, preferably the presence of or a modulated level, preferably a higher level of one or more of 95 and 80 kDa polypeptides in an individual. Such diagnosis may also be done by detecting the absence of or a modulated level, preferably a lower level of a 160 kDa desmoglein I polypeptide in an individual.

The diagnosis may also be achieved by assaying the relative abundance of the 80 kDa, 95 kDa and 160 kDa polypeptides. The diagnosis may be done by detecting presence of proteolysis of a 160 kDa desmoglein I polypeptide in an individual.

Preferably, the relevant polypeptides are detected in an stratum corneum of an individual, whether in vivo or ex-vivo (e.g., in the form of a tape strip).

Desmoglein 3Proteolysis Profile

Desmoglein 3 (Dsg 3) shows in increase in proteolysis. The bands corresponding to the two proteolysis fragments 55 kDa and 100 kDa are strong in the stratum corneum of eczema skin compared to the stratum corneum of normal skin. Both fragments react with the same antibody directed against the cytoplasmic domain. Another band of 80 kDa which appears as a proteolysis product of Dsg 3 is weaker in normal compared to eczematous stratum corneum.

We therefore disclose the diagnosis of a Group I disease or susceptibility to a Group I disease (preferably eczema or susceptibility to eczema) in an individual, by detecting any of the changes described above, preferably the presence of or a modulated level, preferably a higher level of any one, pair or all of a 55 kDa desmoglein 3 polypeptide, an 80 kDa desmoglein 3 polypeptide and a 100 kDa desmoglein 3 polypeptide in an individual.

Preferably, the relevant polypeptides are detected in an stratum corneum of an individual, whether in vivo or ex-vivo (e.g., in the form of a tape strip).

Plakoglobin Proteolysis Profile

Antibody to plakoglobin revealed three bands 85, 75 and 70 kDa The 70 and 75 kDa are proteolytic forms of the native protein (85 kDa) which is cleaved by proteases such as SCCE and SCTE during keratinocytes differenciation. The 70 kDa is very weak in eczematic skin (both lesional and non-lesional) and is almost absent in the normal epidermis. 85 and 75 kDa are very strong in eczema skin compared to normal. Eczema and normal skin have two different profiles. The 85 and 75 kDa bands can be use for diagnosis of eczema.

We therefore disclose the diagnosis of a Group I disease or susceptibility to a Group I disease (preferably eczema or susceptibility to eczema) in an individual, by detecting any of the changes described above, preferably the absence of or a modulated level, preferably a lower level of a 70 kDa plakoglobin polypeptide in an individual. We also disclose the diagnosis of a Group I disease or susceptibility to a Group I disease (preferably eczema or susceptibility to eczema) in an individual, by detecting the presence of or a modulated level, preferably a higher level of an 85 kDa plakoglobin and/or a 75kDa plakoglobin polypeptide.

The diagnosis may also be achieved by assaying the relative abundance of the 85 kDa, 75 kDa and 70 kDa polypeptides.

Preferably, the relevant polypeptides are detected in an stratum corneum of an individual, whether in vivo or ex-vivo (e.g., in the form of a tape strip).

Desmocollin 1 Proteolysis Profile

Desmocollin 1 (DGIV/V) is strongly expressed in the suprabasal layers of the normal epidermis.

Antibody to desmoscollin revealed three Dsc1 isoforms in normal epidermis at molecular weigh 70-80; 60-70 and 50-60 kDa. The 50-60 kDa band is strongly expressed in eczema skin including lesional and non-lesional skin, suggesting an increase proteolysis of the desmocollin 1 in eczema skin. The high expression of 50-60 kDa band could be used for proteomic diagnosis of disease with defective skin barrier such as eczema.

We therefore disclose the diagnosis of a Group I disease or susceptibility to a Group I disease (preferably eczema or susceptibility to eczema) in an individual, by detecting any of the changes described above, preferably the presence of or a modulated level, preferably a higher level of a 50-60 kDa Desmocollin 1 polypeptide in an individual.

The diagnosis may also be achieved by assaying the relative abundance of the 70-80 kDa, 60-70 kDa and 50-60 kDa polypeptides.

Preferably, the relevant polypeptides are detected in an stratum corneum of an individual, whether in vivo or ex-vivo (e.g., in the form of a tape strip).

### Examples D

### Gene Regulation

### Example D1

### Expression of Adhesion Protein, Protease and Protease Inhibitor Genes in Psoriasis Assayed Using Oligonucleotide Arrays

We used Affymetrix oligonucleotide arrays comprising desmosomal and corneodesmosomal adhesion proteins, proteases and protease inhibitor genes. These genes are listed in the Tables below in Examples D2 to D7.

Punch biopsies from involved and uninvolved skin of patients with psoriasis, acne vulgaris, ichtyosis, keratoses pilaris, atopic eczema, Crohn's disease, skin melanoma, squamous cell carcinima, basal cell carcinima, cutaneous lymphoma, skin cancer, malignancy of gastrointestinal tract, malignancy of the lung, are obtained from the skin. Biopsies are also obtained from unrelated control blood donors.

RNA is extracted from each sample and an average of 50, 20 and 15 μg are obtained from lesional, non-lesional and normal skins respectively. Approximately 10 μg is used to prepare biotinylated cRNA and 2 μg is used for hybridization to Affymetrix U95A arrays that contain probes for genes as listed in the Tables.

GENECHIP software is used to reflect the expression level of each gene by converting the image intensities to average difference into the expression level. The expression of genes in normal skin is used as reference and the results are shown in the Tables below.

### Example D2

### Expression of Corneodesmosomal Genes in Psoriasis Assayed Using Oligonucleotide Arrays

The expression of corneodesmosomal genes is assayed as described in psoriatic patients, using psoriatic lesional skin (“involved”) and psoriatic non-lesional skin (“uninvolved”). The expression level of each gene in disease (involved and uninvolved) is compared to its expression in normal skin.

Results are shown in Table D2.1 below. Key: ++: normally expressed; +++: strongly expressed; ++++: highly expressed, +down-regulation.

TABLE D2.1

| | Expression level | | GenBank accession |
|---|---|---|---|
| Gene Name | Involved | Uninvolved | number |
| S/corneodesmosin | ++++ | +++ | GB: AF030130 |
| desoplakin | ++++ | +++ | GB: XM_004463 |
| plakoglobin | ++++ | +++ | GB: NM_002230; |
| | | | GB: NM_021991 |
| desmoglein 1 | ++++ | +++ | GB: XM_008810 |
| desmocollin 1 | ++++ | +++ | GB: MX_008687 |
| envoplakin | ++++ | +++ | GB: XM_008135; U72543 |
| plectin 1 | ++++ | +++ | GB: NM000445 |
| S100A2 | ++++ | +++ | GB: AI539439; M87068 |
| keratin 6A | +++ | ++ | GB: L42611 |
| keratin 17 | +++ | ++ | GB: Z19574 |
| S100A8 | ++++ | ++ | GB: AI126134 |
| S100A7 | ++++ | +++ | GB: AA586894 |
| S100A9 | ++++ | +++ | GB: W72424 |
| SPRR2A | ++++ | +++ | GB: M21302 |
| SPRR1B | ++++ | +++ | GB: M19888 |
| SPRK | ++++ | +++ | GB: AI923984 |
| HCR | ++++ | +++ | GB: BAA81890 |
| SEEK1 | ++++ | +++ | GB: BAA88130 |
| SPR1 | ++++ | +++ | GB: BAB63315 |
| STG | ++++ | +++ | GB: BAA88132 |
| involucrin | ++++ | +++ | GB: NM_005547 |
| annexin A1/ | ++++ | ++++ | GB: X05908 |
| lipocortin | | | |
| collagen, type VI, | +++ | ++ | GB: NM_004369 |
| alpha 3 | | | |
| (COL6A3) | | | |
| trichohyalin | ++++ | +++ | GB: NM_005547 |
| loricrin | ++++ | +++ | GB: XM_048902 |

We therefore provide the diagnosis of a Group II disease or susceptibility to a Group II disease (preferably psoriasis or susceptibility to psoriasis) in an individual, by detecting modulation of expression, preferably up-regulation of expression of a polypeptide or nucleic acid selected from the group consisting of S/corneodesmosin (AF030130); desoplakin (XM_{—}004463); plakoglobin (NM_{—}002230; GB: NM_{—}021991); desmoglein 1 (XM_{—}008810); desmocollin 1 (MX_{—}008687); envoplakin (XM_{—}008135;U72543); plectin 1 (NM000445); S100A2 (AI539439;M87068); keratin 6A (L42611); keratin 17 (Z19574); S100A8 (AI126134); S100A7 (AA586894); S100A9); GB:W72424); SPRR2A); GB:M21302); SPRR1B (M19888); SPRK (AI923984); HCR (BAA81890); SEEK1 (BAA88130); SPR1 (BAB63315); STG (BAA88132); involucrin (NM_{—}005547); annexin A1/lipocortin (X05908); collagen, type VI, alpha 3 (COL6A3) (NM_{—}004369); trichohyalin (NM_{—}005547); and loricrin (XM_{—}048902).

### Example D3

### Expression of Protease Genes in Psoriasis Assayed Using Oligonucleotide Arrays

The expression of protease genes is assayed as described in psoriatic patients, using psoriatic lesional skin (“involved”) and psoriatic non-lesional skin (“uninvolved”). The expression level of each gene in disease (involved and uninvolved) is compared to its expression in normal skin.

Results are shown in Table D3.1 below. Key: ++: normally expressed; +++: strongly expressed; ++++: highly expressed, +down-regulation.

TABLE D3.1

| | | | GenBank |
|---|---|---|---|
| | Expression level | | accession |
| Gene Name | Involved | Uninvolved | number |
| Apoptosis-related cysteine | + | + | GB: NM_012114 |
| protease (CASP14) mRNA | | | |
| Transglutaminase 1 | ++++ | +++ | GB: M98447 |
| (TGM1) | | | |
| TGM2 | ++++ | ++ | XM_009482 |
| TGM4 | +++ | ++ | XM_056203 |
| TGM5 | ++++ | ++ | GB: XM_007529 |
| TGM7 | ++++ | ++ | GB: NM_052955 |
| TGM3 | ++++ | ++ | GB: L10386 |
| phospholipases A(2) | ++++ | ++ | GB: BC013384 |
| CD47 antigen | +++ | ++++ | GB: X69398 |
| Kallilkrein 8 | ++++ | ++ | GB: AB008390 |
| AD024 protein | +++ | +++ | GB: XM_002642 |
| SCCE | + | + | GB: XM_009002 |
| Defensin beta2 | ++++ | ++ | GB: AF0711216 |
| Interferon a inducible | +++ | +++ | GB: X67325 |
| protein 27 | | | |
| Fatty acid binding protein | +++ | +++ | GB: M94856 |
| FABP5 | | | |
| SCTE | ++ | + | GB: XM_009000 |
| kallikrein 1, | ++ | ++ | GB: XM_047300 |
| renal/pancreas/salivary | | | |
| (KLK1) | | | |
| Homo sapiens kallikrein 2, | +++ | +++ | GB: XM_031757 |
| prostatic (KLK2) | | | |
| kallikrein 3, (prostate | ++++ | ++ | GB: XM_031768 |
| specific antigen) (KLK3) | | | |
| kallikrein 6 (neurosin, | ++ | + | GB: XM_055658 |
| zyme) (KLK6) | | | |
| kallikrein 4 (prostase, | +++ | ++ | GB: XM_008997 |
| enamel matrix, prostate) | | | |
| (KLK4) | | | |
| membrane-type serine | +++ | ++ | GB: AF133086 |
| protease 1 | | | |
| Human skin collagenase | + | + | GB: M13509 |
| collagenase MMP-1 | +++ | ++ | GB: LOC116389 |
| collagenase MMP-12 | +++ | + | GB: U78045 |
| collagenase MMP-9 | +++ | ++ | GB: NM_004994 |
| collagenase MMP-3 | +++ | ++ | GB: U78045 |
| collagenase MMP-28 | +++ | ++ | GB: AF219624 |
| caspase 7 | ++ | ++ | GB: BC015799 |
| Caspase 5 | +++ | ++ | GB: NM_004347 |
| Caspase-14 | ++ | ++ | GB: NM_012114 |
| ubiquitin specific protease | ++ | ++ | GB: NM_003481 |
| USP-5 | | | |
| ubiquitin specific protease | ++ | ++ | GB: NM_004651 |
| USP-11 | | | |
| ubiquitin specific protease | ++ | ++ | GB: NM_004505 |
| USP 6 | | | |
| ubiquitin specific protease | +++ | ++ | GB: NM_031907 |
| USP 26 | | | |
| ubiquitin specific protease | +++ | ++ | GB: NM_020886 |
| (USP 28) | | | |
| 26S protease subunit 4 | +++ | ++ | GB: L02426 |
| LILRB1 | | | GB: AF004230 |
| Signal trasducer and | +++ | ++ | GB: 977935 |
| activator of transcription 1, | | | |
| 91 kDa (STAT1) | | | |
| proteasome (prosome, | +++ | ++ | GB: X59417 |
| macropain) subunit 6 | | | |
| (PSMA6) | | | |
| TPS1 | + | + | GB: NM_003293 |
| TPSB1 | ++ | ++ | GB: XM_016204 |
| TPSG1 | + | ++ | GB: XM_008123 |
| protease nexin-II | ++ | ++ | GB: XM_047793 |
| Glia derived nexin | ++ | ++ | GB: P07093 |
| precursor | | | |
| 26S protease regulatory | ++ | ++ | GB: Q92524 |
| subunit S10B | | | |
| PCOLN3 | ++ | ++ | GB: XM_047524 |

We therefore provide the diagnosis of a Group II disease or susceptibility to a Group II disease (preferably psoriasis or susceptibility to psoriasis) in an individual, by detecting modulation of expression, preferably up-regulation of expression of a polypeptide or nucleic acid selected from the group consisting of Transglutaminase 1 (TGM1) (M98447); TGM2 (XM_{—}009482); TGM4 (XM_{—}056203); TGM5 (XM_{—}007529); TGM7 (NM_{—}052955); TGM3 (L10386); phospholipases A(2) (BC013384); CD47 antigen (X69398); Kallilkrein 8 (AB008390); AD024 protein (XM_{—}002642); Defensin beta2 (AF0711216); Interferon a inducible protein 27 (X67325); Fatty acid binding protein FABP5 (M94856); SCTE (XM_{—}009000); kallikrein 1, renal/pancreas/salivary (KLK1) (XM_{—}047300); Homo sapiens kallikrein 2, prostatic (KLK2) (XM_{—}031757); kallikrein 3, (prostate specific antigen) (KLK3) (XM_{—}031768); kallikrein 6 (neurosin, zyme) (KLK6) (XM_{—}055658); kallikrein 4 (prostase, enamel matrix, prostate) (KLK4) (XM_{—}008997); membrane-type serine protease 1 (AF133086); collagenase MMP-1 (LOC16389); collagenase MMP-12 (U78045); collagenase MMP-9 (NM_{—}004994); collagenase MMP-3 (U78045); collagenase MMP-28 (AF219624); caspase 7 (BC015799); Caspase 5 (NM_{—}004347); Caspase-14 (NM_{—}012114); ubiquitin specific protease USP-5 (NM_{—}003481); ubiquitin specific protease USP-11 (NM_{—}004651); ubiquitin specific protease USP 6 (NM_{—}004505); ubiquitin specific protease USP 26 (NM_{—}031907); ubiquitin specific protease (USP 28) (NM_{—}020886); 26S protease subunit 4 (L02426); LILRB1 (AF004230); Signal trasducer and activator of transcription 1, 91 kDa (STAT1) (977935); proteasome (prosome, macropain) subunit 6 (PSMA6) (X59417); TPSB1 (XM_{—}016204);; protease nexin-II (XM_{—}047793); Glia derived nexin precursor (P07093); and 26S protease regulatory subunit S10B; PCOLN3 (XM_{—}047524).

We further provide the diagnosis of a Group II disease or susceptibility to a Group II disease (preferably psoriasis or susceptibility to psoriasis) in an individual, by detecting modulation of expression, preferably down-regulation of expression of a polypeptide or nucleic acid selected from the group consisting of Apoptosis-related cysteine protease (CASP14) mRNA (NM_{—}012114), SCCE (XM_{—}009002), Human skin collagenase (M13509); TPS1 (NM_{—}03293); and TPSG1 (XM_{—}008123).

### Example D4

### Expression of Protease Inhibitor Genes in Psoriasis Assayed Using Oligonucleotide Arrays

The expression of protease inhibitor genes is assayed as described in psoriatic patients, using psoriatic lesional skin (“involved”) and psoriatic non-lesional skin (“uninvolved”). The expression level of each gene in disease (involved and uninvolved) is compared to its expression in normal skin.

Results are shown in Table D4.1 below. Key: ++: normally expressed; +++: strongly expressed; ++++: highly expressed, +down-regulation.

TABLE D4.1

| | Expression level | | GenBank |
|---|---|---|---|
| | | Un- | accession |
| Gene Name | Involved | involved | number |
| SLPI | ++++ | +++ | GB: X04502 |
| SKALP | ++++ | +++ | XM_009524; |
| | | | L10343 |
| CSTA | ++++ | +++ | GB: |
| | | | NM_005213; |
| | | | AA570193 |
| SCCA | ++++ | +++ | GB: S66296 |
| SCCA2 | ++++ | +++ | GB: U19557 |
| plasminogen activator | ++++ | ++ | BG: X04729; |
| inhibitor type 1 | | | X04731 |
| PAI2 | ++++ | + | GB: AF071400 |
| SERPINA5 | ++++ | ++ | GB: NM_000624 |
| hbc750 Human pancreatic | + | ++ | GB: T11141; |
| islet | | | T10920 |
| plasminogen activator | +++ | ++ | GB: L19066 |
| inhibitor type 2 | | | |
| TIMP | ++++ | +++ | GB: D11139 |
| TIMP-1 | +++ | ++ | GB: NM_003254 |
| TIMP-2 | ++++ | ++ | GB: NM_003255 |
| TIMP-3 | ++++ | ++ | GB: E13880 |
| TIMP-4 | ++ | ++ | GB: NM_003256 |
| TIM9a | +++ | +++ | GB: AF150100 |
| TIM9b | +++ | +++ | GB: AF150105 |
| Cystatin A | ++++ | +++ | GB: AA570193 |
| Cystatin M/E | ++++ | +++ | GB: NM_001323 |
| multivalent protease | +++ | ++ | GB: AF422194 |
| inhibitor WFIKKN | | | |
| C1 inhibitor (SERPING1) | +++ | ++ | GB: XM_046218 |
| protease inhibitor, Kunitz | +++ | ++ | GB: XM_032280 |
| type, 2 (SPINT2) | | | |
| serine protease inhibitor, | ++++ | +++ | GB: XM_005539 |
| Kazal type 4 (SPINK4) | | | |
| proteinase inhibitor, clade B | ++++ | +++ | GB: XM_053642 |
| (ovalbumin), member 9 | | | |
| serine (or cysteine) | ++++ | +++ | GB: XM_047984 |
| proteinase inhibitor, clade | | | |
| B (ovalbumin), member 6 | | | |
| eppin-1 (EPPIN) | +++ | ++ | GB: AF286368 |
| eppin-2 (EPPIN) | ++ | ++ | GB: AF286369 |
| eppin-3 (EPPIN) | ++ | +++ | GB: AF286370 |
| Serine protease inhibitor- | ++++ | +++ | GB: NM_020398 |
| like, with Kunitz and WAP | | | |
| domains 1 (eppin) | | | |
| (SPINLW1) | | | |
| sparc/osteonectin, cwcv and | +++ | ++ | GB: NM_004598 |
| kazal-like domains | | | |
| proteoglycan (testican) | | | |
| (SPOCK) | | | |
| protease inhibitor Kunitz | ++++ | +++ | GB: XM_056836 |
| type 1 (SPINT1) | | | |
| PI12 | ++++ | +++ | GB: AH009756 |
| Human immunodeficiency | ++++ | ++ | GB: AB020923 |
| virus type 1 gene for HIV-1 | | | |
| protease | | | |
| Human immunodeficiency | ++++ | ++ | GB: AB020924 |
| virus type 1 gene for HIV-1 | | | |
| protease | | | |
| tissue factor pathway | ++ | ++ | GB: NM_006528 |
| inhibitor 2 (TFPI2) | | | |
| secreted phosphoprotein 2, | ++ | ++ | GB: NM_006944 |
| 24 kD (SPP2) | | | |
| cathepsin F (CTSF | +++ | +++ | GB: NM_003793 |
| serine (or cysteine) | ++++ | +++ | GB: NM_001756 |
| proteinase inhibitor, clade A | | | |
| (alpha-1 antiproteinase, | | | |
| antitrypsin), member 6 | | | |
| (SERPINA6) | | | |
| serine (or cysteine) | ++++ | +++ | GB: NM_006919 |
| proteinase inhibitor, clade B | | | |
| (ovalbumin), member 3 | | | |
| (SERPINB3) | | | |
| Serine (or cysteine) | ++++ | +++ | GB: NM_001085 |
| proteinase inhibitor, clade A | | | |
| (alpha-1 antiproteinase, | | | |
| antitrypsin), member 3 | | | |
| (SERPINA3) | | | |
| Homo sapiens serine (or | ++++ | +++ | GB: XM_008743 |
| cysteine) proteinase | | | |
| inhibitor, clade B | | | |
| (ovalbumin), member 13 | | | |
| serine (or cysteine) | ++++ | +++ | GB: XM_008742 |
| proteinase inhibitor, clade B | | | |
| (ovalbumin), member 5 | | | |
| (SERPINB5 | | | |
| RelA-associated inhibitor | ++++ | +++ | GB: XM_057693 |
| inhibitor of DNA binding 1, | +++ | ++ | GB: XM_046179 |
| dominant negative helix- | | | |
| loop-helix protein (ID1) | | | |
| serine (or cysteine) | ++++ | +++ | GB: XM_054850 |
| proteinase inhibitor, clade E | | | |
| (nexin, plasminogen | | | |
| activator inhibitor type 1), | | | |
| member | | | |
| (SERPINE1) | | | |
| cyclin-dependent kinase | +++ | +++ | GB: NM_004936 |
| inhibitor 2B (p15, inhibits | | | |
| CDK4) (CDKN2B) | | | |
| Similar to cyclin-dependent | ++++ | +++ | GB: BC014469 |
| kinase inhibitor 2B (p15, | | | |
| inhibits CDK4) | | | |
| serine (or cysteine) | ++++ | +++ | GB: XM_008745 |
| proteinase inhibitor, clade B | | | |
| (ovalbumin), member 7 | | | |
| (SERPINB7 | | | |
| protein inhibitor of | ++++ | +++ | GB: NM_016149 |
| activated STAT protein | | | |
| PIASy (PIASY) | | | |
| similar to protein inhibitor | ++++ | +++ | GB: XM_016864 |
| of activated STAT protein | | | |
| PIASy (LOC95830) | | | |
| PKC-potentiated PP1 | ++++ | +++ | GB: AY050668 |
| inhibitory protein | | | |
| (PPP1R14A) | | | |
| inhibitor of DNA binding 3, | ++++ | +++ | GB: NM_002167 |
| dominant negative helix- | | | |
| loop-helix protein (ID3) | | | |
| Clade A (alpha-1 | ++++ | +++ | XM_028358 |
| antiproteinase, antitrypsin) | | | |
| serine (or cysteine) | ++++ | +++ | GB: NM_004353 |
| proteinase inhibitor, clade H | | | |
| (heat shock protein 47), | | | |
| member 1 (SERPINH1) | | | |
| PI13 gene for hurpin (serine | ++++ | +++ | GB: AJ278717 |
| protease inhibitor) | | | |
| protease inhibitor 5 | ++++ | +++ | XM_008742 |
| (maspin) (PI5) | | | |
| PAI-2 | +++ | +++ | A32415 |

We therefore provide the diagnosis of a Group II disease or susceptibility to a Group II disease (preferably psoriasis or susceptibility to psoriasis) in an individual, by detecting modulation of expression, preferably up-regulation of expression of a polypeptide or nucleic acid selected from the group consisting of SLPI (X04502); SKALP (XM_{—}009524; L10343); CSTA (NM_{—}005213; AA570193); SCCA (S66296); SCCA2 (U19557); plasminogen activator inhibitor type 1 (X04729; X0473 1); PAI2 (AF071400); SERPINA5 (NM_{—}000624); plasminogen activator inhibitor type 2 (L19066); TIMP (D11139); TIMP-1 (NM_{—}003254); TIMP-2 (NM_{—}003255); TIMP-3 (E13880); TIMP-4 (NM_{—}003256); TIM9a (AF150100); TIM9b (AF150105); Cystatin A (AA570193); Cystatin M/E (NM_{—}001323); multivalent protease inhibitor WFIKKN (AF422194); C1 inhibitor (SERPING1) (XM_{—}046218); protease inhibitor, Kunitz type, 2 (SPINT2) (XM_{—}032280); serine protease inhibitor, Kazal type 4 (SPINK4) (XM_{—}005539); proteinase inhibitor, clade B (ovalbumin), member 9 (XM_{—}053642); serine (or cysteine) proteinase inhibitor, clade); B (ovalbumin), member 6 (XM_{—}047984); eppin-1 (EPPIN) (AF286368); eppin-2 (EPPIN) (AF286369); eppin-3 (EPPIN) (AF286370); Serine protease inhibitor-like, with Kunitz and WAP domains 1 (eppin) (SPINLW1) (NM_{—}020398); sparc/osteonectin, cwcv and kazal-like domains proteoglycan (testican) (SPOCK) (NM_{—}004598); protease inhibitor Kunitz type 1 (SPINT1) (XM_{—}056836); PI12 (AH009756); Human immunodeficiency virus type 1 gene for HIV-1 protease (AB020923); Human immunodeficiency virus type 1 gene for HIV-1 protease (AB020924); tissue factor pathway inhibitor 2 (TFPI2) (NM_{—}006528); secreted phosphoprotein 2, 24 kD (SPP2) (NM_{—}006944); cathepsin F (CTSF (NM_{—}003793); serine (or cysteine) proteinase inhibitor, clade A (alpha-1 antiproteinase, antitrypsin), member 6 (SERPINA6) (NM_{—}001756); serine (or cysteine) proteinase inhibitor, clade B (ovalbumin), member 3 (SERPINB3) (NM_{—}006919); Serine (or cysteine) proteinase inhibitor, clade A (alpha-1 antiproteinase, antitrypsin), member 3 (SERPINA3) (NM_{—}001085); Homo sapiens serine (or cysteine) proteinase inhibitor, clade B (ovalbumin), member 13 (XM_{—}008743); serine (or cysteine) proteinase inhibitor, clade B (ovalbumin), member 5 (SERPINB5 (XM_{—}008742); RelA-associated inhibitor (XM_{—}057693); inhibitor of DNA binding 1, dominant negative helix-loop-helix protein (ID1) (XM_{—}046179); serine (or cysteine) proteinase inhibitor, clade E (nexin, plasminogen activator inhibitor type 1), member (SERPINE1) (XM_{—}054850); cyclin-dependent kinase inhibitor 2B (p15, inhibits CDK4) (CDKN2B) (NM_{—}004936); Similar to cyclin-dependent kinase inhibitor 2B (p 15, inhibits CDK4) (BC014469); serine (or cysteine) proteinase inhibitor, clade B (ovalbumin), member 7 (SERPINB7 (XM_{—}008745); protein inhibitor of activated STAT protein PIASy (PIASY) (NM_{—}16149); similar to protein inhibitor of activated STAT protein PIASy (LOC95830) (XM_{—}016864); PKC-potentiated PP1 inhibitory protein (PPP1R14A) (AY050668); inhibitor of DNA binding 3, dominant negative helix-loop-helix protein (ID3) (NM_{—}002167); Clade A (alpha-1 antiproteinase, antitrypsin) (XM_{—}028358); serine (or cysteine) proteinase inhibitor, clade H (heat shock protein 47), member 1 (SERPINH1) (NM_{—}004353); PI13 gene for hurpin (serine protease inhibitor) (AJ278717); protease inhibitor 5 (maspin) (PI5) (XM_{—}008742); PAI-2 (A32415).

We further provide the diagnosis of a Group II disease or susceptibility to a Group II disease (preferably psoriasis or susceptibility to psoriasis) in an individual, by detecting modulation of expression, preferably down-regulation of expression of a hbc750 Human pancreatic islet (T11141; T10920) polypeptide or nucleic acid.

### Example D5

### Expression of Corneodesmosomal Genes in Eczema Assayed Using Oligonucleotide Arrays

The expression of corneodesmosomal genes is assayed as described in eczema patients, using eczema lesional skin (“involved”) and eczema non-lesional skin (“uninvolved”). The expression level of each gene in disease (involved and uninvolved) is compared to its expression in normal skin.

Results are shown in Table D5.1 below. Key: ++: normally expressed; +++: strongly expressed; ++++: highly expressed, +down-regulation.

TABLE D5.1

| | Expression level | | GenBank accession |
|---|---|---|---|
| Gene Name | Involved | Uninvolved | number |
| S/corneodesmosin | + | + | GB: AF030130 |
| desoplakin | + | + | GB: XM_004463 |
| plakoglobin | + | + | GB: NM_002230; |
| | | | GB: NM_021991 |
| desmoglein 1 | + | + | GB: XM_008810 |
| desmocollin 1 | + | + | GB: MX_008687 |
| envoplakin | + | + | GB: XM_008135; |
| | | | U72543 |
| plectin 1 | + | ++ | GB: NM000445 |
| S100A2 | + | ++ | GB: AI539439; |
| | | | M87068 |
| keratin 6A | ++ | ++ | GB: L42611 |
| keratin 17 | ++ | ++ | GB: Z19574 |
| S100A8 | + | ++ | GB: AI126134 |
| S100A7 | + | +++ | GB: AA586894 |
| S100A9 | + | ++ | GB: W72424 |
| SPRR2A | + | ++ | GB: M21302 |
| SPRR1B | + | + | GB: M19888 |
| SPRK | + | + | GB: AI923984 |
| HCR | + | + | GB: BAA81890 |
| SEEK1 | + | + | GB: BAA88130 |
| SPR1 | + | + | GB: BAB63315 |
| STG | + | ++ | GB: BAA88132 |
| involucrin | + | + | GB: NM_005547 |
| annexin A1/lipocortin | ++ | ++ | GB: X05908 |
| trichohyalin | + | + | GB: NM_005547 |
| collagen, type VI, | ++ | ++ | GB: NM_004369 |
| alpha 3 | | | |
| (COL6A3) | | | |
| loricrin | + | + | GB: XM_048902 |

We therefore provide the diagnosis of a Group 1 disease or susceptibility to a Group 1 disease (preferably eczema or susceptibility to eczema) in an individual, by detecting modulation of expression, preferably up-regulation of expression of a polypeptide or nucleic acid selected from the group consisting of keratin 6A (L42611); keratin 17 (Z19574); annexin A1/lipocortin (X05908); and collagen, type VI, alpha 3 (COL6A3) (NM_{—}004369) in an individual.

Furthermore, we provide the diagnosis of a Group 1 disease or susceptibility to a Group 1 disease (preferably eczema or susceptibility to eczema) in an individual, by detecting modulation of expression, preferably down-regulation of expression of a polypeptide or nucleic acid selected from the group consisting of S/corneodesmosin (AF030130); desoplakin (XM_{—}004463); plakoglobin (NM_{—}002230; (NM_{—}021991); desmoglein 1 (XM_{—}008810); desmocollin 1 (MX_{—}008687); envoplakin (XM_{—}008135;U72543); plectin 1 (NM000445); S100A2 (AI539439;M87068); S100A8 (AI126134); S100A7 (AA586894); S100A9); GB:W72424); SPRR2A); GB:M21302); SPRR1B (M19888); SPRK (AI923984); HCR(BAA81890); SEEK1 (BAA88130); SPR1 (BAB63315); STG (BAA88132); involucrin (NM_{—}005547); trichohyalin (NM_{—}005547); and loricrin (XM_{—}048902).

### Example D6

### Expression of Protease Genes in Eczema Assayed Using Oligonucleotide Arrays

The expression of protease genes in eczema patients involved skin (eczema lesional skin), eczema uninvolved (eczema non-lesional skin) and normal skin. The expression level of each gene in disease (involved and uninvolved) is compared to its expression in normal skin.

Results are shown in Table D6.1 below. Key: ++: normally expressed; +++: strongly expressed; ++++: highly expressed, +down-regulation.

TABLE D6.1

| | | | GenBank |
|---|---|---|---|
| | Expression level | | accession |
| Gene Name | Involved | Uninvolved | number |
| Apoptosis-related cysteine | +++ | ++ | GB: NM_012114 |
| protease (CASP14) mRNA | | | |
| Transglutaminase 1 | + | + | GB: M98447 |
| (TGM1) | | | |
| TGM2 | + | ++ | XM_009482 |
| TGM4 | + | ++ | XM_056203 |
| TGM5 | ++ | ++ | GB: XM_007529 |
| TGM7 | + | + | GB: NM_052955 |
| TGM3 | + | ++ | GB: L10386 |
| phospholipases A(2) | +++ | ++ | GB: BC013384 |
| CD47 antigen | ++ | ++ | GB: X69398 |
| Kallilkrein 8 | ++++ | ++ | GB: AB008390 |
| AD024 protein | +++ | +++ | GB: XM_002642 |
| SCCE | ++++ | +++ | GB: XM_009002 |
| Defensin beta2 | ++++ | ++ | GB: AF0711216 |
| Interferon a inducible | +++ | +++ | GB: X67325 |
| protein 27 | | | |
| Fatty acid binding protein | +++ | +++ | GB: M94856 |
| FABP5 | | | |
| SCTE | ++++ | +++ | GB: XM_009000 |
| kallikrein 1, | ++++ | ++ | GB: XM_047300 |
| renal/pancreas/salivary | | | |
| (KLK1) | | | |
| Homo sapiens kallikrein 2, | ++++ | + | GB: XM_031757 |
| prostatic (KLK2) | | | |
| kallikrein 3, (prostate | ++++ | ++ | GB: XM_031768 |
| specific antigen) (KLK3) | | | |
| kallikrein 6 (neurosin, | +++ | ++ | GB: XM_055658 |
| zyme) (KLK6) | | | |
| kallikrein 4 (prostase, | ++++ | ++ | GB: XM_008997 |
| enamel matrix, prostate) | | | |
| (KLK4) | | | |
| membrane-type serine | ++ | + | GB: AF133086 |
| protease 1 | | | |
| Human skin collagenase | +++ | + | GB: M13509 |
| collagenase MMP-1 | +++ | + | GB: LOC116389 |
| collagenase MMP-12 | +++ | ++ | GB: U78045 |
| collagenase MMP-9 | ++ | +++ | GB: NM_004994 |
| collagenase MMP-3 | +++ | ++ | GB: U78045 |
| collagenase MMP-28 | +++ | + | GB: AF219624 |
| caspase 7 | ++ | ++ | GB: BC015799 |
| Caspase 5 | ++ | ++ | GB: NM_004347 |
| Caspase-14 | ++ | ++ | GB: NM_012114 |
| ubiquitin specific protease | ++ | ++ | GB: NM_003481 |
| USP-5 | | | |
| ubiquitin specific protease | ++ | ++ | GB: NM_004651 |
| USP-11 | | | |
| ubiquitin specific protease | ++ | ++ | GB: NM_004505 |
| USP 6 | | | |
| ubiquitin specific protease | + | ++ | GB: NM_031907 |
| USP 26 | | | |
| ubiquitin specific protease | + | ++ | GB: NM_020886 |
| (USP 28) | | | |
| 26S protease subunit 4 | + | ++ | GB: L02426 |
| LILRB1 | | | GB: AF004230 |
| Signal trasducer and | + | ++ | GB: 977935 |
| activator of transcription 1, | | | |
| 91 kDa (STAT1) | | | |
| proteasome (prosome, | + | ++ | GB: X59417 |
| macropain) subunit 6 | | | |
| (PSMA6) | | | |
| TPS1 | +++ | ++ | GB: NM_003293 |
| TPSB1 | ++++ | ++ | GB: XM_016204 |
| TPSG1 | ++ | ++ | GB: XM_008123 |
| protease nexin-II | ++ | ++ | GB: XM_047793 |
| Glia derived nexin | ++ | ++ | GB: P07093 |
| precursor | | | |
| 26S protease regulatory | ++ | ++ | GB: Q92524 |
| subunit S10B | | | |
| PCOLN3 | ++ | ++ | GB: XM 047524 |

We therefore provide the diagnosis of a Group 1 disease or susceptibility to a Group 1 disease (preferably eczema or susceptibility to eczema) in an individual, by detecting modulation of expression, preferably up-regulation of expression of a polypeptide or nucleic acid selected from the group consisting of Apoptosis-related cysteine protease (CASP14) mRNA (NM_{—}012114); TGM5 (XM_{—}007529); phospholipases A(2) (BC013384); CD47 antigen (X69398); Kallilkrein 8 (AB008390); AD024 protein (XM_{—}002642); SCCE (XM_{—}009002); Defensin beta2 (AF0711216); Interferon a inducible protein 27 (X67325); Fatty acid binding protein FABP5 (M94856); SCTE (XM_{—}009000); kallikrein 1, renal/pancreas/salivary (KLK1) (XM_{—}047300); Homo sapiens kallikrein 2, prostatic (KLK2) (XM_{—}031757); kallikrein 3, (prostate specific antigen) (KLK3) (XM_{—}031768); kallikrein 6 (neurosin, zyme) (KLK6) (XM_{—}055658); kallikrein 4 (prostase, enamel matrix, prostate) (KLK4) (XM_{—}008997); membrane-type serine protease 1 (AF133086); Human skin collagenase (M13509); collagenase MMP-1 (LOC116389); collagenase MMP-12 (U78045); collagenase MMP-9 (NM_{—}004994); collagenase MMP-3 (U78045); collagenase MMP-28 (AF219624); caspase 7 (BC015799); Caspase 5 (NM_{—}004347); Caspase-14 (NM_{—}012114); ubiquitin specific protease USP-5 (NM_{—}003481); ubiquitin specific protease USP-11 (NM_{—}004651); ubiquitin specific protease USP 6 (NM_{—}004505); TPS1 (NM_{—}003293); TPSB1 (XM_{—}016204); TPSG1 (XM_{—}008123); protease nexin-II (XM_{—}047793); Glia derived nexin precursor (P07093); 26S protease regulatory subunit S10B (Q92524); and PCOLN3 (XM_{—}047524).

[symbol]

expression of a polypeptide or nucleic acid selected from the group consisting of

We further provide the diagnosis of a Group 1 disease or susceptibility to a Group 1 disease (preferably eczema or susceptibility to eczema) in an individual, by detecting modulation of expression, preferably down-regulation of expression of a polypeptide or nucleic acid selected from the group consisting of Transglutaminase 1 (TGM1) (M98447); TGM2 (XM_{—}009482); TGM4 (XM_{—}056203); TGM7 (NM_{—}052955); TGM3 (L10386); ubiquitin specific protease USP 26 (NM_{—}031907); ubiquitin specific protease (USP 28) (NM_{—}020886); 26S protease subunit 4 (L02426); LILRB1 (AF004230); Signal trasducer and activator of transcription 1, 91 kDa (STAT1) (977935); and proteasome (prosome, macropain) subunit 6 (PSMA6) (X59417).

### Example D7

### Expression of Protease Inhibitor Genes in Eczema Assayed Using Oligonucleotide Arrays

The expression of protease inhibitor genes in eczema patients involved skin (eczema lesional skin), eczema uninvolved (eczema non-lesional skin) and normal skin. The expression level of each gene in disease (involved and uninvolved) is compared to its expression in normal skin.

Results are shown in Table D7.1 below. Key: ++: normally expressed; +++: strongly expressed; ++++: highly expressed, +down-regulation.

TABLE D7.1

| | Expression level | | GenBank |
|---|---|---|---|
| | | Un- | accession |
| Gene Name | Involved | involved | number |
| SLPI | + | ++ | GB: X04502 |
| SKALP | + | + | XM_009524; |
| | | | L10343 |
| CSTA | + | + | GB: |
| | | | NM_005213; |
| | | | AA570193 |
| SCCA | + | + | GB: S66296 |
| SCCA2 | + | + | GB: U19557 |
| plasminogen activator | + | ++ | BG: X04729; |
| inhibitor type 1 | | | X04731 |
| PAI2 | + | ++ | GB: AF071400 |
| SERPINA5 | + | + | GB: NM_000624 |
| hbc750 Human pancreatic | ++ | ++ | GB: T11141; |
| islet | | | T10920 |
| plasminogen activator | ++ | ++ | GB: L19066 |
| inhibitor type 2 | | | |
| TIMP | + | ++ | GB: D11139 |
| TIMP-1 | + | + | GB: NM_003254 |
| TIMP-2 | + | + | GB: NM_003255 |
| TIMP-3 | + | ++ | GB: E13880 |
| TIMP-4 | ++ | ++ | GB: NM_003256 |
| TIM9a | ++ | + | GB: AF150100 |
| TIM9b | + | + | GB: AF150105 |
| Cystatin A | + | + | GB: AA570193 |
| Cystatin M/E | + | + | GB: NM_001323 |
| multivalent protease | +++ | ++ | GB: AF422194 |
| inhibitor WFIKKN | | | |
| C1 inhibitor (SERPING1) | + | ++ | GB: XM_046218 |
| protease inhibitor, Kunitz | + | + | GB: XM_032280 |
| type, 2 (SPINT2) | | | |
| serine protease inhibitor, | + | ++ | GB: XM_005539 |
| Kazal type 4 (SPINK4) | | | |
| proteinase inhibitor, clade B | + | + | GB: XM_053642 |
| (ovalbumin), member 9 | | | |
| serine (or cysteine) | + | + | GB: XM_047984 |
| proteinase inhibitor, clade | | | |
| B (ovalbumin), member 6 | | | |
| eppin-1 (EPPIN) | +++ | ++ | GB: AF286368 |
| eppin-2 (EPPIN) | ++ | ++ | GB: AF286369 |
| eppin-3 (EPPIN) | ++ | ++ | GB: AF286370 |
| Serine protease inhibitor- | + | ++ | GB: NM_020398 |
| like, with Kunitz and WAP | | | |
| domains 1 (eppin) | | | |
| (SPINLW1) | | | |
| sparc/osteonectin, cwcv and | ++ | ++ | GB: NM_004598 |
| kazal-like domains | | | |
| proteoglycan (testican) | | | |
| (SPOCK) | | | |
| protease inhibitor Kunitz | + | + | GB: XM_056836 |
| type 1 (SPINT1) | | | |
| PI12 | ++ | +++ | GB: AH009756 |
| Human immunodeficiency | ++ | ++ | GB: AB020923 |
| virus type 1 gene for HIV-1 | | | |
| protease | | | |
| Human immunodeficiency | + | ++ | GB: AB020924 |
| virus type 1 gene for HIV-1 | | | |
| protease | | | |
| tissue factor pathway | + | ++ | GB: NM_006528 |
| inhibitor 2 (TFPI2) | | | |
| secreted phosphoprotein 2, | ++ | ++ | GB: NM_006944 |
| 24 kD (SPP2) | | | |
| cathepsin F (CTSF) | + | + | GB: NM_003793 |
| serine (or cysteine) | + | + | GB: NM_001756 |
| proteinase inhibitor, clade A | | | |
| (alpha-1 antiproteinase, | | | |
| antitrypsin), member 6 | | | |
| (SERPINA6) | | | |
| serine (or cysteine) | + | + | GB: NM_006919 |
| proteinase inhibitor, clade B | | | |
| (ovalbumin), member 3 | | | |
| (SERPINB3) | | | |
| Serine (or cysteine) | + | + | GB: NM_001085 |
| proteinase inhibitor, clade A | | | |
| (alpha-1 antiproteinase, | | | |
| antitrypsin), member 3 | | | |
| (SERPINA3) | | | |
| Homo sapiens serine (or | + | + | GB: XM_008743 |
| cysteine) proteinase | | | |
| inhibitor, clade B | | | |
| (ovalbumin), member 13 | | | |
| serine (or cysteine) | + | ++ | GB: XM_008742 |
| proteinase inhibitor, clade B | | | |
| (ovalbumin), member 5 | | | |
| (SERPINB5 | | | |
| RelA-associated inhibitor | + | ++ | GB: XM_057693 |
| inhibitor of DNA binding 1, | + | ++ | GB: XM_046179 |
| dominant negative helix- | | | |
| loop-helix protein (ID1) | | | |
| serine (or cysteine) | + | + | GB: XM_054850 |
| proteinase inhibitor, clade E | | | |
| (nexin, plasminogen | | | |
| activator inhibitor type 1), | | | |
| member | | | |
| (SERPINE1) | | | |
| cyclin-dependent kinase | ++ | ++ | GB: NM_004936 |
| inhibitor 2B (p15, inhibits | | | |
| CDK4) (CDKN2B) | | | |
| Similar to cyclin-dependent | + | +++ | GB: BC014469 |
| kinase inhibitor 2B (p15, | | | |
| inhibits CDK4) | | | |
| serine (or cysteine) | + | + | GB: XM_008745 |
| proteinase inhibitor, clade B | | | |
| (ovalbumin), member 7 | | | |
| (SERPINB7 | | | |
| protein inhibitor of | + | ++ | GB: NM_016149 |
| activated STAT protein | | | |
| PIASy (PIASY) | | | |
| similar to protein inhibitor | + | ++ | GB: XM_016864 |
| of activated STAT protein | | | |
| PIASy (LOC95830) | | | |
| PKC-potentiated PP1 | + | + | GB: AY050668 |
| inhibitory protein | | | |
| (PPP1R14A) | | | |
| inhibitor of DNA binding 3, | + | ++ | GB: NM_002167 |
| dominant negative helix- | | | |
| loop-helix protein (ID3) | | | |
| Clade A (alpha-1 | + | + | XM_028358 |
| antiproteinase, antitrypsin) | | | |
| serine (or cysteine) | + | ++ | GB: NM_004353 |
| proteinase inhibitor, clade H | | | |
| (heat shock protein 47), | | | |
| member 1 (SERPINH1) | | | |
| PI13 gene for hurpin (serine | + | ++ | GB: AJ278717 |
| protease inhibitor) | | | |
| protease inhibitor 5 | + | + | XM_008742 |
| (maspin) (PI5) | | | |
| PAI-2 | + | + | A32415 |

We therefore provide the diagnosis of a Group 1 disease or susceptibility to a Group 1 disease (preferably eczema or susceptibility to eczema) in an individual, by detecting modulation of expression, preferably down-regulation of expression of a polypeptide or nucleic acid selected from the group consisting of hbc750 Human pancreatic islet (T1141; T10920); TIMP-4 (NM_{—}003256); TIM9a (AF150100); plasminogen activator inhibitor type 2 (L19066); multivalent protease inhibitor WFIKKN (AF422194); eppin-1 (EPPIN) (AF286368); eppin-2 (EPPIN) (AF286369); eppin-3 (EPPIN) (AF286370); sparc/osteonectin, cwcv and kazal-like domains proteoglycan (testican) (SPOCK) (NM_{—}004598); PI12 (AH009756); Human immunodeficiency virus type 1 gene for HIV-1 protease (AB020923); secreted phosphoprotein 2, 24 kD (SPP2) (NM_{—}006944); and cyclin-dependent kinase inhibitor 2B (p15, inhibits CDK4) (CDKN2B) (NM_{—}004936).

We therefore provide the diagnosis of a Group 1 disease or susceptibility to a Group 1 disease (preferably eczema or susceptibility to eczema) in an individual, by detecting modulation of expression, preferably up-regulation of expression of a polypeptide or nucleic acid selected from the group consisting of SLPI (X04502); SKALP (XM_{—}009524; L10343); CSTA (NM_{—}005213; AA570193); SCCA (S66296); SCCA2 (U19557); plasminogen activator inhibitor type 1 (X04729; X04731); PAI2 (AF071400); SERPINA5 (NM_{—}000624); TIMP (D11139); TIMP-1 (NM_{—}003254); TIMP-2 (NM_{—}003255); TIMP-3 (E13880); TIM9b (AF150105); Cystatin A (AA570193); Cystatin M/E (NM_{—}001323); C1 inhibitor (SERPING1) (XM_{—}046218); protease inhibitor, Kunitz type, 2 (SPINT2) (XM_{—}032280); serine protease inhibitor, Kazal type 4 (SPINK4) (XM_{—}005539); proteinase inhibitor, clade B (ovalbumin), member 9 (XM_{—}053642); serine (or cysteine) proteinase inhibitor, clade B (ovalbumin), member 6 (XM_{—}047984); Serine protease inhibitor-like, with Kunitz and WAP domains 1 (eppin) (SPINLW1) (NM_{—}020398); protease inhibitor Kunitz type 1 (SPINT1) (XM_{—}056836); Human immunodeficiency virus type 1 gene for HIV-1 protease (AB020924); tissue factor pathway inhibitor 2 (TFPI2) (NM_{—}006528); cathepsin F (CTSF) (NM_{—}003793); serine (or cysteine) proteinase inhibitor, clade A (alpha-1 antiproteinase, antitrypsin), member 6 (SERPINA6) (NM_{—}001756); serine (or cysteine) proteinase inhibitor, clade B (ovalbumin), member 3 (SERPINB3) (NM_{—}006919); Serine (or cysteine) proteinase inhibitor, clade A (alpha-1 antiproteinase, antitrypsin), member 3 (SERPINA3) (NM_{—}001085); Homo sapiens serine (or cysteine) proteinase inhibitor, clade B (ovalbumin), member 13 (XM_{—}008743); serine (or cysteine) proteinase inhibitor, clade B (ovalbumin), member 5 (SERPINB5 (XM_{—}008742); RelA-associated inhibitor (XM_{—}057693); inhibitor of DNA binding 1, dominant negative helix-loop-helix protein (ID1) (XM_{—}046179); serine (or cysteine) proteinase inhibitor, clade E (nexin, plasminogen activator inhibitor type 1), member (SERPINE1) (XM_{—}054850); Similar to cyclin-dependent kinase inhibitor 2B (p15, inhibits CDK4) (BC014469); serine (or cysteine) proteinase inhibitor, clade B (ovalbumin), member 7 (SERPINB7 (XM_{—}008745); protein inhibitor of activated STAT protein PIASy (PIASY) (NM_{—}016149); similar to protein inhibitor of activated STAT protein PIASy (LOC95830) (XM_{—}016864); PKC-potentiated PP1 inhibitory protein (PPP1R14A) (AY050668); inhibitor of DNA binding 3, dominant negative helix-loop-helix protein (ID3) (NM_{—}002167); Clade A (alpha-1 antiproteinase, antitrypsin) serine (or cysteine) proteinase inhibitor, clade H (heat shock protein 47), member 1 (SERPINH1) (NM_{—}04353); PI13 gene for hurpin (serine protease inhibitor) (AJ278717); protease inhibitor 5 (maspin) (PI5) (XM_{—}008742); and PAI-2 (A32415).

### Example D8.

### Semi-Quantitative RT-PCR

RNA Extraction from the Epidermis

RNA extraction is carried out according to the manufacturers instructions using the RNeasy kit (QIAGEN). RNAs are extracted from proliferative and differentiated keratinocytes, and also from normal epidermis and psoriatic lesional and non lesional epidermis. The quantification is performed by using three different dilutions of RT to perform a PCR for each sample.

RT-PCR of Corneodesmosomal, Protease and Protease Inhibitor in Normal, Psoriatic, and Eczematous Skin

The expression level of various corneodesmosomal, protease and protease inhibitor genes is assayed by RT-PCR on normal, psoriatic and eczematous skin (involved and uninvolved). Expression levels in involved and uninvolved skin from individuals are compared to the corresponding expression level in normal, undiseased skin. The corneodesmosomal, protease and protease inhibitor genes assayed are those listed in Tables D2.1 and D5.1; D3.1 and D6.1; and D4.1 and D7.1 respectively.

Results

RT-PCR results show broadly the expression changes as detailed in the above Examples. The results of expression level studies of the various corneodesmosomal, protease and protease inhibitor genes assayed by oligonucleotide arrays is therefore confirmed by direct assay of level of expressed message.

### Examples E

### Treatment

### Example E1

### Corneodesmosome Density Assayed Using Tape Strips

Preparation of Tape Strips for Transmission Electron Microscopy

Tape strips are obtained according to a method described by Guerrin et al, 1998.

Small pieces of tape strips from control patients along with patients suffering from the skin conditions eczema, psoriasis, dermatitis and psoriasis are fixed for 2 h at 4° C. in Karnovsky fixative (2% paraformaldehyde, 2.5% gluteraldehyde in 0.1M phosphate buffer, pH 7.4). The samples are then washed three times in 0.1M phosphate buffer (pH7.4) containing 10% sucrose for 30 minutes each at 4° C.

Secondary fixation is carried out in aqueous 2% osmium tetroxide solution for 1 hour at room temperature. Dehydration is through a graded series of ethanol at room temperature (75% ethanol for 15 minutes; 95% ethanol for 15 minutes; 2 washes of 100% ethanol for 15 minutes; 100% ethanol dried over anhydrous copper sulphate for 15 minutes).

For resin embedding, samples are first placed in the intermediate solvent, propylene oxide, for two changes of 15 minutes duration. This is followed by infiltration in a 50:50 mixture of propylene oxide:araldite resin overnight at room temperature. The samples are placed in full strength araldite resin for 6-8 hours at room temperature. The biopsies are embedded in fresh araldite resin for 48 hours at 60° C. Araldite resin for embedding consisted of a 50:50 mixture of CY212 araldite:dodecenyl succinic anhydride (DDSA) hardener and 1 drop of n-benzyldimethylamine (BDMA) accelerator per 1 ml of resin mixture.

Ultrathin sections (70-90 nm) are cut on an ultramicrotome. Sections are stained for 5 minutes with 3% Uranyl Acetate in 50% ethanol followed by staining with Reynold's Lead Citrate for 2 minutes. The sections are examined using a Philips CM10 Transmission Electron Microscope at an accelerating voltage of 80 Kv. Electron micrographs are recorded on Agfa Scientific 23D56 EM film.

Corneodesmosome density (area occupied by corneodesmosomes divided by total area of micrograph) is measured on electron micrographs of tape strips of each condition.

Treatment of Patients Stratum Corneum with Protease

Tape strips of 3×3 mm from normal and diseased stratum corneum are incubated with 22-50 nM recombinant SCCE or SCTE in 10 ×concentrated proteolysis buffer (10 mM sodium phosphate buffer, pH 7.2, 0.15 M NaCl) for 1-6 h at 37° C. The reactions are stopped and the strips fixed for electronic microscopy analysis for corneodesmosome counting as described above.

Results

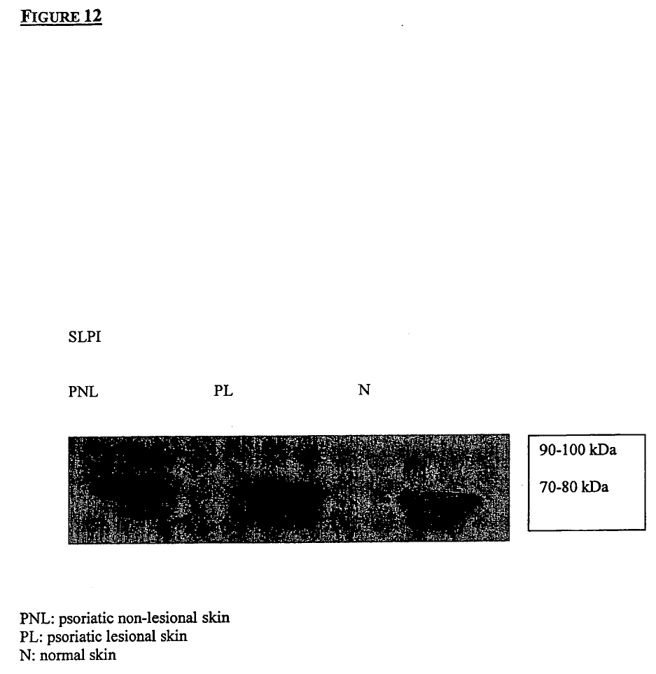

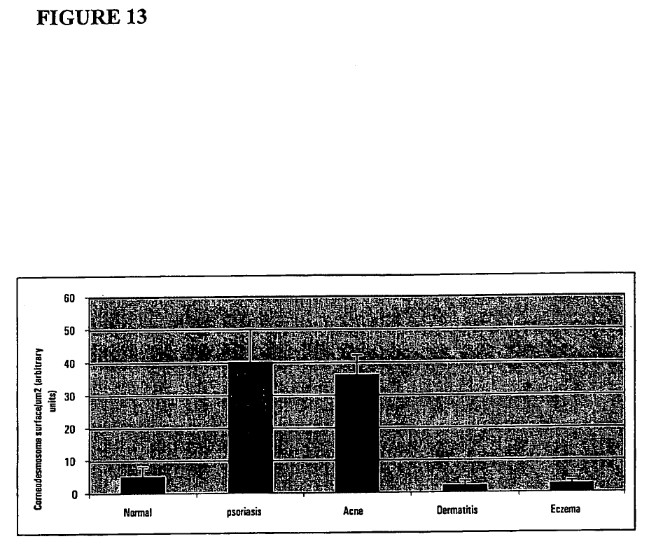

The results of this experiment are shown in FIG. 13.

The density of corneodesmosomes in the upper stratum corneum of patients with psoriasis and acne is significantly increased compared with normal skin. The density of corneodesmosomes in the stratum corneum of dermatitis and eczema patients is significantly decreased.

### Example E2

### Treatment of Tape Strips from Psoriatic Skin with Proteases

Tape strips from normal and psoriatic individuals are obtained and treated as described. Corneodesmosome density is assayed as before.

Results

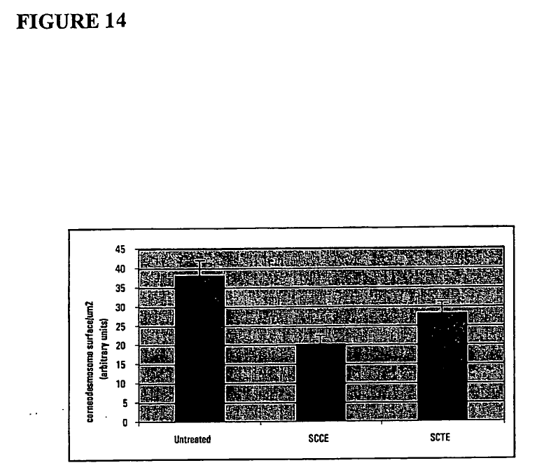

The results of this experiment are shown in FIG. 14.

The number of corneodesmosomes is found to decrease significantly in the treated stratum corneum compared to untreated stratum corneum. These experiments demonstrate that there is direct relationship between corneodesmosome number and proteolysis by SCCE or SCTE.

Discussion

Using proteases (e.g. SCCE/SCTE) we demonstrate reduction of the number of corneodesmosomes in the stratum corneum of patients with increased skin barrier (i.e., Group II disease). We therefore propose a new treatment to promote the formation of a normal skin barrier in patients with increased stratum corneum cohesion.

### Example E3

### Treatment of Skin Biopsies from Psoriatic Patients with Protease

Recruitment

Patients with psoriasis are identified and recruited through a dermatology clinic at the Royal Hallamshire Hospital. Patients are selected if they are over the age of forty, have evidence of active psoriasis and are not taking systemic antipsoriatic agents. Patients with a history of rheumatic fever, prosthetic heart valve replacement or joint replacement are automatically excluded. Full informed written consent is obtained from each patient with MREC approval (MREC/98/4/018). Topical treatment of the biopsy site is omitted for two weeks prior to the procedure.

Procedure

All biopsies are carried out by a dermatologist in an operating theatre. Aseptic technique is employed and sterile/disposable equipment used throughout the procedure. Two skin biopsies are taken from the lower back of each patient, one from an area of active plaque psoriasis and the other from an uninvolved site at least 10 cm away. The lower back is cleaned with antiseptic solution and local anaesthetic introduced to the intended areas of biopsy. Ellipses approximately 1.5 cm by 0.5 cm are excised from both lesional and non-lesional skin and placed in a container with PBS solution. The wounds are sutured using catgut for deep sutures and ethilon for superficial sutures (employing two or three stitches per wound). After the operation the wounds are covered with a sterile dressing and the patient observed for 30 minutes before being allowed home. Sutures are removed two weeks later.

Protease Treatment

The following procedures are performed under aseptic conditions: The psoriatic skin biopsy is cut into 2 pieces using a sterile scalpel. One half of the biopsy is placed in 2 ml of 0.25% chymotrypsin (diluted from a 10×stock in skin maintenance medium, Skinethic) in one well of a 24-well plate. The other half is incubated in 2 ml of maintenance medium with no additions. The well plate is incubated at 37° C. for 16 hours. Both biopsy segments are then rinsed through two changes of PBS and fixed for electron microscopy as below.

Processing for Electron Microscopy

The skin biopsy samples are fixed in Karnovsky fixative (2% paraformaldehyde, 2.5% gluteraldehyde in 0.1M phosphate buffer, pH 7.4) for 2 hours at 4° C. The samples are then washed three times in 0.1M phosphate buffe (pH7.4) containing 10% sucrose for 30 minutes each at 4° C.

Secondary fixation is carried out in aqueous 2% osmium tetroxide solution for 1 hour at room temperature. Dehydration is through a graded series of ethanol at room temperature(75% ethanol for 15 minutes; 95% ethanol for 15 minutes; 2 washes of 100% ethanol for 15 minutes; 100% ethanol dried over anhydrous copper sulphate for 15 minutes).

For resin embedding, samples are first placed in the intermediate solvent, propylene oxide, for two changes of 15 minutes duration. This is followed by infiltration in a 50:50 mixture of propylene oxide:araldite resin overnight at room temperature. The samples are placed in full strength araldite resin for 6-8 hours at room temperature. The biopsies are embedded in fresh araldite resin for 48 hours at 60° C. Araldite resin for embedding consisted of a 50:50 mixture of CY212 araldite:dodecenyl succinic anhydride (DDSA) harderner and 1 drop of n-benzyldimethylamine (BDMA) accelerator per 1 ml of resin mixture.

Ultrathin sections (70-90 nm) are cut on an ultramicrotome. Sections are stained for 5 minutes with 3% Uranyl Acetate in 50% ethanol followed by staining with Reynold's Lead Citrate for 2 minutes. The sections are examined using a Philips CM10 Transmission Electron Microscope at an accelerationg voltage of 80 Kv. Electron micrographs are recorded on Agfa Scientia 23D56 EM film.

Results

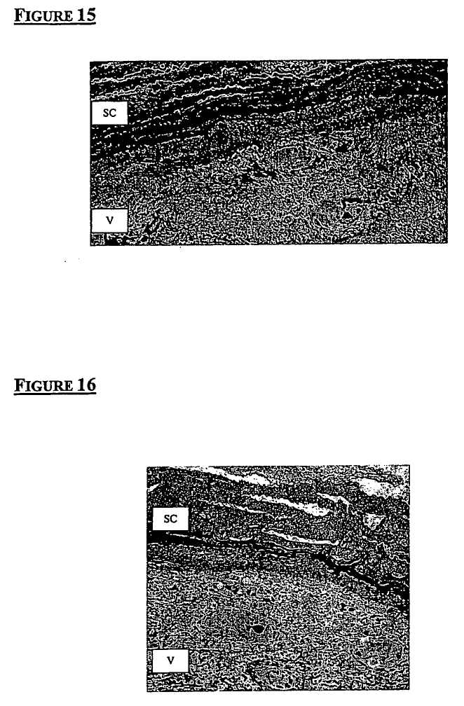

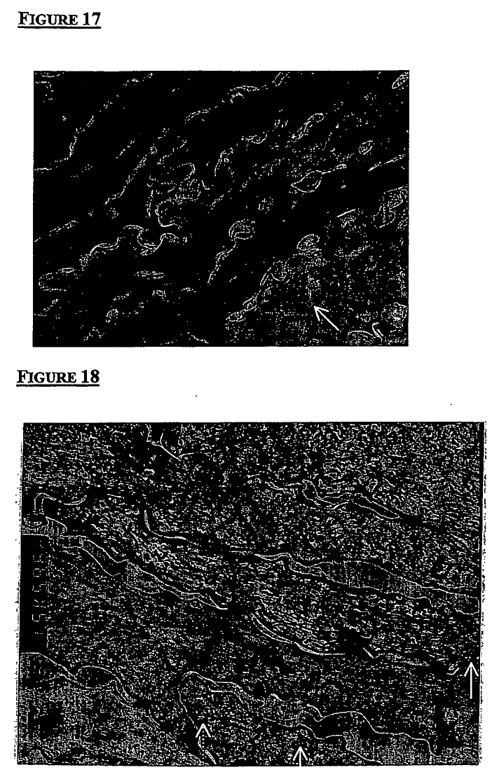

The results are shown in FIG. 15, FIG. 16, FIG. 17 and FIG. 18.

FIG. 15 shows a untreated psoriatic biopsy. FIG. 16 shows that a psoriatic biopsy treated for 16 hours with 0.25% chymotrypsin produces splitting of the layers of corneocytes (acantholysis). This is caused by degradation of the corneodesmosomes by chymotrypsin. FIG. 17 shows a section of an untreated psoriatic biopsy (stratum corneum). Numerous corneodesmosomes can be seen, some of these are indicated by the white arrows. FIG. 18 shows that in a stratum corneum of psoriatic biopsy treated for 16 hours with 0.25% chymotrypsin, far fewer corneodesmosomes are visible after protease treatment (solid white arrows). Some remnants of degraded corneodesmosomes can also be seen (dashed white arrows).

Discussion

Comparing psoriatic lesional biopsies treated with chymotrypsin and the control (untreated lesional psoriatic skin), there is a marked splitting of the epidermal cell layers (acantholysis) in the chymotrypsin treated samples. This is present within the lower part of the stratum corneum and at the junction of the stratum corneum and the viable epidermis. The breakdown of the skin barrier may be used for the treatment of diseases with increased barrier function (i.e., Group II diseases), for example psoriasis (as detailed here) and acne.

### Example E4

### Treatment of Skin Equivalents with Proteases

Materials

Reconstituted human epidermis cultures and maintenance medium are purchased from Skinethic Tissue Culture Laboratories, Nice. Chymotrypsin is purchased from Sigma Chemicals.

Methods

1 ml of room temperature SkinEthic maintenance medium is added to each well of 6-well tissue culture plates (one well per skin equivalent). The cell culture inserts containing the skin equivalents are removed from the agarose transportation medium using sterile forceps and placed in the wells ensuring no air bubbles are formed. The well plates are placed in a humid incubator at 37° C., 5% CO2. After 24 hours the medium is changed and the appropriate treatment is applied. 200 μl of each treatment is applied to the surface of the skin equivalent as follows: Control—buffered salt solution; 10% foetal bovine serum; 0.25% chymotrypsin; 6 μM Peptide 643.

All reagents are made up to the correct concentration in a buffered salt solution comprising the following in 1 litre: 0.142 g Na₂HPO₄, 1.802 g Glucose, 7.149 g HEPES, 0.224 g KCl, 7.597 g NaCl, pH 7.4.

Treatments are allowed to act on the cultures for 16 hours at 37° C.

Processing for Light Microscopy

The skin equivalents and membranes are cut from the cell culture insert using a scalpel. The equivalents are fixed in 10% formalin (3.7% formaldehyde) in phosphate buffered saline for 24 hours at 4° C. Samples are washed in 3 changes of PBS. The PBS is the decanted from the samples which are dehydrated in 3×5 minute changes of 70% ethanol, 2×5 minute changes of 95% ethanol and 2×5 minute changes of 100% ethanol. The samples are then placed in xylene for 5 minutes and then molten paraffin for infiltration for 5 minutes. The samples are occasionally agitated during each of the steps. The equivalents are then oriented in more molten paraffin in an embedding mould and allowed to set for 1 hour.

5 μm sections are taken of each block and collected on glass microscope slides. The slides are dried at 50° C. overnight.

Haematoxylin and Eosin Staining for Histology

The slides are racked and placed in xylene for 2×5 minute washes to remove any unwanted paraffin. The slides are then rehydrated by placing them for 5 minutes in each of the following; 2×100% ethanol, 1×95% ethanol, 1×70% ethanol. The slides are then rinsed in running tap water for 1 minute. Staining is with Gill's haematoxylin (2 minutes) followed by rinsing in water for 2 minutes then placing in 1% eosin for 5 minutes and rinsing briefly in water. The samples are dehydrated once more by placing in 70% ethanol for 30 seconds, 95% ethanol for 30 seconds and 2 changes of 100% ethanol for 1 and 2 minutes each. The slides are then placed in xylene for 1 minute before mounting coverslips with DPX mountant for microscopy.

Slides are examined under light microscope at using the 40×objective lens and typical images are captured onto the PC using Synoptics Acquis Pro.

Results

Chymotrypsin (0.25%) degrades the barrier of the stratum corneum. The layers appear to be “looser” and are beginning to lift away from the viable cell layer. Chymotrypsin is therefore shown to degrade the corneodesmosomes.

Similar effects are observed when the skin equivalent is treated with SCCE or SCTE. Proteases such as chymotrypsin, SCCE and SCTE may therefore be used for treatment of diseases such as psoriasis and acne where the stratum corneum thickness is increased. These enzymes shown here decrease the thickness of the stratum corneum barrier by degrading corneodesmosomes which connect the cell layers.

### Example E5

### Treatment of Skin Equivalents with SLPI Protease Inhibitor Peptides

Materials

Day 17 reconstituted human epidermis cultures and maintenance medium are purchased from Skinethic Tissue Culture Laboratories, Nice. NUNC tissue culture plasticware is obtained from Life Technologies.

A number of peptides corresponding to regions of proteins important in skin barrier formation are synthesised using standard techniques. The peptides are as shown in Table E5.1 below.

Peptides 643, 651 and 653 correspond to different regions of the same protein (SLPI). Tumour Necrosis Factor-α (TNF-α) and Interleukin-1β (IL-1β) are purchased from Calbiochem.

TABLE E5.1

| PEPTIDE | | ORIGINAL |
|---|---|---|
| NAME | SEQUENCE | PROTEIN |
| Peptide 641 | NH2-met-glu-asn-ser-leu-gly-pro- | Desmocollin 1 |
| | phe-pro-gln-cys-COOH | |
| Peptide 642 | NH2-ser-gly-lys-arg-asp-lys-ser- | Desmoplakin |
| | glu-glu-val-gln-cys-COOH | |
| Peptide 643 | NH2-cys-val-ser-pro-val-lys-ala- | Secretory Leukocyte |
| | COOH | Protease Inhibitor |
| | | (SLPI) |
| Peptide 651 | NH2-leu-asp-pro-val-asp-COOH | Secretory Leukocyte |
| | | Protease Inhibitor |
| | | (SLPI) |
| Peptide 653 | NH2-leu-asp-pro-val-asp-thr-pro- | Secretory Leukocyte |
| | asn-pro-thr-arg-arg-lys-pro-gly- | Protease Inhibitor |
| | COOH | (SLPI) |
| | | (long form of 651) |

Methods

1 ml of room temperature SkinEthic maintenance medium is added to each well of 6-well tissue culture plates (one well per skin equivalent). The cell culture inserts containing the skin equivalents are removed from the agarose transportation medium using sterile forceps and placed in the wells ensuring no air bubbles are formed. The well plates are placed in a humid incubator at 37° C., 5% CO2. After 24 hours the medium is changed and the appropriate treatment is applied.

All reagents are made up to the correct concentration in a buffered salt solution comprising the following in 1 litre: 0.142 g Na₂HPO₄, 1.802 g Glucose, 7.149 g HEPES, 0.224 g KCl, 7.597 g NaCl, pH 7.4.

200 μl of the following treatments are applied to the surface of the skin equivalents: (1) Control—buffered salt solution; (2) 6 μM Peptide 641; (3) 6 μM Peptide 642; (4) 6 μM Peptide643; (5) 6 μM Peptide 651; (6) 6 μM Peptide 653.

The following treatments are diluted from stock solutions (10 μg/ml TNF-α, 1 μg/m IL-1β) directly into 1 ml of medium and added to the well during the medium change: (7) 2.5 ng/ml TNF-α; and (8) 75 ng/ml IL-1β.

Treatments are allowed to act on the cultures for 16 hours at 37° C.

Results

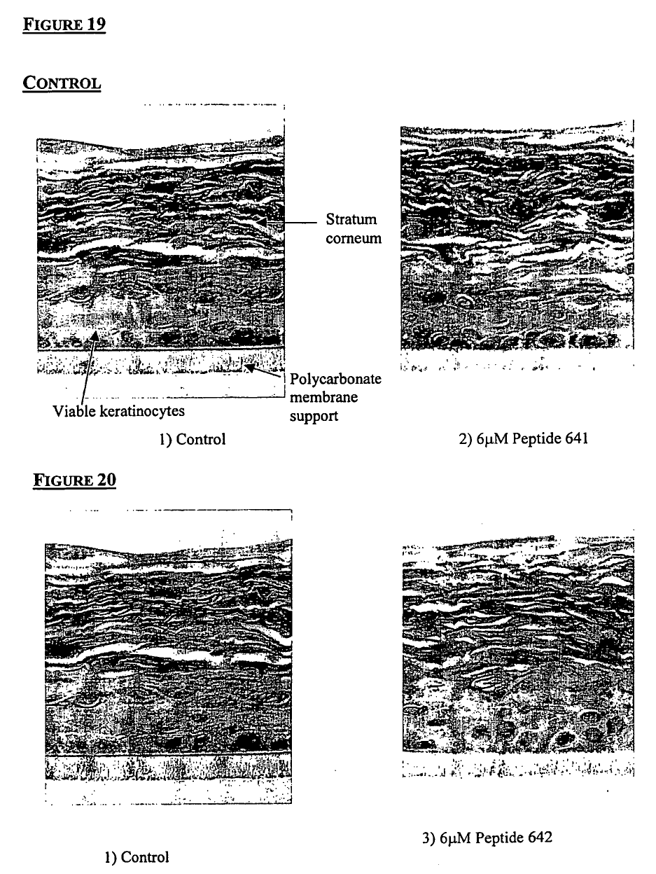

The results are shown in FIGS. 19 to 25.

Serum contains a number of protease inhibitors including inhibitors of trypsin and chymotrypsin. On addition to the skin equivalents, 10% serum is seen to increase the thickness of the stratum corneum.

FIG. 19 shows that addition of 6 μm peptide 641 causes the stratum corneum to become more permeable.

FIG. 20 shows that addition of 6 μM peptide 642 causes the stratum corneum to become more permeable.

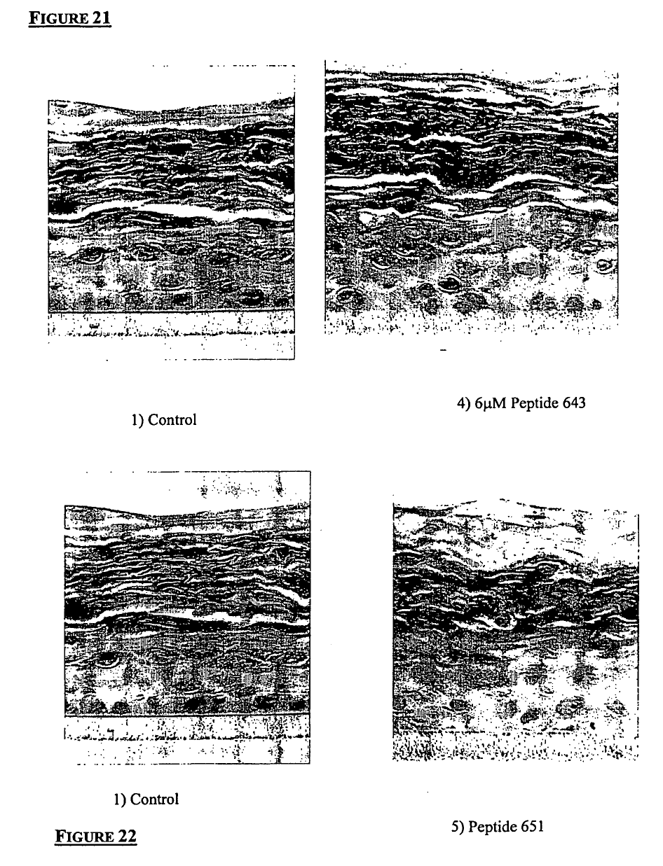

FIG. 21 shows that a more marked effect is observed after addition of peptide 643 to the skin cultures. This peptide mimics a short region of SLPI. Due to its short length (7 amino acids) it is able to penetrate the stratum corneum where it inhibits specifically the protease enzymes including SCCE and SCTE. The overall effect observed is a thickening of the stratum corneum, and therefore an increased skin barrier.

FIG. 22 shows that addition of 6 μM peptide 651 (SLPI) causes an increase in the thickness of the stratum corneum, and therefore an increased skin barrier.

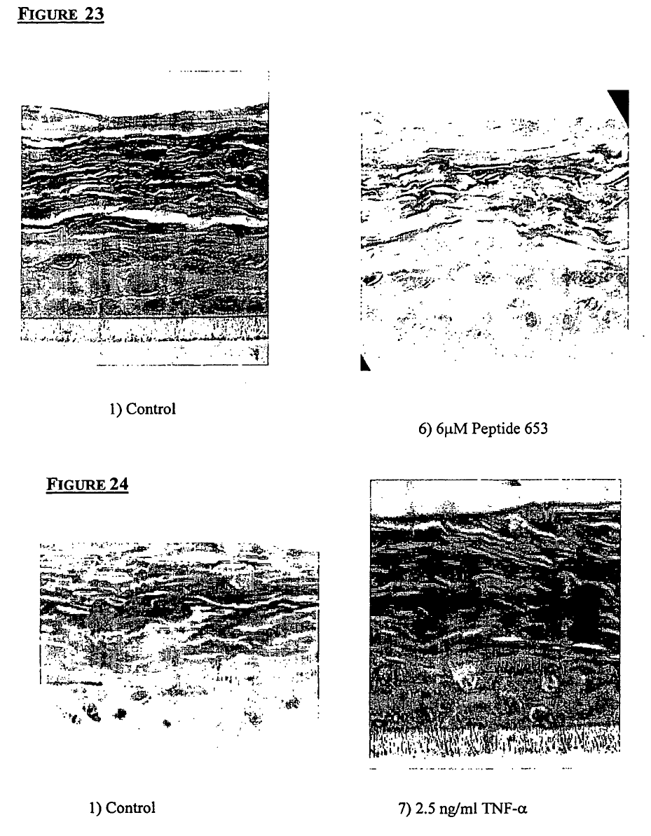

FIG. 23 shows that addition of 6 μM peptide 653 (SLPI) causes an increase in the thickness of the stratum corneum, and therefore an increased skin barrier.

Protease inhibitors, and fragments of these, therefore provide new treatments for diseases with symptoms including impaired skin barrier function (e.g. dermatitis, eczema). The novel peptides included here may be used as a treatment for skin disorders with defective skin barrier due to their small size and penetrability and their effect on improving skin barrier thickness.

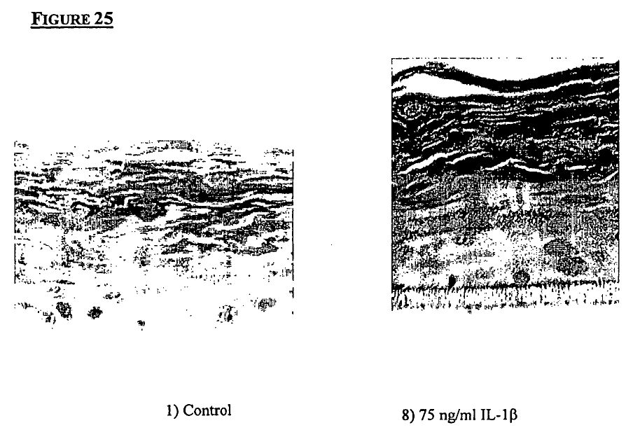

FIG. 24 shows that addition of 2.5 ng/ml TNF-α causes an increase in the thickness of the stratum corneum, and therefore an increased skin barrier.

FIG. 25 shows that addition of 2.5 ng/ml IL-1β causes causes an increase in the thickness of the stratum corneum, and therefore an increased skin barrier.

Discussion

Peptides 641 and 642 mimic regions of desmocollin and desmoplakin, two of the protein components of corneodesmosomes, the cellular structures which are involved in cell cohesion in the stratum corneum. Peptides 641 and 642 mimic regions of the proteins which are involved in cleavage during desquamation.

When skin equivalents are treated with these peptides the texture of the skin barrier altered and appeared to become more permeable. Thus, peptides 641 and 642 may be used as new treatments for diseases with symptoms including increased stratum corneum cohesion (e.g. psoriasis and acne, i.e., any Group II disease).

We therefore provide a method of treatment or prevention of a Group II disease in an individual, preferably psoriasis and/or acne, the method comprising administering an adhesion protein or a fragment thereof, to the individual. Preferably, a fragment of desmocollin or a fragment of desmoplakin is administered; more preferably, a peptide comprising the sequence peptide 641 and/or peptide 642 is administered.

On addition of peptides 643, 651 and 653 to the skin equivalents an increase in stratum corneum thickness is observed. These peptides are designed to mimic regions of SLPI, an inhibitor of the proteases Stratum Corneum Chymotrypsin Enzyme and Stratum Corneum Trypsin Enzyme (SCCE and SCTE respectively). These enzymes degrade the desmosomes in the stratum corneum thus decreasing cell cohesion. The increased thickness of the stratum corneum shows that the peptides 643, 651 and 653 are inhibiting the proteases. The most marked effect is observed after addition of peptide 643 to the skin cultures. This peptide mimics a short region of SLPI. Due to its short length (7 amino acids) it is probably able to penetrate the stratum corneum where it inhibits specifically the protease enzymes SCCE and SCTE.

TNFa and IL-1β may also be used to treat diseases with decrease adhesion such as eczema and dermatitis because they are activators of protease inhibitors such as SLPI.

We therefore provide a method of treatment or prevention of a Group I disease in an individual, preferably eczema and/or dermatitis, more preferably atopic eczema and/or dermatitis herpetiformis, the method comprising administering a protease inhibitor or a fragment thereof, to the individual. Preferably, a fragment of SLPI is administered; more preferably, a peptide comprising the sequence peptide 643, and/or peptide 651 and/or peptide 653 is administered.

The complete list of peptides derived from the SLPI GenBank sequence (X04502) that mimic the inhibitor effect of SLPI is as follow: CGKS (SB7a) and CGKS CVSPVKA (SB7b); KIIDGA; GDKIIDGA; GDKIID; KII; KIID; KIIDG; KIIDGA; LDPVD (651); KRDLK (652); LDPVDTPNP (653); LDPVDTPNPTRRKPG (654); CGKSCVSPVKA (644); CVSPVKA (643). Any of these peptides may therefore be used in the treatment or prevention of a Group I disease.

### Example E6

### Treatment of Skin Equivalents with Other Protease Inhibitors

Methods

Skin equivalent (+ and − inhibitor) is cultured with the inhibitor for 24 h. Skin equivalent are taken the preparation of histological sections. Sections are then analysed by light and electron microscopy.

SLPI

SLPI (500 nM) is added to cultured skin equivalent, and cultured for 24 h. Skin equivalent (+ and − SLPI) are taken the preparation of sections. Sections are then analysed in light and electron microscopy.

Treatment with SLPI is seen to increase the thickness of the stratum corneum, and to enhance the skin barrier. SLPI can therefore be used to treat any Group I disease, such as eczema and dermatitis.

Anti-SCCE Antibodies

SCCE antibody is added is added to cultured skin equivalent, and cultured for 24 h. Skin equivalent (+ and − SCCE antibodies) are taken the preparation of sections. Sections are then analysed in light and electron microscopy.

Treatment with anti-SCCE antibody is seen to increase the thickness of the stratum corneum, and to enhance the skin barrier. Anti-SCCE antibody can therefore be used to treat any Group I disease, such as eczema and dermatitis.

Anti-SCTE Antibodies

SCTE antibody is added is added to cultured skin equivalent, and cultured for 24 h. Skin equivalent (+ and − SCTE antibodies) are taken the preparation of sections. Sections are then analysed in light and electron microscopy.

Treatment with anti-SCTE antibody is seen to increase the thickness of the stratum corneum, and to enhance the skin barrier. Anti-SCTE antibody can therefore be used to treat any Group I disease, such as eczema and dermatitis.

### Example E7

### Treatment of Individuals Suffering from Eczema with Protease Inhibitor

Method

SPLI protease inhibitor is extracted from normal human skin of a volunteer. Alternatively, a nucleic acid sequence encoding the protease inhibitor from the individual is cloned using PCR, or library screening. The individual's protease inhibitor sequence is cloned into an expression vector, and recombinant protease inhibitor expressed and purified using conventional and known means.

The inhibitor is then formulated in an emollient cream base. Two fingertip units of this formulation are applied to an area of the volunteer's skin on the arm measuring 2×2 cm. The area is covered with a plastic capsule which is secured in place with a cotton bandage. The cream is reapplied to the same area each day for 14 consecutive days.

On day 14 the cup is removed and a 1×0.5 cm ellipse skin biopsy is taken from the treated area and a similar biopsy taken from an untreated area on the opposite arm.

Results

Histological examination of the treated biopsy compared with the control reveals a thickening of the stratum corneum with loosely adherent scales in the superficial part of the stratum corneum. This is reminiscent of the features of the upper part of a psoriatic plaque. Within the viable epidermis there is parakeratosis and thickening. These features are also similar to those seen in a psoriatic lesion.

Discussion

The thickening of the skin in response to the addition of the SPLI protease inhibitor is therefore shown to be a useful treatment for diseases with symptoms including impaired skin barrier function (e.g. eczema, dermatitis).

### Examples F

### Transgenic Organisms

### Example F1

### Transgenic Mice Over-Expressing Corneodesmosin

We over-expressed the corneodesmosin gene in mouse in order to generate transgenic mice with abnormal skin barrier.

Generation of Corneodesmosin Fragment

cDNA is generated from mRNA isolated from the epidermis using set of primers including forward 5′ccgtgcagtccgagatg3′ and reverse 5′gatatagtgtatgtgcttg3′ which contain 5′UTR the whole ORF and 3′UTR. PCR products (1659 bp) are purified (as described above). The purified products are used as template using modified primers. These primers contain NotI site in their 5′extremity.

PCR products are purified from 1% agarose gel incubated with NotI restriction enzyme 4 hrs at 37° C. Digested products are inserted into involucrin expression cassette and transgene fragment is excised from the parent plasmids and purified. Purified fragments are suspended in sterile PBS at a concentration of 5 mg/ml for oocyte injection.

Involucrin Expression Cassette

The involucrin expression cassette includes the 3.7-kb involucrin sequences (2.5 kb of involucrin promoter), a simian virus 40 (SV40) intron, and an SV40 polyadenytion sequence as described by Carroll et al, 1993 and 1996. Briefly, the 3740 bp Hind III fragment from −2500 to +1240 (numbers are based on their sequence distance upstream (−) and downstream (+) of transcriptional initiation site (+1)), containing distal region, proximal region TATAA, exon 1, intron 1 of involucrin gene including sites donor and acceptor of intron 1 (Carroll et al, 1993).

Animal Phenotypes

The mice over expressing corneodesmosin have thickened skin with a scaly surface. These changes are most prominent on the limbs, ears and head. Histology of the lesional skin reveals a acanthosis of the epidermal ridges which are elongated and club shaped at the bases. There is also parakeratosis and hyperkertosis within the epidermis. The stratum corneum is thickened with a scaly surface. These features are similar to some changes seen in psoriatic skin.

A diffuse alopecia develops in the corneodesmosin mice. This is more obvious after the second month.

Histology and Immunohistochimestry

Tissues from transgenic and trangene-negative mice matched according to sex and body site are used for all tissue studies. For histological analysis, tissues are fixed overnight in formol-saline, embedded in paraffin, sectioned, and stained with hematoxylin and eosin. Chloroacetate esterase is used as a histochemical marker for neutrophils. TUNEL is performed on paraffin sections of normal and tragenic mice (see Carroll et al, 1995). The skin is thick and histology sections of dorsal skin of mice show a significant increase of the thickness of the epidermis. The skin barrier is enhanced in these animals as is observed in psoriatic skin.

Imunnohistochemistry is performed with antibodies specific for mouse corneodesmosin (1:40 dilution). Primary antibody is detected by secondary antitbody using avidin DH and biotinylated staining system (Vectasatain ABC kit, USA). Staining shows that there is a high expression of corneodesmosin in the skin of transgenic animals compared to matched controls. This over-expression of the corneodesmosin is mainly seen in the suprabasal layers of the transgenic animal skins.

### Example F2

### Transgenic Mice Over-Expressing SCCE

We over-expressed SCCE in mouse in order to generate transgenic mice with severe abnormal (defective) skin barrier.

Different SCCE haplotypes are amplified using RNA from disease epidermis using set of primers including forward (5′ To 3′): CGG GCT CCA TGG CAA GAT C and reverse (5′ To 3′): GCG TCC TCA CTC CTG TGC which contain 5′UTR the whole ORF and 3′UTR. PCR products are purified (as described above). The purified products are used as template using modified primers. These primers contain NotI site in their 5′extrimity. PCR products are purified from 1% agarose gel incubated with NotI restriction enzyme 4 hrs at 37° C. Digested products are inserted into involucrin expression cassette and the transgene fragment is excised from the parent plasmids and purified. Purified fragments are suspended in sterile PBS at a concentration of 5 mg/ml for oocyte injection.

Animal Phenotypes

Adult mice over-expressing the SCCE have abnormalities in the skin barrier. Animals over-expressing SCCE in the supralayers present widespread skin blistering. The skin barrier defect is more pronounced in these animals compared to animals over-expressing SCCE non-disease alleles.

Histology and Immunohistochemistry

Tissues from transgenic and transgene-negative mice are matched and treated as described above.

The skin is thick and histology sections of dorsal skin of mice show a significant increase of the thickness of the epidermis. The skin barrier is impaired in these animals as is observed in eczematic skin.

Histology of blisters in mice with a defective skin barrier shows loss of cell adhesion in the superficial layers of the epidermis.

Imunnohistochemistry is performed with antibodies specific for mouse SCCE (1:40 dilution). Primary antibody is detected by secondary antibody using avidin DH and biotinylated staining system (Vectastain ABC kit, USA). Staining shows that there is a high expression of SCCE in the skin of transgenic animals compared to matched controls. This over-expression of the SCCE is mainly seen in the suprabasal layers of the transgenic animal skins.

The animals over expressing SCCE in the suprabasal layers have a dry flaky skin. Histological examination of the skin reveals splits in the epidermis. This acantholysis is similar to that seen in the skin equivalent cultures treated with chymotrypsin (seee treatment section in this study). The splits occurr within the granular layer, between the granular and spinous layers and between the cornified and granular layers. There are also splits within the stratum corneum.

The epidermis of the SCCE transgenic (+/+) mice is thicker than that of litter mates. This thickening is found at all body sites. We tested the integrity of the epidermal barrier in the SCCE transgenic mice using dye penetration assays (Hardman et al., 1998; Marshall et al., 2000). The SCCE +/+ transgenic mice show numerous dark spots indicating localized loss of barrier function.

### Example F3

### Transgenic Mice Over-Expressing SLPI

cDNA is generated from mRNA isolated from the epidermis using set of primers including forward 5′ctcctgccttcaccatgaag3′ and reverse 5′cagagcctcctccatatg3′ which contain 5′UTR the whole ORF and 3′UTR. PCR products are purified (as described above).

The purified products are used as template using modified primers. These primers contain NotI site in their 5′extrimity. PCR products are purified from 1% agarose gel incubated with NotI restriction enzyme 4 hrs at 37° C. Digested products are inserted into involucrin expression cassette and the transgene fragment is excised from the parent plasmids and purified. Purified fragments are suspended in sterile PBS at a concentration of 5 mg/ml for oocyte injection.

Animal Phenotypes

Adult mice over-expressing the SLPI have abnormalities in the skin. SLPI is expressed mainly suprabasal layers of the epidermis in the transgenic animal; this indicates that the transgenic mice show phenotypes similar to psoriatic skin.

Histology and Immunohistochemistry

Tissues from transgenic and transgene-negative mice matched according to sex and body site are used for all tissue studies. For histological analysis, tissues are fixed overnight in formol-saline, embedded in paraffin, sectioned, and stained with hematoxylin and eosin. Chloroacetate esterase is used as a histochemical marker for neutrophils. TUNEL is performed on paraffin sections of normal and transgenic mice (see Carroll et al, 1995). The skin is thick and histology sections of dorsal skin of mice shows a significant increase of the thickness of the epidermis. The skin barrier is enhanced in these animals as is observed in psoriatic skin.

Imunnohistochemistry is performed with antibodies specific for mouse SLPI (1:40 dilution). Primary antibody is detected by secondary antitbody using avidin DH and biotinylated staining system (Vecta stain ABC kit, USA). Staining shows that there is a high expression of SLPI in the skin of transgenic animals compared to matched controls. This over-expression of the SLPI is mainly seen in the suprabasal layers of the transgenic animal skins.

### Annex A: Cystatin A Reference Sequences

CystA.1 (Cystatin A sequence 1) Sequence

Position 1 is the start of Exon 1. The exons are underlined.

| | |
|---|---|
| −1400 | TTTTATAGTT |
| −1350 | CATCAAGTCA CCGTGCCCAG CCTCCAATTA TTTTTATATT TGTATGTGTA |
| −1300 | AATGAATGTC TCTGAAGGAT ACAGAAGAAG TTGGTGCCAA TAATTGTCTC |
| −1250 | TGGAGAGTAG AAGGGTGTCT GAGGAATTAA GTGAGTGAAT AATTCCTTAT |
| −1200 | ATTGTAAAGC CTGGTTCCTT CCAAACATTT TTATCCTGTG TATGTATTAT |
| −1150 | ATATTCCCAA AATTGAATAA AATAATATAC ATAAATATTC ACACAATGTG |
| −1100 | GCCATTTTGC TTCTAGATAA GTTGTCTGCA GAAGCCCTCA CTCATGTAAA |
| −1050 | AAGGAGAAAA AATGCAACGT TGTTTGTAAG AATGAAACAC CAGAAACAAC |
| −1000 | GAAAATCGTC ATCAATAGAG AAATGAGTAT GTAAAATATG AACTAGAATA |
| −950 | TAGCAATTAG ATAGGTAGAC TAAATAGATA GGTCTAGCAT AAAATAGCAT |
| −900 | ATCACAGTTA AAGGAAATGA ACAGGATTGA TCTATCTGTG GATTGATCTA |
| −850 | TGTGTATATC AACATAGAAA GGCTCAAAAA CATGTTGAAT ACAAAAAGGA |
| −800 | ACATAACATA GAATATATTT AGCATACAAT TTAAGTGAAA TTTTAAAGAC |
| −750 | ACACCAAGTA AGACATATTT TAATTTTAAA GACACACATA TAAAAATGGC |
| −700 | CTGGAAGGAT ATTAATTCAC CATGTACTTT GCCTTCTGGA AGGCAAGGTT |
| −650 | GCGTGGGGTG TGGGGCTTCC TCCATATCTG TAATATTTTA TTTCCTAAAA |
| −600 | ATAACTACAA AAATAAAAAA CAGCAAGCAA ATATGACAAA AGGGTTAAAA |
| −550 | GTTTTAATTC TGAGTGATAG AAATATAGAT GTTTGTTATT TTATTCTTTG |
| −500 | TGTTTTACCG TATGTTAAAC ATTTCCAGAT ATTTAAATAA GAGTAAAGAA |
| −450 | GACACATCCA GCCAAGGTCC TCCAGATAGA TCCTTTTGCT TTCTTTCTAA |
| −400 | AGTCAAGTAA ATTCTAAACT AACCTTGACA TTATTAGTAA GTTTTGCTTT |
| −350 | AAAAAAAATA AAATTTTGTG TTAGAAGTTT TAAAACATTT GGAAATTCTA |
| −300 | GTTGCGGCTT CAGATTTCAT AATTCAGATG ATGCAACAGG ATGGAACCAT |
| −250 | TGTCAAAGAG AATGCAGGGA CGTTTGATGC TTGTTAGGAC ATGACTCCTG |
| −200 | TACTTGCCCA TTTGTTCATC CTCCAACCCC TCTTTCTTCC AAATTCCATG |
| −150 | TAGCATATTC TCTCCAGGAA GCAAGAAGAC TTGCCTGGCG GCATACTCAT |
| −100 | TTTCCCCATG CCTCTTTGCT GTTTGTGGAA AATAAAGCAT TCTATAGGCG |
| −50 | GAGCTAGTGA ACGCCTCTTT TAAAACACGA GTCTCCACAC TTCCCTGTTC |
| 1 | ACTTTGGTTC CAGCATCCTG TCCAGCAAAG AAGCAATCAG CCAAAATGAT |
| 51 | ACCTGGAGGC TTATCTGAGG CCAAACCCGC CACTCCAGAA ATCCAGGAGA |
| 101 | TTGTTGATAA GGTGAGTTGA TGCCATTCAG GAAAAAGTCT GAGCCAAAAT |
| 151 | CTTGATTCAT AAGTTGTCCC TGTGGAAAGG CTGTGGTTGA ACATGAAAAA |
| 201 | CAGAAGTTTA GGATGTTTGT GGCTTTGTTC AGGTTTCTTT ACAACTAAGA |
| 251 | TGAAATAAGA GTTTTTCAGA TGAAGCCTCA AATAATCCCC AAACTGTGAG |
| 301 | TGTGACCTTG AGCAAATCAC ATTTCTTCTC TGAGCTTCAT TTTCTCCATG |
| 351 | GGTAAAAGAA TTTGTATTGT ATCTTCTCTA AAGCTCTTAC AAGATATGAT |
| 401 | CTAGAAATAC TAATCATGTA TNCCGTNCAG CAGCCTTTAA AAA |

CystA.2 (Cystatin A sequence2) Sequence

Position 1 is the start of the 3′ section of intron 1. There is a gap in the published sequence between the 5′ and 3′ sections of intron 1, so two sequences are used here.

| | |
|---|---|
| 1 | ACATTTAAAC CCAACCCATA TCCCTCCATG GTAGACAGAA TGATGGCCCT |
| 51 | CCAAAGATGT CCATGTCCTA ATCCCTGGAA CCTGTGACTA GGTTCCTTTA |
| 101 | CACAGCAAAA GAGACTTTGC AGGTGTGATT AAGTTAAGGC TCTGGAGATG |
| 151 | GAGAGGTTAT CCTGGATTAT CCAGGTAAGC CCATGCAATC ACAGGGTCTT |
| 201 | TATAAGAATT ATACCCAAAT ACTGCACTCT AGTTAGTATA TTGTTTTCCA |
| 251 | CAGGGGTATA GGTCAACAAT TCTGAAATTA TTTTATGTAT ATGCTAGAAC |
| 301 | TGAGAAAATA GGCGAATACA TTGCAAATAA TAAGAACCAG GATTCTTCCT |
| 351 | CAGTGTTTGA GAAAGGAGTT ATAATAAGGA AAAGGGAAAA GCTAAATGAG |
| 401 | TCCTGTGGTA TTTGATTTAT TTTATTATTT ATTTATTTAT TTATTTATTT |
| 451 | ATGTATTTTG AGGCAGACTT TCACTCTTGT TGCCCAGGCT GGAGTGCAAT |
| 501 | GGTGTGATCT CGGCTCACTG CAACCTCCGC CTCCCAGGTT CAAGCAATTC |
| 551 | TCCTGCCTCA GCCTCCTGAG TAGCTGGAAT TACAGACACC CACCATTGCG |
| 601 | CCCAGCTAAT TTTTGTATTT TTAGTAGAGA CGGGGTTTCA TCATGTTGGT |
| 651 | CACACTGGTC TCAAACTCCT GACCTCAAGT GATCCACCCG CCTCTGCCTC |
| 701 | CCAAAGTGCT GGGATTACAG GCATGAGCCA CCACACCCGG CCGTGGTATT |
| 751 | TGATTTTAAT CAGAGGAATA CACACACACA CACACACACA CACACACACA |
| 801 | CACACACGTA CACACACATA TATAATGTAT ATATATAATG ACACACAGAT |
| 851 | AAATGTGTGT ATATATGCAC ACACATACAC AAATGCGTAA AGATGTGTGC |
| 901 | ATGTGTGAGT ACACATATTA TTTTGTAGCC CTTTCCACTG AGTGGGCCTA |
| 951 | GAAGCAATGA CATTTAGTAA CAAGAGACAC AGCACCCAAA TCTTGGGTCT |
| 1001 | TAAATACCGT TCTCCAATAA AAGGAGCAGG GACTCCTTGG AGAAATAGTT |
| 1051 | GATTCCAGTG AGGAGGCAAG GAAACTTCAA GATAAACCTG GAACATCTGA |
| 1101 | CTCTACTTTT GTAGAGTCAG AAAGTAAGGA AGTGCTTATA AAATGATGGA |
| 1151 | GGCATGTTGA AAGAACACAG GCATCAGGTG AAGGAGCTCC CAACGGCCAA |
| 1201 | ATCTAGGGCA TTTGAGTAAC AAAATAGAGT AATGAATTGT AATCCACAGA |
| 1251 | GTAAAAGAAA TATCCATGAG TCATATTAAT ATAAAATAAG TAATTGACTA |
| 1301 | CATAAAGAGG AGAGAGGGAA CAGCTCTTCC TTATGGCAGA ATTCCAGATA |
| 1351 | ATAAATGCAC AAGAAATGAT TTTTTAATTA CCATTTGGCC AACACCACAA |
| 1401 | TTAATCATTG TTGTAGGCAA GAACCATCAA TGGATGCTAA AATTCATGTA |
| 1451 | TGAAAGTATG GCGAGAAACA AGACTCCTCA CAAGACACTT ACTAATTACA |
| 1501 | AAGGGGAAAA AAATGGTAAC TTTACCGTGG AGAAACCTGG CAGACATCCT |
| 1551 | AAGTGATCAA AATTAACATC ACCAGTAATG AAACGTAGCT CCTGATAGGA |
| 1601 | TGCACTTAAG CACAACATCA CGTTTGTGCT ATCCTTACAA ACATGTATAA |
| 1701 | TACTAACATA AGAGGAGATC ACACAAACCT AAATTGAGAG ACATTCTACC |
| 1751 | ATTTAACAGG CCAGTATTCT TCAAAAACAT CAAGGCCATG AACAAAAAGG |
| 1801 | AATGAACCCA GATTGTAGGA GACTGAGGAG AAATGACAAC TAAATGCAAT |
| 1851 | GTAGGATCCT GCATGAGATC CTGGAGCAGA AAAGAGACAT TTTCAGGAAA |
| 1901 | ACTGGCAAAA TTCTGATAAG GTTTAGAGAC TACTTAATAG TATTATACCA |
| 1951 | TGTTAATTTC CTCATTTTGG TGATTATACT ATGTTATGTA AAACGGCAAA |
| 2001 | ATTAAGGGAA CTGTAAATGA TATAAGAGAA TTCATTATAC TAATTTTACA |
| 2051 | ACTTTTAAGT CTGAAATCAT TTTAAAATAA TGTTTTAATC AAGTAGTAAT |
| 2101 | TATTGCTCCA CTGATTTTTA TTAATCAAAG ATACATATAA CTCTTAGAGT |
| 2151 | TTTAGCAGTC AAAACGCTCC TATATTGCAT TACCACACTG AGTTGATGTG |
| 2201 | AATTCAGCCT AAAGCAACAA AATTTGACTT TTTAACCTAT GTAATTGTAT |
| 2251 | TAAAACAGGT GTTTTCTATT GAAATAATAA AAATAGTTCA TAGATTTCAT |
| 2301 | TACCATCTTT GAAGACTTTT AGGAGGATGA GGTTCCCAGA TGGGTACATT |
| 2351 | GCATACATGG AGTCTAATAT AGCTTTGATT ATTTGTTTCC TCTTTTCTTT |
| 2401 | TCTTTAGGTT AAACCACAGC TTGAAGAAAA AACAAATGAG ACTTATGGAA |
| 2451 | AATTGGAAGC TGTGCAGTAT AAAACTCAAG TTGTTGCTGG AACAAATTAC |
| 2501 | TACATTAAGG TTAGAGTTCA GCACCTACTT TAGCGCCAAA AGATGTATTT |
| 2551 | CTCATTTTAT GTAAAATATT CCCTGATTTC CCTACCACAT AATTCCTTCC |
| 2601 | TTACGGTTGT CCTAAATTAA GATGCCCAGC ATGATTCCTT CCAAGTGGTG |
| 2651 | GACTCTCAAA TTTGGTAGAT GATGTGACAC CATTTTTTTT CTTGTGAATT |
| 2701 | CAGAAAGTTT TATTACATTG ATTCTTGATT TCTGGAAATT GTATAGAAAA |
| 2751 | AGATTTCATA ACAGTTCAGG GCATGCTGGA TTTGTGGCTG TGCTGCTGCT |
| 2801 | TAGGTAAGGA GGGAGGATCA CGTCTCATCT GCCTGAAGGT GTGGGGCTGG |
| 2851 | ACCACATGGT ATCTTGAAGC TGATTCTACC TCAAAGCTGT ATGATTCTCT |
| 2901 | ATGTCTAAAT TAAATAAATT AAAGTAACGA CTCAAATCAT TAGCTGGGAA |
| 2951 | AATAATTTAA ACCTTTTTTG CCCTTCAATT AATATAATGT AAATACTATT |
| 3001 | TTGTAACTCA AATTAGATTT AACTTGTGGC ACTCTAGGCA TAGTTTAAAA |
| 3051 | GAGTGGAGTA TTTAAAATAG TTTAAAAGTT AATTTTATAG CTCTGTCAAA |
| 3101 | AAATTACACA ATGCATTGAT CGAGTTCTAA TTGAAGTTAT CAAGAGGAAA |
| 3151 | CCAGTTGAAC AAGTCAACCA TGAATATTTG AGAGCCATGT TTGTCCTTAT |
| 3201 | ATATTAAGAA GATGATAATG CTGATTTTAA AAATAAAAAC AAATATTCAA |
| 3251 | GTATATTTAC TTAATCTTTA CTTCACTATT TGACCAGGGC ACAGTGTGGC |
| 3301 | TTATTTCTCT TTAACAAATA TTTCTATTAA AATATAAACA TTTTAAAAGA |
| 3351 | TTTTTTCAGC AAAAAAAAAA AAAAAAGTAA GTGAAGTAAG TGGAGCTAGT |
| 3401 | CAGCTCAGCA TAAGAAAGTT TCAGTACAGA GCTATGTACA CAGATCAGCC |
| 3451 | CTGTTCTGCC AAATGTCAGC TCCTAGTAAC GACCAGGTCC CGGGGAGACA |
| 3501 | CATGTGAAAG AGGACCCTGC TGTATCCTCA GAAAACAATG GCCCCCTTTC |
| 3551 | CTCCATTCTC AATCTGCTTT CTTCATACGC TGGAAAAGAA AGTTTTCCAG |
| 3601 | TTAAAAATGA TTATGTGCAA AGATAGGAAA CATTCCATCA ACATTAACAT |
| 3651 | ATTTAATAAT TTATGATTAA TTCAAATGCA TAAATTTCAC ATATTACCAG |
| 3701 | CTCACATGTT TCTTCAACAG TTCATCAGAT AACTATCTTG AAAATTTCTG |
| 3751 | CTTGAAAATT TGTTCCCTTG ACCACCCTTT TGCCCTCTCT TAATCAGTCT |
| 3801 | CCTCTCTCTC TCTCATCTTT TCTTCCTTCT GCTATCAAAC TTTTCCTACT |
| 3851 | GGATCTCAGC CACCGATCCC AGTTCCCTTT TACTTCCTCG TAGTCTGGCT |
| 3901 | GTTGATCCCT TTGCTCTGAG GCACTCTAGA TTTAAGGTCT TGCCAGTGAT |
| 3951 | GTGACCTTCT CTATGTATTT CAAGTACCTA TCAAGAGGTA GGTGGTAGAA |
| 4001 | TGGAAGGACC ACAAGCTTAG GTGTCAGAGT GTCCTGGGTT TGAACCCTTG |
| 4051 | TTCAATTTGT TCTATGGGAA GCTCCTCCTC CTCTCTGAGC CTTCATTCCC |
| 4101 | TTATCTGCAC AATGAGGGTA ATAATCTACT TCGCAGCGTG TTGTGAGGAA |
| 4151 | TAAATAAGCT GGAAATTTAT TGAGCACTTA TAATTCACTA TGCACTATTC |
| 4201 | TAAGAACAGG GCTTATCTCA TTTAATCCTC ACAACAAATC TATGAGATAA |
| 4251 | GTACAATCAC TTCTCTTGGG TTACCAACGA AAAAACTGAG TTCCAGGGTG |
| 4301 | GTGAAGAAAC TAAAAAGATC ACACAACTAC AAGAGCAGAG TCAGGATTTG |
| 4351 | AACCCAGATA GACTGAGCTT AACTACTGGC TGTGCTGCCT CTAATATAAA |
| 4401 | GCACAAAATA AGAGCTTTTA CAAGGTTACA CTGGCCTGGT GTGGTGGCTT |
| 4451 | ATACCTGTAA TCCGAACACT TTGGGAGGCC AAGGTGGGAG GATAGCTTGA |
| 4501 | AGCCAGGAGT TTGAGATCAT TCTGGGCAAC ATAGCAAGAC CCTGCCTCTA |
| 4551 | CAAAGAAAAT GTTTTAATTA CCTAGGCATG GTGATGCACA CCAGTAGTCC |
| 4601 | TAGCTACTTG GGAAGCTGAG GTAGGAGGAT CACTTGAGCC GAGGAGTTTG |
| 4651 | AGGTTGCAGT GAGCTGCGAT CGCACCACTG CACTGTGAGC CAGGCCTCAT |
| 4701 | TCCCCGATCA GTACCCCCCA AAAATGTTAC ATTGTAGAGT GAAAAGAATG |
| 4751 | TAGATGCTAG AGGCTAACAG ATCTGGGTTT GAATATTGGC TGTGCCACTG |
| 4801 | ACTAGCTGAG AGATTTATGG AAAATCACTT AATCTCTCCT ACTCTGCTTC |
| 4851 | CACGTCTGTA AAAATTTCAT TGCTCCACTT TTCTTCAGGC CTATAATATA |
| 4901 | GGTTAATATA ATCATTTATA TAAAATGTTC ATCATAGTGT CTGGCTCACA |
| 4951 | GTAAACATTT GATATATGGC ATTTGTTAAA ATTAGGATAG GAAGTGACAT |
| 5001 | CAGAAGCACA ATAAATATTT GTATAAGACA AAGCATTTAT TGTCTCCAGC |
| 5051 | AAGAACCAAA GTAAAAATTC TTACCATAAT TTTCCAGGTC TCAGATTCAT |
| 5101 | GTCCAAACTA CTGCTTCTGC TTCAACCTTC CTACCAATGA CTTCCTAGTA |
| 5151 | AAACCCCTTA GCTTTTTTTT TTCTTTTCTT TTTTGAGACG GAGTTTCACT |
| 5201 | CTGTCACTCA GGCTGGAGTG CAGTGGCACA ATCTCGGCTC TCTGCGACCT |
| 5251 | CCACCTCCCG GATTCAAGCG ATTCTCCTGC TTCAGCCTCC CAAGTAGCTG |
| 5301 | GGATTACAGA TGTGCACGAC CACACCCAGC TAATTTTGTA TTTTTAGTAG |
| 5351 | AGACCAGATT TCACCATGTT GGCCAGGTTT GTCTCAAACT CCTGACCTCA |
| 5401 | GGTGATCCAC CCACCTTGGC CTCCCAAAGT GCTGGGATTA TAGGCGTGAG |
| 5451 | CCACAGTGCC CGGCTGGCCC CTTCACTTTA GAAGGGAGGG TGTCCGCCCC |
| 5501 | CTGGGCTCTG CTCAATCCTA CCAGTGGGTG TTTATAAAGT AGGTTCTGAT |
| 5551 | ATAGGTATGG GAACCCTGCA TCCCAAATTT TCTAGGGCAA TTCTGATTTT |
| 5601 | CTTCTCCCTT ATCAGACTGT ATGGCAAACA AAGTGTCCCA AGTTGAGAGT |
| 5651 | CAGAAAACAG TATCACCAAA AATATAGGCT ATCACTTGTT TTCTTGCTCC |
| 5701 | ATGCATCTTT GAAAATAAAA TGCTGTCCTT TGTGCCCAGA TTATACTAAA |
| 5751 | AAAAATAACA AAATAAACCT AGGACCTATG CCTCTTGCCA TGCCATTAGT |
| 5801 | CTACAATTTT TTTTTTTTTT TAAGACAAAC TCTTGCTCTA TCACCCAGGC |
| 5851 | TGGAGTACAG TGGCATGATC TCGGCTCACT GCAACCTTCT CCTCCTGGGT |
| 5901 | TCAAGTGATT CTCCTGCCTC AGCCTCTAGA GTAGCTGGGA TTACAGGCAT |
| 5951 | GCACCACCAT GCCCGGCTAA TTTTTTGTAT TTTTAGTAGA GACGGGATTT |
| 6001 | CACCATGCTG GCCAGGCTTG TCTTGAACTC CTGACCTCGT GATCCCCCTG |
| 6051 | CCTCAGCCTC CCAAAGTGCT AGGATTACAG GTGGGAGCCA CGGCACCCAG |
| 6101 | CCTAGTCTGC TCTTTTTCTC CTAAAATAAG GTGGTGGATT TATGAATACA |
| 6151 | AAGAGTCTAA GAATGGTGGA CTAGGTCTAG CAATGCTGTT CCTCAGCAGC |
| 6201 | TTTTTGGACA GAAGTCTTTG TAGACCTGTG GCTCTCTCAC TTGATGTAGA |
| 6251 | CCCATTTGAA TGAATCTCCT TTTGCTTTCT CTTTCTTTAA TATTTTTCAG |
| 6301 | GTACGAGCAG GTGATAATAA ATATATGCAC TTGAAAGTAT TCAAAAGTCT |
| 6351 | TCCCGGACAA AATGAGGACT TGGTACTTAC TGGATACCAG GTTGACAAAA |
| 6401 | ACAAGGATGA CGAGCTGACG GGCTTTTAGC AGCATGTACC CAAAGTGTTC |
| 6451 | TGATTCCTTC AACTGGCTAC TGAGTCATGA TCCTTGCTGA TAAATATAAC |
| 6501 | CATCAATAAA GAAGCATTCT TTTCCAAAGA AATTATTTCT TCAATTATTT |
| 6551 | CTCATTTATT GTATTAAGCA GAAATTACCT TTTCTTTCTC AAAATCAGTG |
| 6601 | TTATTGCTTT AGAGTATAAA CTCCATATAA ATTGATGGCA ATTGGAAATC |
| 6651 | TTATAAAAAC TAGTCAAGCC TAATGCAACT GGCTAAAGGA TAGTACCACC |
| 6701 | CTCACCCCCA CCATAGGCAG GCTGGATCGT GGACTATCAA TTCACCAGCC |
| 6751 | TCCTTGTTCC CTGTGGCTGC TGATAACCCA ACATTCCATC TCTACCCTCA |
| 6801 | TACTTCAAAA TTAAATCAAG TATTTTACAA AGTGTGTGTG TGTGTGTGTG |
| 6851 | TGTGTA |

### Annex B: SLPI Reference Sequences

SLPI reference sequence. Position 1 on this sequence corresponds to position 1 of NCBI M7444.

| | |
|---|---|
| 1 | GAATTCCAAG CATGAAGATA ATGAGTCAAG AGCTTGGAGT TTGTAGCTAG |
| 51 | ATGAGCTTTG GTTGAATTTT ATTTTATTTT ATTTTTTTAA GACAGGGTAT |
| 101 | CGCTCTGTCC CCCAAGCTGG AATGCAGTGG CACAATCATG GCTCACTGCA |
| 151 | GCCTCAAACT CCTGGGCTAA AGCGATCCTC CTGGCTCAGC CTCCCAAGTA |
| 201 | GCTGGGACTA CAGGCATACG TACGTCATCA TGCCTGGCTG ATTTTTTACA |
| 251 | TTTTTTTGTA GAGATGGGGT CTCAATATGT GGCCAGGGCT GGTCTCAAAC |
| 301 | TCCTACTCTC AAGGAATCCA TACACCTCAG CCTCCTGGGC AGCTGAGACA |
| 351 | GCAAGTGTGC GACCCTACAC TCAGCTATGG GCTGAATTTT AGAGATAATG |
| 401 | GTCGCTCTCT TTATAATTAG AAGCAACCTA TGCAGACTGG GTAGCAAATA |
| 451 | GAATGGGTTT AATTTTTTGC TGTCATGTGA GATCTGTAAG GGATTTTGGG |
| 501 | GAATTTTAGG AAGCAATCCT CTAAGATCTC AAATTATCTC ACAGCTAAAT |
| 551 | GTAGATTACA GTGACTGATG AGCTGCTTTC CCCCTTTATC TCAGATTCAT |
| 601 | TTCAATTCTC TTTAGTGGGA AGGGATACTA TTCATTTGTT CTTTTCATTC |
| 651 | AGAGTCCCTT CATGCCCTTA ATTTCATAAC CCTCTGAGAA GGGCTGACTT |
| 701 | GTTAGTATCA TTTCATTTCA CAGCTGAGAC AACTGAGCTC CAGAGAGATT |
| 751 | TGTGGAGAGC GGAGCTCTTC TTCAGCTTTC ATTTGTGAGT GCTTTTCCTG |
| 801 | TGTCAGGCAC AGAACAGGCA CTGGGGATAT AACGGTGTAA ATATTTCAGG |
| 851 | GAACTAAGTA TCAGTTGGTT GAACGAGCTG AACTTTTGAG AAAGAAACTG |
| 901 | CATTGAGTAA TCAGCAGAGT TTCACAATGC CTGAGAGTCC AGTAATGTGA |
| 951 | GAATCAGAAT TAGCAATGTG AGAATAGAAT GTATTGCACA AAGTCTCAGC |
| 1001 | AGGGAGTCTG TGTCTGGTTT TAGTTCCAGG TCCGGGTAGC ACCTTTGCAA |
| 1051 | TTGACCACTT CTTCCCTCTC TCCACCTATA AGGCTAATGG CCTGGGATCT |
| 1101 | TGTGATGTTT AGGGCTCAGA TGGACACTGA GATGGCCTCT TTAATCAACC |
| 1151 | AACTTCCCAG GCCAATCTCT TCCCTTTCTT TTCTGATAGT TGCTGTGTTG |
| 1201 | GCCTCATAGC CTTACCTGGC ATAGGAAAGA TAAACAATCT CCTTGGTGTC |
| 1251 | AGGATTTCTG GTCTCTGGCT ACGTTTCCTG CTTATGCAAT AGTAGCTGGG |
| 1301 | AGAGGCCGAA AGAATTCTGG TGGGGCCACA CCCACTGGTG AAAGAATAAA |
| 1351 | TAGTGAGGTT TGGCATTGGC CATCAGAGTC ACTCCTGCCT TCACCATGAA |
| 1401 | GTCCAGCGGC CTCTTCCCCT TCCTGGTGCT GCTTGCCCTG GGAACTCTGG |
| 1451 | CACCTTGGGC TGTGGAAGGC TCAGGGCTCT AGATGGACAC TGAGACGGCC |
| 1501 | TCTTTAATCA ACCAACTTCC CAGGCCAATC TCTTCCCTTT CTTTTCTCGA |
| 1551 | TAGTTGCTGT GTTTGGCCTC ATAGCCTTAC CTGGCATAGG AAAGATAAAC |
| 1601 | AATCTCCTTG GTGTCAGGAT TTCTGGTTTT TGGTTAGGGT TTCCTGCTTA |
| 1651 | TGCAATAGTA GCTGGGAGAG GCCCGAAAGA ATTCTGGTGG GGCCACACCC |
| 1701 | ACTGGTGAAA GAATAAATAG TGAGGTTTGG CATTGGCCAT CAGAGTCACT |
| 1751 | CCTGCCTTCA CCATGAAGTC CAGCGGCCTC TTCCCCTTCC TGGTGCTGCT |
| 1801 | TGCCCTGGGA ACTCTGGCAC CTTGGGCTGT GGAAGGCTCT GGAAAGTGTA |
| 1851 | AGTTGGAGTC ACTGTCTAAT CTGGGCTGCA GGGTCAGAGG TG |

### References

Allen M H, Veal C, Faassen A, Powis S H, Vaughan R W, Trembath R C, Barker J N W N. (1999). A non-HLA gene within the MHC in psoriasis. 353; 1589-1590.

Becker K G, Simon R M, Bailey-Wilson J E, Freidlin B, Biddison W E, McFarland H F, Trent J M. (1998). Clustering of non-major histocompatibility complex susceptibility candidate loci in human autoimmune diseases. Proc Natl Acad Sci USA. 95(17):9979-84.

Brattsand M, Egelrud T. (1999). Protein purification, molecular cloning, and expression of a human stratum corneum trypsin-like serine protease with possible function in desquamation. J Biol Chem. 274(42):30033-40.

Carroll J M, Romero M R, Watt F M. (1995). Suprabasal integrin expression in the epidermis of transgenic mice results in developmental defects and a phenotype resembling psoriasis. Cell. 1995 83(6):957-968.

Chapman and Walsh (1991). Desmosomes, corneosomes and desquamation. An ultrastructural study of adult pig epidermis. Arch Dermatol Res 282; 304-310.

Coleman, R., Trembath, R. C., Harper, J. I. (1997) Genetic studies of atopic and atopic dermatitis. Br. J. Dermatol. 136: 1-5

Cookson W O, Ubhi B, Lawrence R, Abecasis G R, Walley A J, Cox H E, Coleman R, Leaves N I, Trembath R C, Moffatt M F, Harper J I. (2001). Genetic linkage of childhood atopic dermatitis to psoriasis susceptibility loci. Nat Genet. 27(4):372-3.

Cork, M. J. (1997) The importance of skin barrier function. J. Derm Treatment. 8, S7-S13.

Daser A, Koez K, Batjer N, Jung M, Ruschendorf F, Goltz M, Ellerbrok H, Renz H, Walter J, Paulsen M. (2000). Genetics of atopy in a mouse model: polymorphism of the IL-5 receptor α chain. Immunogenetics 51: 632-638.

Egelrud T. (1993). Purification and preliminary characterization of stratum corneum chymotryptic enzyme: a proteinase that may be involved in dequamation. J Invest dermatol 101, 200-204.

Egelrud, T. (2000) Desquamation in the stratum corneum. Acta. Derm. Venerol. Supp. 208, 44-45.

Ekholm I E, Brattsand M, Eglrud T. (2000). Stratum corneum tryptic enzyme in normal epidermis: a missing link in the desquamation process. J Invest Dermatol, 114, 56-63.

Ekholm, E. and Egelrud, T. (1998) The expression of stratum corneum chymotrytic enzyme in human anagen hair follicles: further evidence for its involvement in desquamation-like processes. Brit. J. Derm. 139, 585-590.

Elias, P. M. (1983) Epidermal lipids, barrier function and desquamation. J Invest. Dermatol. 80, (6) 44-49.

Franzke C- W, Baici A, Bartels J, Christophers E, Wiedow O. (1996). Antileukoprotease inhibits statum corneum chymotryptic enzyme. Evidence for regulative function in desquamation. J Biol Chem 271 (36); 21886-21890.

Gan L, Lee I, Smith R, Argonza-Barrett R, Lei H, McCuaig J, Moss P, Paeper B, Wang K. (2000). Sequencing and expression analysis of the serine protease gene cluster located in chromosome 19q13 region. Gene. 17;257(1):119-130.

Guerrin M, Vincent C, Simon M, Tazi Ahnini R, Fort M, Serre G. (2001). Identification of six novel polymorphisms in the human corneodesmosin gene. Tissue Antigens. 57(1):32-8.

Guerrin, M., Simon, M., Montézin, M., Haftek, M., Vincent, C. and Serre, G. (1998) Expression cloning of human corneodesmosin proves its identity with product of the S gene and allows improved characterisation of its processing during keratinocyte differentiation. J. Biol. Chem., 273 22640-22647.

Hansson L, Stromqvist M, Backman A, Wallbrandt P, Carlstein A, Egelrud T. (1994). Cloning, expression, and characterization of stratum corneum chymotryptic enzyme. A skin-specific human serine proteinase. J Biol Chem. 269(30):19420-6.

Ishihara M, Yamaguata N, Ohno S, Naruse T, Ando A, Kawata H, Ozawa A, Ohkido M, Mizuki N, Shiina T, Ando H, Inoko H. Genetic polymorphisms in the keratin-like S gene within the human major histocompatibility complex and association on the susceptibility to psoriasis vulgaris. (1996). Tissue Antigens, 48: 182-186.

Jenisch S, Koch S, Henseler T, Nair R P, Elder J T, Watts C E, Westphal E, Voorhees J J, Christophers E, Kronke M. (1999). Corneodesmosin gene polymorphism demonstrates strong linkage disequilibrium with HLA and association with psoriasis. Tissue Antigens, 54: 439-449.

Landmann, L. (1998) The epidermal permeability barrier. Anat. Embryol. (Berl.). 172, 1-13.

Lee Y A, Wahn U, Kehrt R, Tarani L, Businco L, Gustafsson D, Andersson F, Oranje A P, Wolkertstorfer A, v Berg, A, Hoffmann U, Kuster W, Wienker T, Ruschendorf F, Reis A. (2001). A major susceptibility locus for atopic dermatitis maps to chromosome 3q21. Nat Genet 26(4):470-3.

Li, A., Hopkin, M. (1997) Atopic phenotype in subjects with variants of the β subunit of the high affinity IgE receptor. Thor 52: 654-655.

M. Guerrin, C. Vincent, M. Simon, R. Tazi Ahnini, M. Fort, and G. Serre. (2000). Identification of six novel polymorphisms in the human corneodesmosin gene. Tissue Antigens, in press.

Menton and Eisen (1971). Structure and organisation of mammalian stratum corneum. J Utrastruct. Res 35: 247-264

Murphy R, Williams H C, Duff G W, Cork M J. Total and specific IGE in atopic dermatitis. Some considerations for linkage analysis (submitted, 2000).

North A J, Bardsley W G, Hyam J, Bomslaeger E A, Cordingley H C, Trinnaman B, Halzfeld M, Green K J, Magee A I, Garrod D R. Molecular map of the desmosomal plaque. Journal of Cell Science 112, 4325-4336 (1999).

Ober C, Tsalenko A, Parry R, Cox N J. A second-generation genome-wide screen for asthma-susceptibility alleles in a founder population. (2000). Am J Hum Genet; 67(5): 1154-1162.

Potter S, Mitchell M D, Hansen W R, Marvin K W. (2000). NF-IL6 and CRE elements principally account for both basal and interleukin-1 beta-induced transcriptional activity of the proximal 528 bp of the PGHS-2 promoter in amnion-derived AV3 cells: evidence for involvement of C/EBP beta. Mol Hum Reprod. Sep; 6(9):771-8.

Serre, G., Mils, V., Hafftek, M., Vincent, C., Croute, F., Reano, A., Ouhayoun, J- P., Bettinger, S., Soleilhavoup, J- P. (1991) J. Invest. Dermatol. 97: 1061-1072

Shirakawa, T., Li, A., Dubowitz, M., et al., (1994) Association between atopic and variants of the β subunit of the high afinity immunoglobulin E receptor. Nat. Genet. 7: 125-130

Simon, M., Montézin, M., Guerrin, M., Durieux, J. J. and Serre, G. (1997) Characterisation and purification of human corneodesmosin, an epidermal basic glycoprotein associated with corneocyte-specific modified desmosomes. J. Biol. Chem., 272, 31770-31776.

Sundberg J P, France M, Boggess D, Sundberg B A, Jenson A B, Beamer W G, Shultz L D. (1997). Development of and progression of psoriasiform dermatitis and systemic lesions in the flaky skin (fsn) mouse mutant. Pathobiology, 65: 271-286.

Takahashi h, Asano K, Kinouchi M, Ishida-Yamamoto A, Wueppers K D, Lizuka H. Structure and transcriptional regulation of the human cystatin A gene. (1998). J Biol Chem 273; 17375-80.

Tazi Ahnini R, Camp N J, Mee J B, Duff G W, Cork M, di Giovine F S (1999a). Genetic Association between the corneodesmosin (MHC) S Gene and Susceptibility to Psoriasis. Hum. Mol. Genet. 8 (6): 1135-1140.

Tazi Ahnini R, di Giovine F S, Cox A, Keohane and Cork M J. The corneodesmosin (MHC S) gene in Guttate psoriasis. Lancet 1999 14;354:597

Tsukada J, Saito K, Waterman W R, Webb A C, Auron P E. (1994). Transcription factors NF-IL6 and CREB recognize a common essential site in the human prointerleukin 1 beta gene. Mol Cell Biol. 11:7285-7297.

Williams H C, Burney P G, Hay R J et al. The UK working party's diagnostic criteria for atopic dermatitis I: derivation of a minimum set of discrimination for atopic dermatitis. Br J Dermatol 1994; 131:383-396.

Yamazaki M, Ishidoh K, Kominami E, Ogawa H. (1997). Genomic structure of human cystatin A. DNA Seq. 8(1-2):71-76.

Zhou, Y. and Chaplin, D. D. (1993) Identification in the HLA class I region of a gene expressed late in keratinocyte differentiation. Proc. Natl. Acad Sci. USA, 90,9470-9473.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of diagnosis of a disease, or susceptibility to a disease associated with abnormal cell-cell adhesion between epithelial cells, the method comprising detection of a mutation in a nucleic acid encoding an adhesion protein, a protease, or a protease inhibitor of an individual.

2. A method of diagnosis of a Group I disease or susceptibility to a Group I disease in an individual, the method comprising detecting a presence or absence of a polymorphism in an adhesion protein, protease or protease inhibitor polypeptide, or a nucleic acid encoding such, in which the polymorphism is associated with a Group I disease.

3. An method according to claim 1 or claim 2, in which the adhesion protein is selected from the group consisting of adhesion proteins shown in Tables D2.1 and 5.1, preferably corneodesmosin, desmoglein I, desmoglein 3, plakoglobin, desmoplakin, desmocollin I, envoplakin, a proline-rich protein, preferably a small proline-rich protein (SPRR), SPRR2A, SPRR1B, SPRK, SPRR2E, SPRR2F, SPRR2B, SPRR2D, SPRR2C, SPRR2G, SPRR1A, SPRR3, SPRR4, involucrin, or loricrin.

4. A method according to any preceding claim, in which the protease is selected from the group consisting of proteases shown in Tables D3.1 and 6.1, preferably stratum corneum chymotryptic enzyme (SCCE) or stratum corneum tryptic enzyme (SCTE).

5. A method according to any preceding claim, in which the protease inhibitor is selected from the group consisting of protease inhibitors shown in Tables D4.1 and 7.1, preferably Secretory Leukoprotease Inhibitor (SLPI), elafin protease inhibitor 3 (PI3 or SKALP) or cystatin A (CSTA).

6. A method according to any preceding claim, in which the Group I disease is selected from the group consisting of: atopic eczema, sebarrhoeic eczema, irritant contact dermatitis, allergic contact dermatitis, lung atopic asthma, post viral asthma, branchial hyper-reactivity, chronic obstruction pulmonary disease, Crohn's disease, ulcerative colitis, coeliac disease, peptic ulceration, impetigo, viral warts, Molluslum Contagiosum, bacterial meningitis, viral meningitis, peptic ulceration associated with penetration of Helicobacteria pylori, skin melanoma, squamous cell carcinoma, basal cell carcinoma, cutaneous lymphoma, a skin cancer, a malignancy of the gastrointestinal tract and a malignancy of the lung.

7. A method according to any preceding claim, in which the disease comprises a Group I disease, and the method comprises detecting any one or more of: (a) the presence of a T at position +1243 of a corneodesmosin nucleic acid; (b) the absence of a Hph1 restriction enzyme site at position +1243 of a corneodesmosin nucleic acid; (c) the presence of a leucine (L) residue at position 394 of a corneodesmosin polypeptide (L20815); and (d) a mutation in a corneodesmosin nucleic acid which leads to (c), of an individual.

8. A method according to any preceding claim, in which the disease comprises a Group I disease, and the method comprises detecting any one or more of: (a) the presence of a T at position +1243 of a corneodesmosin nucleic acid; (b) the presence of a leucine (L) residue at position 394 of a corneodesmosin polypeptide (L20815); and (c) a mutation in a corneodesmosin nucleic acid which leads to (b), of an individual.

9. A method according to any preceding claim, in which the disease comprises a Group I disease, and the method comprises detecting any one or more of the following nucleotides or any one or more of the following amino acids at the relevant positions of a corneodesmosin nucleic acid or polypeptide:

| | Nucleotide Position | | | | | | | |
|---|---|---|---|---|---|---|---|---|
| | 442 | 468 | 619 | 1215 | 1236 | 1243 | 1515 | 1593 |
| Nucleic acid (s) | A | AGT | T | A | T | T | G | T |
| Residue | 127 | 137 | 186 | 385 | 392 | 394 | 485 | 511 |
| Position (1) | | | | | | | | |
| Residue | 143 | 153 | 202 | 401 | 408 | 410 | 501 | 527 |
| Position (2) | | | | | | | | |
| Residue | D | S/— | F | S | S | L | D | D/N |

in which “Residue Position (1)” refers to the numbering of the sequence with accession number L20815, and “Residue Position (2)” refers to the numbering of the sequence with accession number AF030130.

10. A method according to any preceding claim, in which the disease comprises a Group I disease, and the method comprises detecting a CD5 corneodesmosin allele or a CD6 corneodesmosin allele, as described in Jenisch et al (1999), Tissue Antigens, 54: 439-449, in an individual.

11. A method according to any preceding claim, in which the disease comprises a Group I disease, and the method comprises detecting any one or more of: (a) the presence of a T at position 180 of a corneodesmosin nucleic acid; (b) the presence of an F at position 40 of a corneodesmosin polypeptide having accession number L20815; (c) the presence of an F at position 56 of a corneodesmosin polypeptide having accession number AF030130; and (d) a mutation in a corneodesmosin nucleic acid which leads to (b) or (c), of an individual.

12. A method according to any preceding claim, in which the disease comprises a Group I disease, and the method comprises detecting any one or more of: (a) the presence of a T at position 619 of a corneodesmosin nucleic acid; (b) the presence of an F at position 186 of a corneodesmosin polypeptide; and (c) a mutation in a corneodesmosin nucleic acid which leads to (b), of an individual.

13. A method according to any preceding claim, in which the disease comprises a Group I disease, and the method comprises detecting the presence of an AACCAACC sequence in an SCCE nucleic acid of an individual, preferably at positions corresponding to positions 7634-7637 in an SCCE genomic sequence (GB: AF166330).

14. A method according to any preceding claim, in which the disease comprises a Group I disease, preferably atopic eczema the method comprising detecting the presence of one or more polymorphisms selected from the group consisting of: (a) the presence of a T residue at position 280 of a SLPI nucleic acid; (b) the presence of a G residue at position 292/293 of a SLPI nucleic acid; (c) the presence of a C residue at position 1235/1236 of a SLPI nucleic acid; (d) the absence of a C or A residue at position 1384/1385 of a SLPI nucleic acid; and (d) a polymorphism in a SLPI polypeptide corresponding to any of the above.

15. A method according to any preceding claim, in which the disease comprises a Group I disease, preferably eczema, more preferably atopic eczema, the method comprising detecting the presence of one or more polymorphisms selected from the group consisting of: the absence of AC at positions 122 and 121 of a cystatin A nucleic acid, the absence of a G residue at position 110 of a cystatin A nucleic acid, the presence of a t residue at position 85 of a cystatin A nucleic acid, the presence of a G residue at position 73 of a cystatin A nucleic acid, absence of an A residue at position 72 of a cystatin A nucleic acid, the absence of a T at position 60 of a cystatin A nucleic acid, the absence of a C at position 15 of a cystatin A nucleic acid, the absence of an A residue at position 14 of a cystatin A nucleic acid, the absence of a C residue at position 13 of a cystatin A nucleic acid, the absence of a C residue at position 6 of a cystatin A nucleic acid, the absence of a T residue at position 5 of a cystatin A nucleic acid, the absence of a G residue at position 4 of a cystatin A nucleic acid, and the absence of a G residue at position 7 of a cystatin A nucleic acid, in which the position numbering is made with reference to the cystatin A sequence CystA.1.

16. A method of diagnosis of a disease, preferably a skin disease, preferably a skin inflammatory disease, preferably eczema, the method comprising detecting the presence of a G residue in a TRE-2 region of a cystatin A nucleic acid.

17. A method of diagnosis of a Group I disease or susceptibility to a Group I disease in an individual, the method comprising detecting the presence, absence or a modulated level of an adhesion protein, protease or protease inhibitor, or a fragment thereof, in an individual.

18. A method according to any preceding claim, in which the method comprises detecting any one or more of: (a) relative abundance of the 36 kDa, 46-43 kDa and 52-56 kDa corneodesmosin polypeptides; (b) the presence of or an elevated level of one or more 36, 46-43 kDa corneodesmosin polypeptides; (c) the absence of or a modulated level, preferably a lower level of 52-56 kDa corneodesmosin polypeptides in an individual.

19. A method according to any preceding claim, in which the method comprises detecting any one or more of: (a) relative abundance of the 80 kDa, 95 kDa and 160 kDa desmoglein I polypeptides; the presence of or an elevated level of one or more of 95 and 80 kDa polypeptides; (c) a reduced level of a 160 kDa desmoglein I polypeptide; (d) proteolysis of a 160 kDa desmoglein I polypeptide in an individual.

20. A method according to any preceding claim, in which the method comprises detecting the presence of or an elevated level of any one or more of a 55 kDa desmoglein 3 polypeptide, an 80 kDa desmoglein 3 polypeptide and a 100 kDa desmoglein 3 polypeptide in an individual.

21. A method according to any preceding claim, in which the method comprises detecting any one or more of: (a) relative abundance of the 85 kDa, 75 kDa and 70 kDa plakoglobin polypeptides; (b) the absence of or a reduced level of a 70 kDa plakoglobin polypeptide; (c) the presence of or an elevated level of an 85 kDa plakoglobin and/or a 75 kDa plakoglobin polypeptide in an individual.

22. A method according to any preceding claim, in which the method comprises detecting any one or more of: (a) relative abundance of the 70-80 kDa, 60-70 kDa and 50-60 kDa Desmocollin 1 polypeptides; and (b) the presence of or an elevated level of a 50-60 kDa Desmocollin 1 polypeptide in an individual.

23. A method according to any preceding claim, in which the polypeptide or fragment is detected in an epidermis of an individual, preferably ex-vivo in the form of a skin biopsy, or in the stratum corneum of an individual, preferably in the form of a tape strip.

24. A method according to any preceding claim, which comprises detecting up-regulation of expression of an adhesion protein polypeptide or nucleic acid selected from the group consisting of: keratin 6A (L42611); keratin 17 (Z19574); annexin A1/lipocortin (X05908); and collagen, type VI, alpha 3 (COL6A3) (NM_{—}004369).

25. A method according to any preceding claim, which comprises detecting down-regulation of expression of an adhesion protein polypeptide or nucleic acid selected from the group consisting of: S/corneodesmosin (AF030130); desoplakin (XM_{—}004463); plakoglobin (NM_{—}002230; (NM_{—}021991); desmoglein 1 (XM_{—}008810); desmocollin 1 (MX_{—}008687); envoplakin (XM_{—}008135;U72543); plectin 1 (NM000445); S100A2 (AI539439;M87068); S100A8 (AI126134); S100A7 (AA586894); S100A9); GB:W72424); SPRR2A); GB:M21302); SPRR1B (M19888); SPRK (AI923984); HCR (BAA81890); SEEK1 (BAA88130); SPR1 (BAB63315); STG (BAA88132); involucrin (NM_{—}005547); trichohyalin (NM_{—}005547); and loricrin (XM_{—}048902).

26. A method according to any preceding claim, which comprises detecting up-regulation of expression of an protease polypeptide or nucleic acid selected from the group consisting of: Apoptosis-related cysteine protease (CASP14) mRNA (NM_{—}012114); TGM5 (XM_{—}007529); phospholipases A(2) (BC013384); CD47 antigen (X69398); Kallilkrein 8 (AB008390); AD024 protein (XM_{—}002642); SCCE (XM_{—}009002); Defensin beta2 (AF0711216); Interferon a inducible protein 27 (X67325); Fatty acid binding protein FABP5 (M94856); SCTE (XM_{—}009000); kallikrein 1, renal/pancreas/salivary (KLK1) (XM_{—}047300); Homo sapiens kallikrein 2, prostatic (KLK2) (XM_{—}031757); kallikrein 3, (prostate specific antigen) (KLK3) (XM_{—}031768); kallikrein 6 (neurosin, zyme) (KLK6) (XM_{—}055658); kallikrein 4 (prostase, enamel matrix, prostate) (KLK4) (XM_{—}008997); membrane-type serine protease 1 (AF133086); Human skin collagenase (M13509); collagenase MMP-1 (LOC116389); collagenase MMP-12 (U78045); collagenase MMP-9 (NM_{—}004994); collagenase MMP-3 (U78045); collagenase MMP-28 (AF219624); caspase 7 (BC015799); Caspase 5 (NM_{—}004347); Caspase-14 (NM 012114); ubiquitin specific protease USP-5 (NM_{—}003481); ubiquitin specific protease USP-11 (NM_{—}004651); ubiquitin specific protease USP 6 (NM_{—}004505); TPS1 (NM_{—}003293); TPSB1 (XM_{—}016204); TPSG1 (XM_{—}008123); protease nexin-II (XM_{—}047793); Glia derived nexin precursor (P07093); 26S protease regulatory subunit S10B (Q92524); and PCOLN3 (XM_{—}047524).

27. A method according to any preceding claim, which comprises detecting down-regulation of expression of an protease polypeptide or nucleic acid selected from the group consisting of: Transglutaminase 1 (TGM1) (M98447); TGM2 (XM_{—}009482); TGM4 (XM_{—}056203); TGM7 (NM_{—}052955); TGM3 (L10386); ubiquitin specific protease USP 26 (NM_{—}031907); ubiquitin specific protease (USP 28) (NM_{—}020886); 26S protease subunit 4 (L02426); LILRB1 (AF004230); Signal trasducer and activator of transcription 1, 91 kDa (STAT1) (977935); and proteasome (prosome, macropain) subunit 6 (PSMA6) (X59417).

28. A method according to any preceding claim, which comprises detecting down-regulation of expression of an protease inhibitor polypeptide or nucleic acid selected from the group consisting of: hbc750 Human pancreatic islet (T11141; T10920); TIMP4 (NM_{—}003256); TIM9a (AF150100); plasminogen activator inhibitor type 2 (L19066); multivalent protease inhibitor WFIKKN (AF422194); eppin-1 (EPPIN) (AF286368); eppin-2 (EPPIN) (AF286369); eppin-3 (EPPIN) (AF286370); sparc/osteonectin, cwcv and kazal-like domains proteoglycan (testican) (SPOCK) (NM_{—}004598); P112 (AH009756); Human immunodeficiency virus type 1 gene for HIV-1 protease (AB020923); secreted phosphoprotein 2, 24 kD (SPP2) (NM_{—}006944); and cyclin-dependent kinase inhibitor 2B (p 15, inhibits CDK4) (CDKN2B) (NM_{—}004936).

29. A method according to any preceding claim, which comprises detecting up-regulation of expression of an protease inhibitor polypeptide or nucleic acid selected from the group consisting of: SLPI (X04502); SKALP (XM_{—}009524; L10343); CSTA (NM_{—}005213; AA570193); SCCA (S66296); SCCA2 (U19557); plasminogen activator inhibitor type 1 (X04729; X04731); PAI2 (AF071400); SERPINA5 (NM_{—}000624); TIMP (D11139); TIMP-1 (NM_{—}003254); TIMP-2 (NM_{—}003255); TIMP-3 (E13880); TIM9b (AF150105); Cystatin A (AA570193); Cystatin M/E (NM_{—}001323); C1 inhibitor (SERPING1) (X_{—}046218); protease inhibitor, Kunitz type, 2 (SPINT2) (XM_{—}032280); serine protease inhibitor, Kazal type 4 (SPINK4) (XM_{—}005539); proteinase inhibitor, clade B (ovalbumin), member 9 (XM_{—}053642); serine (or cysteine) proteinase inhibitor, clade B (ovalbumin), member 6 (XM_{—}047984); Serine protease inhibitor-like, with Kunitz and WAP domains 1 (eppin) (SPINLW1) (NM_{—}020398); protease inhibitor Kunitz type 1 (SPINT1) (XM_{—}056836); Human immunodeficiency virus type 1 gene for HIV-1 protease (AB020924); tissue factor pathway inhibitor 2 (TFPI2) (NM_{—}006528); cathepsin F (CTSF) (NM_{—}003793); serine (or cysteine) proteinase inhibitor, clade A (alpha-1 antiproteinase, antitrypsin), member 6 (SERPINA6) (NM_{—}001756); serine (or cysteine) proteinase inhibitor, clade B (ovalbumin), member 3 (SERPINB3) (NM_{—}006919); Serine (or cysteine) proteinase inhibitor, clade A (alpha-1 antiproteinase, antitrypsin), member 3 (SERPINA3) (NM_{—}001085); Homo sapiens serine (or cysteine) proteinase inhibitor, clade B (ovalbumin), member 13 (XM_{—}008743); serine (or cysteine) proteinase inhibitor, lade B (ovalbumin), member 5 (SERPINB5 (XM_{—}008742); RelA-associated inhibitor (XM_{—}057693); inhibitor of DNA binding 1, dominant negative helix-loop-helix protein (ID1) (XM_{—}946179); serine (or cysteine) proteinase inhibitor, clade E (nexin, plasminogen activator inhibitor type 1), member (SERPINE1) (XM_{—}054850); Similar to cyclin-dependent kinase inhibitor 2B (p15, inhibits CDK4) (BC014469); serine (or cysteine) proteinase inhibitor, clade B (ovalbumin), member 7 (SERPINB7 (XM_{—}008745); protein inhibitor of activated STAT protein PIASy (PIASY) (NM_{—}016149); similar to protein inhibitor of activated STAT protein PIASy (LOC95830) (XM_{—}016864); PKC-potentiated PP1 inhibitory protein (PPP1R14A) (AY050668); inhibitor of DNA binding 3, dominant negative helix-loop-helix protein (ID3) (NM_{—}002167); Clade A (alpha-1 antiproteinase, antitrypsin) serine (or cysteine) proteinase inhibitor, clade H (heat shock protein 47), member 1 (SERPINH1) (NM_{—}004353); PI13 gene for hurpin (serine protease inhibitor) (AJ278717); protease inhibitor 5 (maspin) (PI5) (XM_{—}008742); and PAI-2 (A32415).

30. A method of treatment or prophylaxis of a Group I disease, the method comprising up-regulating the expression and/or activity of an adhesion protein responsible for adhesion between the cells, or down-regulating the proteolysis of the adhesion protein.

31. A method according to the preceding claim, in which the expression and/or activity of the adhesion protein is up-regulated at the transcriptional or the translational level, or both.

32. A method according to any preceding claim, in which the expression, activity and/or breakdown of a protease involved in proteolysis of the adhesion protein is down-regulated.

33. A method according to any preceding claim, in which the expression and/or activity of a protease inhibitor responsible for inhibiting the activity of a protease involved proteolysis of the adhesion protein is up-regulated, and/or in which the breakdown of the protease inhibitor is down-regulated.

34. A method according to the preceding claim, in which proteolysis of the adhesion protein is reduced by one or more of the following: administration of a protease inhibitor or a fragment thereof; administration of an antagonist of a protease or a fragment thereof; administration of an agonist of a protease inhibitor; reducing the expression of a protease; reducing the activity of a protease; increasing the expression of a protease inhibitor; increasing the activity of a protease inhibitor.

35. A method of treatment or prophylaxis of a Group I disease, the method comprising administering to a patient suffering or likely to suffer from such a disease a therapeutically effective amount of a non-disease associated form of an adhesion protein, protease or protease inhibitor, or a fragment thereof.

36. A method according to any preceding claim, which comprises administration of a protease inhibitor, or a fragment thereof, capable of inhibiting protease activity.

37. A method according to the preceding claim, which comprises administration of a fragment of SLPI, preferably a peptide selected from the group consisting of: CGKS (SB7a) and CGKS CVSPVKA (SB7b); KIIDGA; GDKIIDGA; GDKIID; KII; KIID; KIIDG; KIIDGA; LDPVD (651); KRDLK (652); LDPVDTPNP (653); LDPVDTPNPTRRKPG (654); CGKSCVSPVKA (644); CVSPVKA (643), most preferably Peptide 643, Peptide 651 or Peptide 653.

38. A method according to any preceding claim, which comprises administration of TNF-α and/or IL-β.

39. A method according to any preceding claim, in which the Group I disease is eczema, preferably atopic eczema, or dermatitis, preferably dermatitis herpetiformis.

40. A method of diagnosis of a Group II disease or susceptibility to a Group II disease in an individual, the method comprising detecting a presence or absence of a polymorphism in an adhesion protein, protease or protease inhibitor polypeptide, or a nucleic acid encoding such, in which the polymorphism is associated with a Group II disease.

41. A method according to any preceding claim, in which the disease comprises a Group II disease, and the method comprises detecting any one or more of the following nucleotides or any one or more of the following amino acids at the relevant positions of a corneodesmosin nucleic acid or polypeptide:

| | Nucleotide Position | | | | | | | |
|---|---|---|---|---|---|---|---|---|
| | 442 | 468 | 619 | 1215 | 1236 | 1243 | 1515 | 1593 |
| Nucleic acid (s) | G | AGT | T | A | T | C | G | T |
| Residue | 127 | 137 | 186 | 385 | 392 | 394 | 485 | 511 |
| Position (1) | | | | | | | | |
| Residue | 143 | 153 | 202 | 401 | 408 | 410 | 501 | 527 |
| Position (2) | | | | | | | | |
| Residue | S | S | F | S | S | S | D | D |

in which “Residue Position (1)” refers to the numbering of the sequence with accession number L20815, and “Residue Position (2)” refers to the numbering of the sequence with accession number AF030130.

42. A method according to any preceding claim, in which the disease comprises a Group II disease, and the method comprises detecting a CD2 corneodesmosin allele, as described in Jenisch et al (1999), Tissue Antigens, 54: 439-449, in an individual.

43. A method according to any preceding claim, in which the disease comprises a Group II disease, and the method comprises detecting any one or more of: (a) the presence of a C at position 180 of a corneodesmosin nucleic acid; (b) the presence of an L at position 40 of a corneodesmosin polypeptide having accession number L20815; and (c) the presence of L at position 56 of a corneodesmosin polypeptide having accession number AF030130; and (d) a mutation in a corneodesmosin nucleic acid which leads to (b) or (c), of an individual.

44. A method according to any preceding claim, in which the disease comprises a Group II disease, and the method comprises detecting any one or more of: (a) the presence of a C at position 619 of a corneodesmosin nucleic acid of an individual; (b) the presence of an S at position 186 of a corneodesmosin polypeptide of an individual; and (c) a mutation in a corneodesmosin nucleic acid which leads to (b), of an individual.

45. A method according to any preceding claim, in which the disease comprises a Group II disease, and the method comprises detecting the absence of an AACCAACC sequence in an SCCE nucleic acid of an individual, preferably at positions corresponding to positions 7634-7637 in an SCCE genomic sequence (GB: AF166330).

46. A method according to any preceding claim, in which the disease comprises a Group II disease, preferably acne, in an individual, the method comprising detecting the presence of a G residue at position 1300 of SLPI or the presence of a C residue at position 1418 of SLPI, or both.

47. A method according to any preceding claim, in which the disease comprises a Group II disease, preferably psoriasis, in an individual, the method comprising detecting the presence of one or more polymorphisms selected from the group consisting of: (a) the presence of a G residue at position 291 of a SLPI nucleic acid; (b) the absence of a G residue at position 292/293 of a SLPI nucleic acid; (c) the presence of a G residue at position 1325 of a SLPI nucleic acid; (d) the presence of a C residue at position 1418 of a SLPI nucleic acid; and (d) a polymorphism in a SLPI polypeptide corresponding to any of the above.

48. A method according to any preceding claim, in which the disease comprises a Group II disease, preferably acne, in an individual, the method comprising detecting any one or more polymorphisms selected from the group consisting of: the presence of a G residue at position 110 1, the presence of a C residue at position 96 of a cystatin A nucleic acid, the presence of an A residue at position 71 of cystatin A, the presence of a G residue at position 20 of a cystatin A nucleic acid, the presence of a T residue at position 17 of a cystatin A nucleic acid, the presence of a C residue at position 13 of a cystatin A nucleic acid, the presence of a T residue at position 10 of a cystatin A nucleic acid, the presence of a T residue at position 8 of a cystatin A nucleic acid, the absence of a G residue at position 73 of a cystatin A nucleic acid, the absence of a TG at positions 76 and 77 of a cystatin A nucleic acid, and the absence of a C residue at position 6 of a cystatin A nucleic acid, in which the position numbering is made with reference to the cystatin A sequence CystA.1.

49. A method according to any preceding claim, in which the disease comprises a Group II disease, preferably psoriasis, in an individual, the method comprising detecting the presence of one or more polymorphisms selected from the group consisting of: the absence of a G residue at position 270 of CystA.1, the presence of a G residue at position 73 of CystA.1, the absence of a C residue at position 15 of CystA.1, the absence of a C residue at position 6 of CystA.1, the absence of a G residue at position 4 of CystA.1, and the absence of a G residue at position 7 of CystA.1, in which the position numbering is made with reference to the cystatin A sequence CystA.1.

50. A method according to any preceding claim, in which the Group II disease is acne, preferably acne vulgaris or psoriasis, preferably psoriasis vulgaris.

51. A method of diagnosis of a Group II disease or susceptibility to a Group II disease in an individual, the method comprising detecting the presence, absence or a modulated level of an adhesion protein, protease or protease inhibitor, or a fragment thereof, in an individual.

52. A method of diagnosis of a Group II disease or susceptibility to a Group II disease according to any preceding claim, in which the method comprises detecting any one or more of: (a) relative abundance of the 36 kDa, 46-43 kDa and 52-56 kDa corneodesmosin polypeptides; (b) an elevated level of one or more 52-56 kDa corneodesmosin polypeptides; and (c) a reduced level of one or both of 36 kDa and 46-43 kD corneodesmosin polypeptides, in an individual.

53. A method of diagnosis of a Group II disease or susceptibility to a Group II disease according to any preceding claim, in which the method comprises detecting any one or more of: (a) relative abundance of the 80 kDa, 95 kDa and 160 kDa desmoglein I polypeptides; (b) an elevated level or presence of a 160 kDa desmoglein I polypeptide; and (c) a reduced level or absence of one or both of a 95 and 80 kDa desmoglein I polypeptide; and (d) absence of proteolysis of a 160 kDa desmoglein I polypeptide in an individual.

54. A method of diagnosis of a Group II disease or susceptibility to a Group II disease according to any preceding claim,in which the method comprises detecting a reduced level or absence of any one or more of a 55 kDa desmoglein 3 polypeptide, an 80 kDa desmoglein 3 polypeptide and a 100 kDa desmoglein 3 polypeptide, in an individual.

55. A method of diagnosis of a Group II disease or susceptibility to a Group II disease according to any preceding claim, in which the method comprises detecting any one or more of: (a) relative abundance of the 85 kDa, 75 kDa and 70 kDa plakoglobin polypeptides; (b) presence of or an elevated level of a 70 kDa plakoglobin polypeptide; and (c) absence of or a reduced level of an 75 kDa plakoglobin polypeptide, in an individual.

56. A method of diagnosis of a Group II disease or susceptibility to a Group II disease according to any preceding claim, in which the method comprises detecting any one or more of: (a) relative abundance of 75-80 kDa, 190-250 and/or 120-180 desmoplakin polypeptides; (b) absence of or an reduced level of either or both of 190-250 kDa and 120-180 kDa desmoplakin polypeptides; and (c) lack of proteolysis of a 85 kDa desmoplakin polypeptide in an individual.

57. A method of diagnosis of a Group II disease or susceptibility to a Group II disease according to any preceding claim,in which the method comprises detecting any one or more of: (a) relative abundance of the 70-80 kDa, 60-70 kDa and 50-60 kDa Desmocollin 1 polypeptides; (b) and absence of or a reduced level of a 50-60 kDa Desmocollin 1 polypeptide in an individual.

58. A method of diagnosis of a Group II disease or susceptibility to a Group II disease according to any preceding claim, in which the method comprises detecting any one or more of: (a) relative abundance of the 124-209 kDa, 100-120 kDa, 60-80 kDa and 50-55 kDa envoplakin polypeptides; (b) the presence of or an elevated level of an 60-80 and/or 50-55 kDa envoplakin polypeptide; and (c) the absence of or a reduced level of an 124-209 kDa and/or an 100-120 kDa envoplakin polypeptide in an individual.

59. A method of diagnosis of a Group II disease or susceptibility to a Group II disease according to any preceding claim,in which the method comprises detecting any one or more of: (a) relative abundance of the 80-90 kDa and 70-75 kDa polypeptides; (b) the presence of or an elevated level of an 124-209 kDa SCCE polypeptide; (b) the absence of or a reduced level of a 80-90 kDa SCCE polypeptide in an individual.

60. A method of diagnosis of a Group II disease or susceptibility to a Group II disease according to any preceding claim, in which the method comprises detecting any one or more of: (a) relative abundance of the 90-100 kDa and 20 kDa polypeptides; (b) the presence of or an elevated level of an 90-100 kDa SLPI polypeptide; (c) the absence of or a reduced level of a 20 kDa SLPI polypeptide in an individual.

61. A method of diagnosis of a Group II disease or susceptibility to a Group II disease according to any preceding claim, which comprises detecting up-regulation of expression of an adhesion protein polypeptide or nucleic acid selected from the group consisting of: S/corneodesmosin (AF030130); desoplakin (XM_{—}004463); plakoglobin (NM_{—}002230; GB: NM_{—}021991); desmoglein 1 (XM_{—}008810); desmocollin 1 (MX_{—}008687); envoplakin (XM_{—}008135;U72543); plectin 1 (NM000445); S100A2 (AI539439;M87068); keratin 6A (L42611); keratin 17 (Z19574); S100A8 (AI126134); S100A7 (AA586894); S100A9); GB:W72424); SPRR2A); GB:M21302); SPRR1B (M19888); SPRK (AI923984); HCR (BAA81890); SEEK1 (BAA88130); SPR1 (BAB63315); STG (BAA88132); involucrin (NM_{—}005547); annexin A1/lipocortin (X05908); collagen, type VI, alpha 3 (COL6A3) (NM_{—}004369); trichohyalin (NM_{—}005547); and loricrin (XM_{—}048902).

62. A method of diagnosis of a Group II disease or susceptibility to a Group II disease according to any preceding claim, which comprises detecting up-regulation of expression of an protease polypeptide or nucleic acid selected from the group consisting of: Transglutaminase 1 (TGM1) (M98447); TGM2 (XM_{—}009482); TGM4 (XM_{—}056203); TGM5 (XM_{—}007529); TGM7 (NM_{—}052955); TGM3 (L10386); phospholipases A(2) (BC013384); CD47 antigen (X69398); Kallilkrein 8 (AB008390); AD024 protein (XM_{—}002642); Defensin beta2 (AF0711216); Interferon a inducible protein 27 (X67325); Fatty acid binding protein FABP5 (M94856); SCTE (XM_{—}009000); kallikrein 1, renal/pancreas/salivary (KLK1) (XM_{—}047300); Homo sapiens kallikrein 2, prostatic (KLK2) (XM_{—}031757); kallikrein 3, (prostate specific antigen) (KLK3) (XM_{—}031768); kallikrein 6 (neurosin, zyme) (KLK6) (XM_{—}055658); kallikrein 4 (prostase, enamel matrix, prostate) (KLK4) (XM_{—}008997); membrane-type serine protease 1 (AF133086); collagenase MMP-1 (LOC116389); collagenase MMP-12 (U78045); collagenase MMP-9 (NM_{—}004994); collagenase MMP-3 (U78045); collagenase MMP-28 (AF219624); caspase 7 (BC015799); Caspase 5 (NM_{—}004347); Caspase-14 (NM_{—}012114); ubiquitin specific protease USP-5 (NM_{—}003481); ubiquitin specific protease USP-11 (NM_{—}004651); ubiquitin specific protease USP 6 (NM_{—}004505); ubiquitin specific protease USP 26 (NM_{—}031907); ubiquitin specific protease (USP 28) (NM_{—}020886); 26S protease subunit 4 (L02426); LILRB1 (AF004230); Signal trasducer and activator of transcription 1, 91 kDa (STAT1) (977935); proteasome (prosome, macropain) subunit 6 (PSMA6) (X59417); TPSB1 (XM_{—}016204);; protease nexin-II (XM_{—}047793); Glia derived nexin precursor (P07093); and 26S protease regulatory subunit S10B; PCOLN3 (XM_{—}047524).

63. A method of diagnosis of a Group II disease or susceptibility to a Group II disease according to any preceding claim, which comprises detecting down-regulation of expression of an protease polypeptide or nucleic acid selected from the group consisting of: Apoptosis-related cysteine protease (CASP14) mRNA (NM_{—}012114), SCCE (XM_{—}009002), Human skin collagenase (M13509); TPS1 (NM_{—}003293); and TPSG1 (XM_{—}008123).

64. A method of diagnosis of a Group II disease or susceptibility to a Group II disease according to any preceding claim, which comprises detecting up-regulation of expression of an protease inhibitor polypeptide or nucleic acid selected from the group consisting of: SLPI (X04502); SKALP (XM_{—}009524; L10343); CSTA (NM_{—}005213; AA570193); SCCA (S66296); SCCA2 (U19557); plasminogen activator inhibitor type 1 (X04729; X04731); PAI2 (AF071400); SERPINA5 (NM_{—}000624); plasminogen activator inhibitor type 2 (L19066); TIMP (D11139); TIMP-1 (NM_{—}003254); TIMP-2 (NM_{—}003255); TIMP-3 (E13880); TIMP-4 (NM_{—}003256); TIM9a (AF150100); TIM9b (AF150105); Cystatin A (AA570193); Cystatin M/E (NM_{—}001323); multivalent protease inhibitor WFIKKN (AF422194); C1 inhibitor (SERPING1) (XM_{—}046218); protease inhibitor, Kunitz type, 2 (SPINT2) (XM_{—}032280); serine protease inhibitor, Kazal type 4 (SPINK4) (XM_{—}005539); proteinase inhibitor, clade B (ovalbumin), member 9 (XM_{—}053642); serine (or cysteine) proteinase inhibitor, clade); B (ovalbumin), member 6 (XM_{—}4047984); eppin-1 (EPPIN) (AF286368); eppin-2 (EPPIN) (AF286369); eppin-3 (EPPIN) (AF286370); Serine protease inhibitor-like, with Kunitz and WAP domains 1 (eppin) (SPINLW1) (NM_{—}020398); sparc/osteonectin, cwcv and kazal-like domains proteoglycan (testican) (SPOCK) (NM_{—}004598); protease inhibitor Kunitz type 1 (SPINT1) (XM_{—}056836); PI12 (AH009756); Human immunodeficiency virus type 1 gene for HIV-1 protease (AB020923); Human immunodeficiency virus type 1 gene for HIV-1 protease (AB020924); tissue factor pathway inhibitor 2 (TFPI2) (NM_{—}006528); secreted phosphoprotein 2, 24 kD (SPP2) (NM_{—}006944); cathepsin F (CTSF (NM_{—}003793); serine (or cysteine) proteinase inhibitor, clade A (alpha-1 antiproteinase, antitrypsin), member 6 (SERPINA6) (NM_{—}001756); serine (or cysteine) proteinase inhibitor, clade B (ovalbumin), member 3 (SERPINB3) (NM_{—}006919); Serine (or cysteine) proteinase inhibitor, clade A (alpha-1 antiproteinase, antitrypsin), member 3 (SERPINA3) (NM_{—}001085); Homo sapiens serine (or cysteine) proteinase inhibitor, clade B (ovalbumin), member 13 (XM_{—}008743); serine (or cysteine) proteinase inhibitor, clade B (ovalbumin), member 5 (SERPINB5 (XM_{—}008742); RelA-associated inhibitor (XM_{—}057693); inhibitor of DNA binding 1, dominant negative helix-loop-helix protein (ID1) (XM046179); serine (or cysteine) proteinase inhibitor, clade E (nexin, plasminogen activator inhibitor type 1), member (SERPINE1) (XM_{—}054850); cyclin-dependent kinase inhibitor 2B (p15, inhibits CDK4) (CDKN2B) (NM_{—}004936); Similar to cyclin-dependent kinase inhibitor 2B (p15, inhibits CDK4) (BC014469); serine (or cysteine) proteinase inhibitor, clade B (ovalbumin), member 7 (SERPINB7 (XM_{—}008745); protein inhibitor of activated STAT protein PIASy (PIASY) (NM_{—}016149); similar to protein inhibitor of activated STAT protein PIASy (LOC95830) (XM_{—}016864); PKC-potentiated PP1 inhibitory protein (PPP1R14A) (AY050668); inhibitor of DNA binding 3, dominant negative helix-loop-helix protein (ID3) (NM_{—}002167); Clade A (alpha-1 antiproteinase, antitrypsin) (XM_{—}028358); serine (or cysteine) proteinase inhibitor, clade H (heat shock protein 47), member 1 (SERPINH1) (NM_{—}0043 53); PI13 gene for hurpin (serine protease inhibitor) (AJ278717); protease inhibitor 5 (maspin) (PI5) (XM_{—}008742); PAI-2 (A32415).

65. A method of diagnosis of a Group II disease or susceptibility to a Group II disease according to any preceding claim, which comprises detecting down-regulation of expression of hbc750 Human pancreatic islet (T11141; T10920) polypeptide or nucleic acid.

66. A method according to any preceding claim, in which the Group II disease is selected from the group consisting of: psoriasis, ichtyoses, acne vulgaris and keratoses pilaris.

67. A method of treatment or prophylaxis of a Group II disease, the method comprising down-regulating the expression and/or activity of an adhesion protein for adhesion between the cells, or up-regulating the proteolysis of the adhesion protein.

68. A method of treatment or prophylaxis of a Group II disease according any preceding claim, in which the expression and/or activity of the adhesion protein is down-regulated at the transcriptional or the translational level, or both.

69. A method of treatment or prophylaxis of a Group II disease according any preceding claim, in which the expression, activity and/or breakdown of a protease involved in proteolysis of the adhesion protein is up-regulated.

70. A method of treatment or prophylaxis of a Group II disease according any preceding claim, in which the expression and/or activity of a protease inhibitor responsible for inhibiting the activity of a protease involved proteolysis of the adhesion protein is down-regulated, and/or in which the breakdown of the protease inhibitor is up-regulated.

71. A method of treatment or prophylaxis of a Group II disease according any preceding claim, in which the proteolysis of the adhesion protein is increased by one or more of the following: administration of a protease or a fragment thereof; administration of an agonist of a protease; administration of an antagonist of a protease inhibitor; increasing the expression of a protease; increasing the activity of a protease; reducing the expression of a protease inhibitor; reducing the activity of a protease inhibitor.

72. A method of treatment or prophylaxis of a Group II disease, the method comprising administering to a patient suffering or likely to suffer from such a disease a therapeutically effective amount of a non-disease associated form of an adhesion protein, protease or protease inhibitor, or a fragment thereof.

73. A method of treatment or prophylaxis of a Group II disease according to any preceding claim, which comprises administration of an adhesion protein, or a fragment thereof.

74. A method of treatment or prophylaxis of a Group II disease according to the preceding claim, which comprises administration of a fragment of Desmocollin I, preferably a peptide comprising the sequence of Peptide 641, or a fragment of Desmoplakin, preferably a peptide comprising the sequence of Peptide 642.

75. A monoclonal or polyclonal antibody capable of specifically reacting with an adhesion protein, protease or protease inhibitor, preferably a disease associated form of an adhesion protein, protease or protease inhibitor.

76. A method for identifying a molecule capable of capable of binding to an adhesion protein, protease or protease inhibitor, the method comprising contacting an adhesion protein, protease or protease inhibitor polypeptide with a candidate compound and determining whether the candidate compound binds to the adhesion protein, protease or protease inhibitor.

77. A compound identified by a method according to claim 76.

78. A method of identifying a molecule capable of modulating the activity of a protease, the method comprising: (a) providing an adhesion protein; (b) providing a protease; (c) exposing the adhesion protein to the protease in the presence of a candidate molecule; and (d) detecting cleavage or absence of cleavage of the adhesion protein by the protease.

79. Use of a compound identified by a method according to claim 78 to treat a Group II disease, in which the compound is capable of enhancing the cleavage of the adhesion molecule by the protease.

80. Use of a compound identified by a method according to claim 78 to treat a Group I disease, in which the compound is capable of inhibiting the cleavage of the adhesion molecule by the protease.

81. A transgenic, non-human animal expressing a heterologous adhesion protein, protease or protease inhibitor.

82. A transgenic, non-human animal expressing a modulated level, preferably an up-regulated or down-regulated level of an adhesion protein, protease or protease inhibitor.

83. A transgenic, non-human animal which substantially does not express an adhesion protein, protease or protease inhibitor.

84. Use of a transgenic animal according to any preceding claim as a model for a skin disease, preferably a Group I or a Group II disease.
